(19) United States
(12) Patent Application Publication
(10) Pub. No.: US 2017/0280720 A1
(43) Pub. Date: Oct. 5, 2017

(54) ARGININE METHYLTRANSFERASE INHIBITORS AND USES THEREOF

(71) Applicant: EPIZYME, INC., Cambridge, MA (US)

(72) Inventors: Richard CHESWORTH, Concord, MA (US); Lorna Helen MITCHELL, Cambridge, MA (US); Gideon SHAPIRO, Gainesville, FL (US); Oscar Miguel MORADEI, Burlington, MA (US); Kevin Wayne KUNTZ, Woburn, MA (US); Kenneth W. DUNCAN, Westwood, MA (US); Lei JIN, Wellesley, MA (US)

(73) Assignee: Epizyme, Inc., Cambridge (US)

(21) Appl. No.: 15/511,514

(22) PCT Filed: Sep. 17, 2015
(86) PCT No.: PCT/US2015/050675

§ 371 Date: Mar. 15, 2017

Related U.S. Application Data
(60) Provisional application No. 62/051,846, filed on Sep. 17, 2014.

(51) Int. Cl. A01N 47/10; C07D 277/20; C07D 233/54; C07D 209/10; C07D 241/02

(52) U.S. Cl. CPC A01N 47/10; C07D 209/10; C07D 241/02; C07D 233/54; C07D 277/20; A61K 38/45

(57) ABSTRACT

Described herein are compounds of Formula (I), pharmaceutically acceptable salts thereof, and pharmaceutical compositions thereof. Compounds described herein are useful for inhibiting arginine methyltransferase activity. Methods of using the compounds for treating arginine methyltransferase-mediated disorders are also described.

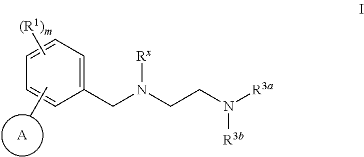

### RELATED APPLICATIONS

The present application claims priority under 35 U.S.C. §119(e) to U.S. provisional patent application, U.S. Ser. No. 62/051,846, filed Sep. 17, 2015, the entire contents of which is incorporated herein by reference.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., arginine methyltransferases), many of which are associated with specific genetic alterations that can cause human disease.

Disease-associated chromatin-modifying enzymes (e.g., arginine methyltransferases) play a role in diseases such as proliferative disorders, autoimmune disorders, muscular disorders, vascular disorders, metabolic disorders, and neurological disorders. Thus, there is a need for the development of small molecules that are capable of inhibiting the activity of arginine methyltransferases.

### DETAILED DESCRIPTION OF CERTAIN EMBODIMENTS

Arginine methyltransferases are attractive targets for modulation given their role in the regulation of diverse biological processes. It has now been found that compounds described herein, and pharmaceutically acceptable salts and compositions thereof, are useful as inhibitors of arginine methyltransferases. Such compounds are of Formula (I):

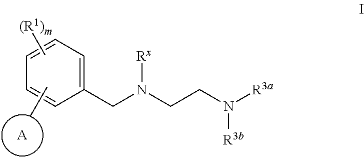

or pharmaceutically acceptable salts thereof, wherein Ring A, R¹, m, R^{3a}, R^{3b}, and R^{x} are as defined herein.

In some embodiments, pharmaceutical compositions are provided which comprise a compound described herein (e.g., a compound of Formula (I)), or a pharmaceutically acceptable salt thereof, and optionally a pharmaceutically acceptable excipient.

In certain embodiments, compounds described herein inhibit activity of an arginine methyltransferase (RMT) (e.g., PRMT1, PRMT3, CARM1, PRMT6, and/or PRMT8). In certain embodiments, methods of inhibiting an arginine methyltransferase are provided which comprise contacting the arginine methyltransferase with an effective amount of a compound of Formula (I), or a pharmaceutically acceptable salt thereof. The RMT may be purified or crude, and may be present in a cell, tissue, or a subject. Thus, such methods encompass inhibition of RMT activity both in vitro and in vivo. In certain embodiments, the RMT is wild-type. In certain embodiments, the RMT is overexpressed. In certain embodiments, the RMT is a mutant. In certain embodiments, the RMT is in a cell. In some embodiments, the RMT is expressed at normal levels in a subject, but the subject would benefit from RMT inhibition (e.g., because the subject has one or more mutations in an RMT substrate that causes an increase in methylation of the substrate with normal levels of RMT). In some embodiments, the RMT is in a subject known or identified as having abnormal RMT activity (e.g., overexpression).

In certain embodiments, methods of modulating gene expression in a cell are provided which comprise contacting a cell with an effective amount of a compound of Formula (I), or a pharmaceutically acceptable salt thereof, or a pharmaceutical composition thereof. In certain embodiments, the cell in culture in vitro. In certain embodiments, cell is in an animal, e.g., a human.

In certain embodiments, methods of modulating transcription in a cell are provided which comprise contacting a cell with an effective amount of a compound of Formula (I), or a pharmaceutically acceptable salt thereof, or a pharmaceutical composition thereof. In certain embodiments, the cell in culture in vitro. In certain embodiments, the cell is in an animal, e.g., a human.

In some embodiments, methods of treating an RMT-mediated disorder (e.g., a PRMT1-, PRMT3-, CARM1-, PRMT6-, or PRMT8-mediated disorder) are provided which comprise administering to a subject suffering from an RMT-mediated disorder an effective amount of a compound described herein (e.g., a compound of Formula (I)), or a pharmaceutically acceptable salt thereof, or a pharmaceutical composition thereof. In certain embodiments, the RMT-mediated disorder is a proliferative disorder. In certain embodiments, compounds described herein are useful for treating cancer. In certain embodiments, compounds described herein are useful for treating breast cancer, prostate cancer, lung cancer, colon cancer, bladder cancer, or leukemia. In certain embodiments, the RMT-mediated disorder is a muscular disorder. In certain embodiments, the RMT-mediated disorder is an autoimmune disorder. In certain embodiments, the RMT-mediated disorder is a neurological disorder. In certain embodiments, the RMT-mediated disorder is a vascular disorder. In certain embodiments, the RMT-mediated disorder is a metabolic disorder.

Compounds described herein are also useful for the study of arginine methyltransferases in biological and pathological phenomena, the study of intracellular signal transduction pathways mediated by arginine methyltransferases, and the comparative evaluation of new RMT inhibitors.

This application refers to various issued patent, published patent applications, journal articles, and other publications, all of which are incorporated herein by reference.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

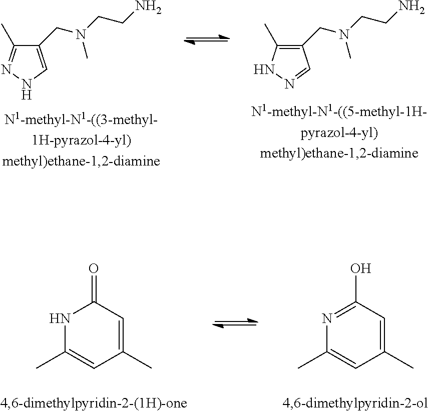

It is to be understood that the following two structures are meant to be equivalent and used interchangeably:

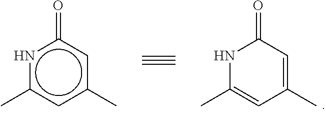

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Radical” refers to a point of attachment on a particular group. Radical includes divalent radicals of a particular group.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms and one or more carbon-carbon double bonds (e.g., 1, 2, 3, or 4 double bonds), and optionally one or more triple bonds (e.g., 1, 2, 3, or 4 triple bonds) (“C₂₋₂₀ alkenyl”). In certain embodiments, alkenyl does not comprise triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms and one or more carbon-carbon triple bonds (e.g., 1, 2, 3, or 4 triple bonds), and optionally one or more double bonds (e.g., 1, 2, 3, or 4 double bonds) (“C₂₋₂₀ alkynyl”). In certain embodiments, alkynyl does not comprise double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Fused” or “ortho-fused” are used interchangeably herein, and refer to two rings that have two atoms and one bond in common, e.g.,

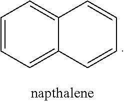

“Bridged” refers to a ring system containing (1) a bridgehead atom or group of atoms which connect two or more non-adjacent positions of the same ring; or (2) a bridgehead atom or group of atoms which connect two or more positions of different rings of a ring system and does not thereby form an ortho-fused ring, e.g.,

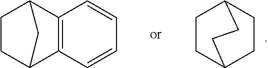 [SMILES: c1ccc2c(c1)C1CCC2C1 ; C1CC2CCC1CC2]

“Spiro” or “Spiro-fused” refers to a group of atoms which connect to the same atom of a carbocyclic or heterocyclic ring system (geminal attachment), thereby forming a ring, e.g.,

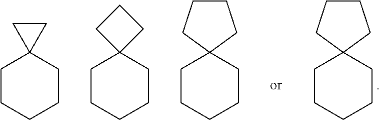

Spiro-fusion at a bridgehead atom is also contemplated.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or is a fused, bridged or spiro-fused ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In certain embodiments, heterocyclyl or heterocyclic refers to a radical of a 3-10 membered non-aromatic ring system having ring carbon atoms and 1-4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms independently selected from nitrogen, oxygen, and sulfur. In some embodiments, the 5-6 membered heterocyclyl has 1-2 ring heteroatoms independently selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-14 membered monocyclic or polycyclic (e.g., bicyclic or tricyclic) 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, heteroaryl refers to a radical of a 5-10 membered monocyclic or bicyclic 4n+2 aromatic ring system having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-14 membered aromatic ring system having ring carbon atoms and 1-4 ring heteroatoms provided in the aromatic ring system, wherein each heteroatom is independently selected from nitrogen, oxygen, and sulfur (“5-14 membered heteroaryl”). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms independently selected from nitrogen, oxygen, and sulfur. In some embodiments, the 5-6 membered heteroaryl has 1-2 ring heteroatoms independently selected from nitrogen, oxygen, and sulfur. In some embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary 5,6-bicyclic heteroaryl groups include, without limitation, any one of the following formulae:

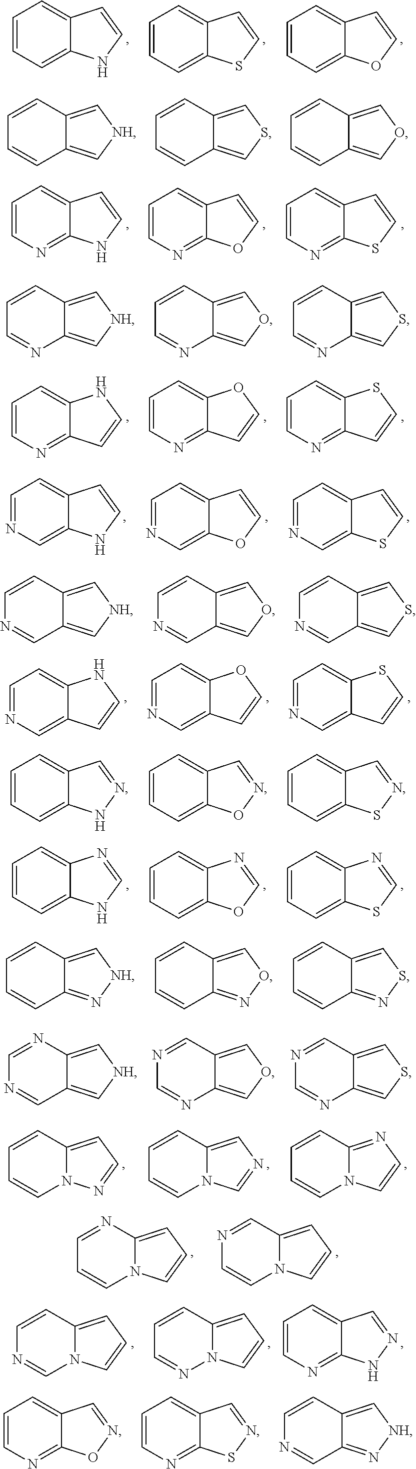

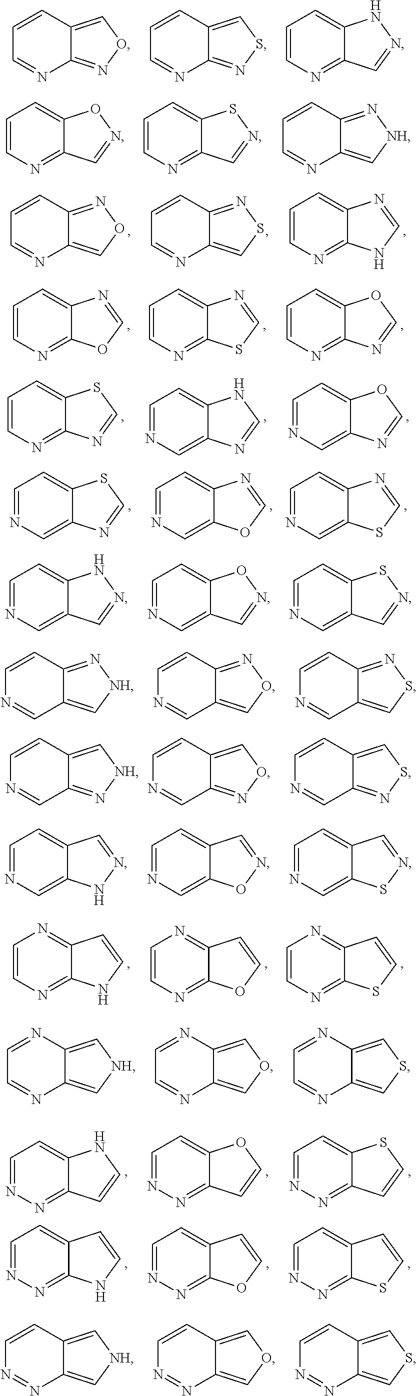

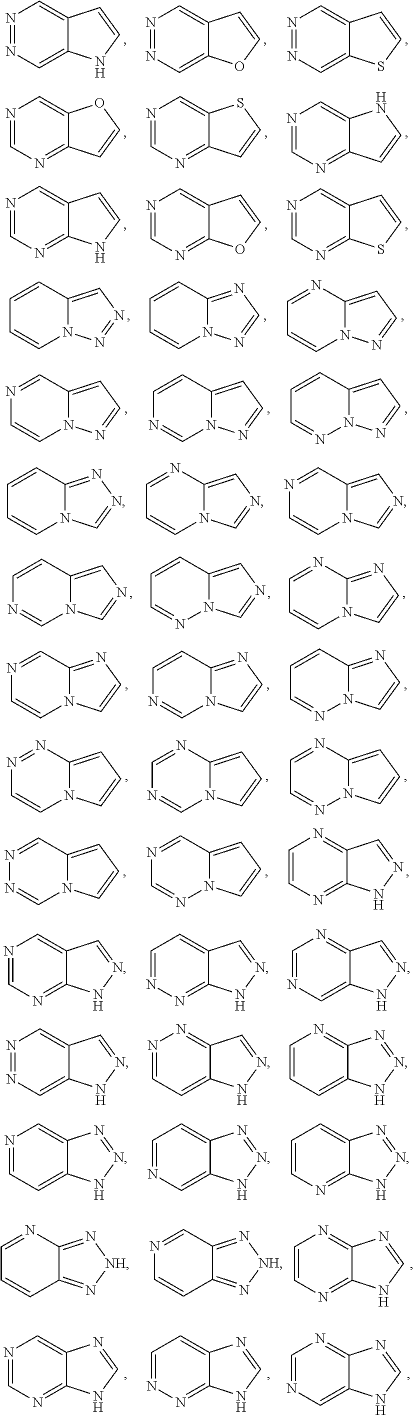

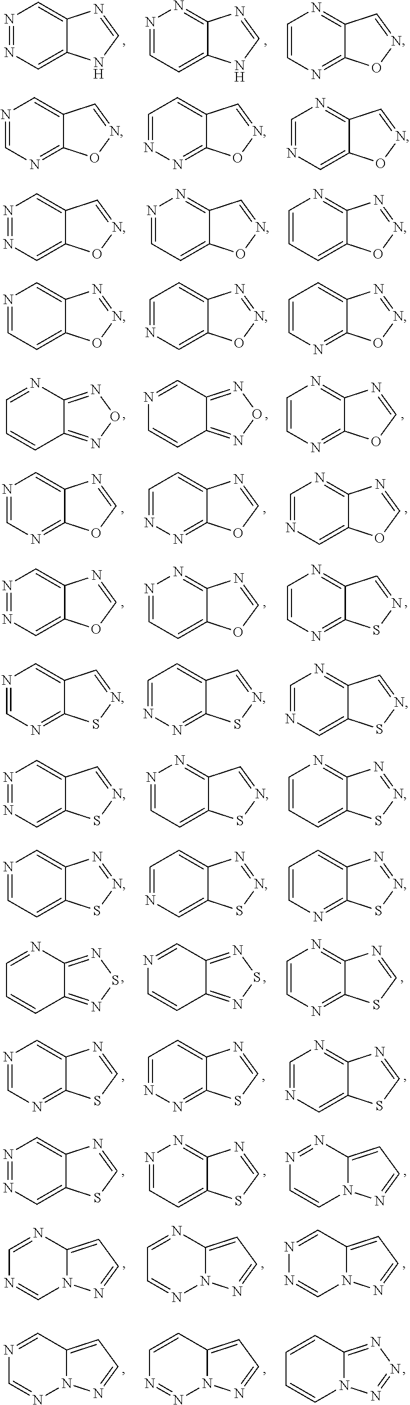

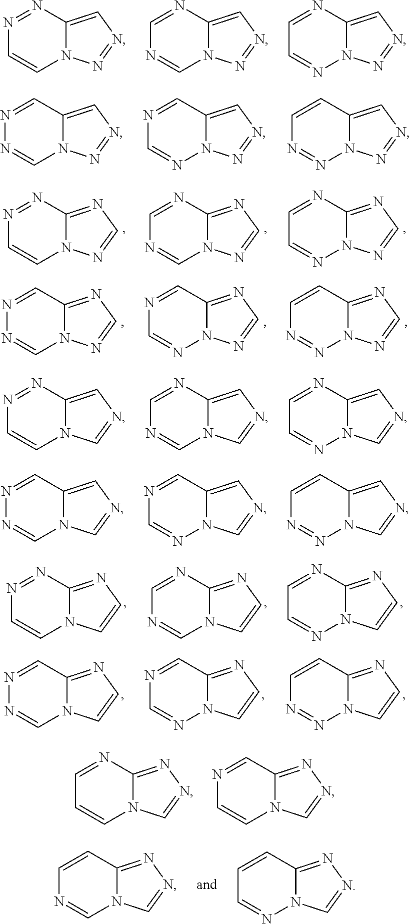

In any of the monocyclic or bicyclic heteroaryl groups, the point of attachment can be any carbon or nitrogen atom, as valency permits.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂ (C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, C(O)₁SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR′, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate, alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, Examples, and claims. The present disclosure is not intended to be limited in any manner by the above exemplary listing of substituents.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

A “subject” to which administration is contemplated includes, but is not limited to, humans (e.g., a male or female of any age group, e.g., a pediatric subject (e.g, infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), rodents (e.g., rats and/or mice), reptiles, amphibians, and fish. In certain embodiments, the non-human animal is a mammal. The non-human animal may be a male or female at any stage of development. A non-human animal may be a transgenic animal.

“Condition,” “disease,” and “disorder” are used interchangeably herein.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

An “effective amount” of a compound refers to an amount sufficient to elicit the desired biological response, e.g., treat the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “methyltransferase” represents transferase class enzymes that are able to transfer a methyl group from a donor molecule to an acceptor molecule, e.g., an amino acid residue of a protein or a nucleic base of a DNA molecule. Methytransferases typically use a reactive methyl group bound to sulfur in S-adenosyl methionine (SAM) as the methyl donor. In some embodiments, a methyltransferase described herein is a protein methyltransferase. In some embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and arginine residues of histone proteins. In certain embodiments, a methyltransferase described herein is a histone-arginine N-methyltransferase.

As generally described above, provided herein are compounds useful as arginine methyltransferase (RMT) inhibitors. In some embodiments, the present disclosure provides a compound of Formula (I):

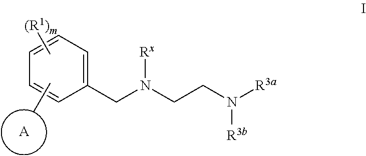

or a pharmaceutically acceptable salt thereof,
wherein

Ring A is optionally substituted aryl, optionally substituted pyridinyl, optionally substituted bicyclic heteroaryl with one, three, or four nitrogen atoms, optionally substituted indazolyl, optionally substituted azaindolyl, or optionally substituted benzoimidazolyl;

m is 0, 1, 2, 3, or 4;

R^{x} is optionally substituted C₁₋₄ alkyl or optionally substituted C₃₋₄ cycloalkyl; and

each of R^{3a} and R^{3b} is independently hydrogen, optionally substituted C₁₋₄ alkyl, or optionally substituted C₃₋₄ cycloalkyl;

each instance of R¹ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;

each instance of R^{A} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl; and

each instance of R^{B} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl;

or two R^{B} taken together with the intervening nitrogen form optionally substituted heterocyclyl;

or R^{A} and R^{B} taken together with the intervening atoms form optionally substituted heterocyclyl; and

provided that the optional substituent on Ring A is not an optionally substituted pyridone.

In certain embodiments, a provided compound is of Formula (I-a):

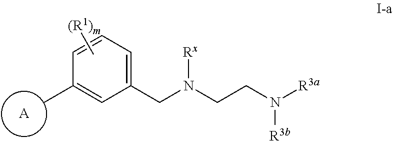

or a pharmaceutically acceptable salt thereof.

In certain embodiments, a provided compound is of Formula (I-b):

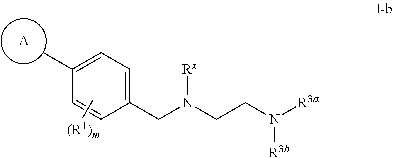

or a pharmaceutically acceptable salt thereof.

As generally defined herein, m is 0, 1, 2, 3, or 4. In certain embodiments, m is 0. In certain embodiments, m is 1. In certain embodiments, m is 2. In certain embodiments, m is 3. In certain embodiments, m is 4.

As generally defined herein, each instance of R¹ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)_{R}^{A}, —C(═O)N(R^{B})₂, —NR^{B}C(═O)R^{A}, —OC(═O)N(R^{B})₂, —NR^{B}C(═O)OR^{A},

—NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R¹ is independently hydrogen, halogen, —CN, —NO₂, —N₃, or optionally substituted alkyl. In certain embodiments, each instance of R¹ is independently hydrogen, halogen, or optionally substituted C₁₋₆ alkyl. In certain embodiments, R¹ is independently hydrogen. In certain embodiments, R¹ is independently halogen (e.g. F, Cl, Br, or I). In certain embodiments, R¹ is optionally substituted alkyl. In certain embodiments, R¹ is substituted alkyl. In certain embodiments, R¹ is unsubstituted alkyl (e.g. methyl or ethyl).

In certain embodiments, a provided compound is of one of the following formulae:

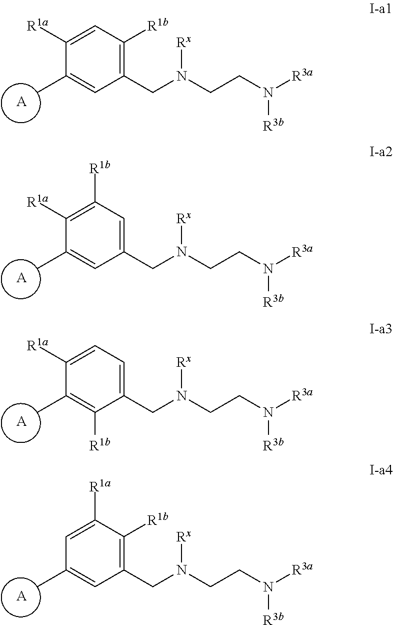

or a pharmaceutically acceptable salt thereof, wherein Ring A, R^{3a}, R^{3b}, and IV are as described herein; and each instance of R^{1a} and R^{1b} is independently hydrogen, halogen, or optionally substituted C₁₋₆ alkyl.

In certain embodiments, a provided compound is of one of the following formulae:

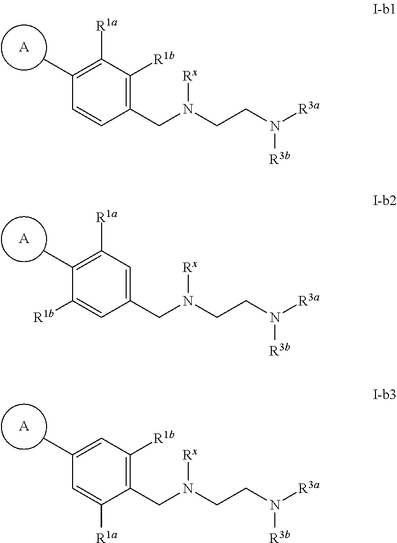

or a pharmaceutically acceptable salt thereof, wherein Ring A, R^{3a}, R^{3b}, and IV are as described herein; and each instance of R^{1a} and R^{1b} is independently hydrogen, halogen, or optionally substituted C₁₋₆ alkyl.

In certain embodiments, R^{1a} is halogen (e.g. F, Cl, Br, or I). In certain embodiments, R^{1a} is optionally substituted C₁₋₆ alkyl. In certain embodiments, R^{1a} is unsubstituted C₁₋₆ alkyl (e.g. methyl or ethyl).

In certain embodiments, R^{1b} is hydrogen. In certain embodiments, R^{1b} is halogen (e.g. F, Cl, Br, or I). In certain embodiments, R^{1b} is optionally substituted C₁₋₆ alkyl. In certain embodiments, R^{1b} is unsubstituted C₁₋₆ alkyl (e.g. methyl or ethyl).

In certain embodiments, R^{1a} is hydrogen and R^{1b} is halogen (e.g. F, Cl, Br, or I). In certain embodiments, R^{1a} is unsubstituted C₁₋₆ alkyl (e.g. methyl or ethyl) and R^{1b} is hydrogen. In certain embodiments, R^{1a} is optionally substituted C₁₋₆ alkyl and R^{1b} is hydrogen. In certain embodiments, R^{1b} is hydrogen and R^{1a} is halogen (e.g. F, Cl, Br, or I). In certain embodiments, R^{1b} is unsubstituted C₁₋₆ alkyl (e.g. methyl or ethyl) and R^{1a} is hydrogen. In certain embodiments, R^{1b} is optionally substituted C₁₋₆ alkyl and R^{1a} is hydrogen.

As generally defined herein, IV is optionally substituted C₁₋₄ alkyl or optionally substituted C₃₋₄ cycloalkyl. In certain embodiments, IV is optionally substituted C₁₋₄ alkyl. In certain embodiments, IV is substituted C₁₋₄ alkyl. In certain embodiments, IV is unsubstituted C₁₋₄ alkyl (e.g. methyl or ethyl). In certain embodiments, R^{x} is optionally substituted C₃₋₄ cycloalkyl (e.g. cyclopropyl).

As generally defined herein, R^{3a} is hydrogen, optionally substituted C₁₋₄ alkyl, or optionally substituted C₃₋₄ cycloalkyl. In certain embodiments, R^{3a} is hydrogen. In certain embodiments, R^{3a} is optionally substituted C₁₋₄ alkyl. In certain embodiments, R^{3a} is substituted C₁₋₄ alkyl. In certain embodiments, R^{3a} is unsubstituted C₁₋₄ alkyl (e.g. methyl or ethyl). In certain embodiments, R^{3a} is optionally substituted C₃₋₄ cycloalkyl (e.g. cyclopropyl).

As generally defined herein, R^{3b} is hydrogen, optionally substituted C₁₋₄ alkyl, or optionally substituted C₃₋₄ cycloalkyl. In certain embodiments, R^{3a} is hydrogen. In certain embodiments, R^{3b} is optionally substituted C₁₋₄ alkyl. In certain embodiments, R^{3b} is substituted C₁₋₄ alkyl. In certain embodiments, R^{3b} is unsubstituted C₁₋₄ alkyl (e.g. methyl or ethyl). In certain embodiments, R^{3b} is optionally substituted C₃₋₄ cycloalkyl (e.g. cyclopropyl).

As generally defined herein, R^{3a} and R^{3b} are both hydrogen. In certain embodiments, R^{3a} is hydrogen and R^{3b} is optionally substituted C₁₋₄ alkyl. In certain embodiments, R^{3a} is hydrogen and R^{3b} is unsubstituted C₁₋₄ alkyl (e.g. methyl). In certain embodiments, each of R^{3a} and R^{3b} is independently optionally substituted C₁₋₄ alkyl. In certain embodiments, each of R^{3a} and R^{3b} is independently unsubstituted C₁₋₄ alkyl. In certain embodiments, R^{3a} and R^{3b} are both methyl.

As generally defined herein, Ring A is optionally substituted aryl, optionally substituted five-membered heteroaryl, optionally substituted six-membered hereoaryl, or optionally substituted bicyclic heteroaryl. In certain embodiments, Ring A is optionally substituted aryl, optionally substituted pyridinyl, optionally substituted bicyclic heteroaryl with one, two, three, or four nitrogen ring atoms. In certain embodiments, Ring A is optionally substituted aryl, optionally substituted pyridinyl, optionally substituted bicyclic heteroaryl with one, three, or four nitrogen ring atoms, optionally substituted indazolyl, optionally substituted azaindolyl, or optionally substituted benzoimidazolyl. In certain embodiments, Ring A is optionally substituted aryl. In certain embodiments, Ring A is optionally substituted phenyl. In certain embodiments, Ring A is unsubstituted phenyl. In certain embodiments, Ring A is substituted phenyl. In certain embodiments, Ring A is mono-substituted phenyl. In certain embodiments, Ring A is di-substituted phenyl. In certain embodiments, Ring A is tri-substituted phenyl. In certain embodiments, Ring A is tetra-substituted phenyl. In certain embodiments, Ring A is penta-substituted phenyl. In certain embodiments, Ring A is optionally substituted pyridinyl. In certain embodiments, Ring A is optionally substituted bicyclic heteroaryl with one, two, three, or four nitrogen ring atoms. In certain embodiments, Ring A is an optionally substituted 6,5-membered heteroaryl ring or an optionally substituted 5,6-membered heteroaryl ring. In certain embodiments, Ring A is an optionally substituted monocyclic 5-membered heteroaryl ring fused with an optionally substituted monocyclic 6-membered aryl ring. In certain embodiments, Ring A is an optionally substituted monocyclic 5-membered heteroaryl ring fused with an optionally substituted monocyclic 6-membered heteroaryl ring. The point of attachment of Ring A to the phenyl ring in Formula (I) may be at any atom of Ring A, as valency permits. In certain embodiments, Ring A is of one of the following formulae:

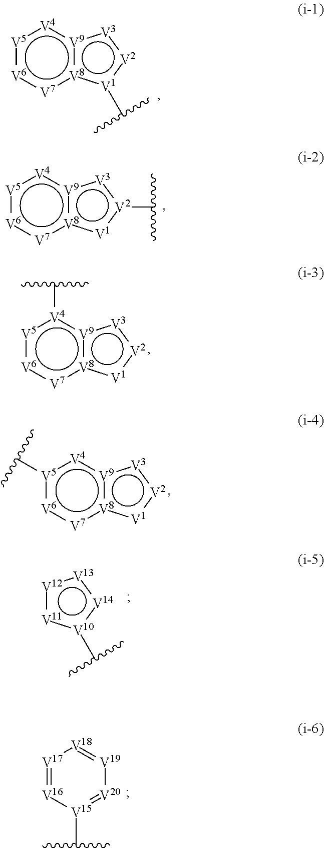

wherein

each of V¹, V², V³, V⁴, V⁵, V⁶, V⁷, V⁸, V⁹, V¹⁰, V¹¹, V¹², V¹³, V¹⁴, V¹⁵, V¹⁶, V¹⁷, V¹⁸, V¹⁹, and V²⁰ is independently O, S, N, NR^{C}, C, or CR^{CV}, as valency permits;

each instance of R^{NV} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group;

each instance of R^{CV} is independently selected from the group consisting of hydrogen, halogen, —CN, —NO₂, —N₃, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{CVa}, —N(R^{CVa})², —SR^{CVa}, —C(═O)R^{CVa}, C(═O)OR^{CVa}, —OC(═O)R^{CVa}, —C(═O)N(R^{CVa})₂, —NR^{CVa}C(═O)R^{CVa}, —OC(═O)N(R^{CVa})₂, —NR^{CVa}C(═O)OR^{CVa}, —NR^{CVa}C(═O)N(R^{CVa})₂, —S(═O)R^{CVa}, —OS(═O)₂R^{CVa}, —SO₂R^{CVa}, NR^{B}SO₂R^{CVa}, or SO₂N(R^{CVa})₂

wherein each occurrence of R^{CVa} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{CVa} groups are joined to form an optionally substituted heterocyclic ring.

In certain embodiments, Ring A is optionally substituted bicyclic heteroaryl with one nitrogen ring atom. In certain embodiments, Ring A is of one of Formulae (i-1)-(i-4), wherein each of V¹, V², V³, V⁴, V⁵, V⁶, V⁷, V⁸, and V⁹ is independently C, CR^{CV}, N or NR^{NV}, provided that only one of V¹, V², V³, V⁴, V⁵, V⁶, V⁷, V⁸, and V⁹ is N or NR^{NV}. In certain embodiments, Ring A is optionally substituted indolyl. In certain embodiments, Ring A is of one of the following formulae:

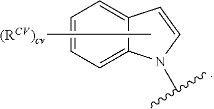

In certain embodiments, Ring A is of one of the following formulae:

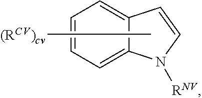

wherein cv is 0, 1, 2, 3, 4, 5, or 6, as valency permits; and the point of attachment is one any carbon ring atom. In certain embodiments, Ring A is optionally substituted isoindolyl. In certain embodiments, Ring A is of the formula

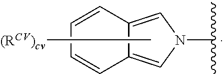

In certain embodiments, Ring A is of the formula

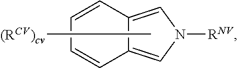

wherein cv is 0, 1, 2, 3, 4, 5, or 6, as valency permits; and the point of attachment is one any carbon ring atom.

In certain embodiments, Ring A is optionally substituted bicyclic heteroaryl with two nitrogen ring atoms. In certain embodiments, Ring A is of one of Formulae (i-1)-(i-4), wherein each of V¹, V², V³, V⁴, V⁵, V⁶, V⁷, V⁸, and V⁹ is independently C, CR^{CV}, N or NR^{NV}, provided that only two of V¹, V², V³, V⁴, V⁵, V⁶, V⁷, V⁸, and V⁹ are N or NR^{NV}. In certain embodiments, Ring A is optionally substituted indazolyl, optionally substituted azaindolyl, or optionally substituted benzoimidazolyl. In certain embodiments, Ring A is optionally substituted indazolyl. In certain embodiments, Ring A is of one of the following formulae:

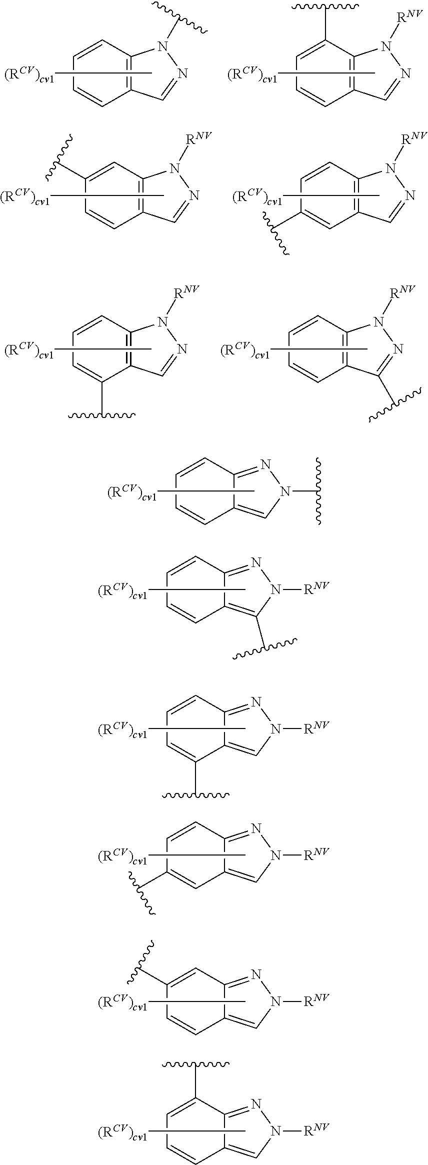

wherein R^{NV} and R^{CV} are as defined herein; and cv1 is 0, 1, 2, 3, or 4, as valency permits. In certain embodiments, Ring A is optionally substituted azaindolyl. In certain embodiments, Ring A is of one of the following formulae:

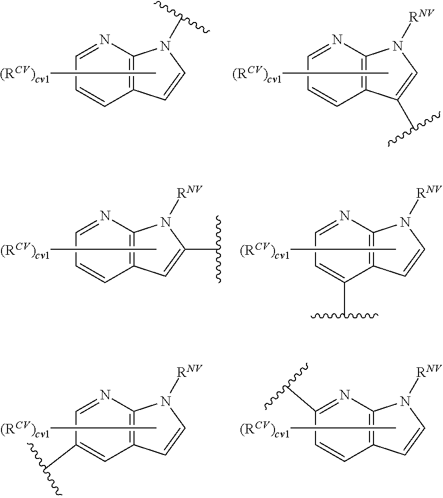

wherein R^{NV} and R^{CV} are as defined herein; and cv1 is 0, 1, 2, 3, or 4, as valency permits. In certain embodiments, Ring A is optionally substituted benzoimidazolyl. In certain embodiments, Ring A is of one of the following formulae:

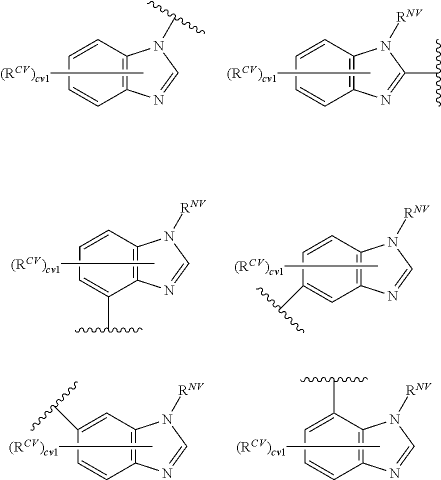

wherein R^{NV} and R^{CV} are as defined herein; and cv1 is 0, 1, 2, 3, or 4, as valency permits.

In certain embodiments, Ring A is optionally substituted bicyclic heteroaryl with three nitrogen ring atoms. In certain embodiments, Ring A is of one of Formulae (i-1)-(i-4), wherein each of V¹, V², V³, V⁴, V⁵, V⁶, V⁷, V⁸, and V⁹ is independently C, CR^{CV}, N or NR^{NV}, provided that only three of V¹, V², V³, V⁴, V⁵, V⁶, V⁷, V⁸, and V⁹ are N or NR^{NV}. In certain embodiments, Ring A is optionally substituted pyrazolo-pyridinyl. In certain embodiments, Ring A is of one of the following formulae:

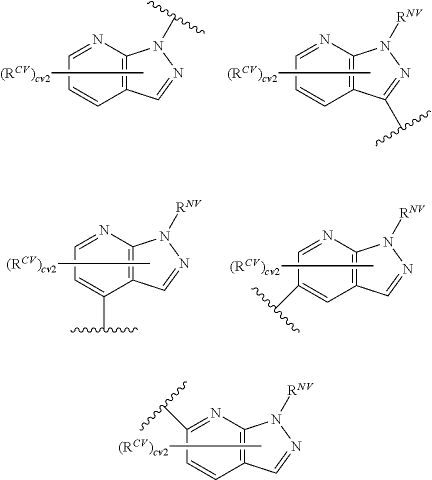

wherein R^{NV} and R^{CV} are as defined herein; and cv2 is 0, 1, 2, or 3, as valency permits. In certain embodiments, Ring A is of one of the following formulae:

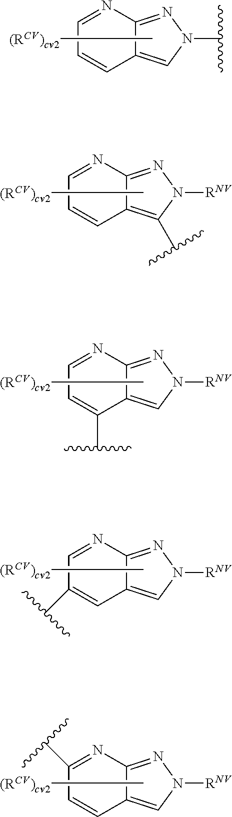

wherein R^{NV} and R^{CV} are as defined herein; and cv2 is 0, 1, 2, or 3, as valency permits. In certain embodiments, Ring A is optionally substituted pyrazolo[1,5-a]pyrimidinyl. In certain embodiments, Ring A is of one of the following formulae:

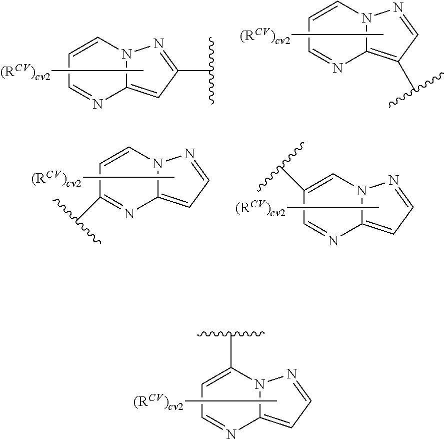

wherein R^{NV} and R^{CV} are as defined herein; and cv2 is 0, 1, 2, or 3, as valency permits.

In certain embodiments, Ring A is optionally substituted bicyclic heteroaryl with four nitrogen ring atoms. In certain embodiments, Ring A is of one of Formulae (i-1)-(i-4), wherein each of V¹, V², V³, V⁴, V⁵, V⁶, V⁷, V⁸, and V⁹ is independently C, CR^{CV}, N or NR^{NV}, provided that only four of V¹, V², V³, V⁴, V⁵, V⁶, V⁷, V⁸, and V⁹ is N or NR^{NV}. In certain embodiments, Ring A is optionally substituted pyrazolo[3,4-d]pyrimidine. In certain embodiments, Ring A is of one of the following formulae:

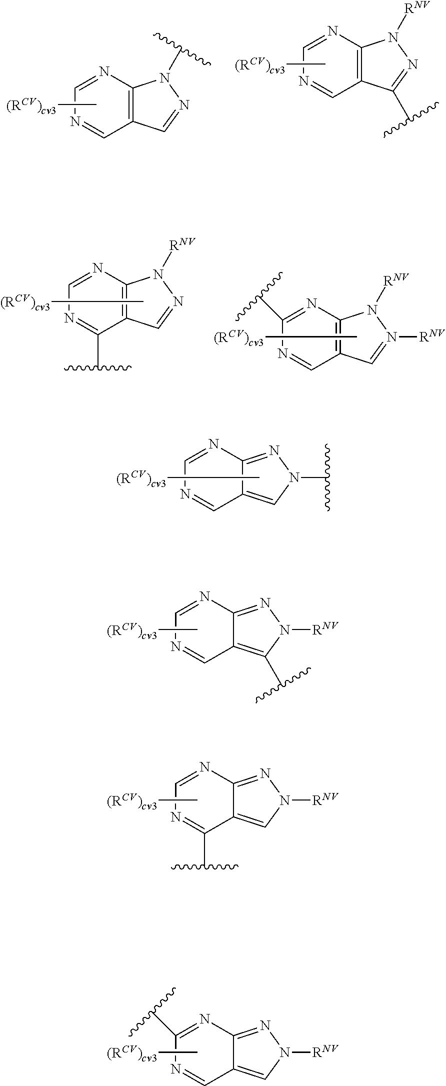

wherein R^{NV} and R^{CV} are as defined herein; and cv3 is 0, 1, or 2, as valency permits.

In certain embodiments, Ring A is optionally substituted phenyl of Formula (A-1):

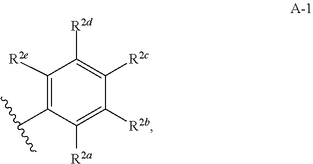

wherein each of R^{2a}, R^{2b}, R^{2c}, R^{2d}, and R^{2e} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂.

In certain embodiments, at least one of R^{2a}, R^{2b}, R^{2c}, R^{2d}, and R^{2e} is hydrogen. In certain embodiments, at least two of R^{2a}, R^{2b}, R^{2c}, R^{2d}, and R^{2e} is hydrogen. In certain embodiments, at least three of R^{2a}, R^{2b}, R^{2c}, R^{2d}, and R^{2e} is hydrogen. In certain embodiments, one of R^{2a}, R^{2b}, R^{2c}, R^{2d}, and R^{2e} is hydrogen. In certain embodiments, two of R^{2a}, R^{2b}, R^{2c}, R^{2d}, and R^{2e} are hydrogen. In certain embodiments, three of R^{2a}, R^{2b}, R^{2c}, R^{2d}, and R^{2e} is hydrogen. In certain embodiments, four of R^{2a}, R^{2b}, R^{2c}, R^{2d}, and R^{2e} is hydrogen. In certain embodiments, at least one of R is a halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, at least two of R^{2a}, R^{2b}, R^{2c}, R^{2d}, and R^{2e} are halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, at least three of R^{2a}, R^{2b}, R^{2c}, R^{2d}, and R^{2e} are halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂.

In certain embodiments, Ring A is of one of the following formulae:

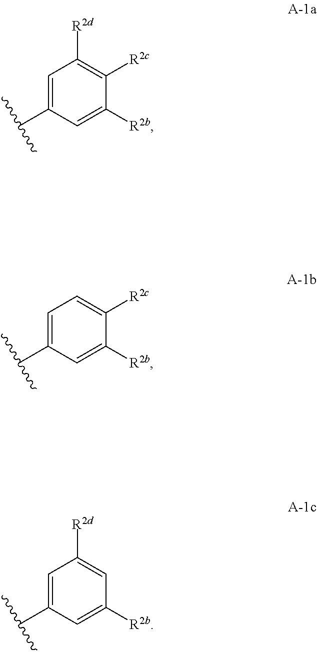

As generally defined herein, R^{2a} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}. In certain embodiments, R^{2a} is hydrogen. In certain embodiments, R^{2a} is halogen (e.g. F, Cl, Br, or I). In certain embodiments, R^{2a} is optionally substituted alkyl.

As generally defined herein, R^{2b} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}. In certain embodiments, R^{2b} is hydrogen. In certain embodiments, R^{2b} is halogen (e.g. F, Cl, Br, or I). In certain embodiments, R^{2b} is optionally substituted alkyl. In certain embodiments, R^{2b} is —N(R^{B})₂ or —C(═O)N(R^{B})₂, wherein R^{B} is as defined herein. In certain embodiments, R^{2b} is —N(R^{B})₂; and each instance of R^{B} is independently hydrogen or optionally substituted alkyl. In certain embodiments, R^{2b} is —NHR^{B} or —N(CH₃)R^{B}, wherein R^{B} is hydrogen or optionally substituted alkyl. In certain embodiments, R^{2b} is —N(CH₃)R^{B}, wherein R^{B} is substituted alkyl. In certain embodiments, R^{2b} is —N(CH₃)R^{B}, wherein R^{B} is —C₁₋₆alkyl-carbocyclyl (e.g. —CH₂-cyclopropyl). In certain embodiments, R^{2b} is —C(═O)N(R^{B})₂, wherein each instance of R^{B} is independently hydrogen, optionally substituted alkyl, or optionally substituted heterocyclyl. In certain embodiments, R^{2b} is —C(═O)NHR^{B} or —C(═O)N(CH₃)R^{B}, wherein R^{B} is independently hydrogen, optionally substituted alkyl, or optionally substituted heterocyclyl. In certain embodiments, R^{2b} is —C(═O)NHR^{B}, wherein R^{B} is optionally substituted heterocyclyl. In certain embodiments, R^{2b} is —C(═O)NHR^{B}, wherein R^{B} is unsubstituted heterocyclyl (e.g. tetrahydropyranyl). In certain embodiments, R^{2b} is —C(═O)NHR^{B}, wherein R^{B} is optionally substituted alkyl. In certain embodiments, R^{2b} is —C(═O)NHR^{B}, wherein R^{B} is substituted alkyl. In certain embodiments, R^{2b} is —C(═O)NHR^{B}, wherein R^{B} is C₁₋₆alkyl-heterocyclyl (e.g. —CH₂-tetrahydropyranyl).

As generally defined herein, R^{2c} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}. In certain embodiments, R^{2c} is hydrogen. In certain embodiments, R^{2c} is halogen (e.g. F, Cl, Br, or I). In certain embodiments, R^{2c} is optionally substituted alkyl. In certain embodiments, R^{2c} is optionally substituted C₁₋₆ alkyl (substituted such as C₁₋₆haloalkyl or unsubstituted such as methyl or ethyl). In certain embodiments, R^{2c} is —C(═O)N(R^{B})₂, wherein each instance of R^{B} is independently hydrogen, optionally substituted alkyl, or optionally substituted heterocyclyl. In certain embodiments, R^{2c} is —C(═O)NHR^{B} or —C(═O)N(CH₃)R^{B}, wherein R^{B} is independently hydrogen, optionally substituted alkyl, or optionally substituted heterocyclyl. In certain embodiments, R^{2c} is —C(═O)NHR^{B}, wherein R^{B} is optionally substituted heterocyclyl. In certain embodiments, R^{2c} is —C(═O)NHR^{B}, wherein R^{B} is unsubstituted heterocyclyl (e.g. tetrahydropyranyl). In certain embodiments, R^{2c} is —C(═O)NHR^{B}, wherein R^{B} is optionally substituted alkyl. In certain embodiments, R^{2c} is —C(═O)NHR^{B}, wherein R^{B} is substituted alkyl. In certain embodiments, R^{2c} is —C(═O)NHR^{B}, wherein R^{B} is C₁₋₆alkyl-heterocyclyl (e.g. —CH₂-tetrahydropyranyl).

As generally defined herein, R^{2d} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}. In certain embodiments, R^{2d} is hydrogen. In certain embodiments, R^{2d} is halogen (e.g. F, Cl, Br, or I). In certain embodiments, R^{2d} is optionally substituted alkyl. In certain embodiments, R^{2d} is —N(R^{B})₂ or —C(═O)N(R^{B})₂, wherein R^{B} is as defined herein. In certain embodiments, R^{2d} is —N(R^{B})₂; and each instance of R^{B} is independently hydrogen, optionally substituted alkyl, or optionally substituted heterocyclyl. In certain embodiments, R^{2d} is —NHR^{B}, —N(CH₃)R^{B}, or —N(C₂H₅)R^{B}, wherein R^{B} is hydrogen, optionally substituted alkyl, or optionally substituted heterocyclyl. In certain embodiments, R^{2d} is —N(CH₃)R^{B} or —N(C₂H₅)R^{B}, wherein R^{B} is substituted alkyl. In certain embodiments, R^{2d} is —N(CH₃)R^{B} or —N(C₂H₅)R^{B}, wherein R^{B} is —C₁₋₆alkyl-carbocyclyl (e.g. —CH₂-cyclopropyl). In certain embodiments, R^{2d} is —N(CH₃)R^{B} or —N(C₂H₅)R^{B}, wherein R^{B} is optionally substituted heterocyclyl (e.g. substituted or unsubstituted tetrahydropyranyl). In certain embodiments, R^{2d} is —C(═O)N(R^{B})₂, wherein each instance of R^{B} is independently hydrogen, optionally substituted alkyl, or optionally substituted heterocyclyl. In certain embodiments, R^{2d} is —C(═O)N(R^{B})₂, wherein each instance of R^{B} is independently optionally substituted alkyl or optionally substituted heterocyclyl. In certain embodiments, R^{2d} is —C(═O)N(R^{B})₂, wherein each instance of R^{B} is independently substituted alkyl or unsubstituted heterocyclyl. In certain embodiments, R^{2d} is —C(═O)N(R^{B})₂, wherein each instance of R^{B} is independently —C₁₋₄alkyl-heterocyclyl (e.g. —CH₂-tetrahydropyranyl) and unsubstituted heterocyclyl (e.g. tetrahydropyranyl). In certain embodiments, R^{2d} is —N(R^{B})₂ or —C(═O)N(R^{B})₂, wherein two R^{B} taken together with the intervening nitrogen form optionally substituted heterocyclyl.

As generally defined herein, R^{2e} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}. In certain embodiments, R^{2e} is hydrogen. In certain embodiments, R^{2e} is halogen (e.g. F, Cl, Br, or I). In certain embodiments, R^{2e} is optionally substituted alkyl.

In certain embodiments, each of R^{2b}, R^{2c}, and R^{2d} is independently of one of the following formulae:

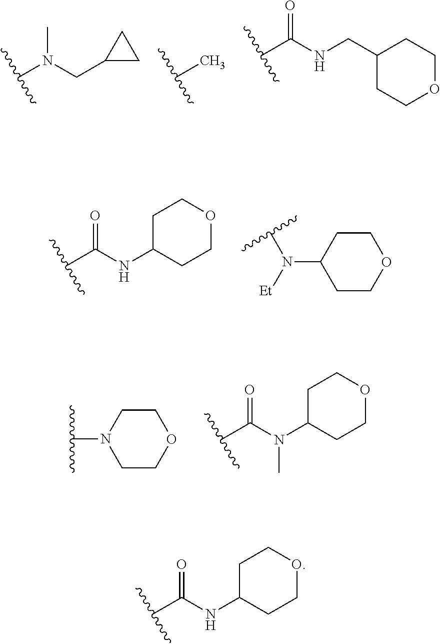

In certain embodiments, Ring A is of the formula:

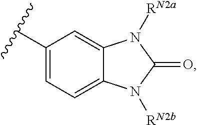

wherein each of R^{N2a} and R^{N2b} is independently hydrogen, optionally substituted alkyl, or optionally substituted aryl. In certain embodiments, R^{N2a} is hydrogen. In certain embodiments, R^{N2a} is optionally substituted phenyl. In certain embodiments, R^{N2b} is hydrogen. In certain embodiments, R^{N2b} is optionally substituted phenyl. In certain embodiments, R^{N2a} is hydrogen and R^{N2b} is optionally substituted phenyl.

In certain embodiments, Ring A is of the formula:

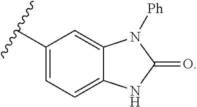 [SMILES: *c1ccc2[nH]c(=O)n(-c3ccccc3)c2c1]

In certain embodiments, Ring A is of the formula:

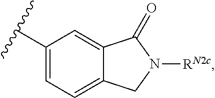

wherein R^{N2c} is optionally substituted alkyl. In certain embodiments, R^{N2c} is unsubstituted C₁₋₆ alkyl (e.g. methyl or ethyl).

In certain embodiments, Ring A is optionally substituted pyridinyl of Formula (A-3)

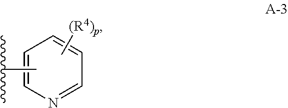

wherein

each instance of R⁴ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and

p is 0, 1, 2, or 3;

or R^{B} and another R⁴ taken together with the intervening atoms form optionally substituted heterocyclyl.

In certain embodiments, p is 0. In certain embodiments, p is 1. In certain embodiments, p is 2. In certain embodiments, p is 3.

In certain embodiments, Ring A is of one of the following formulae:

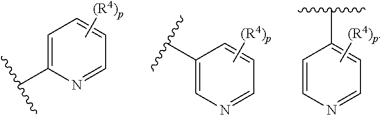

In certain embodiments, Ring A is of one of the following formulae:

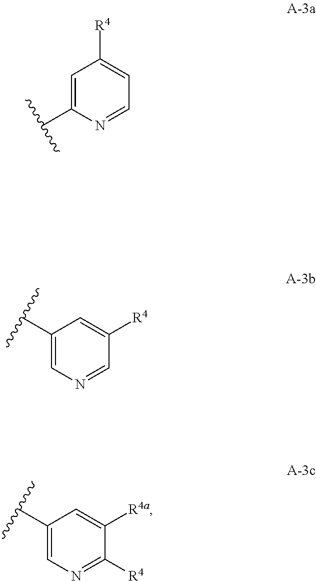

wherein

R^{4a} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂.

As generally defined herein, each instance of R⁴ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R⁴ is independently hydrogen, optionally substituted C₁₋₆ alkyl, —OR^{A}, —NR^{B}C(═O)R^{A}, or —C(═O)N(R^{B})₂, wherein R^{A} and R^{B} are as generally defined herein. In certain embodiments, R⁴ is independently hydrogen, optionally substituted C₁₋₆ alkyl, —OR^{A}, —NR^{B}C(═O)R^{A}, or —C(═O)N(R^{B})₂, wherein R^{A} is hydrogen or optionally substituted alkyl; and R^{B} is hydrogen or optionally substituted alkyl. In certain embodiments, R⁴ is independently hydrogen, optionally substituted C₁₋₆ alkyl, —OR^{A}, —NHC(═O)R^{A}, —C(═O)NHR^{B}, or —C(═O)N(CH₃)R^{B}, wherein R^{A} and R^{B} is as generally defined herein. In certain embodiments, R⁴ is independently hydrogen, optionally substituted C₁₋₆ alkyl, —OR^{A}, —NHC(═O)R^{A}, —C(═O)NHR^{B}, or —C(═O)N(CH₃)R^{B}, wherein R^{A} is hydrogen or optionally substituted alkyl; and R^{B} is optionally substituted —C₁₋₄alkyl-heteroaryl or optionally substituted —C₁₋₄alkyl-phenyl; or R^{A} and R^{B} taken together with the intervening atoms form optionally substituted heterocyclyl; or R^{B} and another R⁴ taken together with the intervening atoms form optionally substituted heterocyclyl.

As generally defined herein, each instance of R^{4a} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R^{4a} is independently hydrogen, optionally substituted C₁₋₆ alkyl, —OR^{A}, —NR^{B}C(═O)R^{A}, or —C(═O)N(R^{B})₂, wherein R^{A} and R^{B} are as generally defined herein. In certain embodiments, R^{4a} is independently hydrogen, optionally substituted C₁₋₆ alkyl, —OR^{A}, —NR^{B}C(═O)R^{A}, or —C(═O)N(R^{B})₂, wherein R^{A} is hydrogen or optionally substituted alkyl; and R^{B} is hydrogen or optionally substituted alkyl. In certain embodiments, R^{4a} is independently hydrogen, optionally substituted C₁₋₆ alkyl, —OR^{A}, —NHC(═O)R^{A}, —C(═O)NHR^{B}, or —C(═O)N(CH₃)R^{B}, wherein R^{A} and R^{B} is as generally defined herein. In certain embodiments, R^{4a} is independently hydrogen, optionally substituted C₁₋₆ alkyl, —OR^{A}, —NHC(═O)R^{A}, —C(═O)NHR^{B}, or —C(═O)N(CH₃)R^{B}, wherein R^{A} is hydrogen or optionally substituted alkyl; and R^{B} is optionally substituted —C₁₋₄alkyl-heteroaryl or optionally substituted —C₁₋₄alkyl-phenyl; or R^{A} and R^{B} taken together with the intervening atoms form optionally substituted heterocyclyl; or R^{B} and another R^{4a} taken together with the intervening atoms form optionally substituted heterocyclyl.

In certain embodiments, Ring A is one of the following formulae:

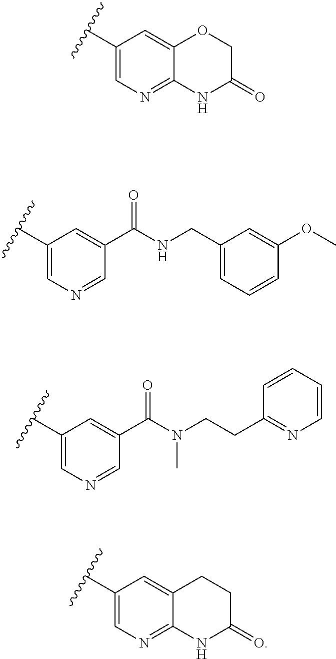 [SMILES: *c1cnc2c(c1)OCC(=O)N2 ; *c1cncc(C(=O)NCc2cccc(OC)c2)c1 ; *c1cncc(C(=O)N(C)CCc2ccccn2)c1 ; *c1cnc2c(c1)CCC(=O)N2]

In certain embodiments, Ring A is of Formula (A-4)

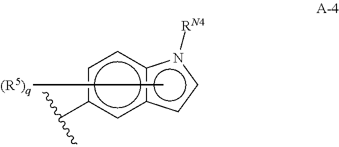

wherein

each instance of R⁵ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;

q is 0, 1, 2, 3, 4, or 5; and

each instance of R^{N4} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;

or R^{B} and another R⁵ taken together with the intervening atoms form optionally substituted heterocyclyl.

In certain embodiments, q is 0. In certain embodiments, q is 1. In certain embodiments, q is 2. In certain embodiments, q is 3. In certain embodiments, q is 4. In certain embodiments, q is 5.

In certain embodiments, Ring A is of Formula (A-4a)

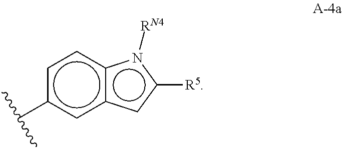

As generally defined herein, R⁵ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R⁵ is hydrogen. In certain embodiments, R⁵ is halogen (e.g. F, Cl, Br, or I). In certain embodiments, R⁵ is optionally substituted C₁₋₆ alkyl. In certain embodiments, R⁵ is unsubstituted C₁₋₆ alkyl (e.g. methyl or ethyl).

As generally defined herein, each instance of R^{N4} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R^{N4} is hydrogen. In certain embodiments, R^{N4} is optionally substituted alkyl (e.g. substituted or unsubstituted methyl). In certain embodiments, R^{N4} is a nitrogen protecting group. In certain embodiments, R^{N4} is a optionally substituted acyl (e.g. acetyl).

In certain embodiments, Ring A is one of the following formulae:

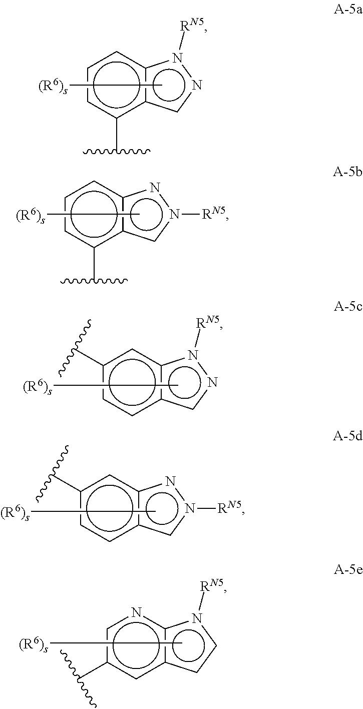

wherein

each instance of R⁶ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and

s is 0, 1, 2, 3, or 4;

each instance of R^{N5} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

In certain embodiments, s is 0. In certain embodiments, s is 1. In certain embodiments, s is 2. In certain embodiments, s is 3. In certain embodiments, s is 4.

In certain embodiments, Ring A is of one of the following formulae:

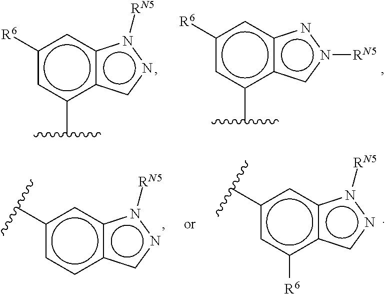

As generally defined herein, each instance of R^{N5} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R^{N5} is hydrogen, optionally substituted alkyl, or —C(═O)NHR^{B}; and R^{B} is as generally defined herein. In certain embodiments, R^{N5} is hydrogen, optionally substituted alkyl, or —C(═O)NHR^{B}; and R^{B} is hydrogen or optionally substituted alkyl. In certain embodiments, R^{N5} is hydrogen. In certain embodiments, R^{N5} is optionally substituted C₁₋₆ alkyl. In certain embodiments, R^{N5} is unsubstituted C₁₋₆ alkyl (e.g. methyl, ethyl, n-propyl, or iso-propyl). In certain embodiments, R^{N5} is substituted C₁₋₆ alkyl.

In certain embodiments, R^{N5} is of Formula (i):

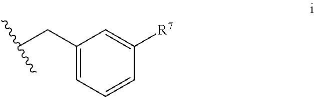

wherein R⁷ is hydrogen, optionally substituted alkyl, —OR^{A}, —C(═O)R^{A}, or —C(═O)N(R^{B})₂; and R^{A} and R^{B} are as generally defined herein.

In certain embodiments, R⁷ is hydrogen or optionally substituted alkyl. In certain embodiments, R⁷ is —OR^{A}, —C(═O)R^{A}, or —C(═O)N(R^{B})₂; and R^{A} and R^{B} are as defined herein. In certain embodiments, R⁷ is —OR^{A}, —C(═O)R^{A}, or —C(═O)N(R^{B})₂; and each instance of R^{A} is optionally substituted alkyl, optionally substituted phenyl, or optionally substituted heterocyclyl; and each instance of R^{B} is independently hydrogen or optionally substituted alkyl; or two R^{B} taken together with the intervening nitrogen form optionally substituted heterocyclyl.

In certain embodiments, R^{N5} is of one of the following formulae:

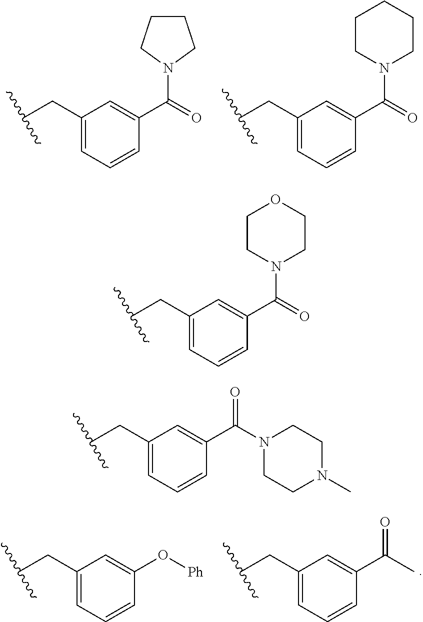

In certain embodiments, R^{N5} is of Formula (ii):

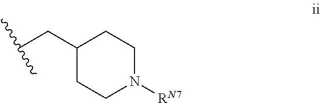

wherein R^{N7} is hydrogen, optionally substituted alkyl, or a nitrogen protecting group.

In certain embodiments, R^{N7} is hydrogen. In certain embodiments, R^{N7} is a nitrogen protecting group. In certain embodiments, R^{N7} is acyl (e.g. acetyl). In certain embodiments, R^{N7} is of one of the following formulae:

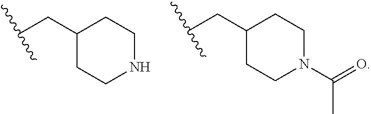 [SMILES: *CC1CCNCC1 ; *CC1CCN(C(C)=O)CC1]

As generally defined herein, each instance of R⁶ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and R^{A} and R^{B} are as generally defined herein. In certain embodiments, R⁶ is hydrogen. In certain embodiments, R⁶ is halogen (e.g. F, Cl, Br, or I). In certain embodiments, R⁶ is optionally substituted C₁₋₆ alkyl. In certain embodiments, R⁶ is unsubstituted C₁₋₆ alkyl (e.g. methyl, ethyl, n-propyl, or isopropyl). In certain embodiments, R⁶ is —C(═O)N(R^{B})₂, wherein R^{B} is as generally defined herein. In certain embodiments, R⁶ is —C(═O)N(R^{B})₂, wherein R^{B} is independently hydrogen or optionally substituted alkyl. In certain embodiments, R⁶ is —C(═O)NHR^{B} or —C(═O)N(CH₃)R^{B}, wherein R^{B} is as generally defined herein. In certain embodiments, R⁶ is —C(═O)NHR^{B} or —C(═O)N(CH₃)R^{B}, wherein R^{B} is independently hydrogen or optionally substituted alkyl. In certain embodiments, R⁶ is —C(═O)NHR^{B} or —C(═O)N(CH₃)R^{B}, wherein R^{B} is substituted alkyl. In certain embodiments, R⁶ is —C(═O)NHR^{B} or —C(═O)N(CH₃)R^{B}, wherein R^{B} is optionally substituted —C₁₋₆alkyl-phenyl. In certain embodiments, R⁶ is —C(═O)NHR^{B} or —C(═O)N(CH₃)R^{B}, wherein R^{B} is one of the following formulae:

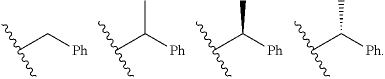

In certain embodiments, R⁶ is —C(═O)NHR^{B} or —C(═O)N(CH₃)R^{B}, wherein R^{B} is optionally substituted —C₁₋₆alkyl-hetero aryl.

In certain embodiments, Ring A is of one of the following formulae:

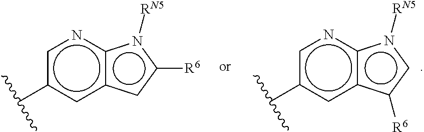

In certain embodiments, R^{N5} is hydrogen and R⁶ is unsubstituted C₁₋₆ alkyl (e.g. methyl).

In certain embodiments, Ring A is of one of the following formulae:

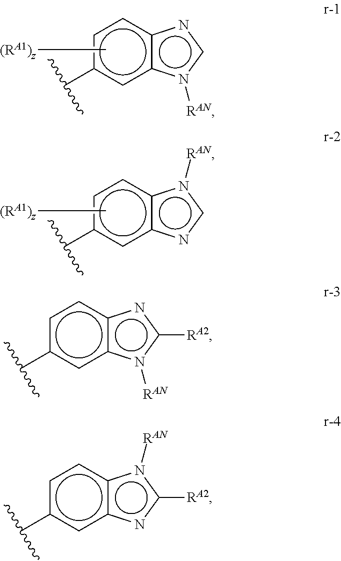

wherein

each instance of R^{A1} and R^{A2} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;

z is 0, 1, 2, or 3;

each instance of R^{A} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group; and

each instance of R^{AN} and R^{B} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;

or two R^{B} taken together with the intervening nitrogen form optionally substituted heterocyclyl;

or R^{A} and R^{B} taken together with the intervening atoms form optionally substituted heterocyclyl.

As generally defined herein, z is 0, 1, 2, or 3. In certain embodiments, z is 0. In certain embodiments, z is 1. In certain embodiments, z is 2. In certain embodiments, z is 3.

As generally defined herein, R^{AN} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R^{AN} is hydrogen, optionally substituted alkyl, optionally substituted aryl, optionally substituted carbocyclyl, or optionally substituted heterocyclyl. In certain embodiments, R^{AN} is hydrogen. In certain embodiments, R^{AN} is optionally substituted alkyl. In certain embodiments, R^{AN} is unsubstituted alkyl (e.g. methyl, ethyl, n-propyl, or isopropyl). In certain embodiments, R^{AN} is substituted alkyl.

In certain embodiments, R^{AN} is of formula:

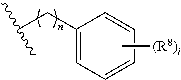

wherein

h is 0, 1, 2, 3, or 4;

i is 0, 1, 2, 3, 4, or 5;

each instance of R⁸ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and

R^{A} and R^{B} are as defined herein.

In certain embodiments, h is 0. In certain embodiments, h is 1. In certain embodiments, h is 2. In certain embodiments, h is 3. In certain embodiments, h is 4.

In certain embodiments, i is 0. In certain embodiments, i is 1. In certain embodiments, i is 2. In certain embodiments, i is 3. In certain embodiments, i is 4. In certain embodiments, i is 5.

In certain embodiments, R^{AN} is of one of the following formulae:

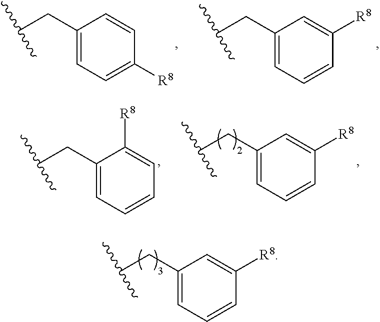

As generally defined herein, R⁸ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R⁸ is hydrogen. In certain embodiments, R⁸ is halogen (e.g. F, Cl, Br, or I). In certain embodiments, R⁸ is —CN. In certain embodiments, R⁸ is optionally substituted alkyl. In certain embodiments, R⁸ is unsubstituted alkyl (e.g. methyl or ethyl). In certain embodiments, R⁸ is —OR^{A}; and R^{A} is as generally defined herein. In certain embodiments, R⁸ is —OR^{A}; and R^{A} is optionally substituted alkyl or an oxygen protecting group. In certain embodiments, R⁸ is —OR^{A}; and R^{A} is substituted alkyl. In certain embodiments, R⁸ is —OR^{A}; and R^{A} is unsubstituted alkyl (e.g. methyl). In certain embodiments, R⁸ is —SO₂R^{A}; and R^{A} is optionally substituted alkyl. In certain embodiments, R⁸ is —SO₂R^{A}; and R^{A} is unsubstituted alkyl (e.g. methyl). In certain embodiments, R⁸ is —C(═O)N(R^{B})₂, wherein each instance of R^{B} is as generally defined herein. In certain embodiments, R⁸ is —C(═O)N(R^{B})₂, wherein each instance of R^{B} is independently hydrogen, optionally substituted alkyl, optionally substituted carbocyclyl, optionally substituted heterocyclyl, optionally substituted aryl, optionally substituted heteroaryl, or a nitrogen protecting group; or two R^{B} taken together with the intervening nitrogen form optionally substituted heterocyclyl. In certain embodiments, R⁸ is —C(═O)NHR^{B} or —C(═O)N(CH₃)R^{B}, wherein R^{B} is independently hydrogen, optionally substituted alkyl, optionally substituted carbocyclyl, optionally substituted heterocyclyl, optionally substituted aryl, optionally substituted heteroaryl, or a nitrogen protecting group. In certain embodiments, R⁸ is —C(═O)NHR^{B} or —C(═O)N(CH₃)R^{B}, wherein R^{B} is hydrogen or optionally substituted alkyl. In certain embodiments, R⁸ is —C(═O)NHR^{B} or —C(═O)N(CH₃)R^{B}, wherein R^{B} is unsubstituted alkyl (e.g. methyl or ethyl). In certain embodiments, R⁸ is —C(═O)NHR^{B} or —C(═O)N(CH₃)R^{B}, wherein R^{B} is substituted alkyl. In certain embodiments, R⁸ is —C(═O)NHR^{B} or —C(═O)N(CH₃)R^{B}, wherein R^{B} is substituted alkyl. In certain embodiments, R⁸ is —C(═O)NHR^{B} or —C(═O)N(CH₃)R^{B}, and R^{B} is optionally substituted —C₁₋₄alkyl-alkoxy, optionally substituted —C₁₋₄alkyl-aryl, optionally substituted —C₁₋₄alkyl-heterocyclyl, or optionally substituted —C₁₋₄alkyl-acyl. In certain embodiments, R⁸ is —C(═O)NHR^{B} or —C(═O)N(CH₃)R^{B}, and R^{B} is of one of the following formulae:

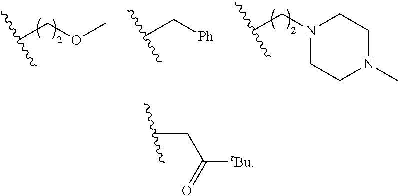

In certain embodiments, R⁸ is —C(═O)NHR^{B} or —C(═O)N(CH₃)R^{B}, and R^{B} is optionally substituted carbocyclyl (e.g. cyclopropyl, cyclopentyl, cyclohexyl, or

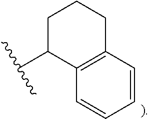 [SMILES: *C1CCCc2ccccc21]

In certain embodiments, R⁸ is —C(═O)NHR^{B} or —C(═O)N(CH₃)R^{B}, and R^{B} is optionally substituted heterocyclyl (e.g. substituted or unsubstituted tetrahydropyranyl). In certain embodiments, R⁸ is —C(═O)N(R^{B})₂, wherein two R^{B} taken together with the intervening nitrogen form optionally substituted heterocyclyl (e.g. optionally substituted pyrrolidine, optionally substituted piperidine, optionally substituted piperazine, optionally substituted azepane, optionally substituted diazepane). In certain embodiments, R⁸ is of one of the following formulae:

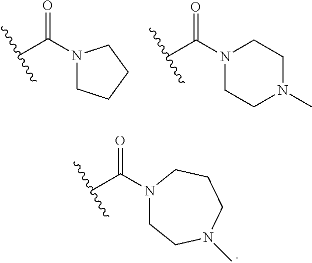

In certain embodiments, R^{AN} is of the formula

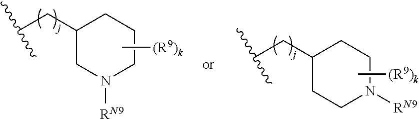

wherein

each instance of j is 0, 1, 2, 3, or 4;

each instance of k is 0, 1, 2, 3, 4, 5, or 6, as valency permits;

each instance of R⁹ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and

each instance of R^{N9} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

In certain embodiments, j is 0. In certain embodiments, j is 1. In certain embodiments, j is 2. In certain embodiments, j is 3. In certain embodiments, j is 4.

In certain embodiments, k is 0. In certain embodiments, k is 1. In certain embodiments, k is 2. In certain embodiments, k is 3. In certain embodiments, k is 4. In certain embodiments, k is 5. In certain embodiments, k is 6.

In certain embodiments, R^{AN} is of one of the following formulae:

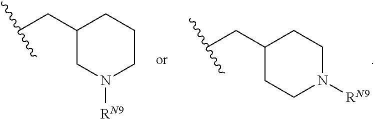

As generally defined herein, each instance of R^{N9} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R^{N9} is hydrogen, optionally substituted alkyl, or a nitrogen protecting group. In certain embodiments, R^{N9} is hydrogen. In certain embodiments, R^{N9} is optionally substituted alkyl. In certain embodiments, R^{N9} is unsubstituted alkyl (e.g. methyl). In certain embodiments, R^{N9} is substituted alkyl. In certain embodiments, R^{N9} is a nitrogen protecting group (e.g. acyl). In certain embodiments, R^{N9} is —C(═O)CH₃. In certain embodiments, R^{N9} is —SO₂—CH₃.

As generally defined herein, each instance of R⁹ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R⁹ is hydrogen. In certain embodiments, R⁹ is optionally substituted alkyl (e.g. substituted or unsubstituted methyl).

In certain embodiments, R^{AN} is optionally substituted carbocyclyl. In certain embodiments, R^{AN} is unsubstituted carbocyclyl (e.g. cyclopropyl, cyclobutyl, or cyclopentyl).

In certain embodiments, R^{AN} is optionally substituted heterocyclyl. In certain embodiments, R^{AN} is substituted tetrahydropyranyl. In certain embodiments, R^{AN} is unsubstituted tetrahydropyranyl. In certain embodiments, R^{AN} is substituted piperidine. In certain embodiments, R^{AN} is unsubstituted piperidine. In certain embodiments, R^{AN} is one of the following formulae:

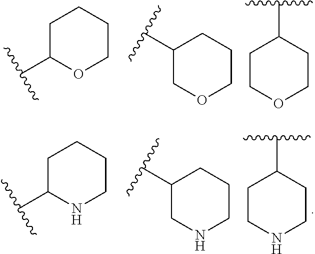

As generally defined herein, R^{A1} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R^{A1} is hydrogen, optionally substituted alkyl, optionally substituted aryl, or optionally substituted heteroaryl. In certain embodiments, R^{A1} is hydrogen. In certain embodiments, R^{A1} is substituted C₁₋₆ alkyl. In certain embodiments, R^{A1} is optionally substituted —C₁₋₄alkyl-aryl. In certain embodiments, R^{A1} is optionally substituted —CH₂-monosubstituted-phenyl. In certain embodiments, R^{A1} is of formula

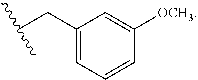 [SMILES: *Cc1cccc(OC)c1]

In certain embodiments, R^{A1} is substituted phenyl (e.g. o-methoxy-phenyl). In certain embodiments, R^{A1} is optionally substituted heteroaryl. In certain embodiments, R^{A1} is substituted quinolinyl. In certain embodiments, R^{A1} is unsubstituted quinolinyl. In certain embodiments, R^{A1} is one of the following formulae:

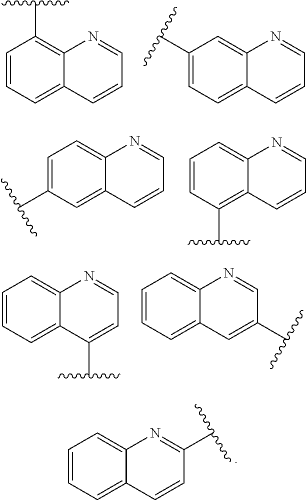

In certain embodiments, Ring A is one of the following formulae

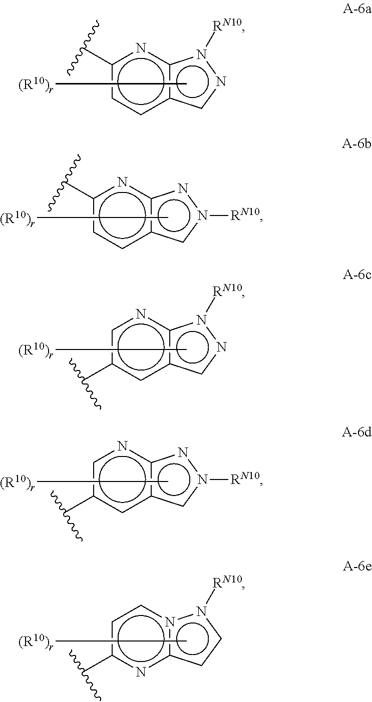

wherein

each instance of R¹⁰ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;

r is 0, 1, 2, 3, or 4, as valency permits; and

each instance of R^{N10} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

In certain embodiments, Ring A is of the formula:

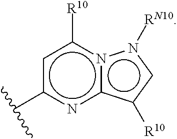

In certain embodiments, Ring A is of the formula:

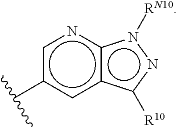

As generally defined herein, each instance of R¹⁰ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, each instance of R¹⁰ is independently optionally substituted C₁₋₆ alkyl or optionally substituted six-membered heterocyclyl. In certain embodiments, R¹⁰ is unsubstituted C₁₋₆ alkyl (e.g. methyl, ethyl, n-propyl, or isopropyl). In certain embodiments, R¹⁰ is unsubstituted six-membered heterocyclyl (e.g. morpholinyl). In certain embodiments, R¹⁰ is of one of the following formulae:

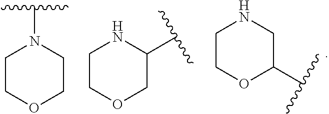

In certain embodiments, R¹⁰ is halogen or optionally substituted carbocyclyl. In certain embodiments, R¹⁰ is halogen (e.g. F, Cl, Br, or I). In certain embodiments, R¹⁰ is optionally substituted C₃₋₆ carbocyclyl. In certain embodiments, R¹⁰ is cyclopropyl.

In certain embodiments, Ring A is of one of the following formulae:

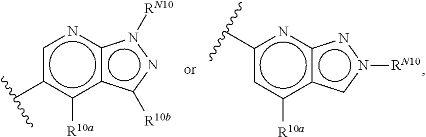

wherein

each of R^{10a} and R^{10b} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and each instance of R^{N10} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

As generally defined herein, each instance of R^{N10} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R^{N10} is hydrogen. In certain embodiments, R^{N10} is optionally substituted C₁₋₆ alkyl. In certain embodiments, R^{N10} is unsubstituted C₁₋₆ alkyl (e.g. methyl, ethyl, n-propyl, isopropyl, or tert-butyl). In certain embodiments, R^{N10} is substituted C₁₋₆ alkyl. In certain embodiments, R^{N10} is R^{N10} is C₁₋₆ haloalkyl. In certain embodiments, R^{N10} is —CH₂—CF₃. In certain embodiments, R^{N10} is —C₁₋₄alkyl-cyano (e.g. —CH₂—CN). In certain embodiments, R^{N10} is —C₁₋₄alkyl-alkenyl (e.g. —CH₂—CH═CH₂). In certain embodiments, R^{N10} is —C₁₋₄alkyl-aryl (e.g. —CH₂-phenyl or —CH₂-o-F-phenyl). In certain embodiments, R^{N10} is —C₁₋₄alkyl-carbocyclyl (e.g. —CH₂-cyclopropyl). In certain embodiments, R^{N10} is —C₁₋₄alkyl-heteroaryl (e.g. —CH₂-pyridinyl). In certain embodiments, R^{N10} is —C₁₋₆alkyl-heterocyclyl (e.g. —CH₂— tetrahydropyranyl). In certain embodiments, R^{N10} is optionally substituted carbocyclyl. In certain embodiments, R^{N10} is cyclobutyl or cyclopropyl. In certain embodiments, R^{N10} is optionally substituted heterocyclyl. In certain embodiments, R^{N10} is substituted tetrahydropyranyl. In certain embodiments, R^{N10} is unsubstituted tetrahydropyranyl. In certain embodiments, R^{N10} is optionally substituted heteroaryl. In certain embodiments, R^{N10} is substituted or unsubstituted pyridinyl. In certain embodiments, R^{N10} is substituted pyridinyl. In certain embodiments, R^{N10} is unsubstituted pyridinyl.

As generally defined herein, R^{10a} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R^{10a} is hydrogen, halogen, —CN, optionally substituted alkyl, optionally substituted alkenyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)N(R^{B})₂, —NR^{B}C(═O)R^{A}, or —NR^{B}C(═O)N(R^{B})₂. In certain embodiments, R^{10a} is hydrogen. R^{10a} is halogen (e.g. F, Cl, Br, or I). In certain embodiments, R^{10a} is CN. In certain embodiments, R^{10a} is optionally substituted C₁₋₆ alkyl. In certain embodiments, R^{10a} is unsubstituted C₁₋₆ alkyl (e.g. methyl, ethyl, n-propyl, or isopropyl). In certain embodiments, R^{10a} is substituted C₁₋₆ alkyl. R^{10a} is C₁₋₆ haloalkyl (e.g. CF₃). In certain embodiments, R^{10a} is —C₁₋₆alkyl-OH. In certain embodiments, R^{10a} is —CH₂OH. In certain embodiments, R^{10a} is of the formula

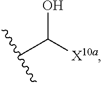

wherein X^{10a} is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, X^{10a} is unsubstituted C₁₋₆ alkyl (e.g. methyl or ethyl). In certain embodiments, X^{10a} is unsubstituted six-membered heteroaryl. In certain embodiments, X^{10a} is substituted or unsubstituted pyridinyl. In certain embodiments, X^{10a} is substituted heterocyclyl. In certain embodiments, X^{10a} is unsubstituted heterocyclyl (e.g. tetrahydropyranyl).

In certain embodiments, R^{10a} is optionally substituted alkenyl. In certain embodiments, R^{10a} is of the formula

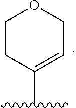 [SMILES: *C1=CCOCC1]

In certain embodiments, R^{10a} is optionally substituted aryl. In certain embodiments, R^{10a} is optionally substituted phenyl. In certain embodiments, R^{10a} is p-OH-phenyl or p-F-phenyl. In certain embodiments, R^{10a} is optionally substituted heterocyclyl. In certain embodiments, R^{10a} is optionally substituted four-membered, five-membered, or six-membered heterocyclyl.

In certain embodiments, R^{10a} is of one of the following formulae:

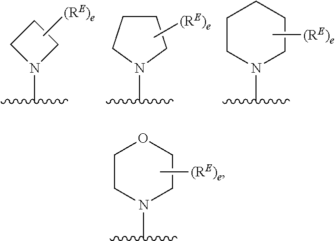

wherein

e is 0, 1, 2, 3, or 4, as valency permits; and

each instance of R^{E} is independently hydrogen, halogen, —CN, —NO₂, —N₃, —OH, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted alkoxy, or optionally substituted amino.

In certain embodiments, e is 0. In certain embodiments, e is 1. In certain embodiments, e is 2. In certain embodiments, e is 3. In certain embodiments, e is 4.

As generally defined herein, R^{E} is independently hydrogen, halogen, —CN, —NO₂, —N₃, —OH, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted alkoxy, or optionally substituted amino. In certain embodiments, R^{E} is hydrogen, halogen, CN or optionally substituted alkyl. In certain embodiments, R^{E} is hydrogen. In certain embodiments, R^{E} is halogen. In certain embodiments, R^{E} is CN. In certain embodiments, R^{E} is unsubstituted alkyl (e.g. methyl or ethyl). In certain embodiments, R^{E} is substituted alkyl. In certain embodiments, R^{E} is CF₃ or methoxy.

In certain embodiments, R^{10a} is one of the following formulae:

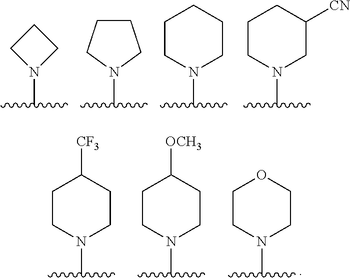

In certain embodiments, R^{10a} is optionally substituted heteroaryl. In certain embodiments, R^{10a} is optionally substituted five-membered heteroaryl. In certain embodiments, R^{10a} is unsubstituted five-membered heteroaryl. In certain embodiments, R^{10a} is thiophenyl, furanyl, thiazolyl, or pyrazolyl. In certain embodiments, R^{10a} is substituted five-membered heteroaryl. In certain embodiments, R^{10a} is one of the following formulae:

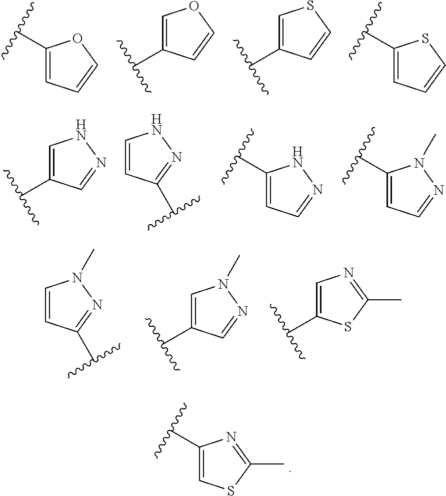

In certain embodiments, R^{10a} is optionally substituted six-membered heteroaryl. In certain embodiments, R^{10a} is substituted or unsubstituted pyridinyl. In certain embodiments, R^{10a} is —OR^{A}; and R^{A} is optionally substituted alkyl, optionally substituted carbocyclyl, optionally substituted heterocyclyl, optionally substituted aryl, or an oxygen protecting group. R^{A} is unsubstituted C₁₋₆ alkyl (e.g. methyl or ethyl). In certain embodiments, R^{10a} is —OR^{A}; and R^{A} is substituted C₁₋₆ alkyl. In certain embodiments, R^{10a} is —OR^{A}; and R^{A} is C₁₋₆ haloalkyl or —C₁₋₆ alkyl-carbocyclyl. In certain embodiments, R^{10a} is —OR^{A}; and R^{A} is —CH₂—CF₃ or —CH₂-cyclopropyl. In certain embodiments, R^{10a} is —OR^{A}; and R^{A} is optionally substituted phenyl. In certain embodiments, R^{10a} is —OR^{A}; and R^{A} is unsubstituted phenyl. In certain embodiments, R^{10a} is —OR^{A}; and R^{A} is optionally substituted heterocyclyl. In certain embodiments, R^{10a} is —OR^{A}; and R^{A} is unsubstituted five-membered heterocyclyl (e.g. tetrahydrofuranyl) or unsubstituted six-membered heterocyclyl (e.g. tetrahydropyranyl). In certain embodiments, R^{10a} is —N(R^{B})₂; and each instance of R^{B} is independently hydrogen, optionally substituted alkyl, optionally substituted carbocyclyl, optionally substituted aryl, optionally substituted heteroaryl, or a nitrogen protecting group. In certain embodiments, R^{10a} is —NHR^{B} or —N(CH₃)R^{B}; and R^{B} is independently hydrogen, optionally substituted alkyl, optionally substituted carbocyclyl, optionally substituted aryl, optionally substituted heteroaryl, or a nitrogen protecting group. In certain embodiments, R^{10a} is —NHR^{B} or —N(CH₃)R^{B}; and R^{B} is hydrogen. In certain embodiments, R^{10a} is —NHR^{B} or —N(CH₃)R^{B}; and R^{B} is unsubstituted C₁₋₆ alkyl (e.g. methyl or ethyl). In certain embodiments, R^{10a} is —NHR^{B} or —N(CH₃)R^{B}; and R^{B} is substituted C₁₋₆ alkyl. In certain embodiments, R^{10a} is —NHR^{B} or —N(CH₃)R^{B}; and R^{B} is optionally substituted —C₁₋₄alkyl-carbocyclyl, optionally substituted —C₁₋₄alkyl-heteroaryl, optionally substituted —C₁₋₄alkyl-heterocyclyl, or optionally substituted —C₁₋₄alkyl-CO₂X^{10B}; and X^{10B} is hydrogen or optionally substituted alkyl.

In certain embodiments, R^{10a} is —NHR^{B} or —N(CH₃)R^{B}; and R^{B} is one of the following formulae:

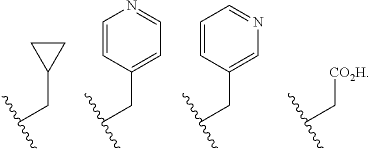

In certain embodiments, R^{10a} is —NHR^{B} or —N(CH₃)R^{B}; and R^{B} is optionally substituted C₃₋₆ carbocyclyl (e.g. substituted or unsubstituted cyclopropyl). In certain embodiments, R^{10a} is —NHR^{B} or —N(CH₃)R^{B}; and R^{B} is optionally substituted heterocyclyl (e.g. substituted or unsubstituted tetrahydropyranyl or substituted or unsubstituted oxetanyl). In certain embodiments, R^{10a} is —NHR^{B} or —N(CH₃)R^{B}; and R^{B} is optionally substituted heteroaryl. In certain embodiments, R^{10a} is —NHR^{B} or —N(CH₃)R^{B}; and R^{B} is optionally substituted thiazolyl

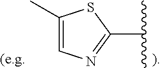 [SMILES: *c1ncc(C)s1]

In certain embodiments, R^{10a} is —NHR^{B} or —N(CH₃)R^{B}; and R^{B} is a nitrogen protecting group. In certain embodiments, R^{10a} is —NHR^{B} or —N(CH₃)R^{B}; and R^{B} is —SO₂—X^{10S}; X^{10S} is optionally substituted alkyl or —N(R^{SB})₂; and each instance of R^{SB} is hydrogen or optionally substituted alkyl. In certain embodiments, R^{10a} is —NHR^{B} or —N(CH₃)R^{B}; and R^{B} is —SO₂—N(CH₃)₂, —SO₂—CH₃, —SO₂—C₂H₅, or —SO₂—CF₃.

In certain embodiments, R^{10a} is —C(═O)N(R^{B})₂; and each instance of R^{B} is independently hydrogen, optionally substituted alkyl, optionally substituted carbocyclyl, optionally substituted aryl, optionally substituted heteroaryl, or a nitrogen protecting group; or two R^{B} taken together with the intervening nitrogen form optionally substituted heterocyclyl. In certain embodiments, R^{10a} is —C(═O)NHR^{B} or —C(═O)N(CH₃)R^{B}. In certain embodiments, R^{B} is hydrogen or optionally substituted C₁₋₆ alkyl. In certain embodiments, R^{B} is unsubstituted C₁₋₆ alkyl (e.g. methyl or ethyl). In certain embodiments, R^{B} is substituted C₁₋₆ alkyl. In certain embodiments, R^{B} is C₁₋₆ haloalkyl (e.g. —CH₂—CF₃). In certain embodiments, R^{B} is optionally substituted —C₁₋₄alkyl-carbocyclyl (e.g. —CH₂-cyclopropyl). In certain embodiments, R^{B} is optionally substituted —C₁₋₄alkyl-heteroaryl. In certain embodiments, R^{B} is one of the following formulae:

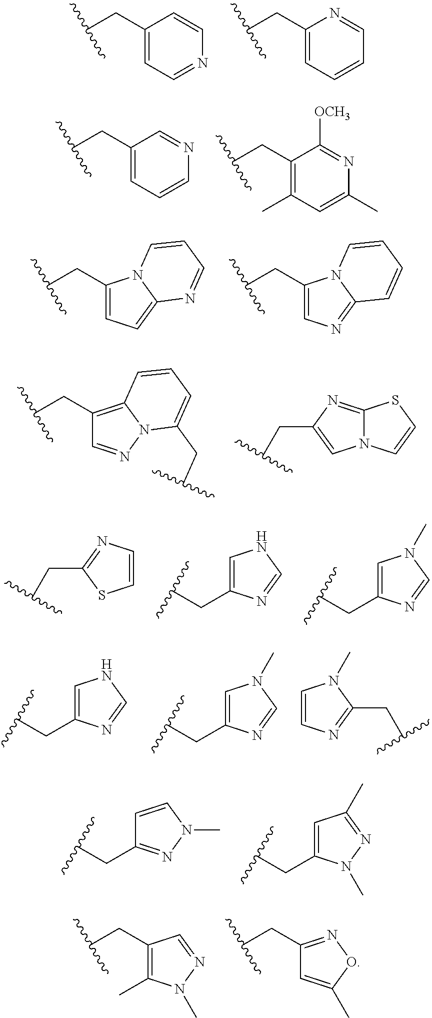

In certain embodiments, R^{B} is optionally substituted —C₁₋₄alkyl-heterocyclyl. In certain
embodiments, R^{B} is one of the following formulae:

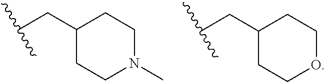

In certain embodiments, R^{B} is optionally substituted —C₁₋₄alkyl-phenyl. In certain embodiments, R^{B} is one of the following formulae:

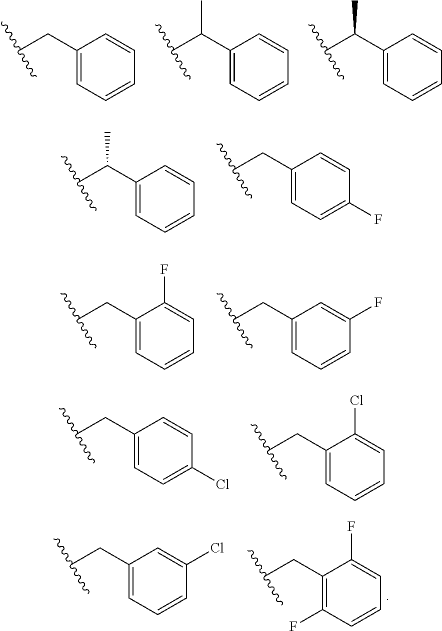

In certain embodiments, R^{B} is optionally substituted carbocyclyl (substituted or unsubstituted cyclopropyl). In certain embodiments, R^{B} is optionally substituted heterocyclyl (substituted or unsubstituted tetrahydrofuranyl or substituted or unsubstituted tetrahydropyranyl).

In certain embodiments, R^{10a} is —C(═O)N(R^{B})₂ and two R^{B} taken together with the intervening nitrogen form optionally substituted heterocyclyl. In certain embodiments, R^{10a} is one of the following formulae:

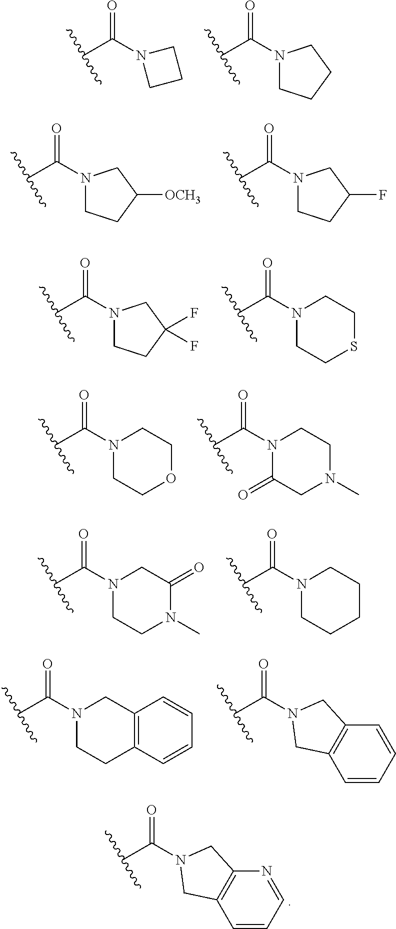

In certain embodiments, R^{10a} is —C(═O)OR^{A}; and R^{A} is optionally substituted alkyl. In certain embodiments, R^{10a} is —C(═O)OC₂H₅. In certain embodiments, R^{10a} is C(═O)R^{A}; and R^{A} is optionally substituted alkyl. In certain embodiments, R^{10a} is —C(═O)C₂H₅.

In certain embodiments, R^{10a} is —NR^{B}C(═O)OR^{A}; and R^{A} is optionally substituted alkyl; and R^{B} is hydrogen or optionally substituted alkyl. In certain embodiments, R^{10a} is —NHC(═O)OR^{A}; and R^{A} is optionally substituted alkyl. In certain embodiments, R^{10a} is —NHC(═O)OR^{A}; and R^{A} is unsubstituted alkyl (e.g. methyl or ethyl). In certain embodiments, R^{10a} is —NHC(═O)OR^{A}; and R^{A} is substituted C₁₋₆ alkyl. In certain embodiments, R^{10a} is —NHC(═O)OR^{A}; and R^{A} is C₁₋₆ alkyl-aryl (e.g. —CH₂-Ph).

In certain embodiments, R^{10a} is —NR^{B}C(═O)N(R^{B})₂; and each instance of R^{B} is independently hydrogen; optionally substituted alkyl; or optionally substituted heterocyclyl. In certain embodiments, R^{10a} is —NHC(═O)N(R^{B})₂ and each instance of R^{B} is independently hydrogen; optionally substituted alkyl; or optionally substituted heterocyclyl. In certain embodiments, R^{10a} is —NHC(═O)NHR^{B} or —NHC(═O)N(CH₃)R^{B}; and R^{B} is unsubstituted C₁₋₆ alkyl (e.g. methyl, ethyl, n-propyl, or isopropyl). In certain embodiments, R^{10a} is —NHC(═O)NHR^{B} or —NHC(═O)N(CH₃)R^{B}; and R^{B} is optionally substituted heterocyclyl (substituted or unsubstituted tetrahydropyranyl).

In certain embodiments, R^{10a} is —C(═O)N(R^{B})₂; and each instance of R^{B} is as generally defined herein. In certain embodiments, R^{10a} is —C(═O)N(R^{B})₂; and each instance of R^{B} is independently hydrogen, optionally substituted alkyl, optionally substituted carbocyclyl, optionally substituted heterocyclyl, optionally substituted heteroaryl. In certain embodiments, R^{10a} is —C(═O)NHR^{B} or —C(═O)N(CH₃)R^{B}; and R^{B} is hydrogen or optionally substituted alkyl. In certain embodiments, R^{10a} is —C(═O)NHR^{B} or —C(═O)N(CH₃)R^{B}; and R^{B}is optionally substituted C₁₋₆alkyl-heteroaryl or optionally substituted C₁₋₆alkyl-heterocyclyl. In certain embodiments, R^{10a} is —C(═O)NHR^{B} or —C(═O)N(CH₃)R^{B}; and R^{B} is of the formula:

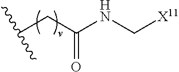

wherein v is 1, 2, 3, 4, 5, 6, or 7; and X¹¹ is optionally substituted heterocyclyl or optionally substituted five-membered heteroaryl. In certain embodiments, X¹¹ is optionally substituted tetrahydropyranyl. In certain embodiments, X¹¹ is optionally substituted pyrazole. In certain embodiments, R^{B} is of one of the following formulae:

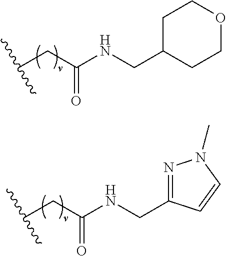

In certain embodiments, v is 1. In certain embodiments, v is 2. In certain embodiments, v is 3. In certain embodiments, v is 4. In certain embodiments, v is 5. In certain embodiments, v is 6. In certain embodiments, v is 7.

As generally defined herein, R^{10b} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂. In certain embodiments, R^{10b} is hydrogen or optionally substituted C₁₋₆ alkyl. In certain embodiments, R^{10b} is hydrogen. In certain embodiments, R^{10b} is optionally substituted C₁₋₆ alkyl. In certain embodiments, R^{10b} is substituted C₁₋₆ alkyl (e.g. C₁₋₆ haloalkyl). In certain embodiments, R^{10b} is unsubstituted C₁₋₆ alkyl (e.g. methyl or ethyl).

In certain embodiments, Ring A is one of the following formulae

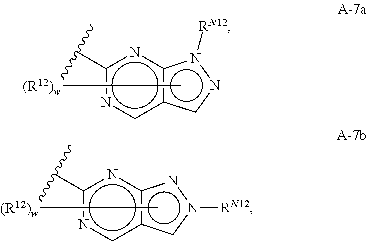

wherein

each instance of R¹² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;

w is 0, 1, or 2; and

each instance of R^{N12} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

In certain embodiments, Ring A is one of the following formulae

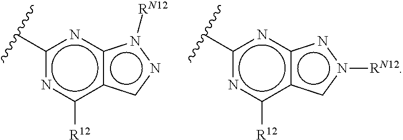

In certain embodiments, w is 0. In certain embodiments, w is 1. In certain embodiments, w is 2.

As generally defined herein, each instance of R^{N12} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R^{N12} is hydrogen. In certain embodiments, R^{N12} is optionally substituted alkyl. In certain embodiments, R^{N12} is unsubstituted C₁₋₆ alkyl (e.g. methyl, ethyl, n-propyl, i-propyl, n-butyl, s-butyl, or t-butyl).

As generally defined herein, each instance of R¹² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and wherein R^{A} and R^{B} are as generally defined herein. In certain embodiments, R¹² is —N(R^{B})₂ or —C(═O)N(R^{B})₂, wherein each instance of R^{B} is independently hydrogen, optionally substituted alkyl, optionally substituted carbocyclyl, optionally substituted heterocyclyl, optionally substituted aryl, or optionally substituted heteroaryl. In certain embodiments, R¹² is —NHR^{B}, —N(CH₃)R^{B}, —C(═O)NHR^{B}, or, —C(═O)N(CH₃)R^{B}. In certain embodiments, R^{B} is hydrogen. In certain embodiments, R^{B} is unsubstituted C₁₋₆ alkyl (e.g. methyl or ethyl). In certain embodiments, R^{B} is substituted C₁₋₆ alkyl (e.g. optionally substituted —C₁₋₆ alkyl-heterocyclyl or optionally substituted —C₁₋₆ alkyl-heteroaryl). In certain embodiments R^{B} is one of the following formulae:

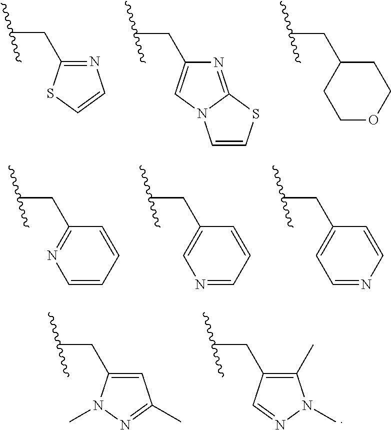

In certain embodiments, R^{B} is optionally substituted carbocyclyl. In certain embodiments, R^{B} is unsubstituted carbocyclyl (e.g. cyclopropyl). In certain embodiments, R^{B} is substituted carbocyclyl. In certain embodiments, R^{B} is optionally substituted heterocyclyl. In certain embodiments, R^{B} is unsubstituted heterocyclyl (e.g. tetrahydropyranyl). In certain embodiments, R^{B} is substituted heterocyclyl.

As generally defined herein, each instance of R^{A} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group. In certain embodiments, R^{A} is hydrogen. In certain embodiments, R^{A} is optionally substituted alkyl. In certain embodiments, R^{A} is unsubstituted alkyl (e.g. methyl or ethyl). In certain embodiments, R^{A} is substituted alkyl (e.g. haloalkyl, alkyl-carboxylate, alkyl-heteroaryl, alkyl-heterocyclyl, or alkyl-carbocyclyl). In certain embodiments, R^{A} is an oxygen protecting group. In certain embodiments, R^{A} is optionally substituted acyl (e.g. acetyl).

As generally defined herein, each instance of R^{B} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R^{B} is hydrogen. In certain embodiments, R^{B} is optionally substituted alkyl. In certain embodiments, R^{B} is unsubstituted alkyl (e.g. methyl or ethyl). In certain embodiments, R^{B} is substituted alkyl (e.g. haloalkyl, alkyl-carboxylate, alkyl-heteroaryl, alkyl-heterocyclyl, or alkyl-carbocyclyl). In certain embodiments, R^{B} is an nitrogen protecting group. In certain embodiments, R^{B} is acyl (e.g. acetyl). In certain embodiments, two R^{B} taken together with the intervening nitrogen form optionally substituted heterocyclyl. In certain embodiments, R^{A} and R^{B} taken together with the intervening atoms form optionally substituted heterocyclyl.

In certain embodiments, the optional substituent on Ring A is not an optionally substituted pyridone. In certain embodiments, the optional substituent directly attached to Ring A is not an optionally substituted pyridone. In certain embodiments, the optional substituent on the substituents directly attached to Ring A is not an optionally substituted pyridone. In certain embodiments, the aforementioned pyridone is of one of following formulae:

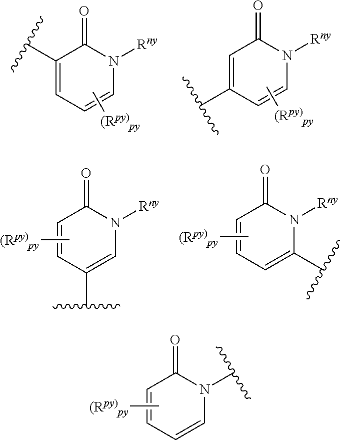

wherein each instance of R^{py} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, hydroxyl, optionally substituted alkoxy, or optionally substituted amino; each instance of R^{ny} is optionally substituted alkyl, optionally substituted alkenyl, optionally substituted alkynyl, optionally substituted carbocyclyl, optionally substituted aryl, optionally substituted heterocyclyl, optionally substituted heteroaryl, or a nitrogen protecting group; and py is 0, 1, 2, or 3 as valency permits. In certain embodiments, py is 1. In certain embodiments, R^{py} is independently halogen or optionally substituted alkyl (e.g. substituted or unsubstituted methyl). In certain embodiments, R^{ny} is hydrogen. In certain embodiments, R^{ny} is a nitrogen protecting group.

In certain embodiments, the direct substituent on Ring A is not one of the following formulae:

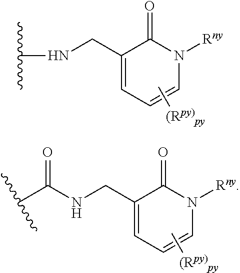

In certain embodiments, the direct substituent on Ring A is not one of the following formulae:

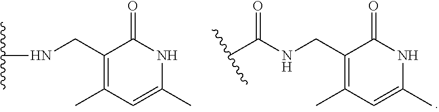 [SMILES: *NCc1c(C)cc(C)[nH]c1=O ; *C(=O)NCc1c(C)cc(C)[nH]c1=O]

In certain embodiments, a provided compound is a compound selected from any one of the compounds provided in Table 1, or a pharmaceutically acceptable salt thereof.

TABLE 1
Exemplified Compounds and Biological Activities

| | | | | | PABP1 |
|---|---|---|---|---|---|
| Cpd | | PRMT1_ | PRMT5_ | CARM1_ | ICW IC₅₀ |
| No | Compound Structure | IC₅₀ (μM) | IC₅₀ (μM) | IC₅₀ (μM) | (μM) |
| 1. | 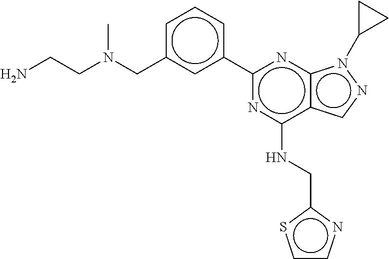 | 0.0193359 | — | 0.00356443 | — |
| 2. | 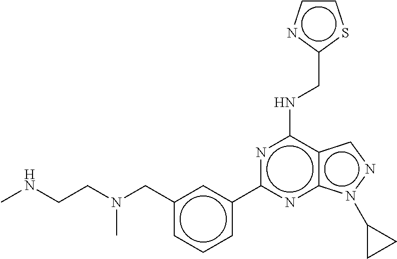 | 0.0216873 | — | 0.0168488 | — |
| 3. | 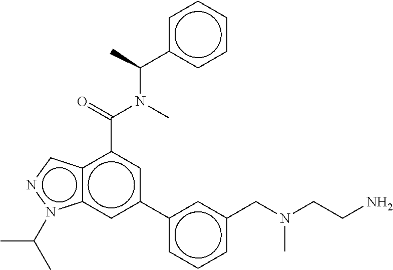 | 0.0266803 | — | 0.0459801 | — |
| 4. | 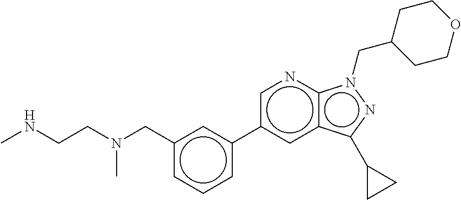 | 0.0348445 | — | 1.40031 | — |
| 5. | 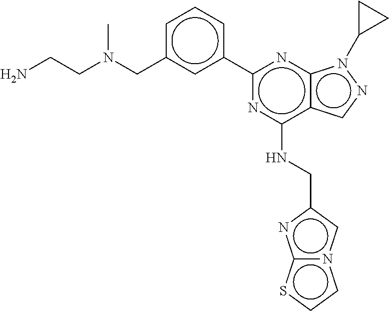 | 0.0368604 | — | 0.0032441 | 20 |
| 6. | 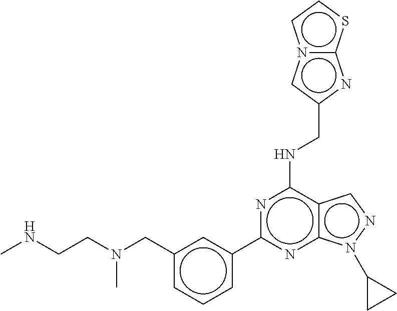 | 0.0369003 | — | 0.023836 | 0.39021525 |
| 7. | 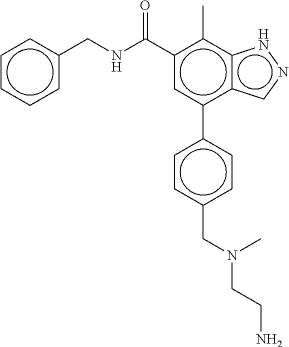 | 0.0451898 | 22.458 | 6.828 | — |
| 8. | 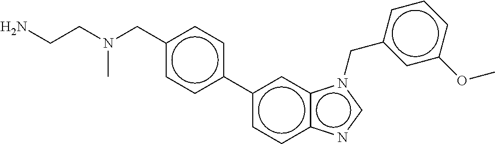 | 0.0470403 | 15.9507 | 3.86688 | — |
| 9. | 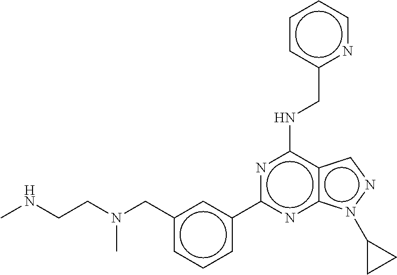 | 0.0473097 | — | 0.00759408 | 20 |
| 10. | 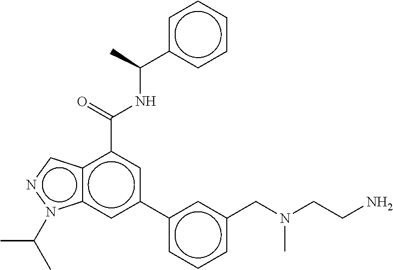 | 0.0484797 | — | 0.0451596 | — |
| 11. | 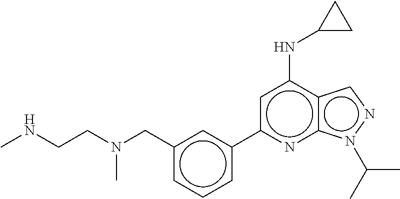 | 0.0515597 | — | 0.0328398 | 3.435765175 |
| 12. | 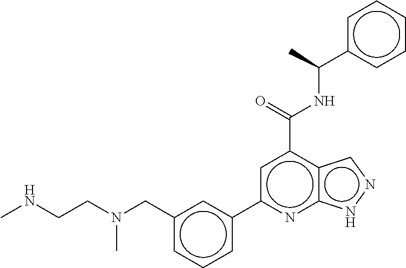 | 0.0593766 | — | 0.0793049 | — |
| 13. | 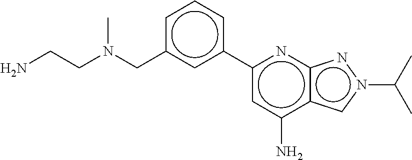 | 0.0661805 | — | 1.71311 | — |
| 14. | 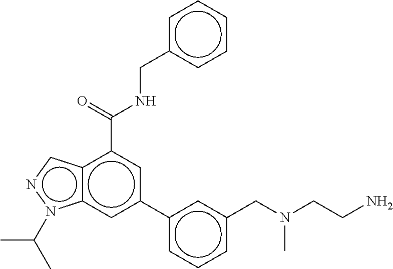 | 0.0691704 | — | 0.135301 | 20 |
| 15. | 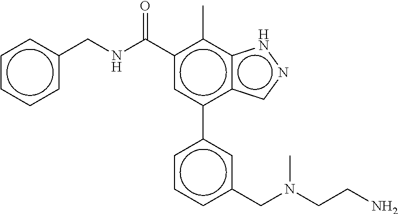 | 0.0696194 | 10.7622 | 0.973218 | — |
| 16. | 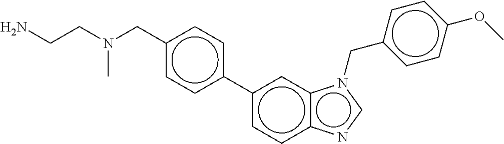 | 0.0696402 | 5.69286 | 4.08207 | — |
| 17. | 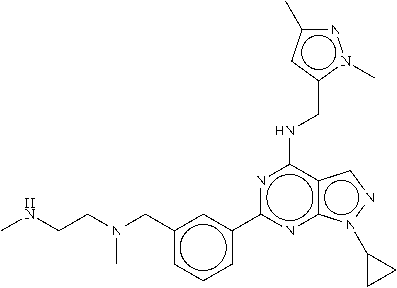 | 0.0696803 | — | 0.00841793 | 20 |
| 18. | 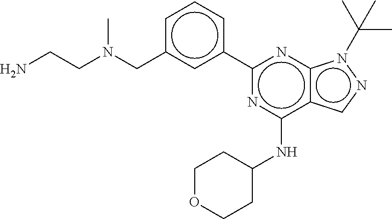 | 0.0728065 | — | 0.0058263 | 0.4419752 |
| 19. | 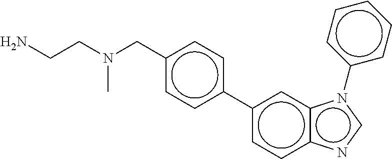 | 0.0822546 | 34.7372 | 12.5888 | — |
| 20. | 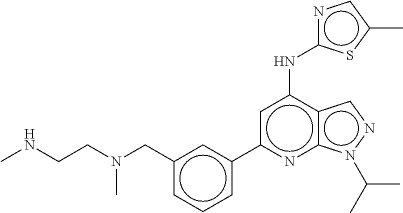 | 0.0849905 | — | 0.0140272 | 3.3299304 |
| 21. | 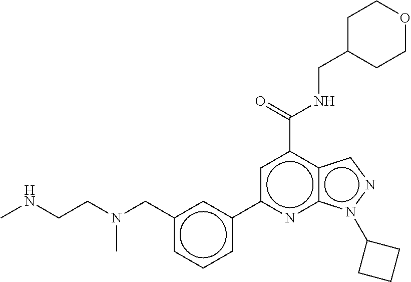 | 0.0890206 | — | 0.0438976 | 7.368296 |
| 22. | 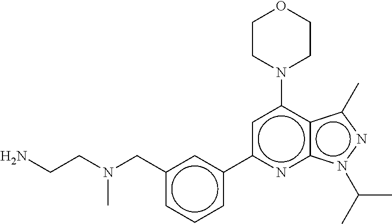 | 0.095181 | — | 0.00896985 | 3.111431875 |
| 23. | 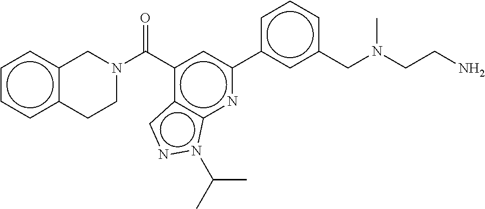 | 0.0970511 | 11.645 | 0.18517 | — |
| 24. | 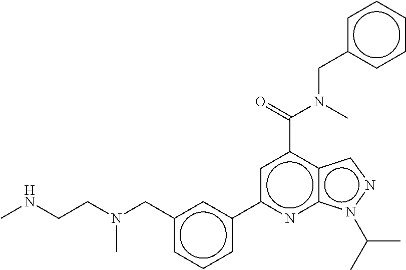 | 0.0974407 | 0.849044 | 0.0713198 | — |
| 25. | 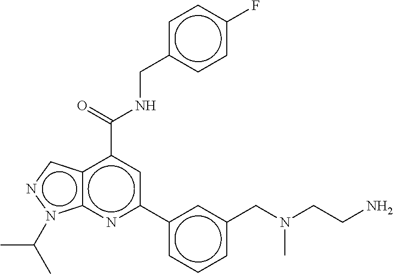 | 0.104841 | — | 0.03292 | — |
| 26. | 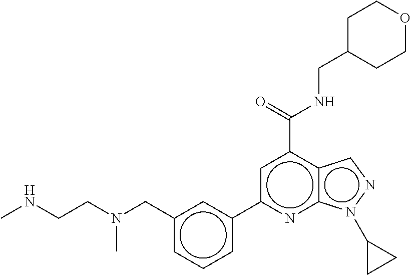 | 0.105621 | — | 0.0265699 | 2.151714333 |
| 27. | 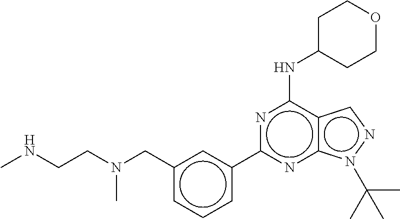 | 0.106085 | — | 0.00425324 | 0.3871179 |
| 28. | 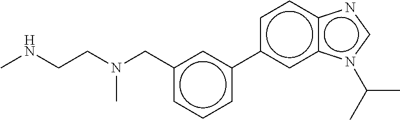 | 0.113839 | — | 12.2163 | — |
| 29. | 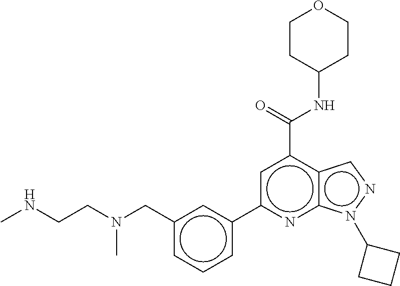 | 0.11397 | — | 0.0924223 | 5.214214 |
| 30. | 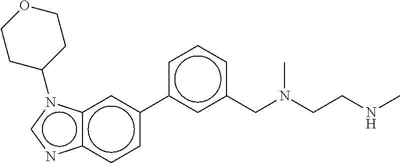 | 0.11834 | — | 0.851942 | — |
| 31. | 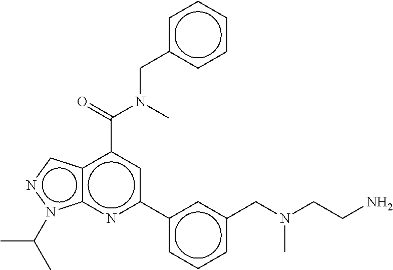 | 0.127729 | 0.857927 | 0.0217183 | — |
| 32. | 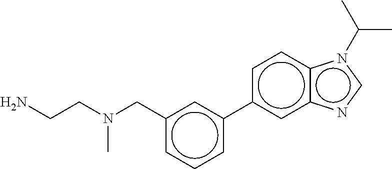 | 0.12795 | — | 7.03575 | — |
| 33. | 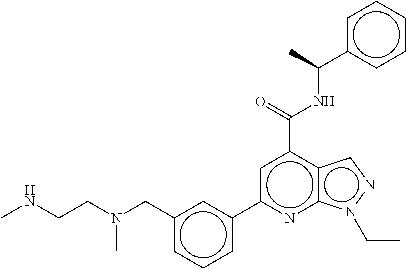 | 0.139869 | — | 0.0797499 | — |
| 34. | 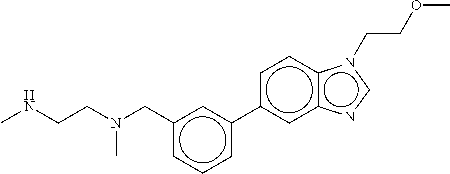 | 0.145399 | — | 10 | — |
| 35. | 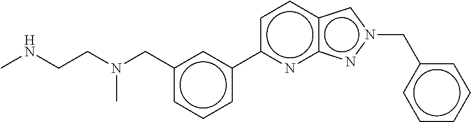 | 0.15196 | — | 4.95713 | 3.0499844 |
| 36. | 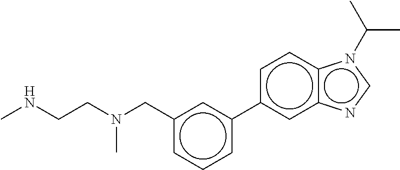 | 0.153308 | 50 | 15.8406 | — |
| 37. | 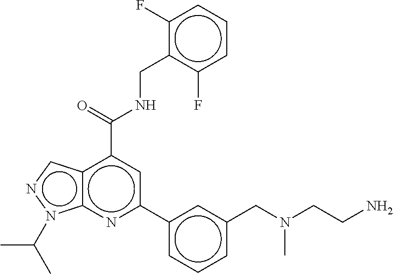 | 0.154099 | — | 0.00547004 | — |
| 38. | 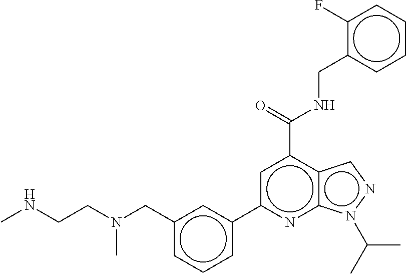 | 0.159221 | — | 0.0186724 | 4.118318 |
| 39. | 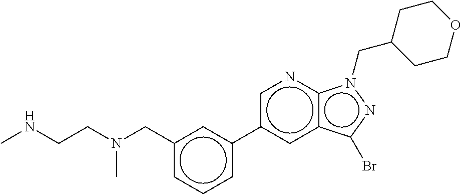 | 0.164309 | — | 6.79298 | 20 |
| 40. | 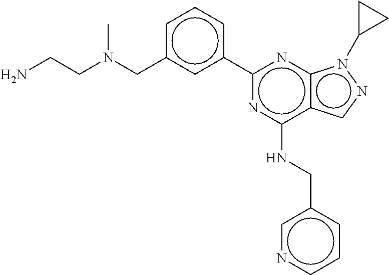 | 0.167989 | — | 0.00713515 | — |
| 41. | 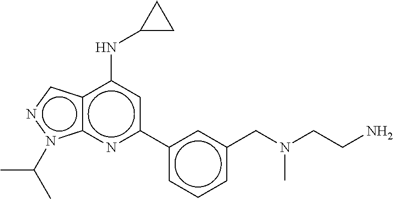 | 0.172347 | — | 0.0134202 | 5.9368516 |
| 42. | 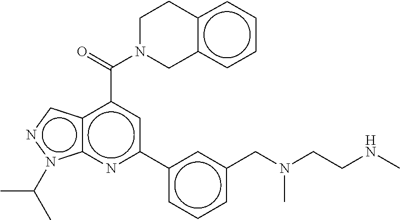 | 0.184778 | — | 0.0840531 | — |
| 43. | 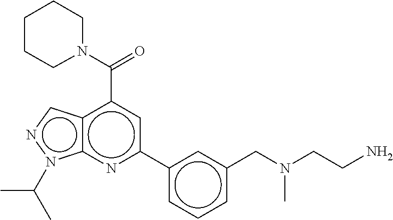 | 0.18756 | — | 0.176332 | 20 |
| 44. | 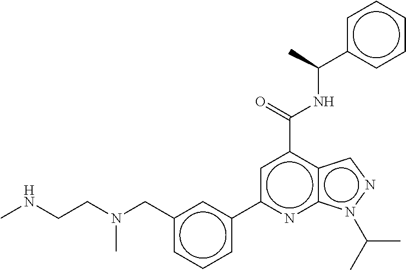 | 0.188665 | 50 | 0.0185834 | — |
| 45. | 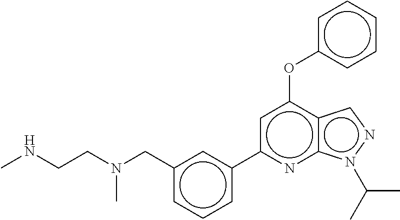 | 0.194281 | — | 0.0841008 | 20 |
| 46. | 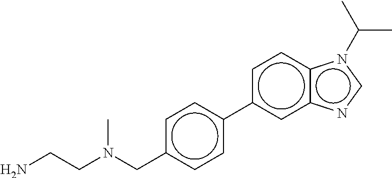 | 0.196308 | — | 10 | — |
| 47. | 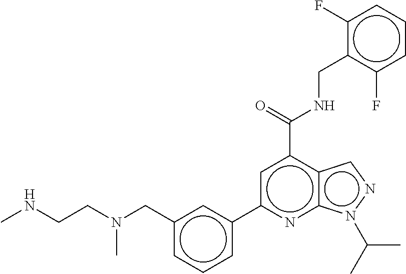 | 0.208959 | — | 0.0223898 | — |
| 48. | 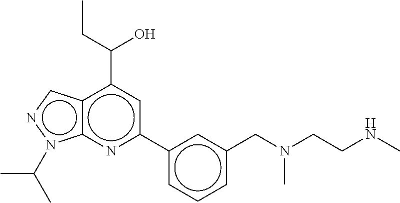 | 0.212452 | — | 0.154421 | — |
| 49. | 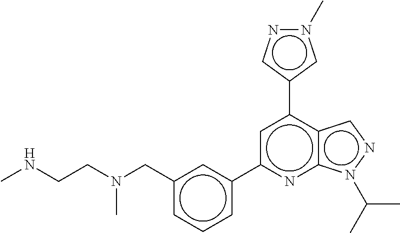 | 0.213162 | — | 0.0124447 | 1.2928301 |
| 50. | 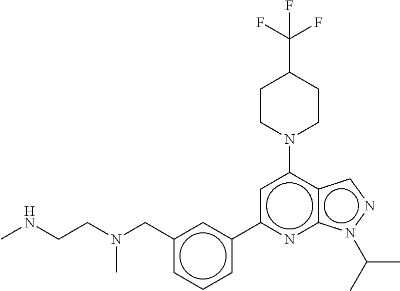 | 0.223131 | — | 0.0275797 | — |
| 51. | 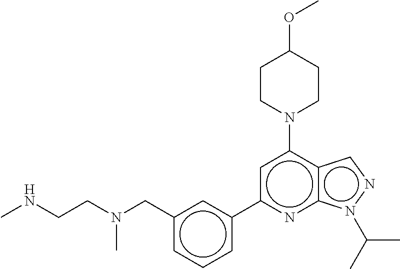 | 0.225918 | — | 0.0167498 | 5.389238417 |
| 52. | 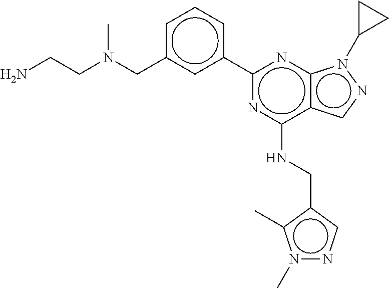 | 0.22616 | — | 0.00403567 | — |
| 53. | 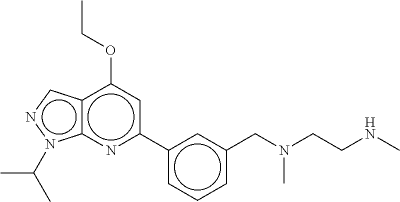 | 0.228481 | — | 0.0198999 | 3.47453335 |
| 54. | 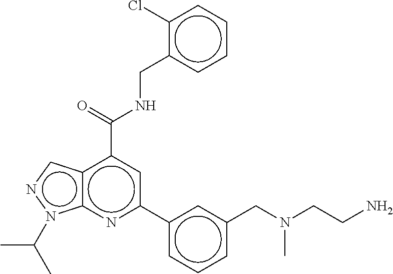 | 0.230553 | — | 0.0260802 | 20 |
| 55. | 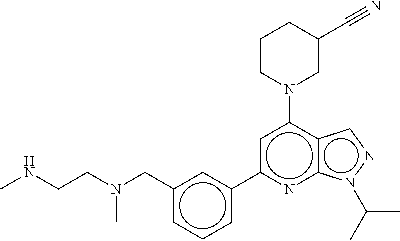 | 0.235538 | — | 0.0354601 | 4.81112185 |
| 56. | 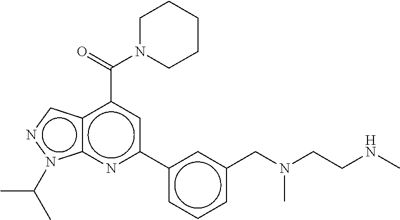 | 0.237509 | — | 0.21921 | 16.833904 |
| 57. | 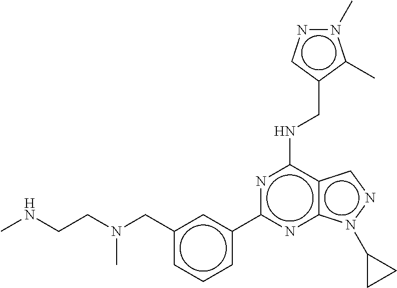 | 0.237608 | — | 0.0184673 | 20 |
| 58. | 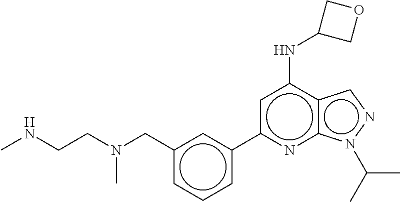 | 0.238798 | — | 0.0221658 | 3.199702225 |
| 59. | 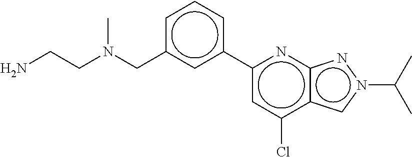 | 0.244236 | — | 0.706985 | 15.48297 |
| 60. | 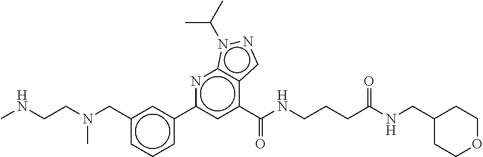 | 0.247543 | — | 0.0172467 | — |
| 61. | 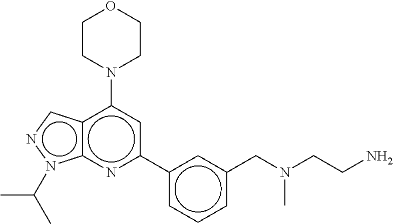 | 0.248788 | — | 0.00521651 | 5.660200483 |
| 62. | 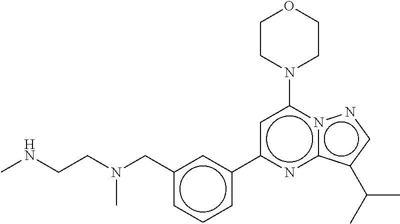 | 0.257935 | — | 0.0208142 | — |
| 63. | 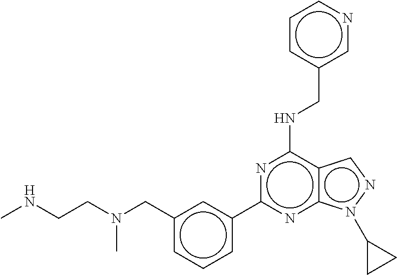 | 0.26199 | — | 0.0169727 | — |
| 64. | 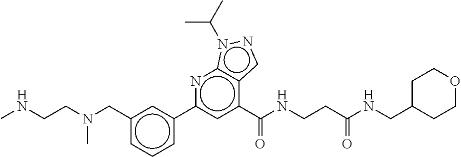 | 0.270817 | — | 0.0249419 | — |
| 65. | 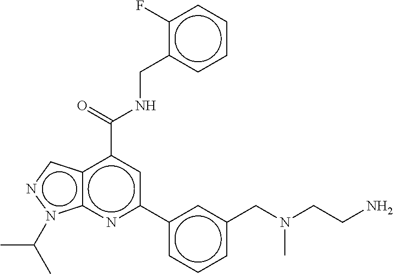 | 0.27145 | — | 0.00838997 | — |
| 66. | 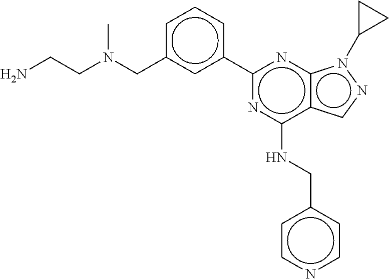 | 0.27429 | — | 0.00631852 | 20 |
| 67. | 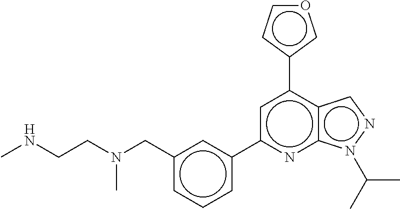 | 0.274739 | — | 0.02813 | — |
| 68. | 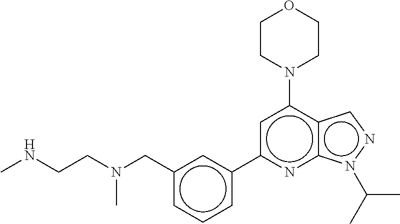 | 0.276905 | — | 0.00984155 | — |
| 69. | 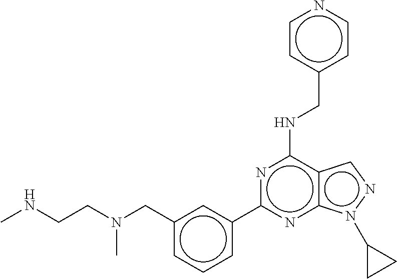 | 0.281988 | — | 0.0195261 | — |
| 70. | 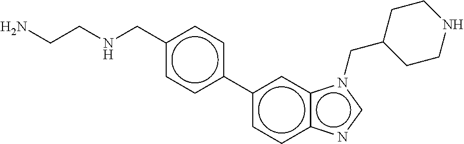 | 0.294958 | 0.421079 | 11.6555 | — |
| 71. | 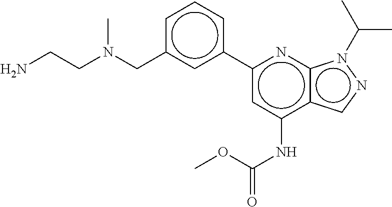 | 0.3021 | — | 0.0106491 | 10.9637801 |
| 72. | 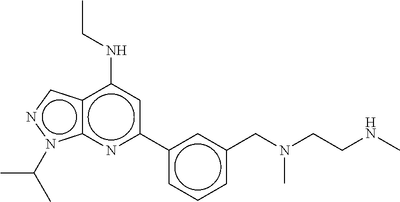 | 0.303103 | — | 0.108969 | 5.36446875 |
| 73. | 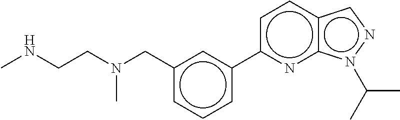 | 0.305971 | 50 | 0.380834 | 20 |
| 74. | 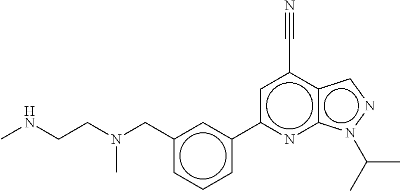 | 0.308506 | — | 0.0384203 | 20 |
| 75. | 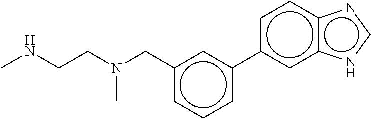 | 0.312198 | — | 10 | — |
| 76. | 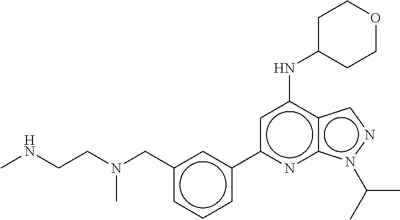 | 0.316563 | — | 0.0131801 | 1.63390022 |
| 77. | 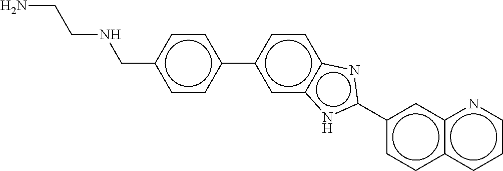 | 0.31749 | 0.25798 | — | — |
| 78. | 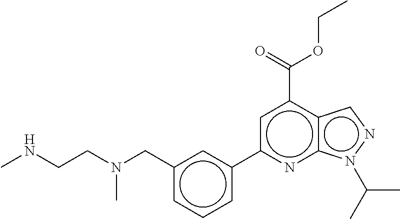 | 0.337451 | — | 0.0179602 | 13.00588865 |
| 79. | 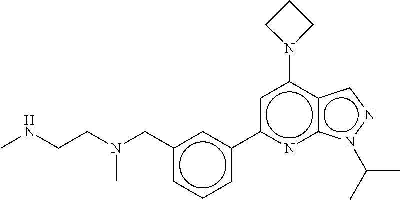 | 0.340612 | — | 0.0145118 | 20 |
| 80. | 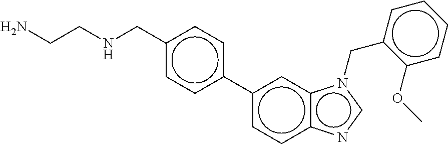 | 0.344937 | 0.16135 | 4.81338 | — |
| 81. | 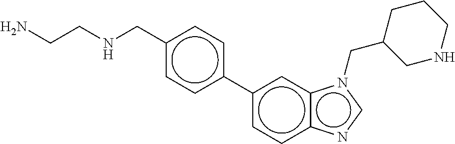 | 0.359154 | 0.321218 | 4.2658 | — |
| 82. | 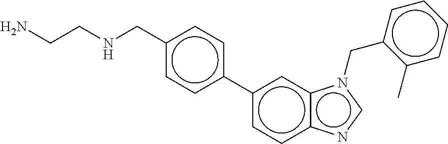 | 0.374878 | 0.235546 | 8.12737 | — |
| 83. | 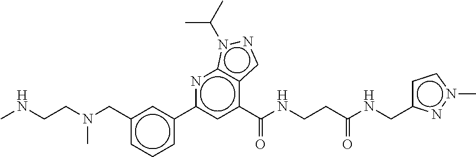 | 0.377799 | — | 0.025691 | — |
| 84. | 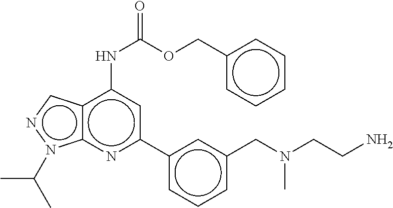 | 0.386688 | 27.6688 | 0.0137129 | 13.29513 |
| 85. | 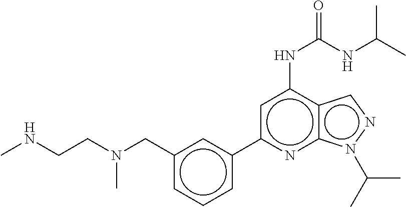 | 0.390994 | — | 0.0139687 | 2.582685583 |
| 86. | 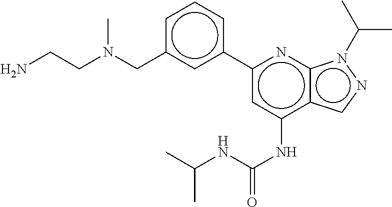 | 0.396972 | — | 0.0081099 | 3.459822333 |
| 87. | 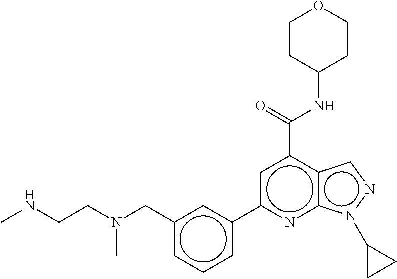 | 0.412278 | — | 0.036831 | 2.1656329 |
| 88. | 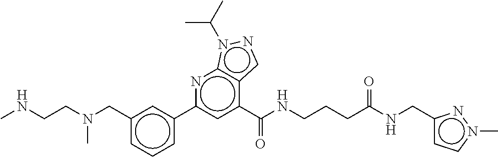 | 0.413343 | — | 0.0164594 | — |
| 89. | 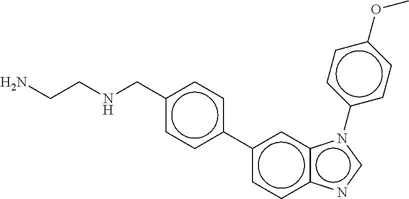 | 0.413933 | 0.417052 | — | — |
| 90. | 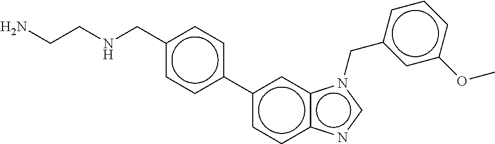 | 0.429932 | 0.125382 | — | — |
| 91. | 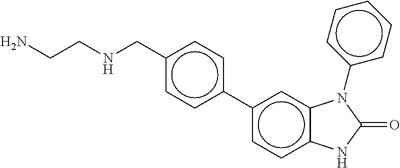 | 0.431301 | 0.501649 | — | — |
| 92. | 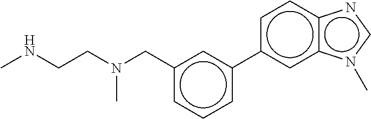 | 0.432126 | — | 6.48754 | — |
| 93. | 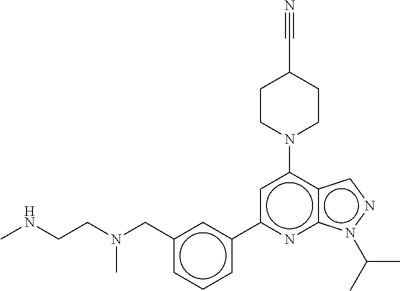 | 0.432673 | — | 0.0105898 | 1.6539789 |
| 94. | 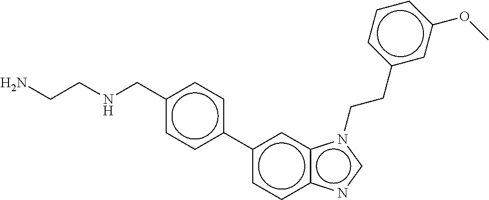 | 0.445995 | 0.0887749 | 4.27376 | — |
| 95. | 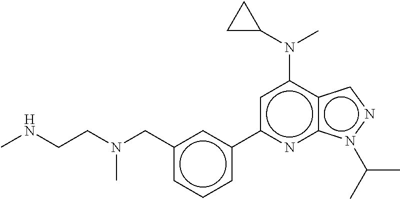 | 0.450952 | 200 | 0.0364805 | 6.802524 |
| 96. | 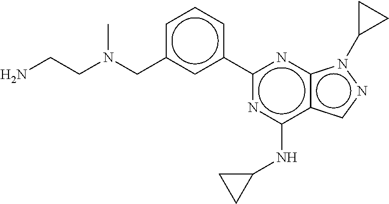 | 0.451149 | — | 0.00841841 | 12.53906635 |
| 97. | 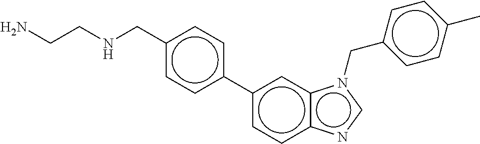 | 0.451643 | 0.0659437 | 8.19276 | — |
| 98. | 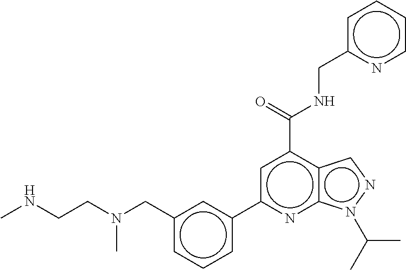 | 0.455754 | — | 0.0324097 | 1.597369575 |
| 99. | 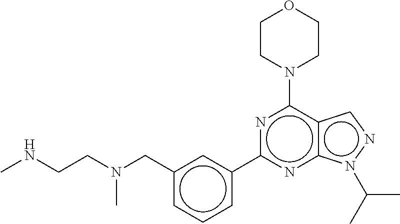 | 0.457028 | — | 0.0113437 | 1.5395674 |
| 100. | 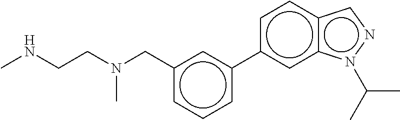 | 0.477035 | 50 | 3.69948 | — |
| 101. | 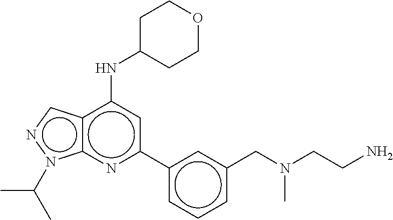 | 0.488424 | — | 0.00511311 | 6.308677033 |
| 102. | 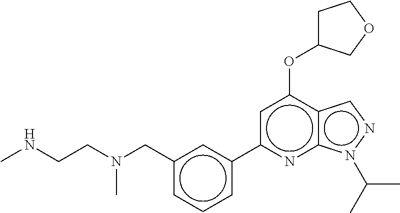 | 0.492334 | — | 0.0223501 | 10.92276495 |
| 103. | 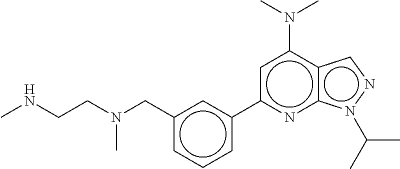 | 0.517107 | — | 0.0576669 | 10.30731495 |
| 104. | 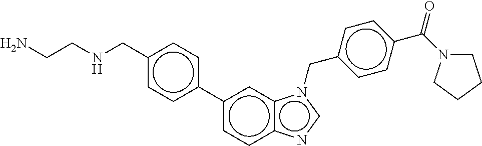 | 0.530726 | 0.100249 | 4.11984 | — |
| 105. | 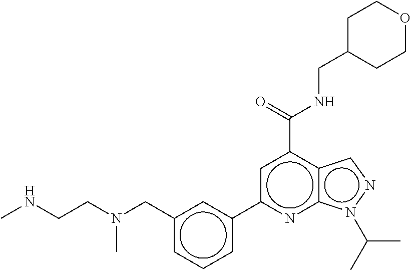 | 0.547823 | — | 0.0180793 | 1.355853267 |
| 106. | 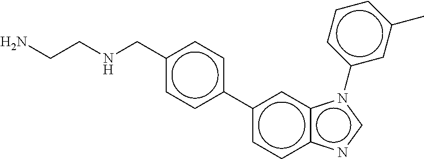 | 0.557982 | 0.177722 | 14.8919 | — |
| 107. | 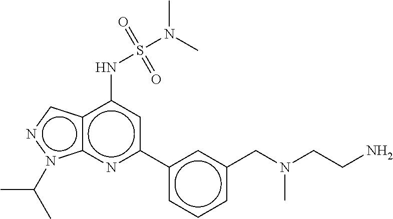 | 0.562245 | — | 0.0257591 | 11.81944003 |
| 108. | 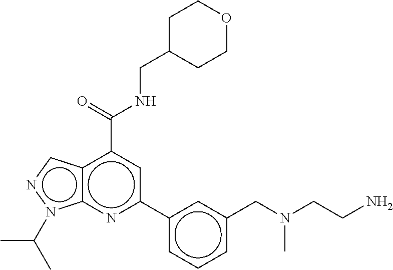 | 0.568297 | — | 0.0103674 | 1.13423494 |
| 109. | 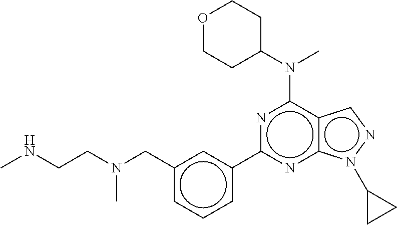 | 0.569876 | — | 0.00501753 | 10.65808715 |
| 110. | 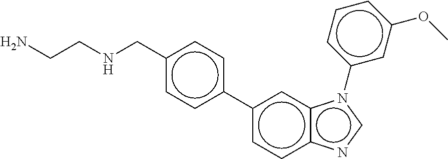 | 0.583231 | 0.272848 | 10.5947 | — |
| 111. | 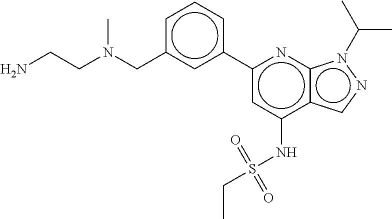 | 0.585221 | — | 0.130569 | 11.92712232 |
| 112. | 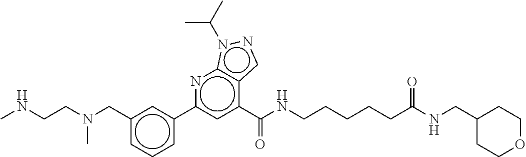 | 0.588458 | — | 0.0238561 | — |
| 113. | 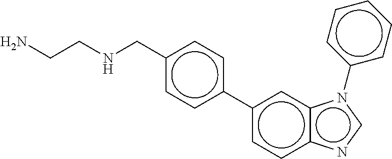 | 0.604151 | 0.366317 | — | — |
| 114. | 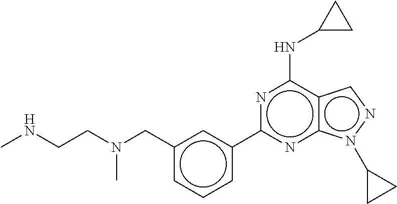 | 0.617164 | — | 0.01795 | 20 |
| 115. | 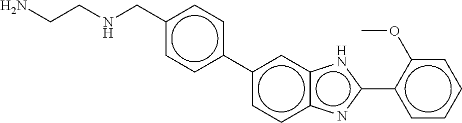 | 0.629405 | 0.34325 | 6.87354 | — |
| 116. | 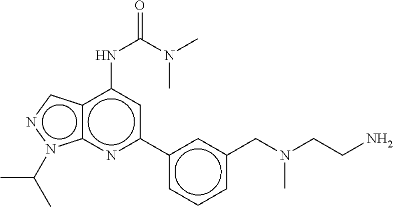 | 0.640288 | — | 0.0234214 | 2.62035085 |
| 117. | 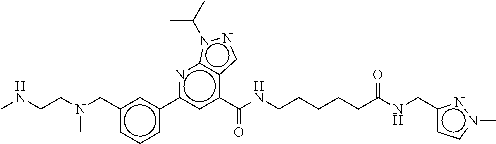 | 0.642178 | — | 0.033424 | — |
| 118. | 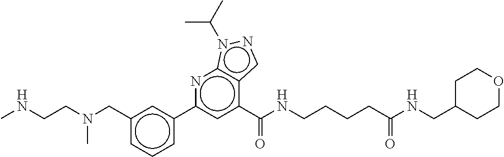 | 0.667775 | — | 0.0179763 | — |
| 119. | 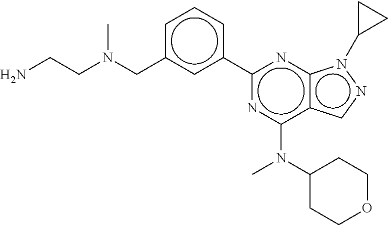 | 0.714234 | — | 0.00477277 | 1.365948925 |
| 120. | 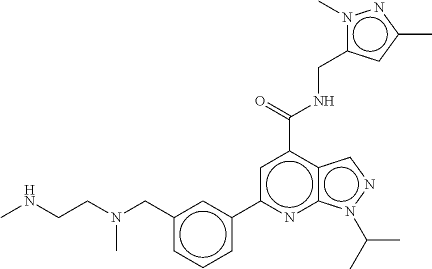 | 0.71839 | — | 0.0355984 | 1.529891425 |
| 121. | 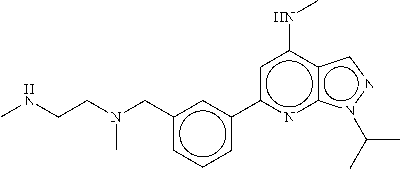 | 0.72078 | — | 0.0702476 | 7.4509711 |
| 122. | 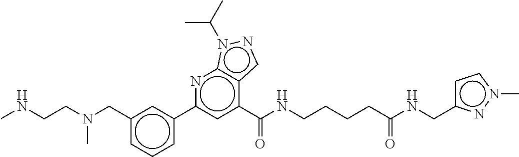 | 0.725221 | — | 0.0135039 | — |
| 123. | 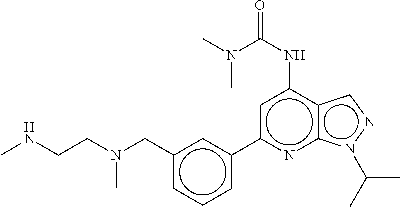 | 0.766311 | — | 0.0295948 | 4.7790361 |
| 124. | 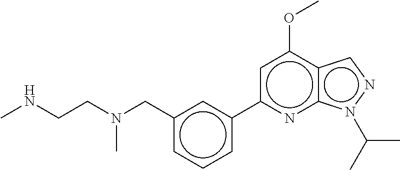 | 0.769556 | — | 0.0754606 | 17.756671 |
| 125. | 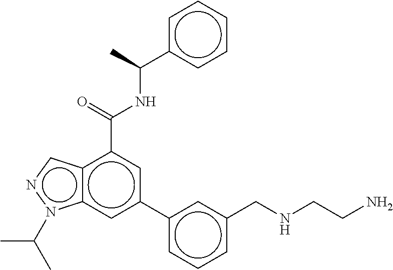 | 0.782619 | — | 0.0758002 | — |
| 126. | 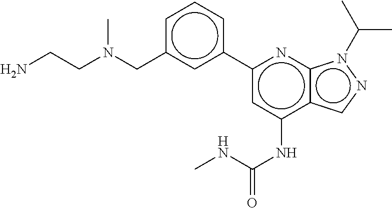 | 0.782943 | — | 0.00796 | 10.76721905 |
| 127. | 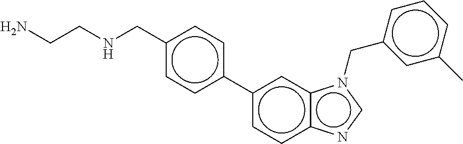 | 0.801863 | 0.0744947 | — | — |
| 128. | 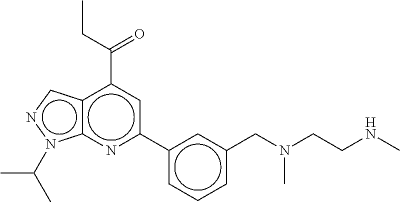 | 0.807979 | — | 0.0307518 | 20 |
| 129. | 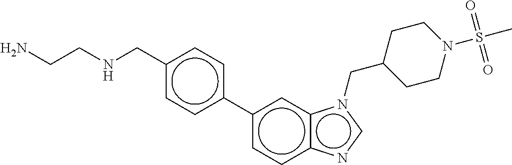 | 0.821448 | 0.121644 | 12.8834 | — |
| 130. | 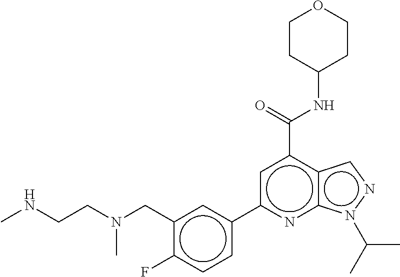 | 0.833471 | — | 0.130789 | 11.4563534 |
| 131. | 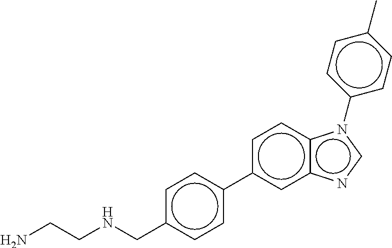 | 0.841454 | 0.370826 | 2.83576 | — |
| 132 | 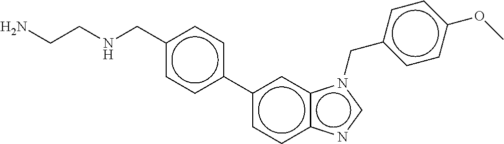 | 0.847657 | 0.0571775 | 7.20875 | — |
| 133. | 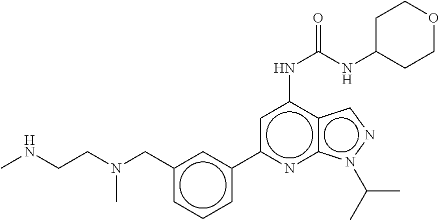 | 0.865965 | — | 0.0119939 | 11.53197265 |
| 134. | 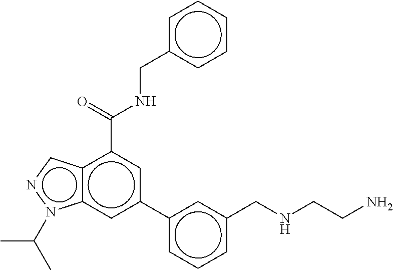 | 0.924571 | — | 0.333903 | — |
| 135. | 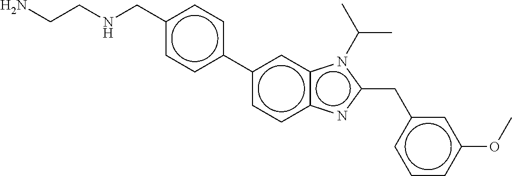 | 0.931537 | 0.168842 | 4.92312 | — |
| 136. | 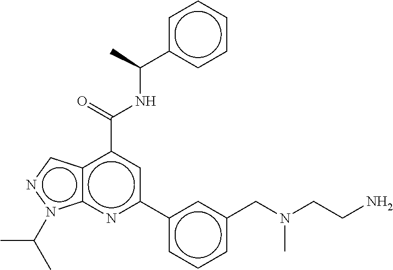 | 0.934308 | 50 | 0.04548 | — |
| 137. | 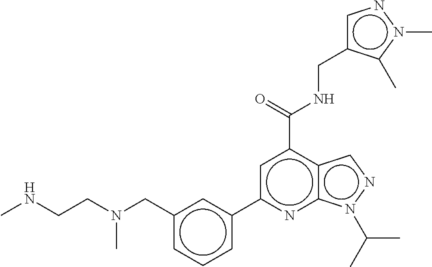 | 0.985191 | — | 0.00606034 | 9.622678667 |
| 138. | 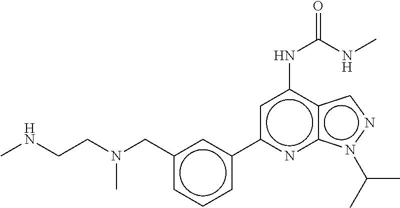 | 1.03588 | — | 0.0186733 | 7.741451917 |
| 139. | 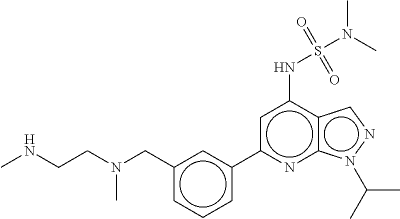 | 1.05051 | — | 0.0373414 | 4.8561333 |
| 140. | 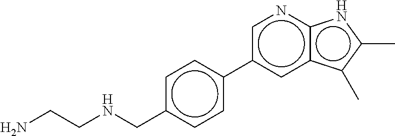 | 1.08689 | 0.128448 | — | 12.53906635 |
| 141. | 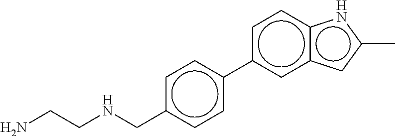 | 1.10581 | 0.32748 | 5.80885 | — |
| 142. | 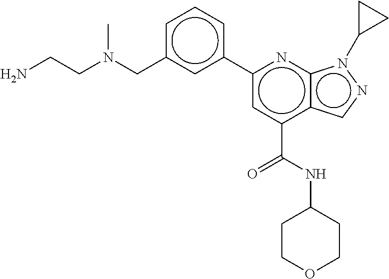 | 1.10676 | — | 0.00943084 | 2.7665276 |
| 143. | 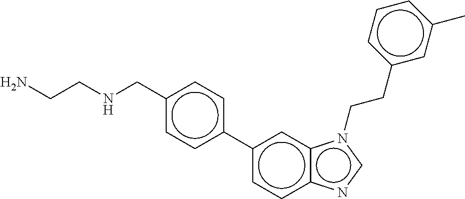 | 1.11143 | 0.281417 | 5.61695 | — |
| 144. | 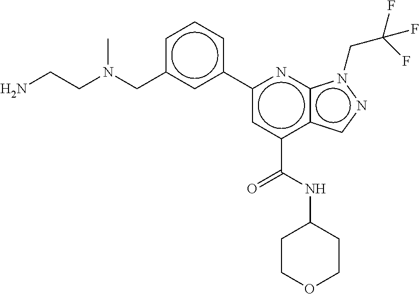 | 1.2007 | — | 0.0267439 | 8.461852 |
| 145. | 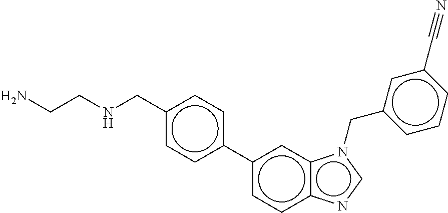 | 1.27186 | 0.0798997 | 8.00314 | — |
| 146. | 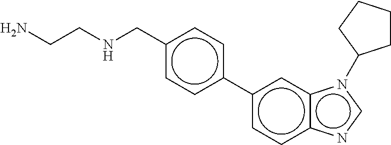 | 1.33254 | 0.173185 | — | — |
| 147. | 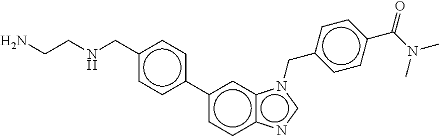 | 1.33257 | 0.129485 | — | — |
| 148. | 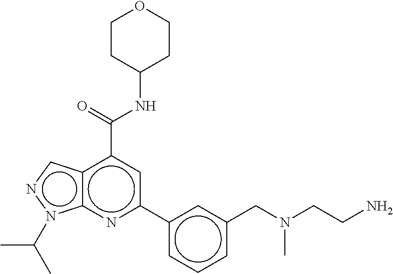 | 1.4777 | — | 0.0109304 | 0.971704767 |
| 149. | 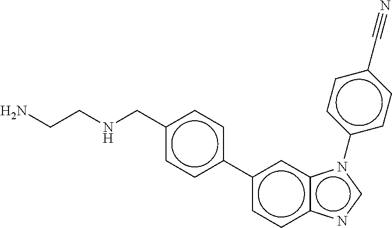 | 1.53208 | 0.238183 | — | — |
| 150. | 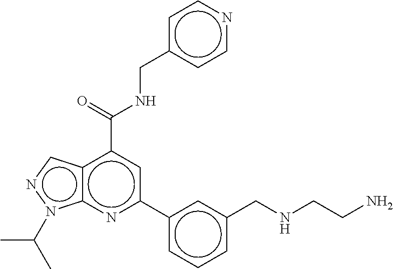 | 1.60291 | 23.9205 | 0.274309 | — |
| 151. | 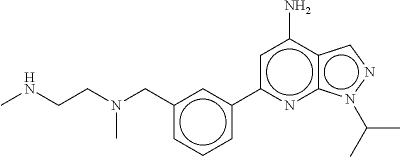 | 1.65982 | — | 0.0920712 | 20 |
| 152. | 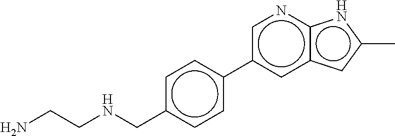 | 1.68854 | 0.31586 | — | — |
| 153. | 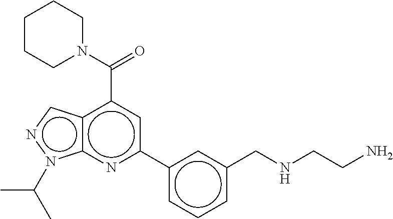 | 1.7071 | — | 0.19455 | 20 |
| 154. | 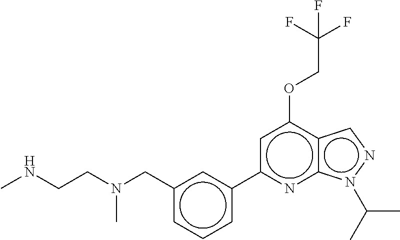 | 2.28934 | — | 0.0469429 | — |
| 155. | 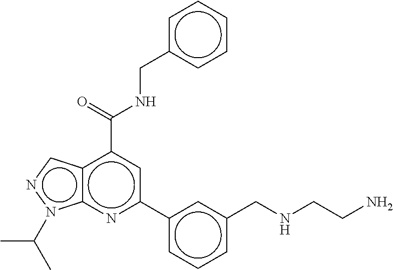 | 2.29975 | — | 0.11466 | — |
| 156. | 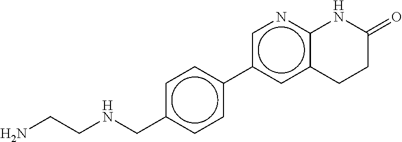 | 2.40536 | 0.338192 | 2.61108 | — |
| 157. | 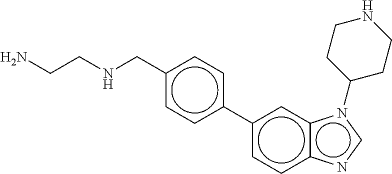 | 2.43338 | 0.417504 | 4.59992 | — |
| 158. | 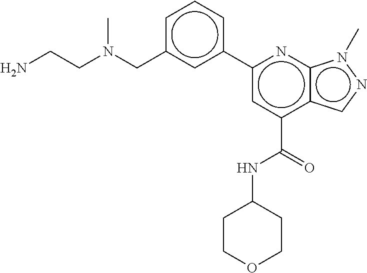 | 3.01912 | — | 0.0385399 | 13.5181072 |
| 159. | 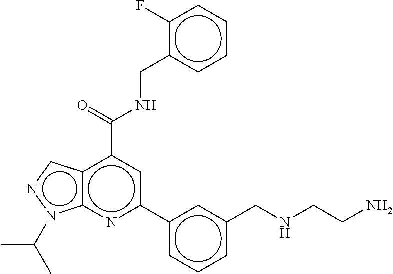 | 3.13733 | — | 0.175239 | 20 |
| 160. | 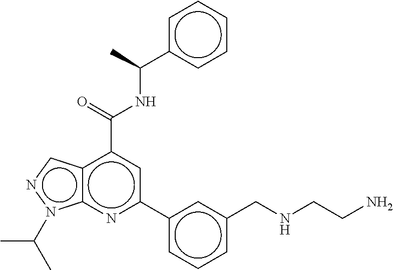 | 4.09508 | 35.874 | 0.0279734 | — |
| 161. | 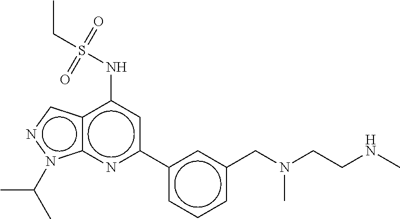 | 4.16956 | — | 0.168889 | 11.3379862 |
| 162. | 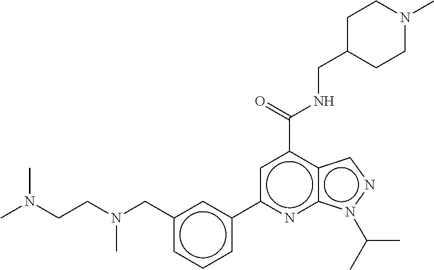 | 4.32195 | 50 | 0.0915694 | — |
| 163 | 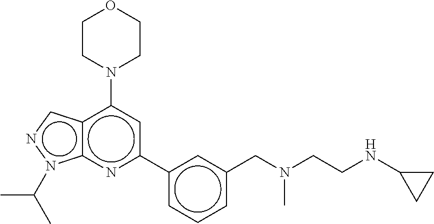 | 4.40569 | — | 0.160797 | 11.5573771 |
| 164. | 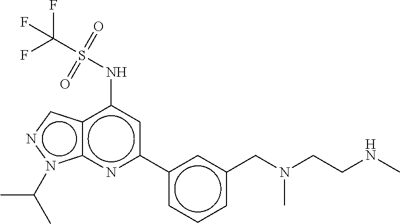 | 4.49925 | — | 0.357981 | 11.89456755 |
| 165. | 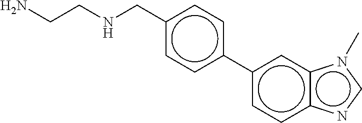 | 4.53044 | 0.250231 | — | — |
| 166. | 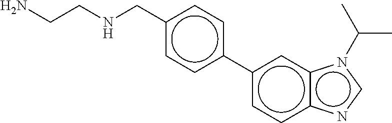 | 5.0946 | 0.404071 | 31.7088 | — |
| 167. | 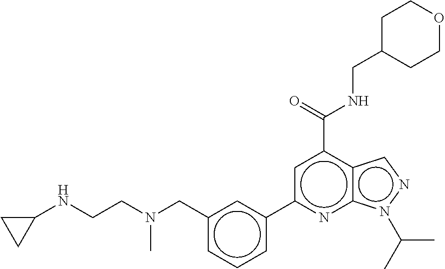 | 5.88613 | — | 0.098199 | 5.136234875 |
| 168. | 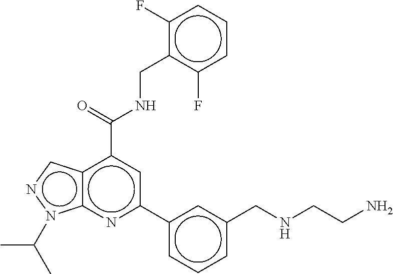 | 7.81196 | — | 0.0233247 | — |
| 169. | 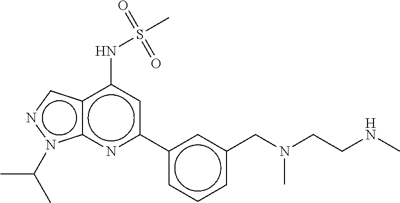 | 10 | — | 0.124892 | 13.34304783 |
| 170. | 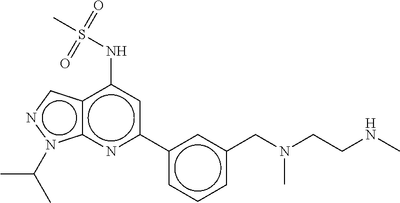 | 10 | — | 0.263743 | 20 |
| 171. | 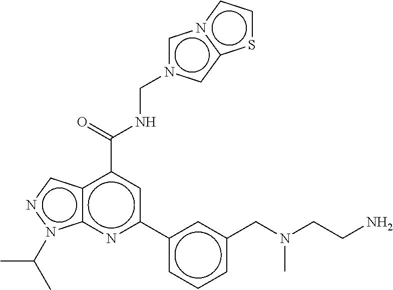 | — | — | 0.00504999 | 0.9520275 |
| 172. | 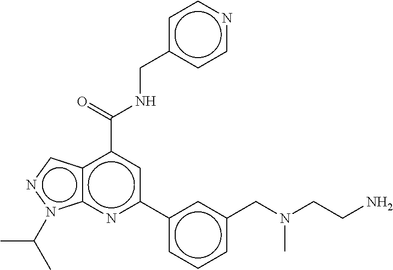 | — | — | 0.00566996 | 3.141475 |
| 173. | 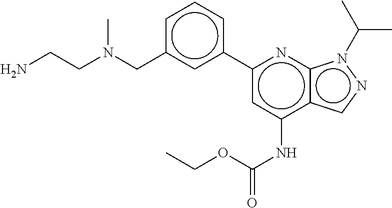 | — | — | 0.0059271 | 3.306424175 |
| 174. | 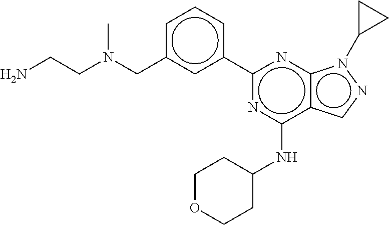 | — | — | 0.00610598 | 5.040449925 |
| 175. | 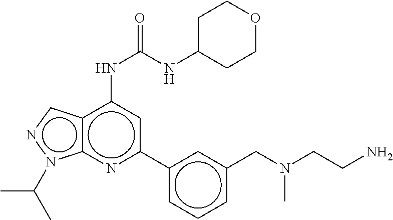 | — | — | 0.00693762 | 11.48881918 |
| 176. | 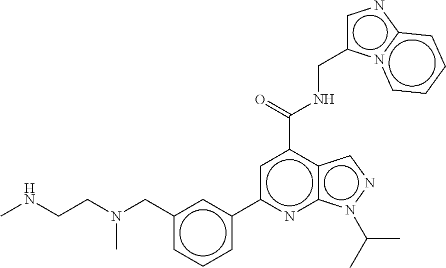 | — | — | 0.00728971 | 0.86519225 |
| 177. | 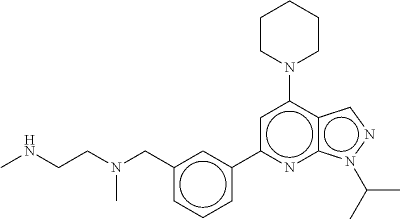 | — | — | 0.00734565 | — |
| 178. | 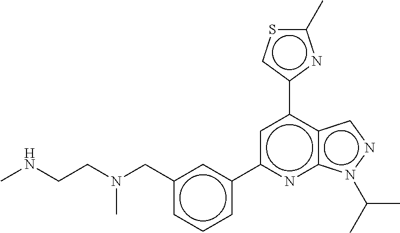 | — | — | 0.00751001 | — |
| 179. | 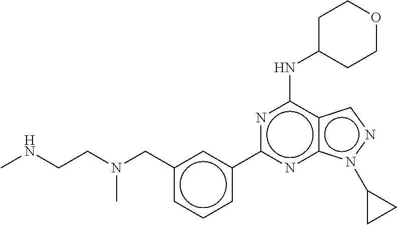 | — | — | 0.00888097 | 4.87229415 |
| 180. | 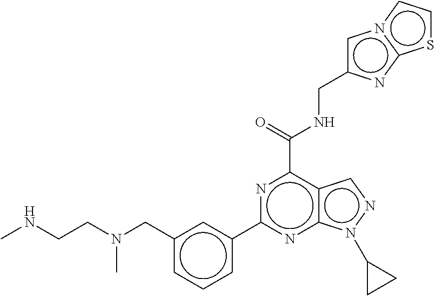 | — | 200 | 0.00890001 | 0.8965305 |
| 181. | 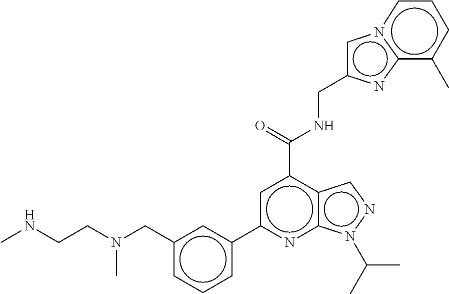 | — | — | 0.00963475 | 0.7326637 |
| 182. | 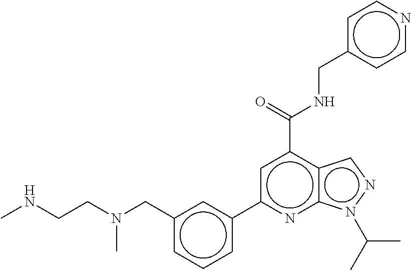 | — | 12.0896 | 0.0102538 | 3.549769133 |
| 183. | 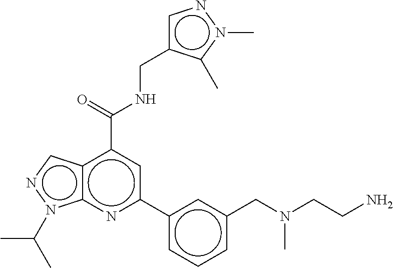 | — | — | 0.0116201 | 1.969586833 |
| 184. | 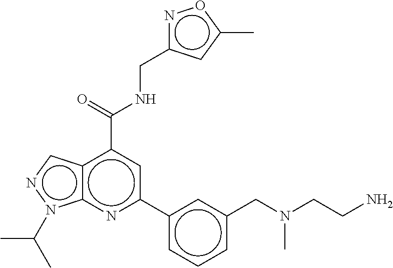 | — | — | 0.0116501 | |
| 185. | 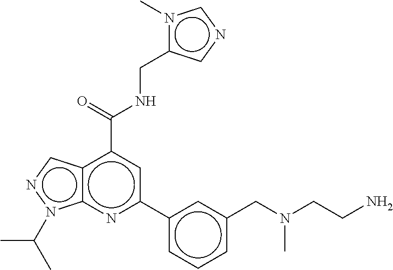 | — | — | 0.0117201 | 10.75364033 |
| 186. | 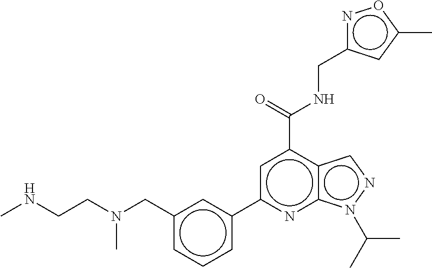 | — | — | 0.0117201 | 1.400954 |
| 187. | 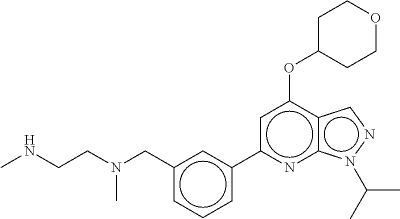 | — | — | 0.0120199 | 6.0942329 |
| 188. | 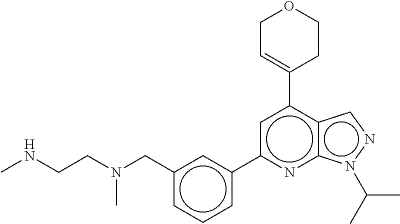 | — | — | 0.01229 | 8.308730733 |
| 189. | 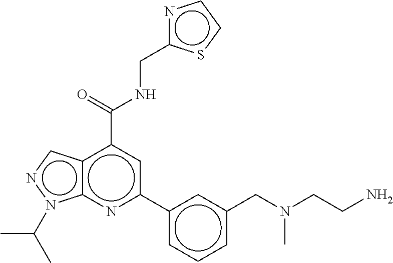 | — | — | 0.0123861 | 1.885007433 |
| 190. | 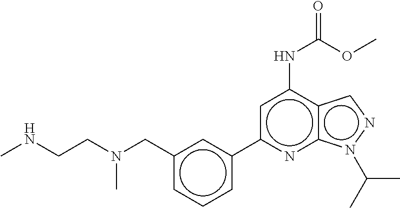 | — | — | 0.0124983 | 2.46110245 |
| 191. | 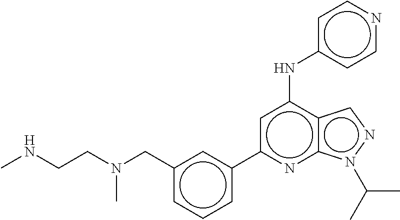 | — | — | 0.0129101 | |
| 192. | 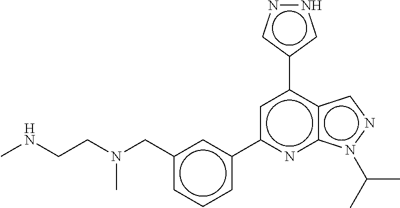 | — | — | 0.01309 | — |
| 193. | 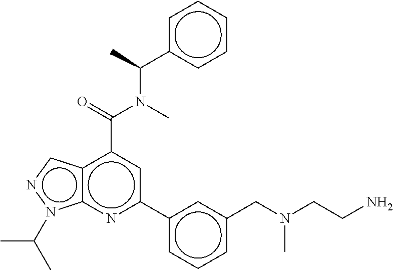 | — | 0.790224 | 0.013487 | — |
| 194. | 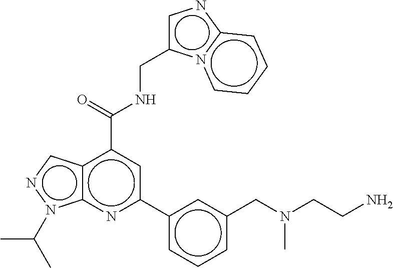 | — | — | 0.0136399 | 1.1990311 |
| 195. | 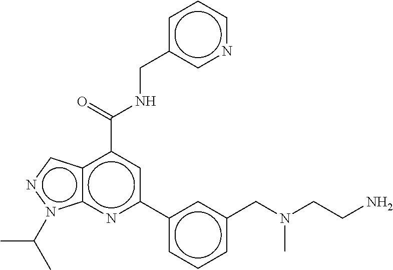 | — | — | 0.0139475 | 1.6583695 |
| 196. | 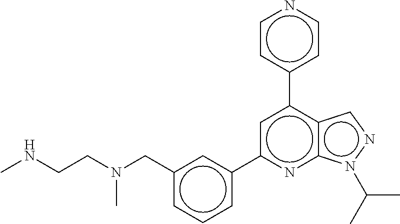 | — | — | 0.0142801 | — |
| 197. | 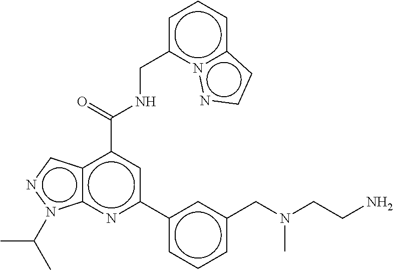 | — | — | 0.01501 | 1.6592784 |
| 198. | 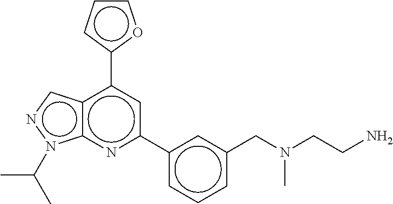 | — | — | 0.01513 | 20 |
| 199. | 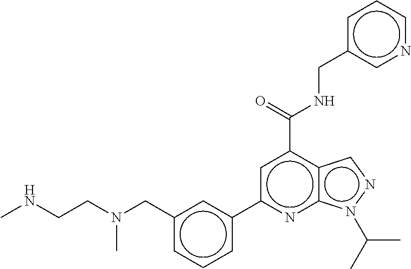 | — | — | 0.015884 | 2.179738833 |
| 200. | 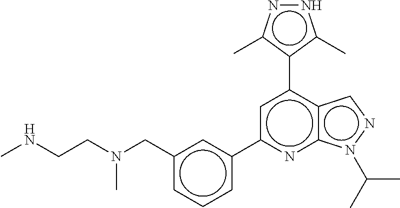 | — | — | 0.0167498 | — |
| 201. | 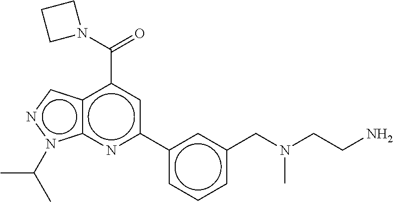 | — | — | 0.0182182 | 3.0499844 |
| 202. | 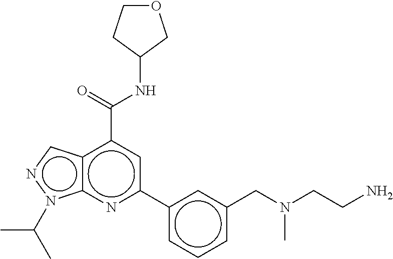 | — | — | 0.0190944 | 1.9911807 |
| 203. | 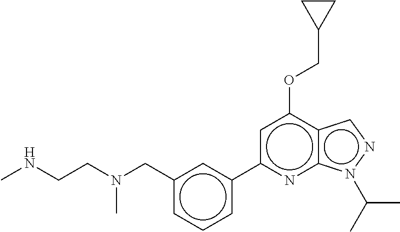 | — | — | 0.0192199 | — |
| 204. | 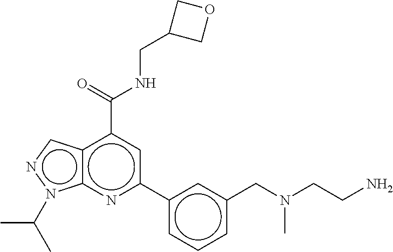 | — | — | 0.0192398 | — |
| 205. | 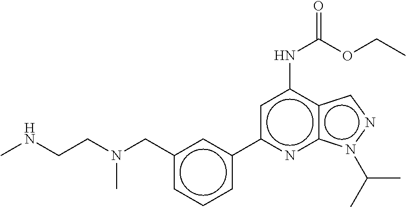 | — | — | 0.019584 | 5.753696425 |
| 206. | 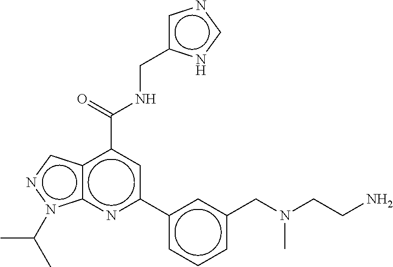 | — | — | 0.0196599 | 20 |
| 207. | 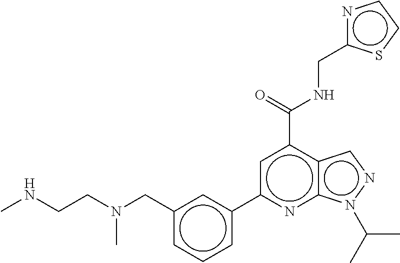 | — | — | 0.0206125 | 1.63757915 |
| 208. | 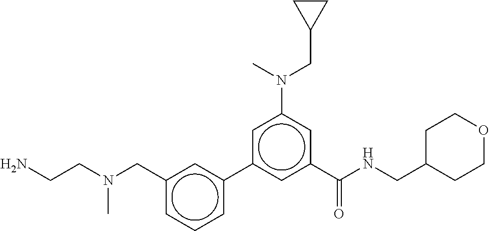 | — | — | 0.02072 | 5.5432166 |
| 209. | 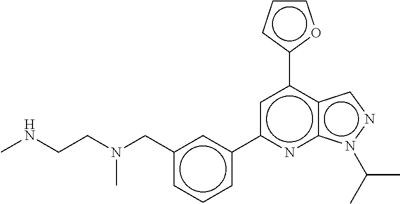 | — | — | 0.0216701 | 20 |
| 210. | 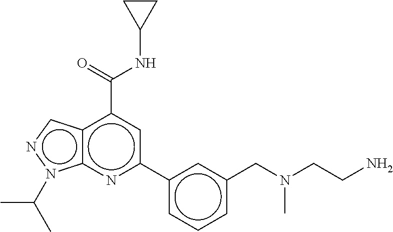 | — | — | 0.0223841 | 2.0046122 |
| 211. | 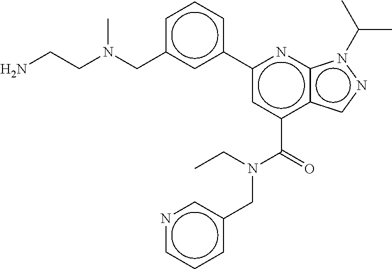 | — | — | 0.0239001 | 3.2522322 |
| 212. | 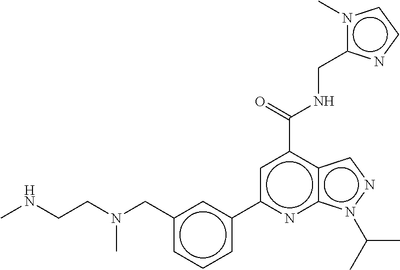 | — | — | 0.0249299 | 1.8005038 |
| 213. | 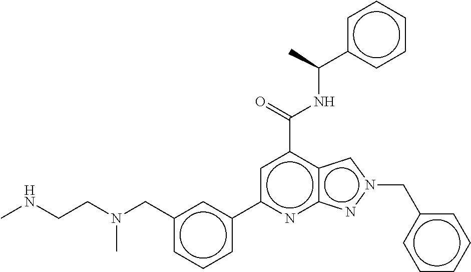 | — | — | 0.0254337 | — |
| 214. | 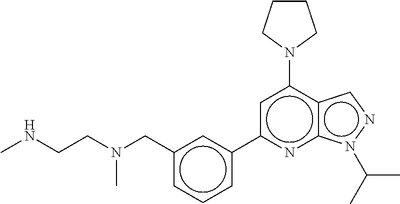 | — | — | 0.0265852 | 20 |
| 215. | 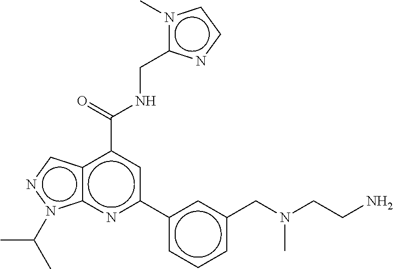 | — | — | 0.02913 | 2.242611433 |
| 216. | 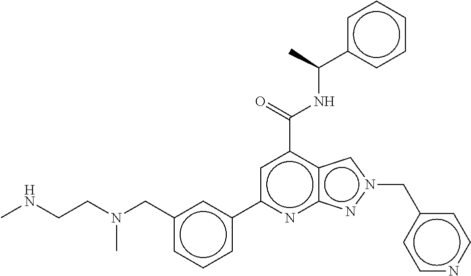 | — | — | 0.0298676 | — |
| 217. | 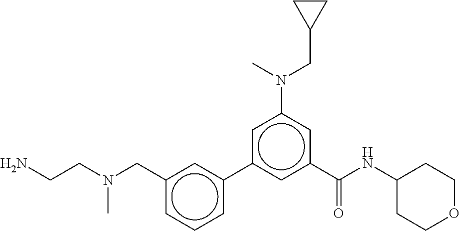 | — | — | 0.0299502 | 5.6910847 |
| 218. | 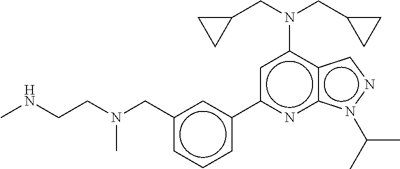 | — | — | 0.0308802 | — |
| 219. | 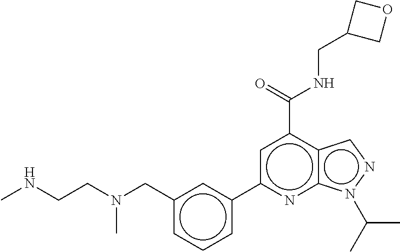 | — | — | 0.0315552 | — |
| 220. | 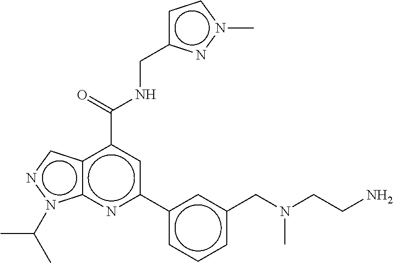 | — | — | 0.0324699 | 1.235610367 |
| 221. | 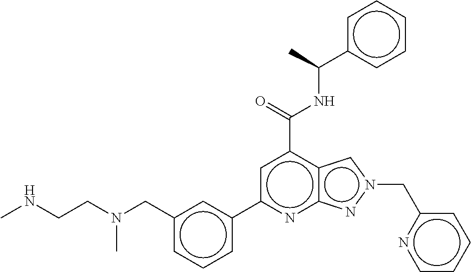 | — | — | 0.0326017 | — |
| 222. | 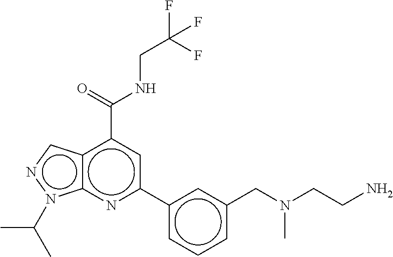 | — | — | 0.0343804 | 3.8175828 |
| 223. | 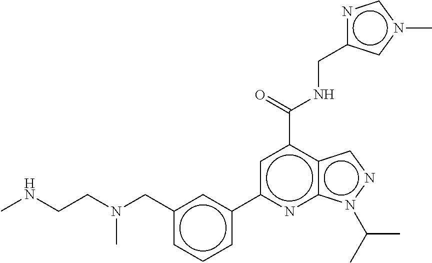 | — | — | 0.03532 | 3.4675366 |
| 224. | 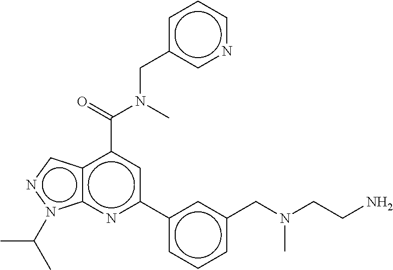 | — | — | 0.0374697 | 8.467277 |
| 225. | 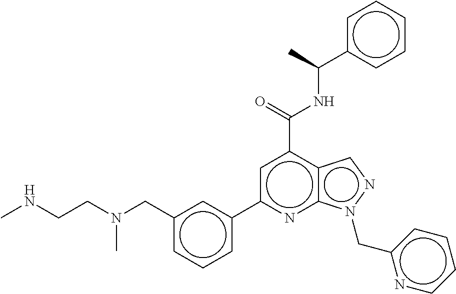 | — | — | 0.0380803 | — |
| 226. | 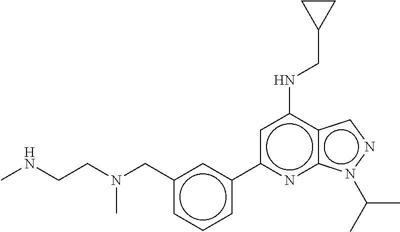 | — | — | 0.0394803 | — |
| 227. | 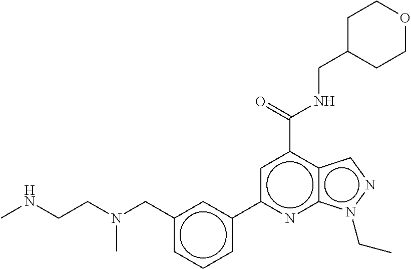 | — | — | 0.0399103 | 4.736308 |
| 228. | 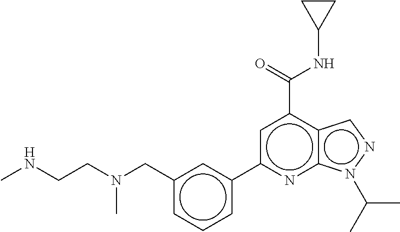 | — | — | 0.0412411 | 1.9124658 |
| 229. | 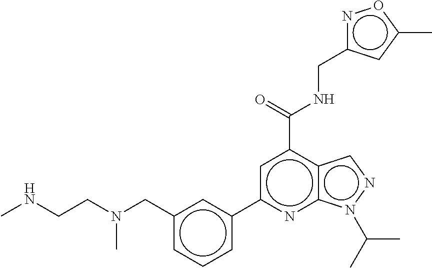 | — | — | 0.0413152 | 2.24534937 |
| 230. | 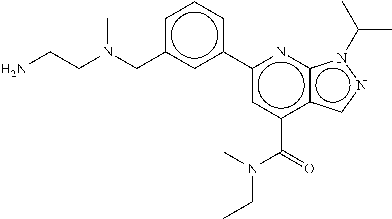 | — | — | 0.04139 | 7.62628 |
| 231. | 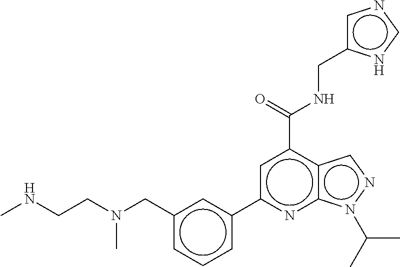 | — | — | 0.0421993 | 20 |
| 232. | 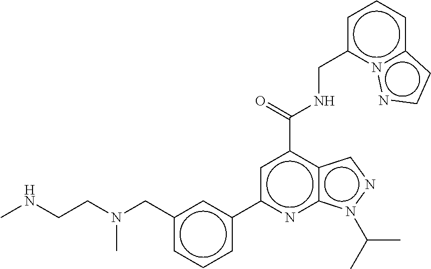 | — | — | 0.0423604 | 1.5062592 |
| 233. | 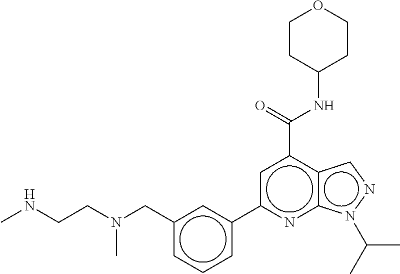 | — | — | 0.0426217 | 0.843111667 |
| 234. | 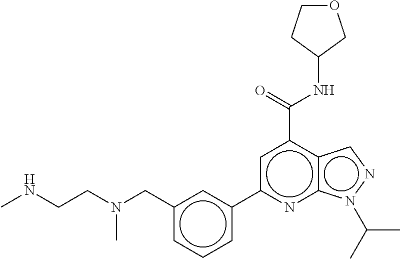 | — | — | 0.0432903 | 2.759729 |
| 235. | 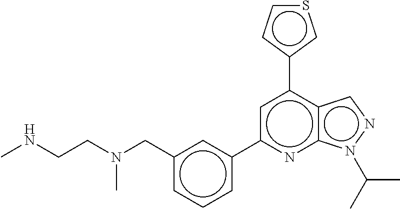 | — | — | 0.04579 | 20 |
| 236. | 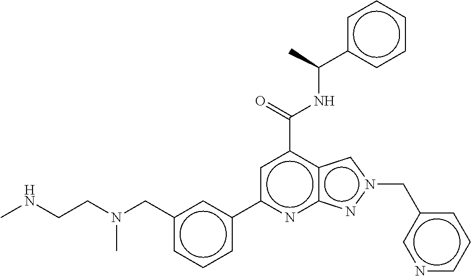 | — | — | 0.0459897 | — |
| 237. | 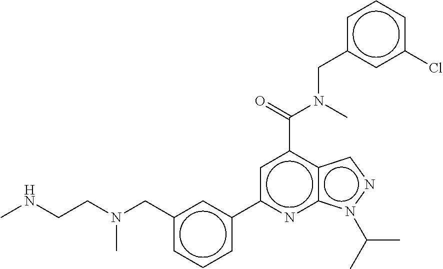 | — | 1.48177 | 0.0480596 | — |
| 238. | 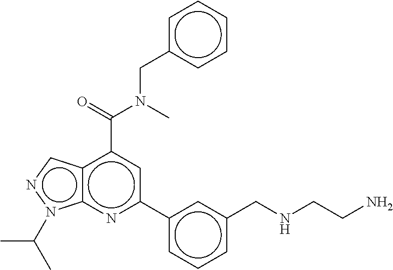 | — | — | 0.0480795 | 20 |
| 239. | 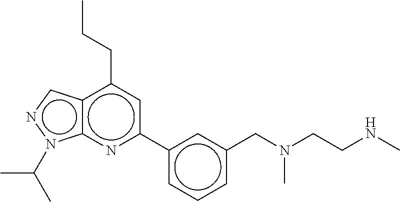 | — | — | 0.04904 | 20 |
| 240. | 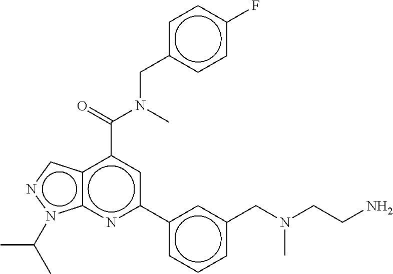 | — | 7.4184 | 0.0496501 | — |
| 241. | 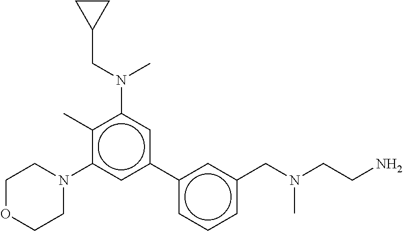 | — | — | 0.0509695 | 5.6910847 |
| 242. | 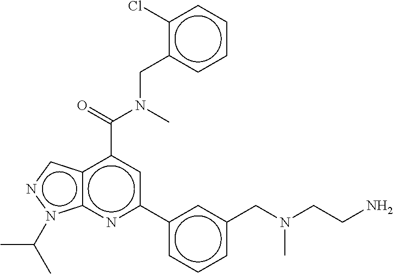 | — | 0.235473 | 0.0519302 | — |
| 243. | 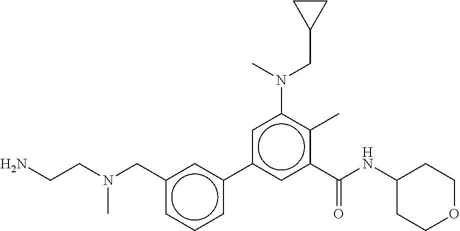 | — | — | 0.0519506 | 20 |
| 244. | 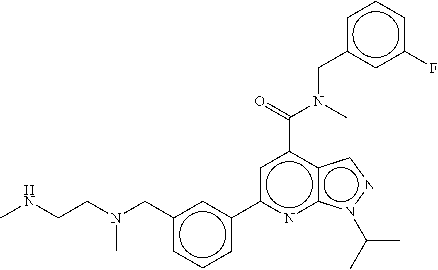 | — | 2.82859 | 0.0530506 | — |
| 245. | 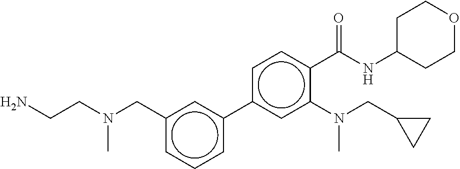 | — | — | 0.0535994 | 20 |
| 246. | 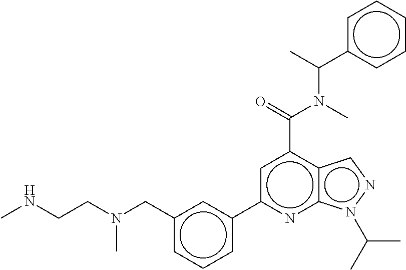 | — | 0.577285 | 0.0543201 | — |
| 247. | 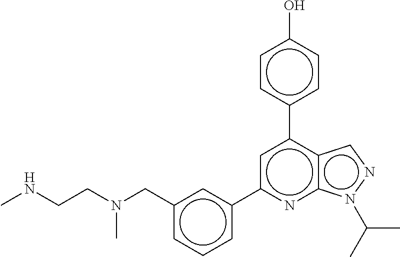 | — | — | 0.0547205 | 20 |
| 248. | 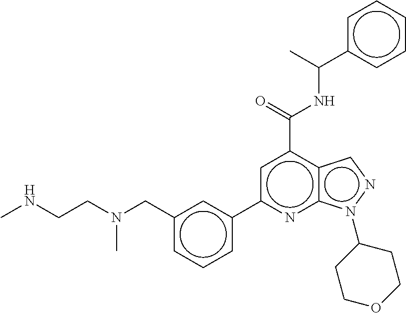 | — | — | 0.0554805 | — |
| 249. | 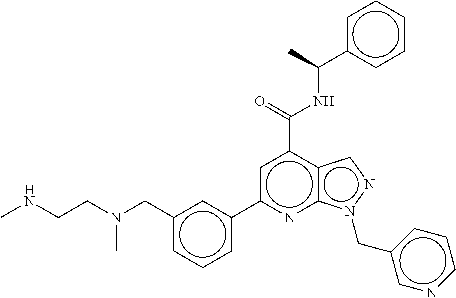 | — | — | 0.0572796 | — |
| 250. | 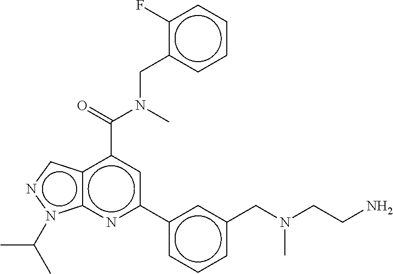 | — | 0.816207 | 0.0582694 | — |
| 251. | 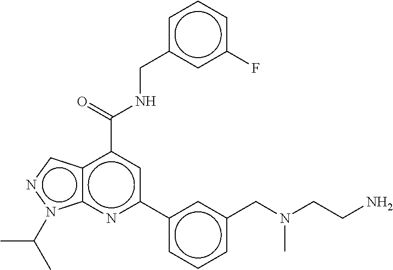 | — | 47.2661 | 0.0591303 | — |
| 252. | 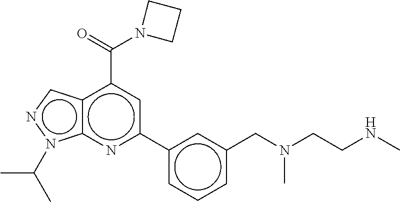 | — | — | 0.0592976 | 20 |
| 253. | 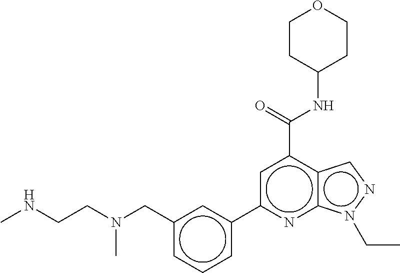 | — | — | 0.0612746 | 6.0942329 |
| 254. | 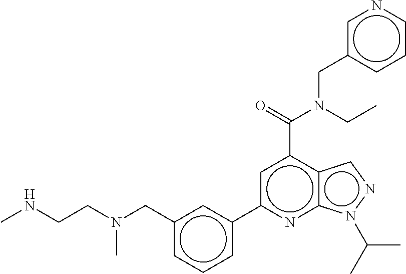 | — | — | 0.0618401 | 5.523701 |
| 255. | 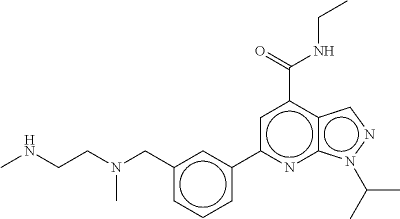 | — | — | 0.0623003 | 3.8669055 |
| 256. | 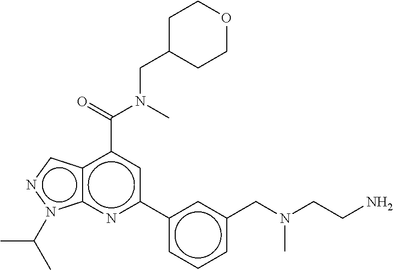 | — | 50 | 0.0630645 | 9.560372 |
| 257. | 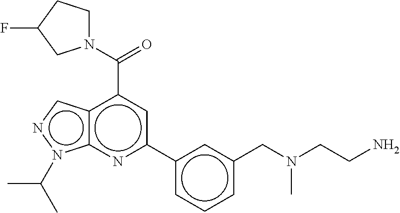 | — | — | 0.0635097 | 10.343119 |
| 258. | 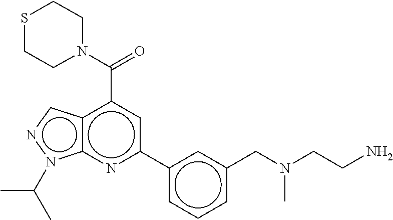 | — | — | 0.0641993 | 18.54362 |
| 259. | 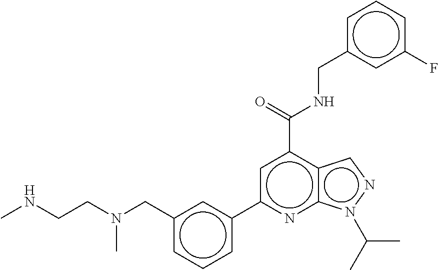 | — | — | 0.06699 | 20 |
| 260. | 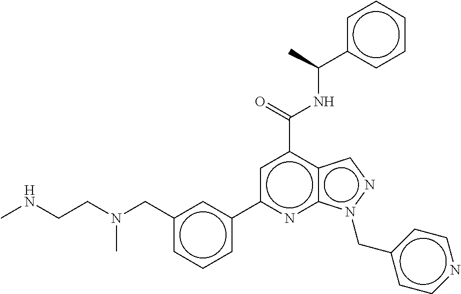 | — | — | 0.0681899 | 20 |
| 261. | 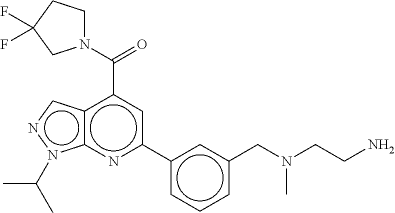 | — | — | 0.0693506 | 20 |
| 262. | 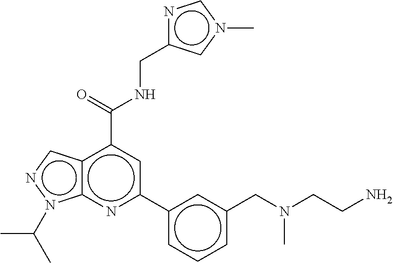 | — | — | 0.0702393 | 10.15098017 |
| 263. | 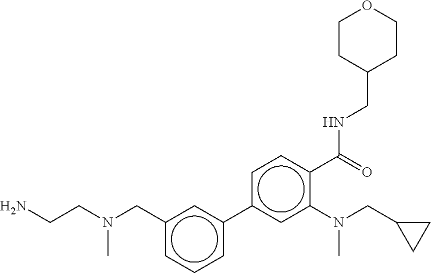 | — | 7.72521 | 0.0721947 | 20 |
| 264. | 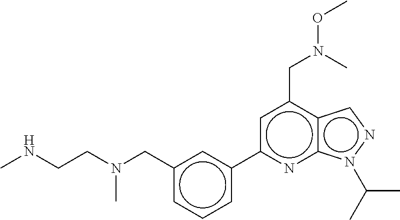 | — | — | 0.0740509 | — |
| 265. | 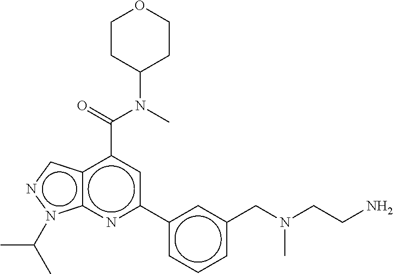 | — | — | 0.0744201 | 10.330071 |
| 266. | 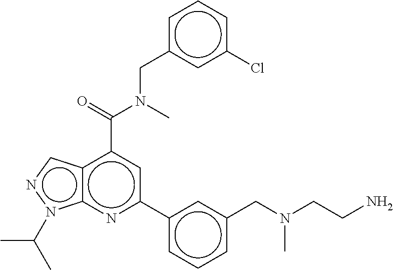 | — | 1.79081 | 0.0748894 | — |
| 267. | 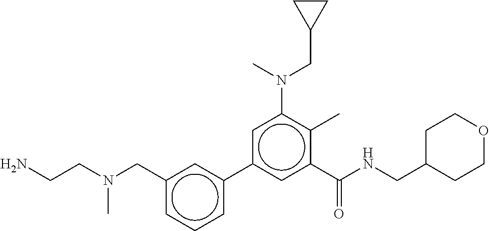 | — | — | 0.0752299 | — |
| 268. | 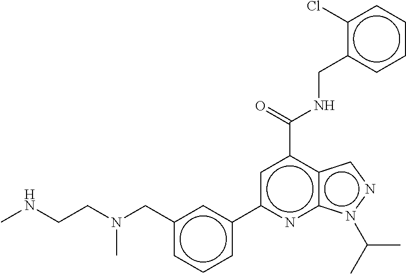 | — | — | 0.0773304 | — |
| 269. | 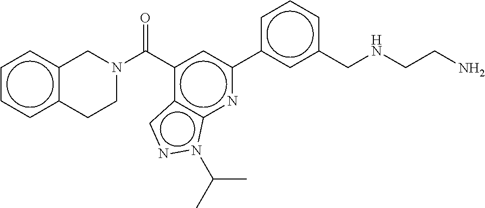 | — | 13.116 | 0.0799209 | — |
| 270. | 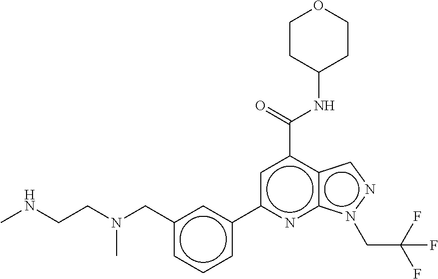 | — | — | 0.0809693 | 5.1425218 |
| 271. | 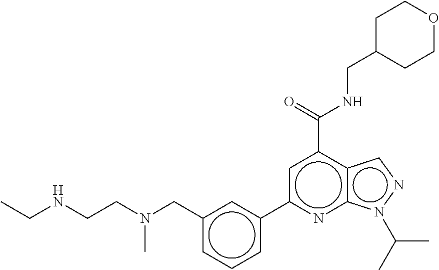 | — | — | 0.0814198 | 20 |
| 272. | 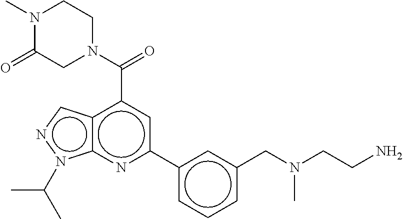 | — | — | 0.0847501 | 20 |
| 273. | 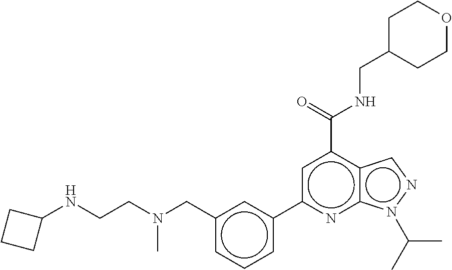 | — | — | 0.0877109 | 14.539342 |
| 274. | 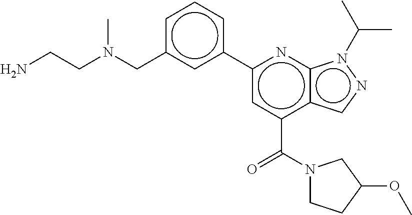 | — | — | 0.0893409 | 20 |
| 275. | 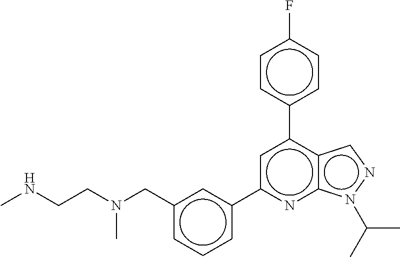 | — | — | 0.0928358 | 20 |
| 276. | 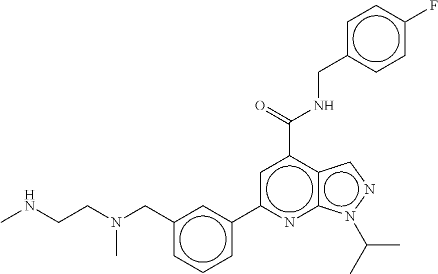 | — | — | 0.09415 | 20 |
| 277. | 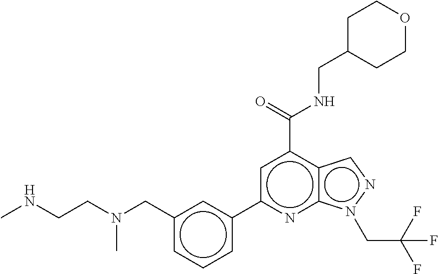 | — | — | 0.105361 | 7.201077 |
| 278. | 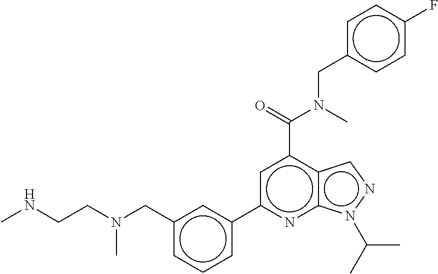 | — | 8.72952 | 0.109769 | — |
| 279. | 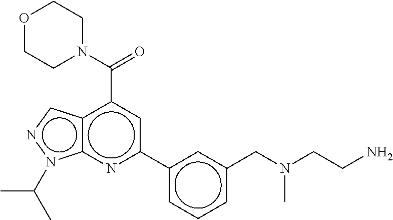 | — | — | 0.112551 | 18.65676267 |
| 280. | 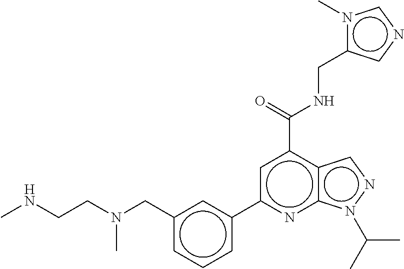 | — | — | 0.113099 | 9.491225 |
| 281. | 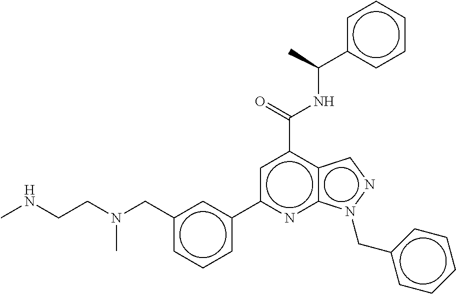 | — | 17.3672 | 0.127571 | — |
| 282. | 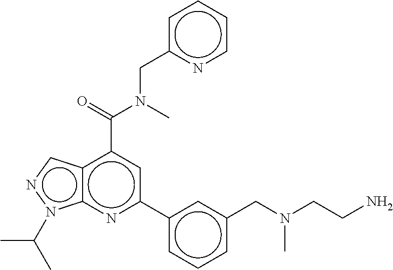 | — | — | 0.13002 | 8.200357767 |
| 283. | 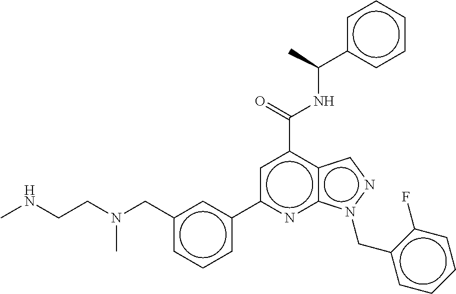 | — | 29.175 | 0.13053 | — |
| 284. | 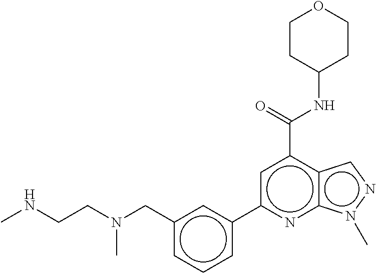 | — | — | 0.136921 | 20 |
| 285. | 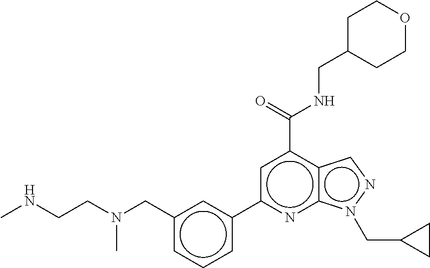 | — | — | 0.138871 | — |
| 286. | 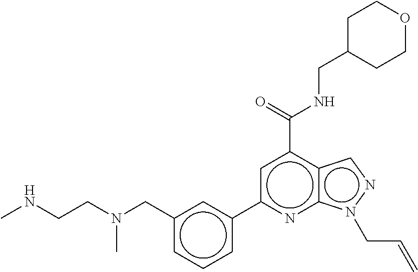 | — | — | 0.148024 | 7.1490376 |
| 287. | 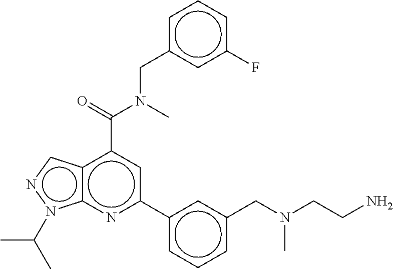 | — | 4.17427 | 0.152929 | — |
| 288. | 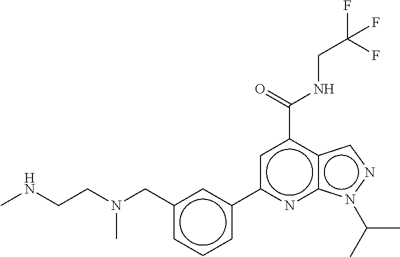 | — | — | 0.158782 | 3.44376665 |
| 289. | 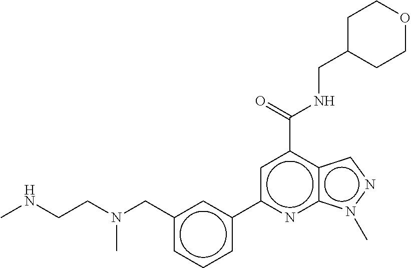 | — | — | 0.162789 | 16.922321 |
| 290. | 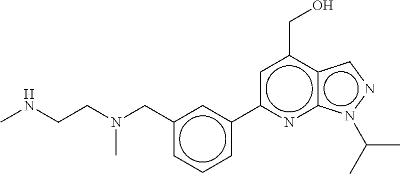 | — | — | 0.167462 | 20 |
| 291. | 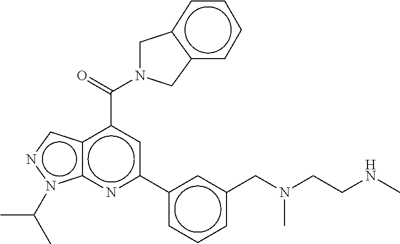 | — | — | 0.167622 | — |
| 292. | 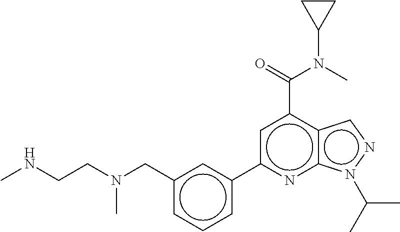 | — | — | 0.17062 | 14.02066033 |
| 293. | 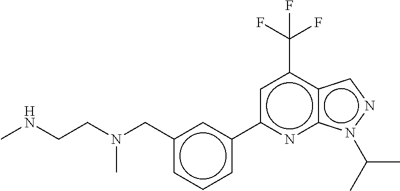 | — | — | 0.173341 | 17.5807455 |
| 294. | 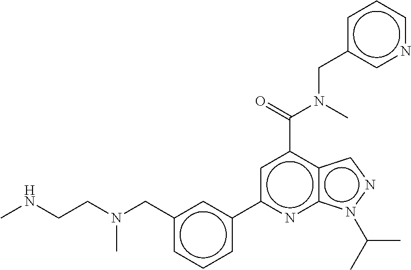 | — | — | 0.177439 | 4.018413317 |
| 295. | 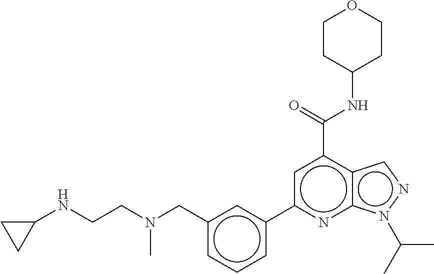 | — | — | 0.180501 | 12.3952038 |
| 296. | 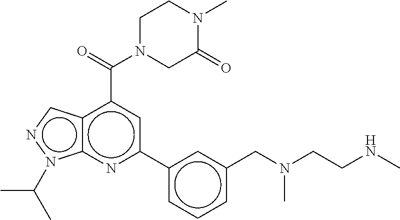 | — | — | 0.182591 | 15.73100625 |
| 297. | 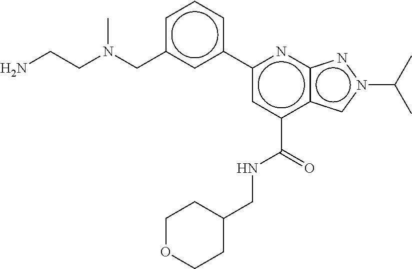 | — | — | 0.189483 | 20 |
| 298. | 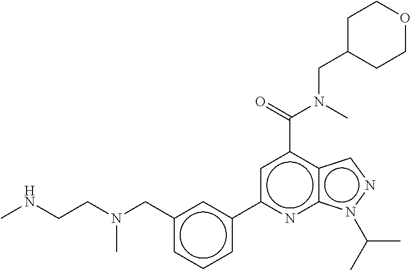 | — | — | 0.200263 | 9.622678667 |
| 299. | 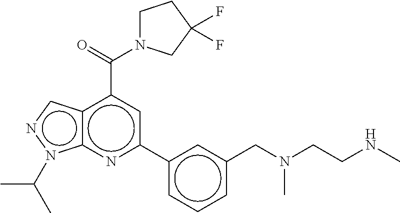 | — | — | 0.203198 | — |
| 300. | 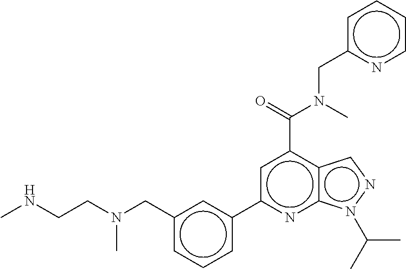 | — | 34.2531 | 0.2054 | 6.3821123 |
| 301. | 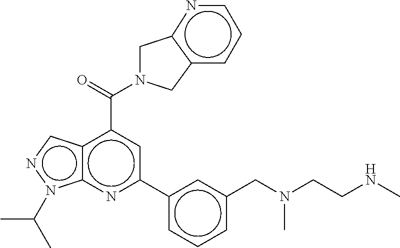 | — | — | 0.209199 | 15.4653755 |
| 302. | 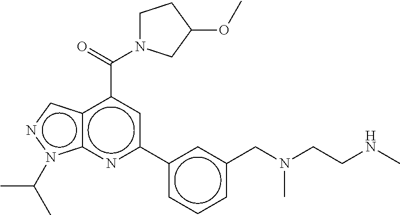 | — | — | 0.233169 | — |
| 303. | 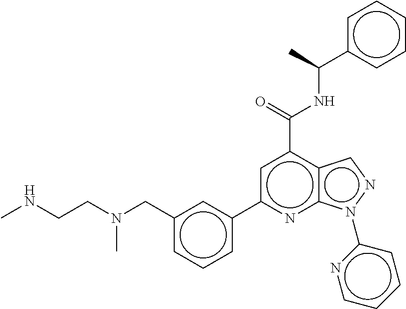 | — | 13.3156 | 0.235277 | — |
| 304. | 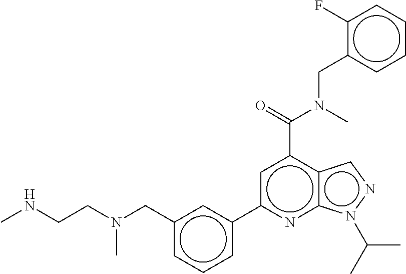 | — | 0.67041 | 0.23947 | — |
| 305. | 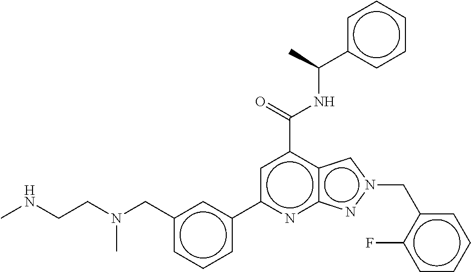 | — | — | 0.240082 | — |
| 306. | 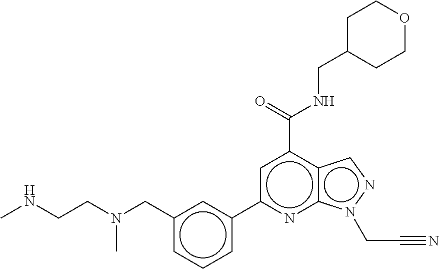 | — | — | 0.242834 | — |
| 307. | 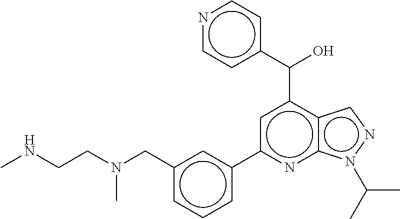 | — | — | 0.24996 | 20 |
| 308. | 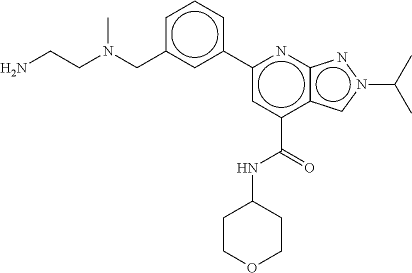 | — | — | 0.253586 | — |
| 309. | 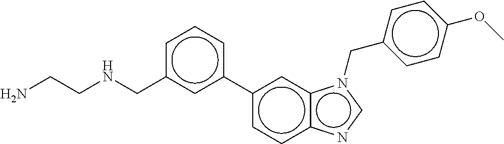 | — | 1.07039 | 0.272302 | — |
| 310. | 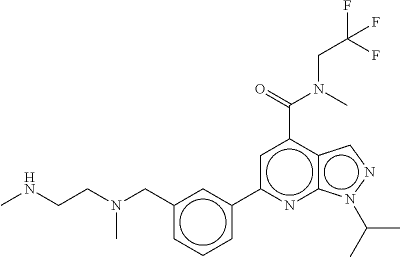 | — | — | 0.283302 | — |
| 311. | 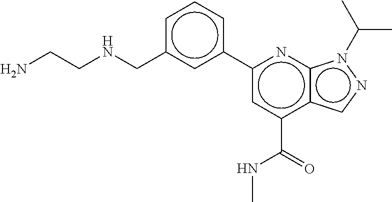 | — | — | 0.293481 | 14.161012 |
| 312. | 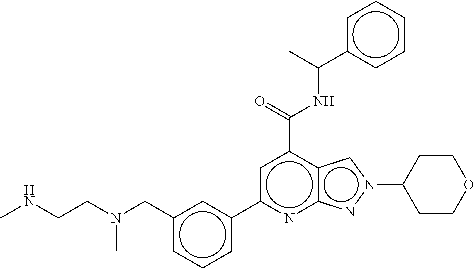 | — | — | 0.310764 | — |
| 313. | 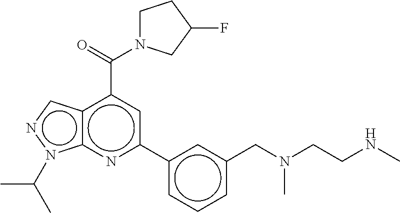 | — | — | 0.311864 | — |
| 314. | 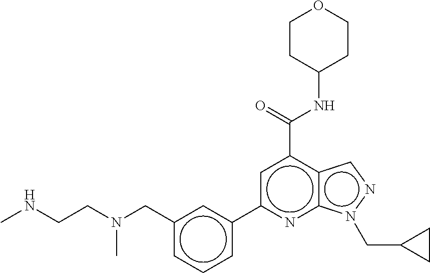 | — | — | 0.334003 | — |
| 315. | 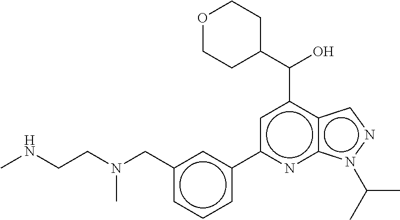 | — | — | 0.368337 | — |
| 316. | 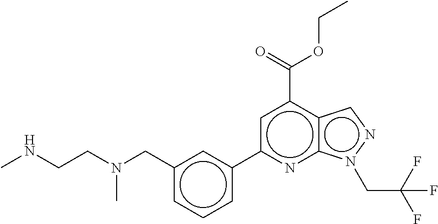 | — | — | 0.379371 | — |
| 317. | 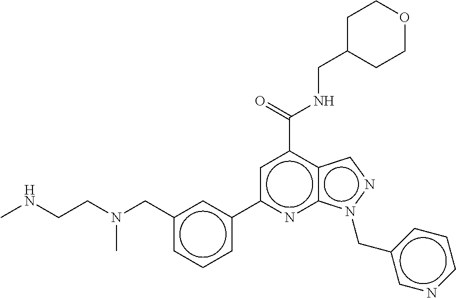 | — | — | 0.385541 | — |
| 318. | 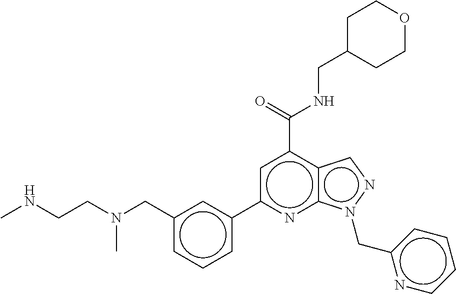 | — | — | 0.397897 | — |
| 319. | 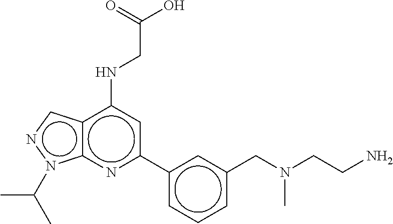 | — | — | 0.401218 | 20 |
| 320. | 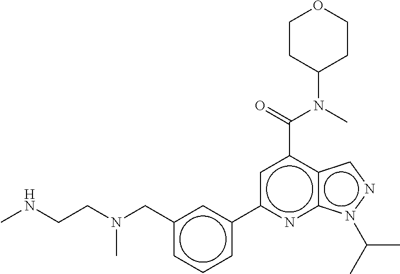 | — | — | 0.410474 | — |
| 321. | 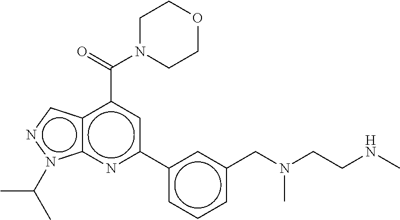 | — | — | 0.453232 | — |
| 322. | 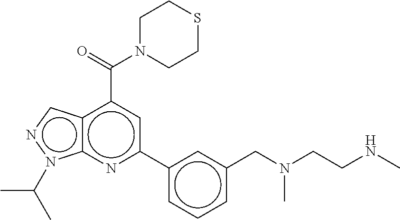 | — | — | 0.456394 | — |
| 323. | 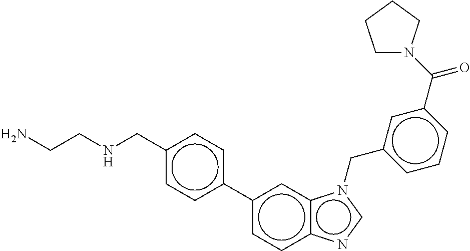 | — | 0.126917 | 0.756102 | — |
| 324. | 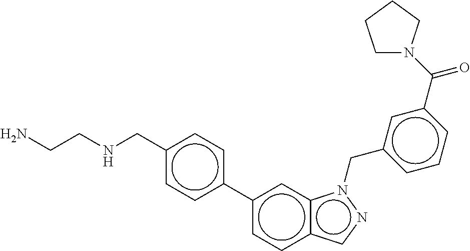 | — | 0.0972748 | 0.886891 | — |
| 325. | 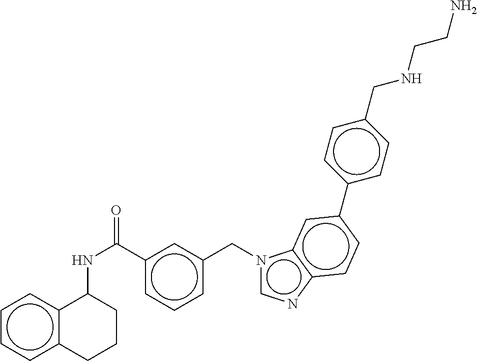 | — | 0.0253402 | 1.02982 | — |
| 326. | 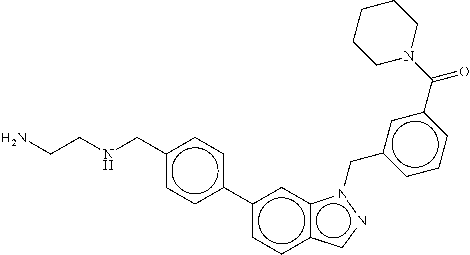 | — | 0.170085 | 1.26675 | — |
| 327. | 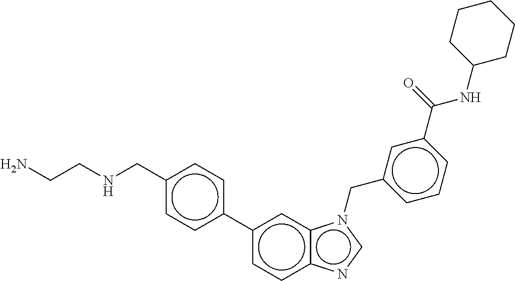 | — | 0.072816 | 1.5968 | — |
| 328. | 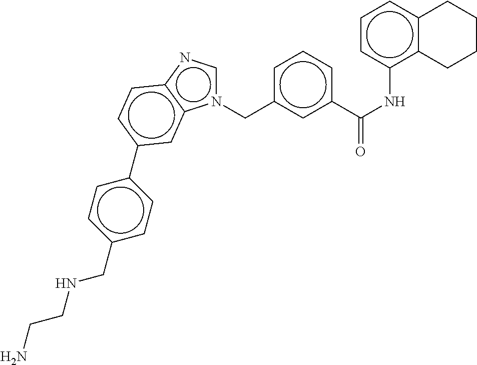 | — | 0.0805175 | 2.19377 | — |
| 329. | 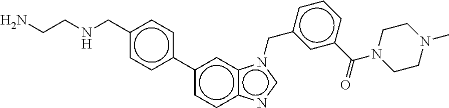 | — | 0.135964 | 2.47708 | — |
| 330. | 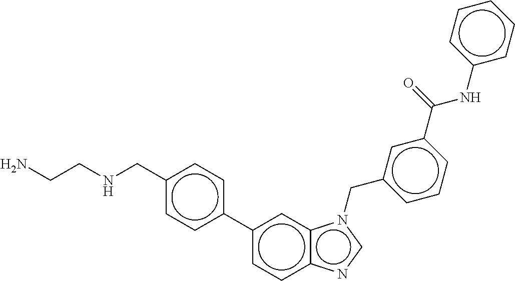 | — | 0.0524095 | 2.71569 | — |
| 331. | 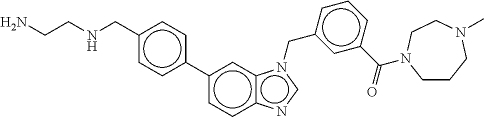 | — | 0.117587 | 2.80796 | — |
| 332. | 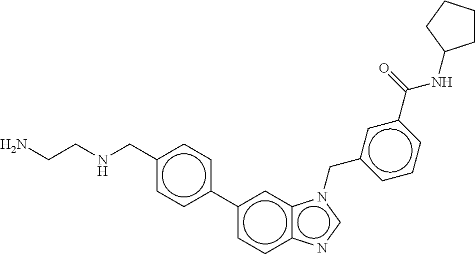 | — | 0.0859301 | 2.84387 | — |
| 333. | 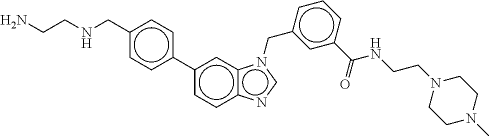 | — | 0.0947088 | 3.0571 | — |
| 334. | 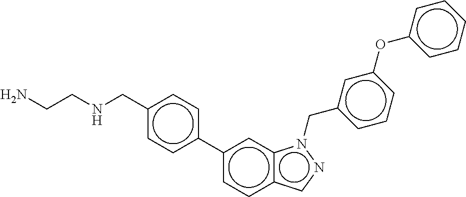 | — | 0.466904 | 3.13322 | — |
| 335. | 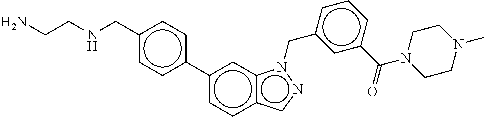 | — | 0.0738066 | 3.51172 | — |
| 336. | 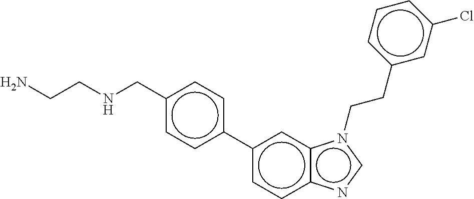 | — | 0.066535 | 5.11741 | — |
| 337. | 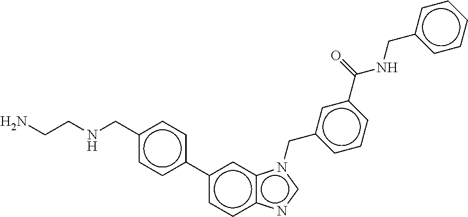 | — | 0.0332581 | 5.34983 | — |
| 338. | 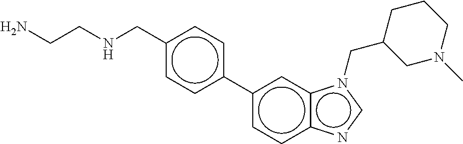 | — | 0.438639 | 5.52606 | — |
| 339. | 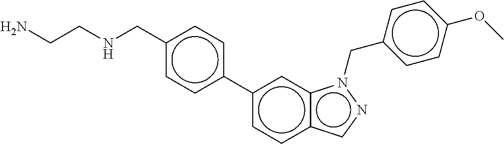 | — | 0.134774 | 5.68159 | — |
| 340. | 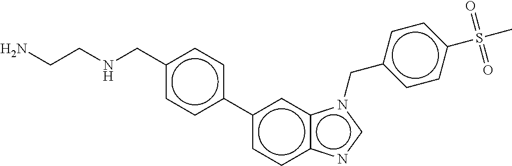 | — | 0.104591 | 5.94292 | — |
| 341. | 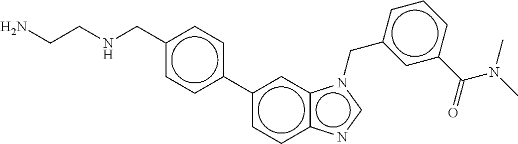 | — | 0.0873233 | 6.07981 | — |
| 342. | 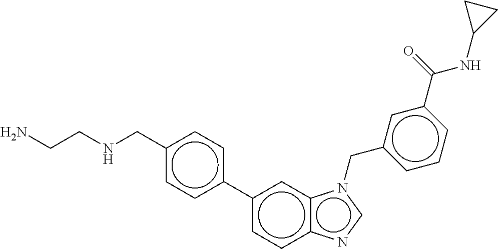 | — | 0.110309 | 6.47754 | — |
| 343. | 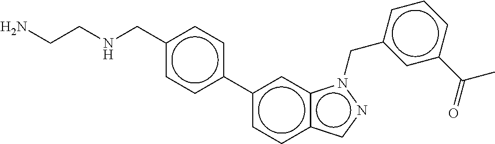 | — | 0.118724 | 8.8869 | — |
| 344. | 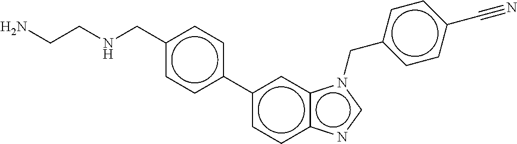 | — | 0.0675718 | 8.95901 | — |
| 345. | 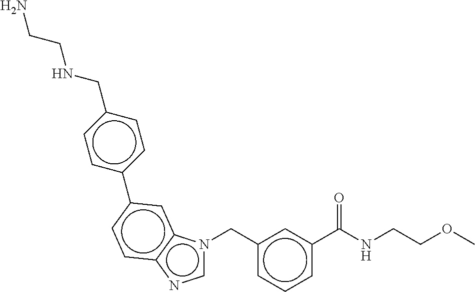 | — | 0.11438 | 9.38923 | — |
| 346. | 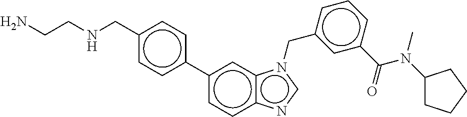 | — | 0.115782 | 10 | — |
| 347. | 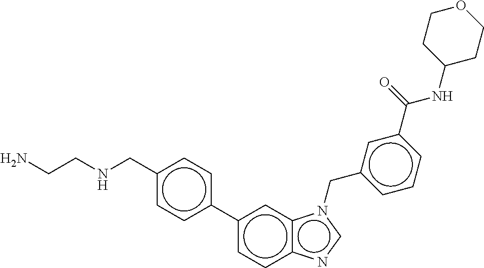 | — | 0.181458 | 10 | — |
| 348. | 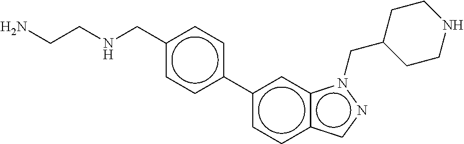 | — | 0.435427 | 10 | — |
| 349. | 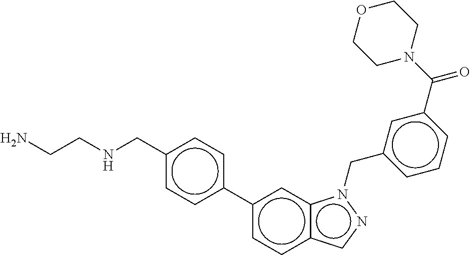 | — | 0.117 | — | — |
| 350. | 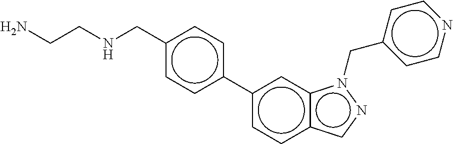 | — | 0.171666 | — | — |
| 351. | 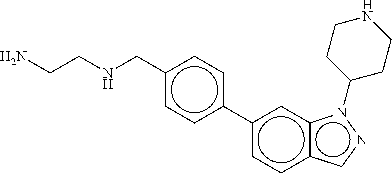 | — | 0.235361 | — | — |
| 352. | 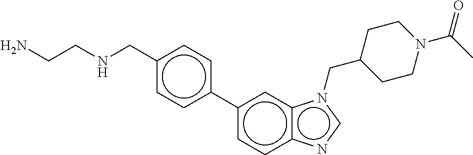 | — | 0.324803 | — | — |
| 353. | 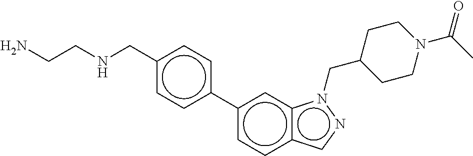 | — | 0.350655 | — | — |
| 354. | 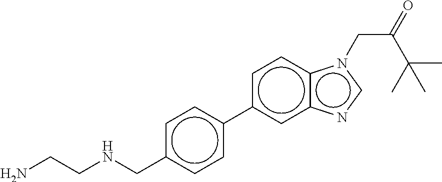 | — | 0.452044 | — | — |
| 355. | 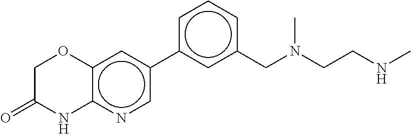 | 0.512295 | — | 10 | 12.8090625 |
| 356. | 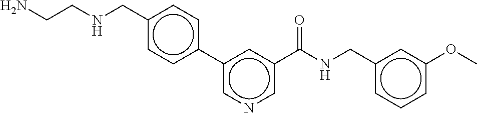 | 0.755032 | 0.376872 | 24.2952 | — |
| 357. | 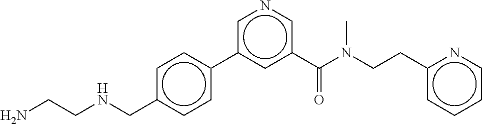 | — | 0.393482 | — | — |
| 358. | 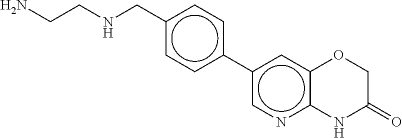 | 0.0784784 | — | 6.29038 | — |

In certain embodiments, a provided compound is not one of the compounds listed in Table 2.

TABLE 2
Biological Evaluation of compounds using the assay as provided in the examples

| | | | | | PABP1 |
|---|---|---|---|---|---|
| Cpd | | PRMT1_ | PRMT5_ | CARM1_ | ICW IC₅₀ |
| No | Compound Structure | IC₅₀ (μM) | IC₅₀ (μM) | IC₅₀ (μM) | (μM) |
| 359. | 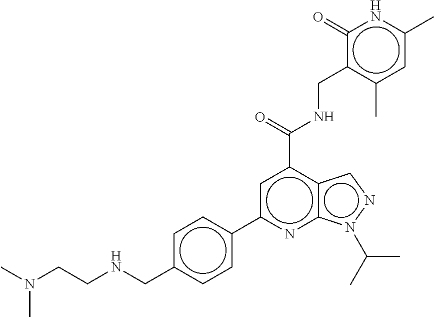 | 4.66387 | — | 50 | — |
| 360. | 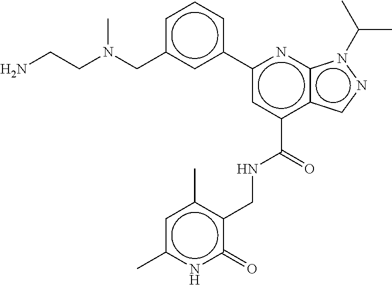 | 0.253817 | 15.4071 | 0.0195413 | 0.933556075 |
| 361. | 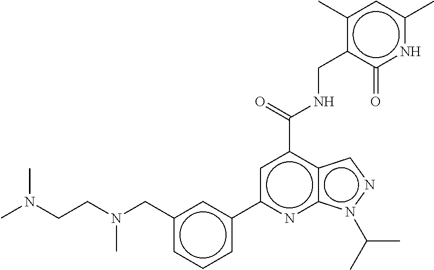 | 4.26531 | 50 | 0.254095 | — |
| 362. | 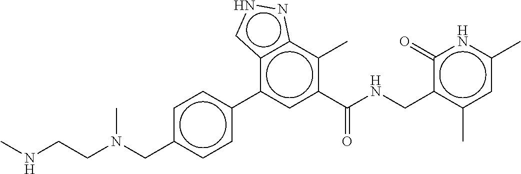 | 0.10445 | — | 1.98408 | 20 |
| 363. | 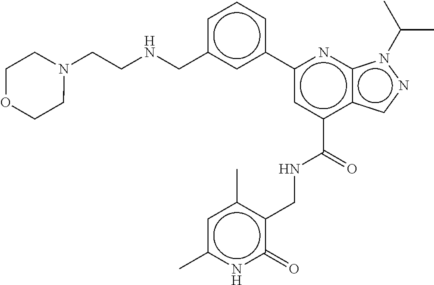 | — | 50 | — | — |
| 364. | 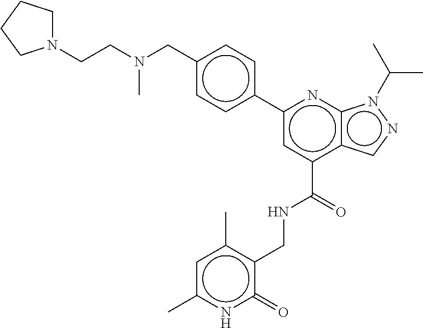 | — | — | 50 | — |
| 365. | 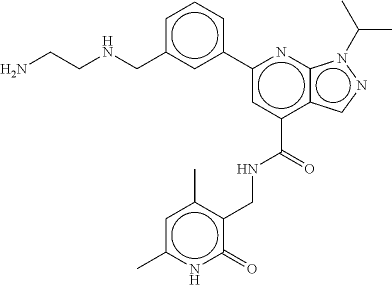 | 7.4879 | 49.0462 | 0.0143998 | — |
| 366. | 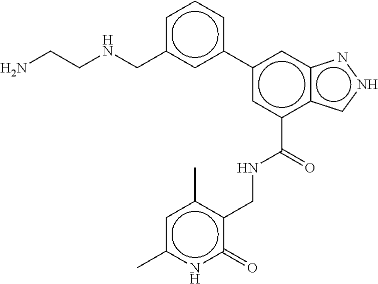 | 2.3979 | 4.19943 | 1.38695 | — |
| 367. | 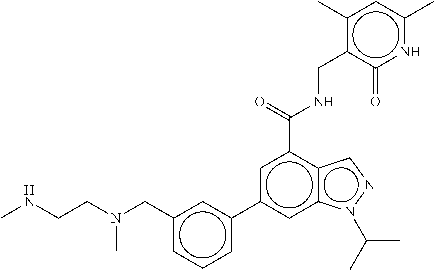 | 0.182751 | — | 0.183878 | 6.477584 |
| 368. | 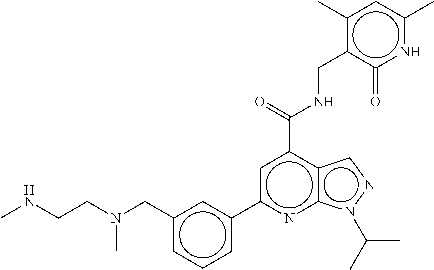 | 0.0865615 | — | 0.00610874 | 1.005110111 |
| 369. | 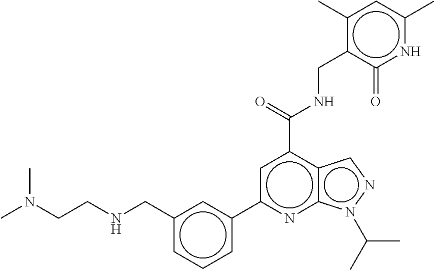 | — | — | 0.501013 | — |
| 370. | 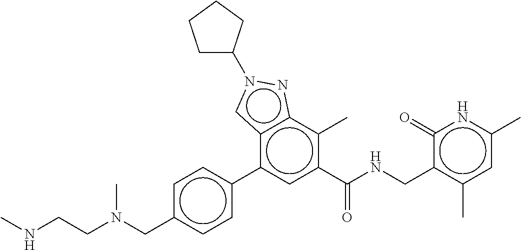 | 0.417504 | 50 | 7.25989 | — |
| 371. | 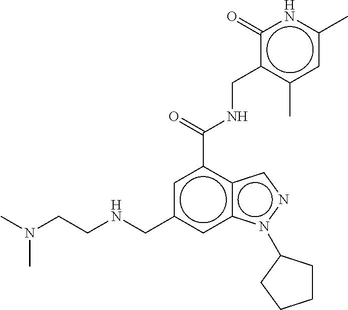 | 11.8016 | 50 | 28.3413 | — |
| 372. | 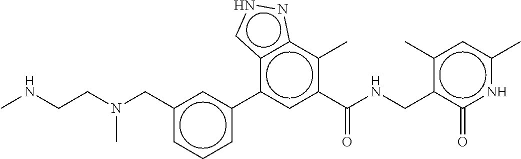 | 0.0595896 | — | 4.30586 | 20 |
| 373. | 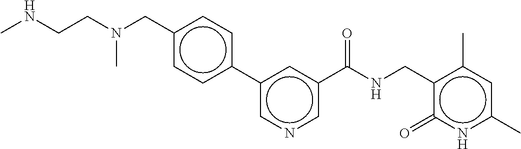 | 0.06473 | 14.7398 | 5.76793 | — |
| 374. | 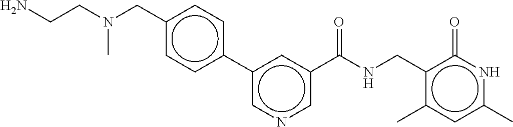 | 0.0730601 | 44.3249 | 9.83181 | — |
| 375. | 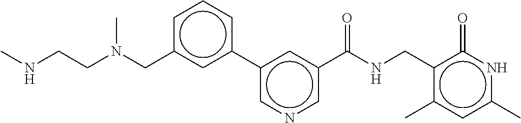 | 0.100691 | 50 | — | — |
| 376. | 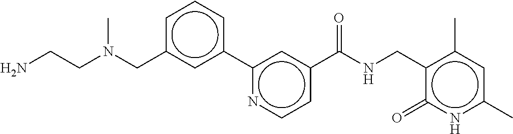 | 0.201711 | 24.3916 | — | — |
| 377. | 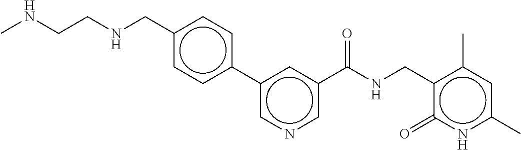 | 0.204202 | 36.4586 | — | — |
| 378. | 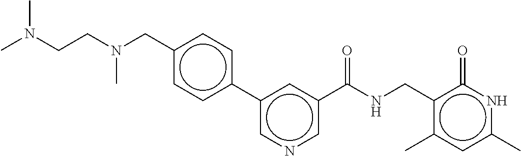 | 0.297228 | 66.6249 | 19.6481 | — |
| 379. | 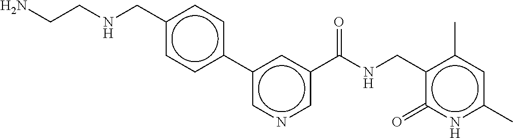 | 0.334958 | 2.12208 | 6.56561 | — |
| 380. | 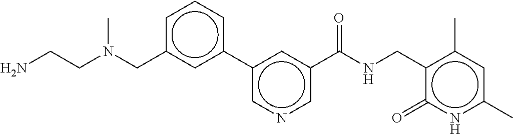 | 0.34004 | 50 | — | — |
| 381. | 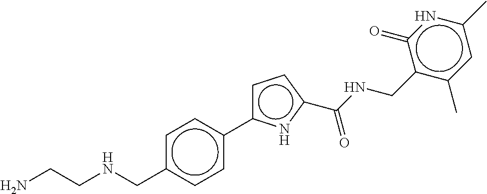 | 0.128139 | 0.399062 | 9.94719 | — |
| 382. | 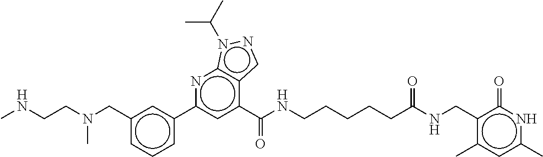 | 0.177317 | — | 0.00900877 | — |
| 383. | 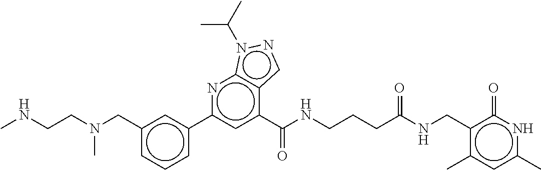 | 0.211997 | — | 0.00839142 | — |
| 384. | 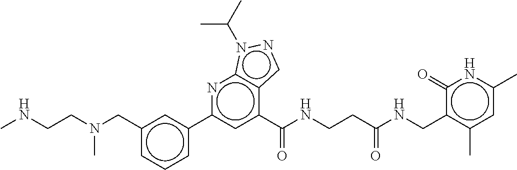 | 0.219528 | — | 0.0187973 | — |
| 385. | 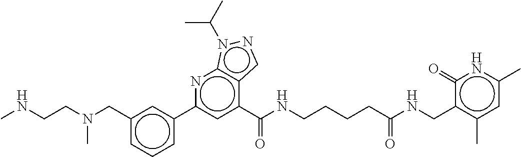 | 0.231948 | — | 0.00711345 | — |
| 386. | 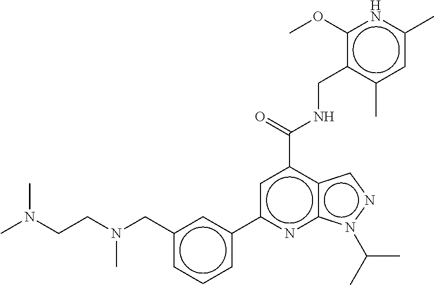 | 1.60635 | — | 0.208728 | — |
| 387. | 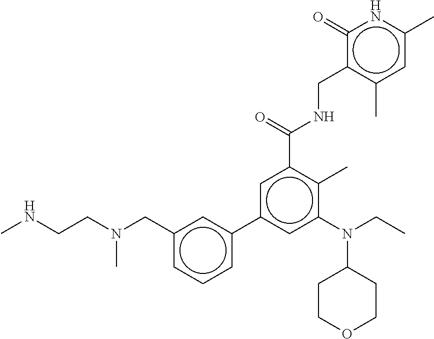 | — | — | 0.0971897 | 10.865537 |
| 388. | 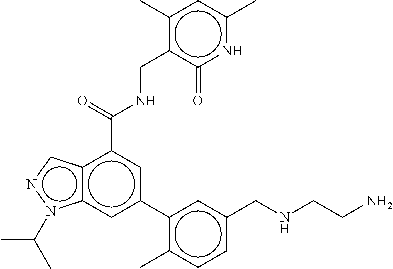 | — | — | 0.148549 | 20 |
| 389. | 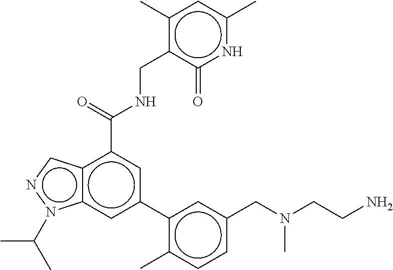 | — | — | 0.232119 | — |
| 390. | 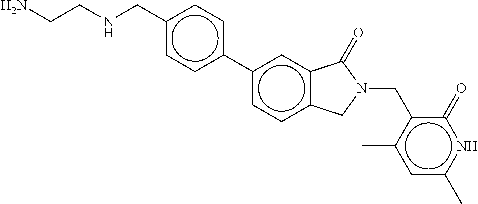 | — | 0.472444 | 17.5283 | — |
| 391. | 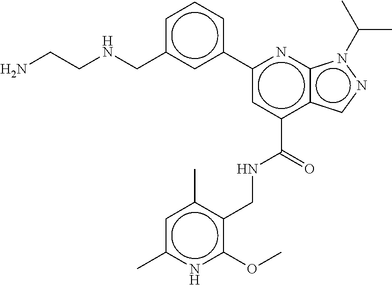 | 1.21551 | 30.4804 | 0.141351 | — |

In certain embodiments, a provided compound is not one of the compounds disclosed in the following patents and patent applications: U.S. Pat. Nos. 8,598,167 and 8,410,088; and International Patent Application Nos: PCT/US2012/033662, PCT/US2013/065126, PCT/US2013/077048, PCT/US2013/077086, PCT/US2014/047238, PCT/US2014/047282, and PCT/2013/065127, all of which are incorporated by references herein.

In certain embodiments, a provided compound inhibits an RMT (e.g., PRMT1, PRMT3, CARM1, PRMT6, and/or PRMT8). In certain embodiments, a provided compound inhibits wild-type PRMT1, PRMT3, CARM1, PRMT6, and/or PRMT8. In certain embodiments, a provided compound inhibits a mutant RMT. In certain embodiments, a provided compound inhibits PRMT1, PRMT3, CARM1, PRMT6, and/or PRMT8, e.g., as measured in an assay described herein. In certain embodiments, the RMT is from a human. In certain embodiments, a provided compound inhibits an RMT (e.g., PRMT1, PRMT3, CARM1, PRMT6, and/or PRMT8) at an IC₅₀ less than or equal to 10 μM. In certain embodiments, a provided compound inhibits an RMT (e.g., PRMT1, PRMT3, CARM1, PRMT6, and/or PRMT8) at an IC₅₀ less than or equal to 1 μM. In certain embodiments, a provided compound inhibits an RMT (e.g., PRMT1, PRMT3, CARM1, PRMT6, and/or PRMT8) at an IC₅₀ less than or equal to 0.1 μM. In certain embodiments, a provided compound inhibits an RMT (e.g., PRMT1, PRMT3, CARM1, PRMT6, and/or PRMT8) at an IC50 less than or equal to 0.01 μM. In certain embodiments, a provided compound inhibits an RMT (e.g., PRMT1, PRMT3, CARM1, PRMT6, and/or PRMT8) in a cell at an EC₃₀ less than or equal to 10 μM. In certain embodiments, a provided compound inhibits an RMT (e.g., PRMT1, PRMT3, CARM1, PRMT6, and/or PRMT8) in a cell at an EC₃₀ less than or equal to 12 μM. In certain embodiments, a provided compound inhibits an RMT (e.g., PRMT1, PRMT3, CARM1, PRMT6, and/or PRMT8) in a cell at an EC₃₀ less than or equal to 3 μM. In certain embodiments, a provided compound inhibits PRMT1 in a cell at an EC₃₀ less than or equal to 12 μM. In certain embodiments, a provided compound inhibits PRMT1 in a cell at an EC₃₀ less than or equal to 3 μM. In certain embodiments, a provided compound inhibits an RMT (e.g., PRMT1, PRMT3, CARM1, PRMT6, and/or PRMT8) in a cell at an EC₃₀ less than or equal to 1 μM. In certain embodiments, a provided compound inhibits an RMT (e.g., PRMT1, PRMT3, CARM1, PRMT6, and/or PRMT8) in a cell at an EC₃₀ less than or equal to 0.1 μM. In certain embodiments, a provided compound inhibits cell proliferation at an EC₅₀ less than or equal to 10 μM. In certain embodiments, a provided compound inhibits cell proliferation at an EC₅₀ less than or equal to 1 μM. In certain embodiments, a provided compound inhibits cell proliferation at an EC₅₀ less than or equal to 0.1 μM.

It will be understood by one of ordinary skill in the art that the RMT can be wild-type, or any mutant or variant.

The present disclosure provides pharmaceutical compositions comprising a compound described herein, e.g., a compound of Formula (I), or a pharmaceutically acceptable salt thereof, as described herein, and optionally a pharmaceutically acceptable excipient. It will be understood by one of ordinary skill in the art that the compounds described herein, or salts thereof, may be present in various forms, such as amorphous, hydrates, solvates, or polymorphs. In certain embodiments, a provided composition comprises two or more compounds described herein. In certain embodiments, a compound described herein, or a pharmaceutically acceptable salt thereof, is provided in an effective amount in the pharmaceutical composition. In certain embodiments, the effective amount is a therapeutically effective amount. In certain embodiments, the effective amount is an amount effective for inhibiting an RMT (e.g., PRMT1, PRMT3, CARM1, PRMT6, and/or PRMT8). In certain embodiments, the effective amount is an amount effective for treating an RMT-mediated disorder (e.g., a PRMT1-, PRMT3-, CARM1-, PRMT6-, and/or PRMT8-mediated disorder). In certain embodiments, the effective amount is a prophylactically effective amount. In certain embodiments, the effective amount is an amount effective to prevent an RMT-mediated disorder.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing a compound described herein (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

In some embodiments, a pharmaceutical composition described herein is sterilized.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds described herein are mixed with solubilizing agents such as Cremophor™, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a provided compound may include ointments, pastes, creams, lotions, gels, powders, solutions, sprays, inhalants and/or patches. Generally, the active ingredient is admixed under sterile conditions with a pharmaceutically acceptable carrier and/or any desired preservatives and/or buffers as can be required. Additionally, the present disclosure encompasses the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A provided pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A provided pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A provided pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In certain embodiments, the additional therapeutically active agent is a compound of Formula (I). In certain embodiments, the additional therapeutically active agent is not a compound of Formula (I). In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, an additional therapeutically active agent is prednisolone, dexamethasone, doxorubicin, vincristine, mafosfamide, cisplatin, carboplatin, Ara-C, rituximab, azacitadine, panobinostat, vorinostat, everolimus, rapamycin, ATRA (all-trans retinoic acid), daunorubicin, decitabine, Vidaza, mitoxantrone, or IBET-151.

Also encompassed by the present disclosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

Compounds and compositions described herein are generally useful for the inhibition of RMT (e.g., PRMT1, PRMT3, CARM1, PRMT6, and/or PRMT8). In some embodiments, methods of treating an RMT-mediated disorder in a subject are provided which comprise administering an effective amount of a compound described herein (e.g., a compound of Formula (I)), or a pharmaceutically acceptable salt thereof), to a subject in need of treatment. In certain embodiments, the effective amount is a therapeutically effective amount. In certain embodiments, the effective amount is a prophylactically effective amount. In certain embodiments, the subject is suffering from a RMT-mediated disorder. In certain embodiments, the subject is susceptible to a RMT-mediated disorder.

As used herein, the term “RMT-mediated disorder” means any disease, disorder, or other pathological condition in which an RMT (e.g., PRMT1, PRMT3, CARM1, PRMT6, and/or PRMT8) is known to play a role. Accordingly, in some embodiments, the present disclosure relates to treating or lessening the severity of one or more diseases in which an RMT is known to play a role.

In some embodiments, the present disclosure provides a method of inhibiting an RMT comprising contacting the RMT with an effective amount of a compound described herein (e.g., a compound of Formula (I)), or a pharmaceutically acceptable salt thereof. The RMT may be purified or crude, and may be present in a cell, tissue, or subject. Thus, such methods encompass both inhibition of in vitro and in vivo RMT activity. In certain embodiments, the method is an in vitro method, e.g., such as an assay method. It will be understood by one of ordinary skill in the art that inhibition of an RMT does not necessarily require that all of the RMT be occupied by an inhibitor at once. Exemplary levels of inhibition of an RMT (e.g., PRMT1, PRMT3, CARM1, PRMT6, and/or PRMT8) include at least 10% inhibition, about 10% to about 25% inhibition, about 25% to about 50% inhibition, about 50% to about 75% inhibition, at least 50% inhibition, at least 75% inhibition, about 80% inhibition, about 90% inhibition, and greater than 90% inhibition.

In some embodiments, provided is a method of inhibiting RMT activity in a subject in need thereof (e.g., a subject diagnosed as having an RMT-mediated disorder) comprising administering to the subject an effective amount of a compound described herein (e.g., a compound of Formula (I)), or a pharmaceutically acceptable salt thereof, or a pharmaceutical composition thereof.

In certain embodiments, provided is a method of modulating gene expression in a cell which comprises contacting a cell with an effective amount of a compound of Formula (I), or a pharmaceutically acceptable salt thereof. In certain embodiments, the cell is in culture in vitro. In certain embodiments, the cell is in an animal, e.g., a human. In certain embodiments, the cell is in a subject in need of treatment.

In certain embodiments, provided is a method of modulating transcription in a cell which comprises contacting a cell with an effective amount of a compound of Formula (I), or a pharmaceutically acceptable salt thereof. In certain embodiments, the cell is in culture in vitro. In certain embodiments, the cell is in an animal, e.g., a human. In certain embodiments, the cell is in a subject in need of treatment.

In certain embodiments, a method is provided of selecting a therapy for a subject having a disease associated with an RMT-mediated disorder or mutation comprising the steps of determining the presence of an RMT-mediated disorder or gene mutation in an RMT gene (e.g., a PRMT1, PRMT3, CARM1, PRMT6, and/or PRMT8 gene) or and selecting, based on the presence of an RMT-mediated disorder a gene mutation in the RMT gene a therapy that includes the administration of a provided compound. In certain embodiments, the disease is cancer.

In certain embodiments, a method of treatment is provided for a subject in need thereof comprising the steps of determining the presence of an RMT-mediated disorder or a gene mutation in the RMT gene and treating the subject in need thereof, based on the presence of a RMT-mediated disorder or gene mutation in the RMT gene with a therapy that includes the administration of a provided compound. In certain embodiments, the subject is a cancer patient.

In some embodiments, a compound provided herein is useful in treating a proliferative disorder, such as cancer. For example, while not being bound to any particular mechanism, protein arginine methylation by PRMTs is a modification that has been implicated in signal transduction, gene transcription, DNA repair and mRNA splicing, among others; and overexpression of PRMTs within these pathways is often associated with various cancers. Thus, compounds which inhibit the action of PRMTs, as provided herein, are effective in the treatment of cancer.

In some embodiments, compounds provided herein are effective in treating cancer through the inhibition of PRMT1. For example, PRMT1 overexpression has been observed in various human cancers, including, but not limited to, breast cancer, prostate cancer, lung cancer, colon cancer, bladder cancer, and leukemia. In one example, PRMT1 specifically deposits an asymmetric dimethylarginine (aDMA) mark on histone H4 at arginine 3 (H4R3me2a), and this mark is associated with transcription activation. In prostate cancer, the methylation status of H4R3 positively correlates with increasing tumor grade and can be used to predict the risk of prostate cancer recurrence (Seligson et al., Nature 2005 435, 1262-1266). Thus, in some embodiments, inhibitors of PRMT1, as described herein, are useful in treating cancers associated with the methylation status of H4R3, e.g., prostate cancer. Additionally, the methylarginine effector molecule TDRD3 interacts with the H4R3me2a mark, and overexpression of TDRD3 is linked to poor prognosis for the survival of patients with breast cancer (Nagahata et al., Cancer Sci. 2004 95, 218-225). Thus, in some embodiments, inhibitors of PRMT1, as described herein, are useful in treating cancers associated with overexpression of TDRD3, e.g., breast cancer, as inhibition of PRMT1 leads to a decrease in methylation of H4R3, thereby preventing the association of overexpressed TDRD3 with H4R3me2a. In other examples, PRMT1 is known to have non-histone substrates. For example, PRMT1, when localized to the cytoplasm, methylates proteins that are involved in signal transduction pathways, e.g., the estrogen receptor (ER). The expression status of ER in breast cancer is critical for prognosis of the disease, and both genomic and non-genomic ER pathways have been implicated in the pathogenesis of breast cancer. For example, it has been shown that PRMT1 methylates ERα, and that ERα methylation is required for the assembly of ERα with SRC (a proto-oncogene tyrosine-protein kinase) and focal adhesion kinase (FAK). Further, the silencing of endogenous PRMT1 resulted in the inability of estrogen to activate AKT. These results suggested that PRMT1-mediated ERα methylation is required for the activation of the SRC-PI3K-FAK cascade and AKT, coordinating cell proliferation and survival. Thus, hypermethylation of ERα in breast cancer is thought to cause hyperactivation of this signaling pathway, providing a selective survival advantage to tumor cells (Le Romancer et al., Mol. Cell 2008 31, 212-221; Le Romancer et al., Steroids 2010 75, 560-564). Accordingly, in some embodiments, inhibitors of PRMT1, as described herein, are useful in treating cancers associated with ERα methylation, e.g., breast cancer. In yet another example, PRMT1 has been shown to be involved in the regulation of leukemia development. For example, SRC-associated in mitosis 68 kDa protein (SAM68; also known as KHDRBS1) is a well-characterized PRMT1 substrate, and when either SAM68 or PRMT1 is fused directly to the myeloid/lymphoid leukemia (MLL) gene, these fusion proteins can activate MLL oncogenic properties, implying that the methylation of SAM68 by PRMT1 is a critical signal for the development of leukemia (Cheung et al., Nature Cell Biol. 2007 9, 1208-1215). Accordingly, in some embodiments, inhibitors of PRMT1, as described herein, are useful in treating cancers associated with SAM68 methylation, e.g., leukemia. In still another example, PRMT1 is implicated in leukemia development through its interaction with AE9a, a splice isoform of AML1-ETO (Shia et al., Blood 2012 119:4953-62). Knockdown of PRMT1 affects expression of certain AE9a-activated genes and suppresses AE9a's self-renewal capability. It has also been shown that AE9a recruits PRMT1 to AE9a activated gene promoters, which leads to increased H4 Arg3 methylation, H3 Lys9/14 acetylation, and transcription activated. Accordingly, in some embodiments, inhibitors of PRMT1, as described herein, are useful in treating cancers associated with AML1-ETO, e.g., leukemia. Thus, without being bound by any particular mechanism, the inhibition of PRMT1, e.g., by compounds described herein, is beneficial in the treatment of cancer.

In some embodiments, compounds provided herein are effective in treating cancer through the inhibition of PRMT3. In one example, the DAL1 tumor suppressor protein has been shown to interact with PRMT3 and inhibits its methyltransferase activity (Singh et al., Oncogene 2004 23, 7761-7771). Epigenetic downregulation of DAL1 has been reported in several cancers (e.g., meningiomas and breast cancer), thus PRMT3 is expected to display increased activity, and cancers that display DAL1 silencing may, in some aspects, be good targets for PRMT3 inhibitors, e.g., those described herein. Thus, without being bound by any particular mechanism, the inhibition of PRMT3, e.g., by compounds described herein, is beneficial in the treatment of cancer.

In some embodiments, compounds provided herein are effective in treating cancer through the inhibition of PRMT4, also known as CARM1. For example, PRMT4 levels have been shown to be elevated in castration-resistant prostate cancer (CRPC), as well as in aggressive breast tumors (Hong et al., Cancer 2004 101, 83-89; Majumder et al., Prostate 2006 66, 1292-1301). Thus, in some embodiments, inhibitors of PRMT4, as described herein, are useful in treating cancers associated with PRMT4 overexpression. PRMT4 has also been shown to affect ERα-dependent breast cancer cell differentiation and proliferation (Al-Dhaheri et al., Cancer Res. 2011 71, 2118-2128), thus in some aspects PRMT4 inhibitors, as described herein, are useful in treating ERα-dependent breast cancer by inhibiting cell differentiation and proliferation. In another example, PRMT4 has been shown to be recruited to the promoter of E2F1 (which encodes a cell cycle regulator) as a transcriptional co-activator (Frietze et al., Cancer Res. 2008 68, 301-306). Thus, PRMT4-mediated upregulation of E2F1 expression may contribute to cancer progression and chemoresistance as increased abundance of E2F1 triggers invasion and metastasis by activating growth receptor signaling pathways, which in turn promote an antiapoptotic tumor environment (Engelmann and Pützer, Cancer Res 2012 72; 571). Accordingly, in some embodiments, the inhibition of PRMT4, e.g., by compounds provided herein, is useful in treating cancers associated with E2F1 upregulation. Thus, without being bound by any particular mechanism, the inhibition of PRMT4, e.g., by compounds described herein, is beneficial in the treatment of cancer.

In some embodiments, compounds provided herein are effective in treating cancer through the inhibition of PRMT6. For example, PRMT6 has been reported to be overexpressed in a number of cancers, e.g., bladder and lung cancer (Yoshimatsu et al., Int. J. Cancer 2011 128, 562-573). Thus, in some embodiments, the inhibition of PRMT6, by compounds provided herein, is useful in treating cancers associated with PRMT6 overexpression. In some aspects, PRMT6 is primarily thought to function as a transcriptional repressor, although it has also been reported that PRMT6 functions as a co-activator of nuclear receptors. For example, as a transcriptional repressor, PRMT6 suppresses the expression of thrombospondin 1 (TSP1; also known as THBS1; a potent natural inhibitor of angiogenesis and endothelial cell migration) and p21 (a natural inhibitor of cyclin dependent kinase), thereby contributing to cancer development and progression (Michaud-Levesque and Richard, J. Biol. Chem. 2009 284, 21338-21346; Kleinschmidt et al., PLoS ONE 2012 7, e41446). Accordingly, in some embodiments, the inhibition of PRMT6, by compounds provided herein, is useful in treating cancer by preventing the repression of THBs1 and/or p21. Thus, without being bound by any particular mechanism, the inhibition of PRMT6, e.g., by compounds described herein, is beneficial in the treatment of cancer.

In some embodiments, compounds provided herein are effective in treating cancer through the inhibition of PRMT8. For example, deep-sequencing efforts of cancer genomes (e.g., COSMIC) have revealed that of all the PRMTs, PRMT8 is reported to be the most mutated. Of 106 sequenced genomes, 15 carry mutations in the PRMT8 coding region, and nine of these result in an amino acid change (Forbes et al., Nucleic Acids Res. 2011 39, D945-D950). Because of its high rate of mutation in cancer, PRMT8 is thought to contribute to the initiation or progression of cancer. Thus, without being bound by any particular mechanism, the inhibition of PRMT8, e.g., by compounds described herein, is beneficial in the treatment of cancer.

In some embodiments, compounds described herein are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (e.g., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, a compound provided herein is useful in treating diseases associated with increased levels of circulating asymmetric dimethylarginine (aDMA), e.g., cardiovascular disease, diabetes, kidney failure, renal disease, pulmonary disease, etc. Circulating aDMA is produced by the proteolysis of asymmetrically dimethylated proteins. PRMTs which mediate aDMA methylation include, e.g., PRMT1, PRMT3, PRMT4, PRMT6, and PRMT8. aDMA levels are directly involved in various diseases as aDMA is an endogenous competitive inhibitor of nitric oxide synthase (NOS), thereby reducing the production of nitric oxide (NO) (Vallance et al., J. Cardiovasc. Pharmacol. 1992 20 (Suppl. 12):S60-2). NO functions as a potent vasodilator in endothelial vessels, and as such inhibiting its production has major consequences on the cardiovascular system. For example, since PRMT1 is a major enzyme that generates aDMA, the dysregulation of its activity is likely to regulate cardiovascular diseases (Boger et al., Ann. Med. 2006 38:126-36), and other pathophysiological conditions such as diabetes mellitus (Sydow et al., Vasc. Med. 2005 10 (Suppl. 1):S35-43), kidney failure (Vallance et al., Lancet 1992 339:572-5), and chronic pulmonary diseases (Zakrzewicz et al., BMC Pulm. Med. 2009 9:5). Additionally, it has been demonstrated that the expression of PRMT1 and PRMT3 are increased in coronary heart disease (Chen et al., Basic Res. Cardiol. 2006 101:346-53). In another example, aDMA elevation is seen in patients with renal failure, due to impaired clearance of this metabolite from the circulation (Jacobi et al., Am. J. Nephrol. 2008 28:224-37). Thus, circulating aDMA levels is observed in many pathophysiological situations. Accordingly, without being bound by any particular mechanism, the inhibition of PRMTs, e.g., by compounds described herein, results in the decrease of circulating aDMA, which is beneficial in the treatment of diseases associated with increased levels of circulating aDMA, e.g., cardiovascular disease, diabetes, kidney failure, renal disease, pulmonary disease, etc. In certain embodiments, a compound described herein is useful for treating or preventing vascular diseases.

In some embodiments, a compound provided herein is useful in treating metabolic disorders. For example, PRMT1 has been shown to enhance mRNA levels of FoxO1 target genes in gluconeogenesis, which results in increased hepatic glucose production, and knockdown of PRMT promotes inhibition of FoxO1 activity and thus inhibition of hepatic gluconeogenesis (Choi et al., Hepatology 2012 56:1546-56). Additionally, genetic haploinsufficiency of Prmt1 has been shown to reduce blood glucose levels in mouse models. Thus, without being bound by any particular mechanism, the inhibition of PRMT1, e.g., by compounds described herein, is beneficial in the treating of metabolic disorders, such as diabetes. In some embodiments, a provided compound is useful in treating type I diabetes. In some embodiments, a provided compound is useful in treating type II diabetes.

In some embodiments, a compound provided herein is useful in treating muscular dystrophies. For example, PRMT1, as well as PRMT3 and PRMT6, methylate the nuclear poly(A)-binding protein (PABPN1) in a region located near its C-terminus (Perreault et al., J. Biol. Chem. 2007 282:7552-62). This domain is involved in the aggregation of the PABPN1 protein, and abnormal aggregation of this protein is involved in the disease oculopharyngeal muscular dystrophy (Davies et al., Int. J. Biochem. Cell. Biol. 2006 38:1457-62). Thus, without being bound by any particular mechanism, the inhibition of PRMTs, e.g., by compounds described herein, is beneficial in the treatment of muscular dystrophies, e.g., oculopharyngeal muscular dystrophy, by decreasing the amount of methylation of PABPN1, thereby decreasing the amount of PABPN1 aggregation.

CARM1 is also the most abundant PRMT expressed in skeletal muscle cells, and has been found to selectively control the pathways modulating glycogen metabolism, and associated AMPK (AMP-activated protein kinase) and p38 MAPK (mitogen-activated protein kinase) expression. See, e.g., Wang et al., Biochem (2012) 444:323-331. Thus, in some embodiments, inhibitors of CARM1, as described herein, are useful in treating metabolic disorders, e.g., for example skeletal muscle metabolic disorders, e.g., glycogen and glucose metabolic disorders. Exemplary skeletal muscle metabolic disorders include, but are not limited to, Acid Maltase Deficiency (Glycogenosis type 2; Pompe disease), Debrancher deficiency (Glycogenosis type 3), Phosphorylase deficiency (McArdle's; GSD 5), X-linked syndrome (GSD9D), Autosomal recessive syndrome (GSD9B), Tarui's disease (Glycogen storage disease VII; GSD 7), Phosphoglycerate Mutase deficiency (Glycogen storage disease X; GSDX; GSD 10), Lactate dehydrogenase A deficiency (GSD 11), Branching enzyme deficiency (GSD 4), Aldolase A (muscle) deficiency, β-Enolase deficiency, Triosephosphate isomerase (TIM) deficiency, Lafora's disease (Progressive myoclonic epilepsy 2), Glycogen storage disease (Muscle, Type 0, Phosphoglucomutase 1 Deficiency (GSD 14)), and Glycogenin Deficiency (GSD 15).

In some embodiments, a compound provided herein is useful in treating autoimmune disease. For example, several lines of evidence strongly suggest that PRMT inhibitors may be valuable for the treatment of autoimmune diseases, e.g., rheumatoid arthritis. PRMTs are known to modify and regulate several critical immunomodulatory proteins. For example, post-translational modifications (e.g., arginine methylation), within T cell receptor signaling cascades allow T lymphocytes to initiate a rapid and appropriate immune response to pathogens. Co-engagement of the CD28 costimulatory receptor with the T cell receptor elevates PRMT activity and cellular protein arginine methylation, including methylation of the guanine nucleotide exchange factor Vav1 (Blanchet et al., J. Exp. Med. 2005 202:371-377). PRMT inhibitors are thus expected to diminish methylation of the guanine exchange factor Vav1, resulting in diminished IL-2 production. In agreement, siRNA directed against PRMT5 was shown to both inhibit NFAT-driven promoter activity and IL-2 secretion (Richard et al., Biochem J. 2005 388:379-386). In another example, PRMT1 is known to cooperate with PRMT4 to enhance NFkB p65-driven transcription and facilitate the transcription of p65 target genes like TNFα (Covic et al., Embo. J. 2005 24:85-96). Thus, in some embodiments, PRMT1 and/or PRMT4 inhibitors, e.g., those described herein, are useful in treating autoimmune disease by decreasing the transcription of p65 target genes like TNFα. These examples demonstrate an important role for arginine methylation in inflammation. Thus, without being bound by any particular mechanism, the inhibition of PRMTs, e.g., by compounds described herein, is beneficial in the treatment of autoimmune diseases.

In some embodiments, a compound provided herein is useful in treating neurological disorders, such as amyotrophic lateral sclerosis (ALS). For example, a gene involved in ALS, TLS/FUS, often contains mutated arginines in certain familial forms of this disease (Kwiatkowski et al., Science 2009 323:1205-8). These mutants are retained in the cytoplasm, which is similar to reports documenting the role arginine methylation plays in nuclear-cytoplasmic shuffling (Shen et al., Genes Dev. 1998 12:679-91). This implicates PRMT, e.g., PRMT1, function in this disease, as it was demonstrated that TLS/FUS is methylated on at least 20 arginine residues (Rappsilber et al., Anal. Chem. 2003 75:3107-14). Thus, in some embodiments, the inhibition of PRMTs, e.g., by compounds provided herein, are useful in treating ALS by decreasing the amount of TLS/FUS arginine methylation.

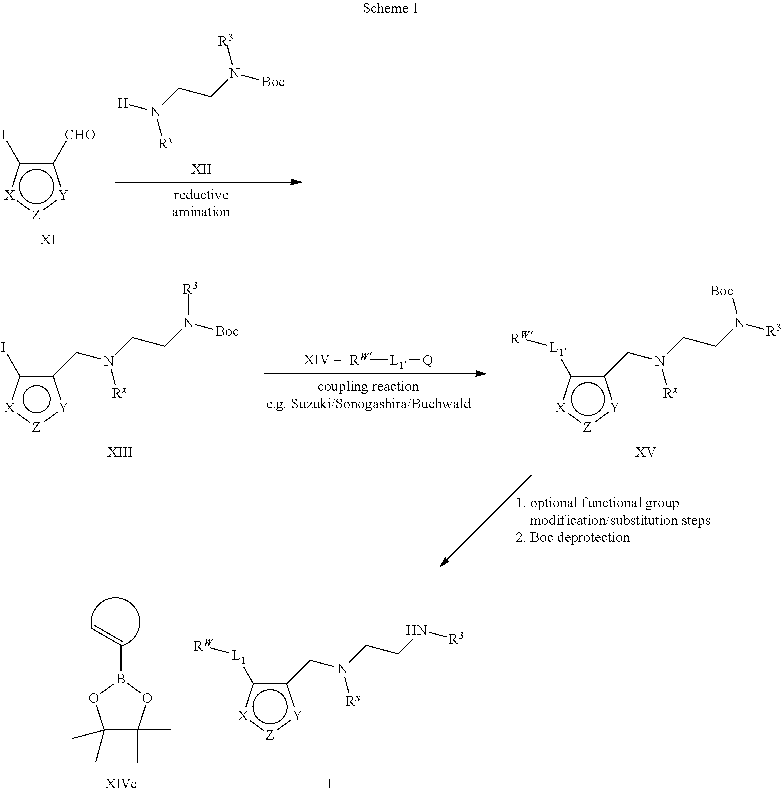

Scheme 1 shows an exemplary general synthesis route to pyrazole compounds of formula I, wherein R^{W′} is either the same as R^{W} or is precursor of R^{W} and L_{1′} is either the same as L₁ or is a precursor of L₁ and R^{W}, L₁, R^{x}, R³, X, Y and Z are as defined above. In the first step iodopyrazole carboxaldehydes of general formula XI are allowed to react with mono-Boc protected ethylenediamines XII under reductive amination conditions (e.g. sodium cyanoborohydride and catalytic acid such as acetic acid) in an appropriate solvent such as methanol to give intermediates of general formula XIII. In certain embodiments, Sonagashira reaction of intermediates of general formula XIII with boronic acids or boronic esters of general formula XIV in which L_{1′} is an acetylene linker and Q is a boronic acid or boronic ester group in the presence of a palladium catalyst (e.g. PdCl₂(dppf)) and a base (e.g. potassium carbonate) in an organic solvent (e.g. toluene) at elevated temperature yields intermediates of general formula XV-a in which L_{1′} is an acetylene linker. Boc deprotection of intermediates of general formula XV-a gives acetylene compounds of formula VI-a. In certain embodiments, Suzuki reaction of intermediates of general formula XIII with boronic acids or boronic esters of general formula XIV in which L_{1′} is a trans-olefin linker and Q is a boronic acid or boronic ester group in the presence of a palladium catalyst (e.g. PdCl₂(dppf)) and a base (e.g. potassium carbonate) in an organic solvent (e.g. toluene) at elevated temperature yields intermediates of general formula XV-b in which L_{1′} is an olefin linker. Boc deprotection of intermediates of general formula XV-b gives olefin compounds of formula VI-b. In certain embodiments, Suzuki reaction of intermediates of general formula XIII with pinacol boranes of general formula XIVc in which L_{1′} is bond, R^{W′} is a heterocycloalkenyl or cycloalkenyl group and Q is a pinacol borane group yields intermediates of general formula XV-c in which L_{1′} is bond and R^{W′} is a heterocycloalkenyl or cycloalkenyl group. In certain embodiments, compounds of formula I wherein L₁ is bond and R^{W} is a heterocyclyl or carbocyclyl group can be prepared by hydrogenation of intermediates of formula XV-c followed by Boc deprotection. In certain embodiments, compounds of formula I where L₁ is —O— can be synthesized from intermediates of general formula XIII by Goldberg reaction with alcohols of formula R^{W}OH followed by Boc deprotection. In certain embodiments, compounds of formula I where L₁ is —N(R^{B})— can be synthesized from intermediates of general formula XIII by palladium catalyzed Buchwald coupling reaction conditions with amines of formula R^{W}N(R^{B})H followed by Boc deprotection. In certain embodiments, compounds of formula I where L₁ is —C(═O)NR^{B}— can be synthesized from intermediates of general formula XIII under known copper catalyzed coupling reaction conditions of amides with aryliodides using copper iodide an amine ligand and a base with amides of formula R^{W}C(═O)NHR^{B} followed by Boc deprotection.

Scheme 1.1 shows an alternative general synthesis route to pyrazole compounds of Formula (I), that involves reversal in the order of the first two steps of the reaction sequence detailed for Scheme 1.0. Thus, in the first step iodopyrazole carboxaldehydes of general formula XI are coupled with compounds or reagents of general formula XIV (e.g. via Suzuki reaction with pinacol boranes of general formula XIVc in which L_{1′} is bond, R^{W′} is a heterocycloalkenyl or cycloalkenyl group and Q is a pinacol borane group) and in a second step the corresponding reductive amination reaction to yield common intermediates of general formula XV is a carried out.

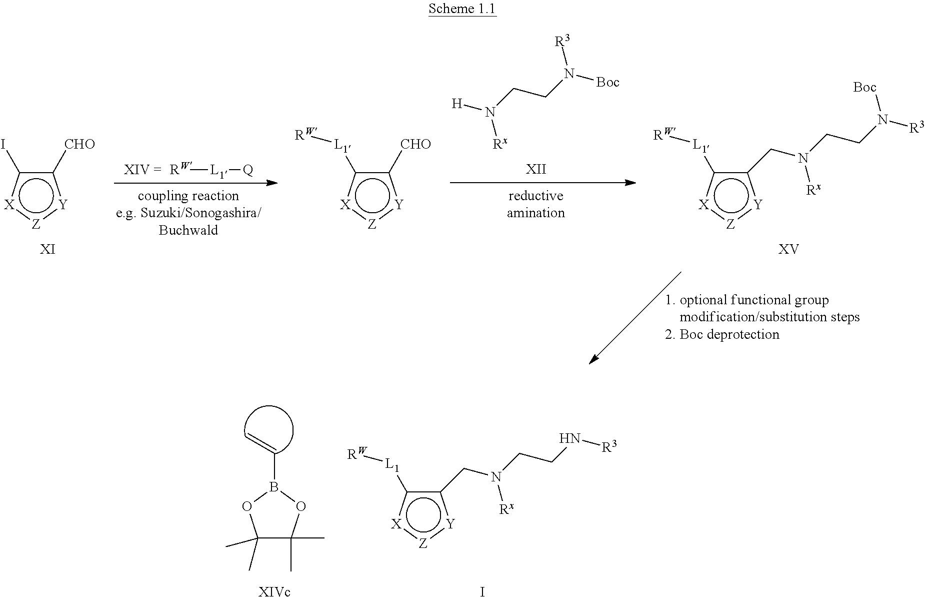

In certain embodiments, iodopyrazole carboxaldehydes of general formula XI may be prepared from suitable known pyrazole compound intermediates by established synthetic chemistry methods. Standard methods include direct iodination of a pyrazole 3-carboxylate and Sandmeyer reaction of a 3-amino pyrazole 4-carboxylate. In certain embodiments, iodopyrazole carboxaldehydes can be derived from iodopyrazole carboxylates by reduction to a hydroxymethyl group followed by oxidation to carboxaldehyde. In certain embodiments, mono-Boc protected ethylenediamines XII can be synthesized by standard methods known in the literature for derivatizing or preparing ethylenediamines. For example intermediates of formula XII may be prepared by treatment of the corresponding unprotected diamine precursors with Boc₂O and purifying the mixture of mono and dibocylated products. In certain embodiments, pyrazole compounds of general formula II can be prepared from iodopyrazole carboxaldehydes of general formula XXI as depicted in Scheme 2. In certain embodiments where R⁴ is hydrogen compounds of general formula II are equivalent to compounds of general formula III which are tautomers. In certain embodiments, R^{4′} is a protecting group such as tetrahydropyranylyl (THP) which maybe cleaved to hydrogen under acidic conditions in the final Boc-deprotection step. In certain embodiments, iodopyrazole carboxaldehydes of general formula XXI can be prepared as depicted in Scheme 3.

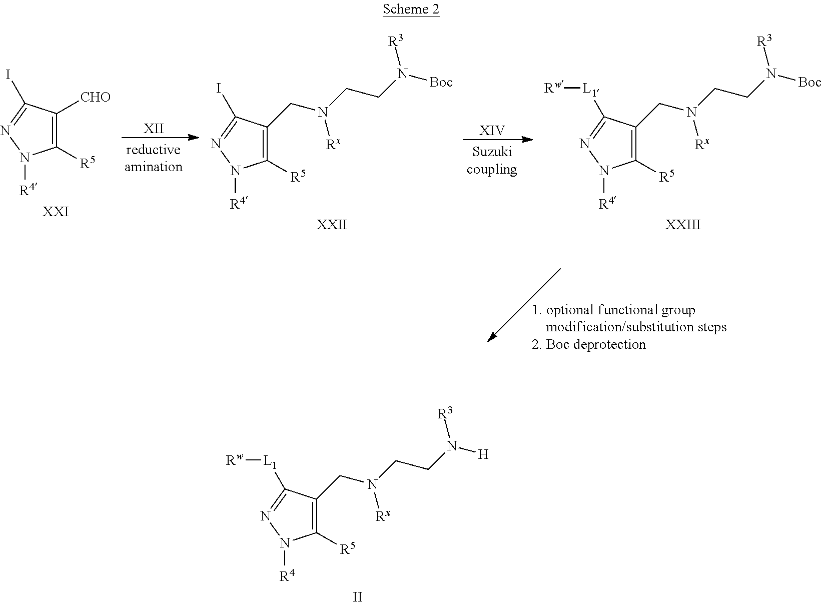

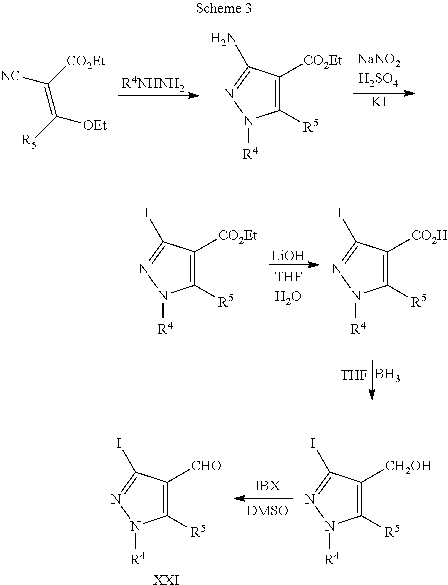

In certain embodiments, iodopyrazole carboxaldehydes of general formula XXI can be prepared as depicted in Scheme 4 which also provides iodopyrazole carboxyaldehydes of general formula XXXI. In certain embodiments, alkylation of intermediates of general formula XXX gives a mixture of pyrazole nitrogen alkylated isomers which are separated by chromatography to give pure isomers XXI and XXXI. In certain embodiments, pyrazole compounds of general formula III can be prepared from iodopyrazole carboxaldehydes of general formula XXXI as depicted in Scheme 5.

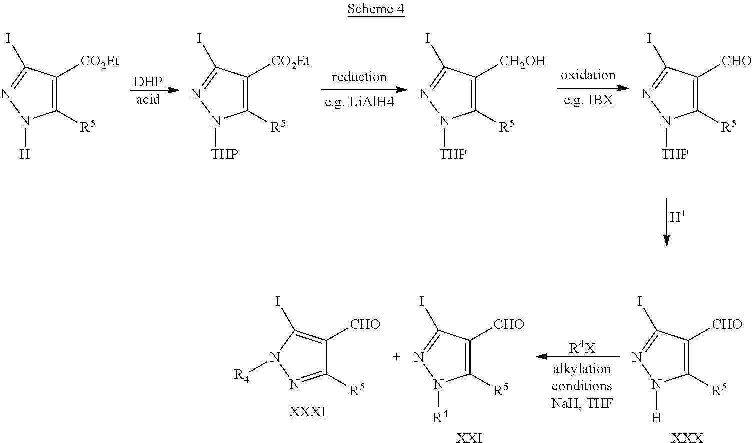

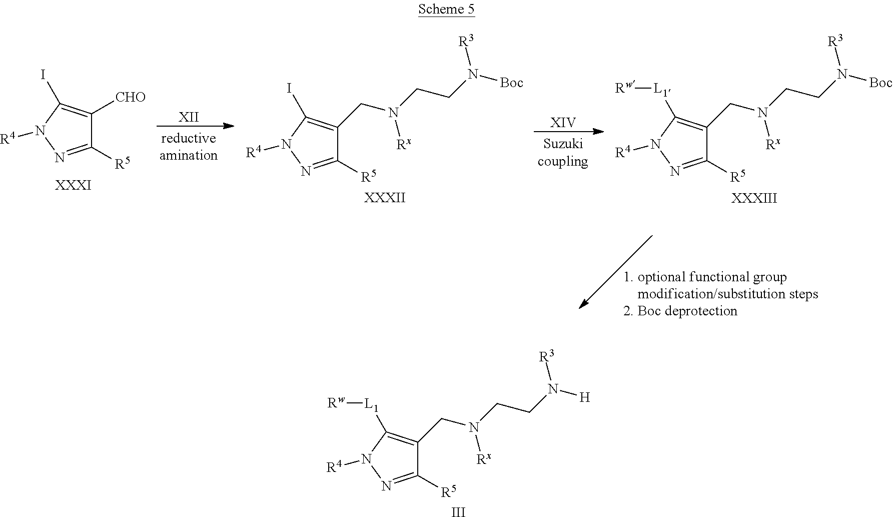

In certain embodiments, pyrazole compounds of general formula IV can be prepared from iodopyrazole carboxaldehydes of general formula XLI as depicted in Scheme 6. In certain embodiments where R⁴ is hydrogen compounds of general formula IV are equivalent to compounds of general formula V which are tautomers. In certain embodiments where R⁴ in compounds of formula IV is hydrogen, R^{4′} in intermediate XLI may be a selected protecting group such as tetrahydropyranyl (THP) which can be cleaved to hydrogen under acidic conditions in the final Boc-deprotection step.

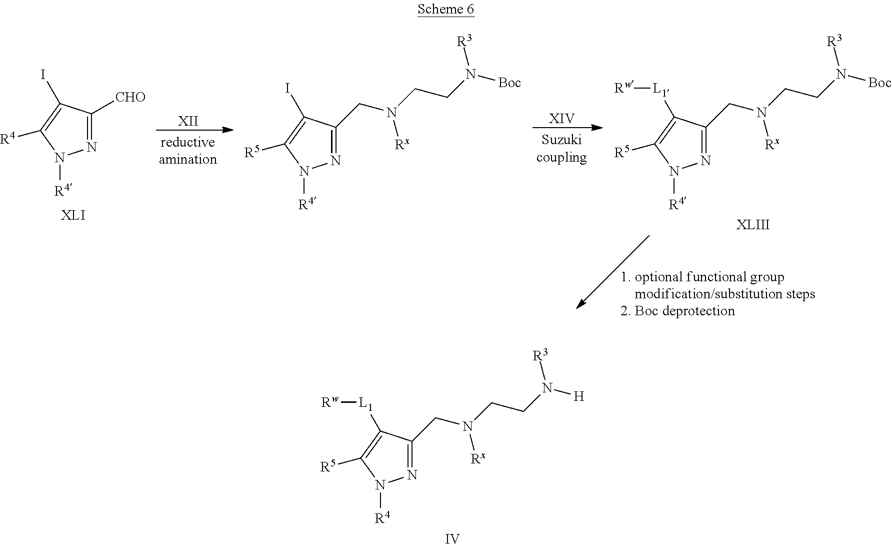

In certain embodiments, iodopyrazole carboxaldehydes of general formula XLI and LI can be prepared as depicted in Scheme 7. In certain embodiments, an R⁴ group of iodopyrazole carboxaldehydes may be introduced by alkylation of intermediates of formula XLVII. This reaction can give a mixture of intermediate compounds of formulas XLI and LI which may be separated by chromatography. In certain embodiments, THP protected intermediates of formula XLVI can be used to prepare compounds of formula IV where R⁴═H as also depicted in Scheme 7.

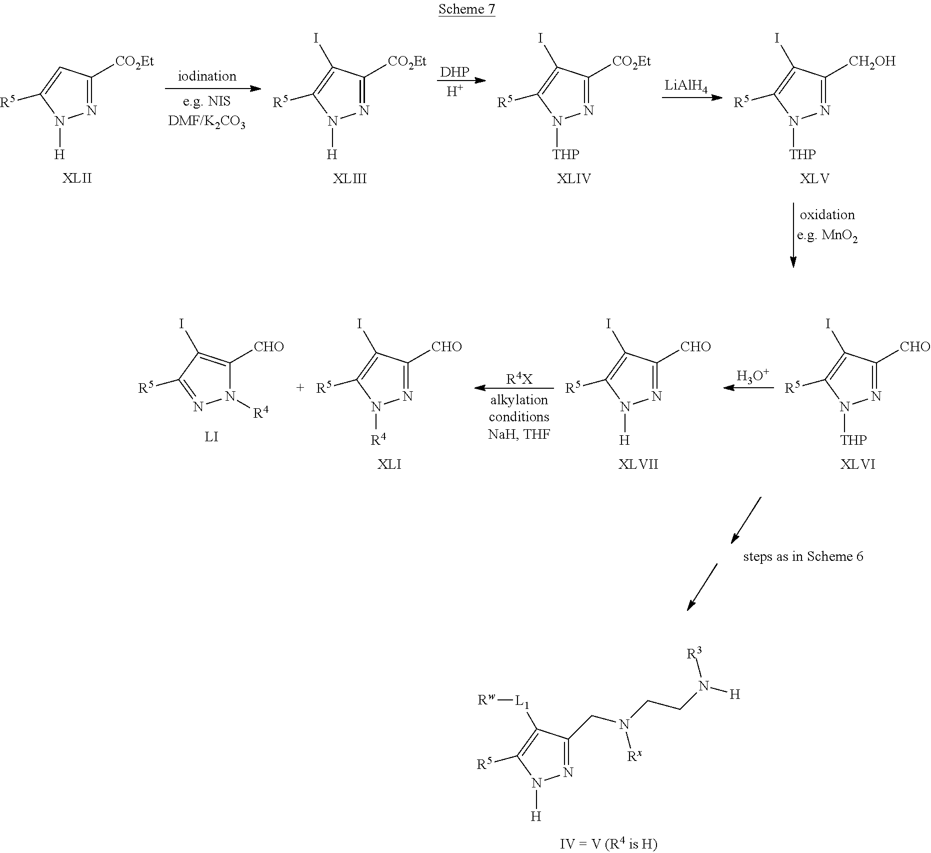

In certain embodiments, pyrazole compounds of general formula V can be prepared from iodopyrazole carboxaldehydes of general formula LI as depicted in Scheme 8.

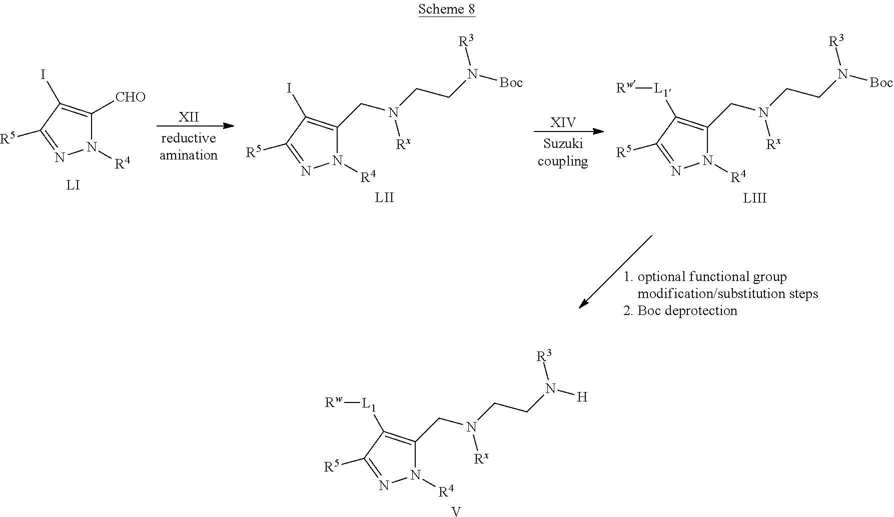

In certain embodiments, boronic acids or esters of general formula XIVa, XIVb and XIVc are commercially available. In certain embodiments, compounds of general formula XIVa, and XIVb can also be prepared from alkenyl bromides and terminal alkynes using standard methods such as treatment with n-BuLi followed by trapping the intermediate lithium species with trimethylborate. In certain embodiments, compounds of general formula XIVc can be prepared from the corresponding cyclic ketones LX via intermediate enol triflates as depicted in Scheme 9.

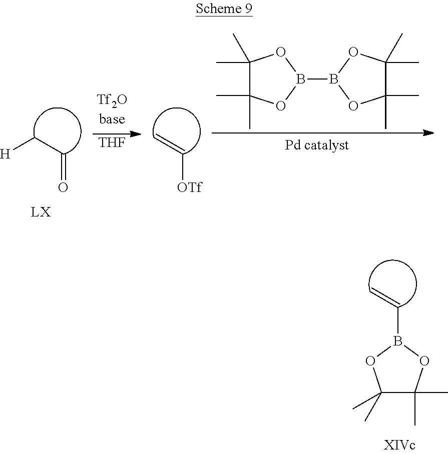

### EXAMPLES

In order that the invention described herein may be more fully understood, the following examples are set forth. It should be understood that these examples are for illustrative purposes only and are not to be construed as limiting this invention in any manner.

### Synthetic Methods

General methods and experimental procedures for preparing and characterizing compounds of the present invention are set forth below. Wherever needed, reactions were heated using conventional hotplate apparatus or heating mantle or microwave irradiation equipment. Reactions were conducted with or without stirring, under atmospheric or elevated pressure in either open or closed vessels. Reaction progress was monitored using conventional techniques such as TLC, HPLC, UPLC, or LCMS using instrumentation and methods described below. Reactions were quenched and crude compounds isolated using conventional methods as described in the specific examples provided. Solvent removal was carried out with or without heating, under atmospheric or reduced pressure, using either a rotary or centrifugal evaporator.

Compound purification was carried out as needed using a variety of traditional methods including, but not limited to, preparative chromatography under acidic, neutral, or basic conditions using either normal phase or reverse phase HPLC or flash columns or Prep-TLC plates. Compound purity and mass confirmations were conducted using standard HPLC and/or UPLC and/or MS spectrometers and/or LCMS and/or GC equipment (e.g., including, but not limited to the following instrumentation: Waters Alliance 2695 with 2996 PDA detector connected with ZQ detector and ESI source; Shimadzu LDMS-2020; Waters Acquity H Class with PDA detector connected with SQ detector and ESI source; Agilent 1100 Series with PDA detector; Waters Alliance 2695 with 2998 PDA detector; AB SCIEX API 2000 with ESI source; Agilent 7890 GC). Exemplified compounds were dissolved in either MeOH or MeCN to a concentration of approximately 1 mg/mL and analyzed by injection of 0.5-10 μL into an appropriate LCMS system using the methods provided in the following table:

| | | | | | | MS | |
|---|---|---|---|---|---|---|---|
| | | | | | | Heat | MS |
| | | | | Flow | | Block | Detector |
| | | Mobile | Mobile | Rate | | Temp | Voltage |
| Method | Column | Phase A | Phase B | (mL/min) | Gradient Profile | (° C.) | (kV) |
| A | Shim-pack | Water/0.05% | ACN/0.05% | 1 | 5% to 100% B in 2.0 | 250 | 1.5 |
| | XR-ODS | TFA | TFA | | minutes, 100% B for | | |
| | 2.2 μm | | | | 1.1 minutes, 100% to | | |
| | 3.0 × 50 mm | | | | 5% B in 0.2 minutes, | | |
| | | | | | then stop | | |
| B | Gemini-NX | Water/ | ACN | 1 | 5% to 100% B in 2.0 | 200 | 0.75 |
| | 3 μm C18 | 0.04% | | | minutes, 100% B for | | |
| | 110A | Ammonia | | | 1.1 minutes, 100% to | | |
| | | | | | 5% B in 0.1 minutes, | | |
| | | | | | then stop | | |
| C | Shim-pack | Water/0.05% | ACN/0.05% | 1 | 5% to 100% B in 2.0 | 250 | 0.85 |
| | XR-ODS | FA | FA | | minutes, 100% B for | | |
| | 1.6 μm | | | | 1.1 minutes, 100% to | | |
| | 2.0 × 50 mm | | | | 5% B in 0.1 minutes, | | |
| | | | | | then stop | | |
| D | Shim-pack | Water/0.05% | ACN/0.05% | 1 | 5% to 100% B in 2.0 | 250 | 0.95 |
| | XR-ODS | TFA | TFA | | minutes, 100% B for | | |
| | 2.2 μm | | | | 1.1 minutes, 100% to | | |
| | 3.0 × 50 mm | | | | 5% B in 0.1 minutes, | | |
| | | | | | then stop | | |
| E | Waters | Water/0.05% | ACN/0.05% | 0.9 | 5% to 100% B in 2.0 | 250 | 1.5 |
| | Xselect C18 | FA | FA | | minutes, 100% B for | | |
| | 3.5 μm | | | | 1.2 minutes, 100% to | | |
| | 3.0 × 50 mm | | | | 5% B in 0.1 minutes, | | |
| | | | | | then stop | | |
| F | Shim-pack | Water/0.05% | ACN/0.05% | 1 | 5% to 80% B in 3.25 | 200 | 0.95 |
| | XR-ODS | TFA | TFA | | minutes, 80% B for | | |
| | 2.2 μm | | | | 1.35 minutes, 80% to | | |
| | 3.0 × 50 mm | | | | 5% B in 0.3 minutes, | | |
| | | | | | then stop | | |
| G | Shim-pack | Water/0.05% | ACN/0.05% | 1 | 5% to 70% B in 2.50 | 200 | 0.95 |
| | XR-ODS | TFA | TFA | | minutes, 70% B for | | |
| | 2.2 μm | | | | 0.70 minutes, 70% to | | |
| | 3.0 × 50 mm | | | | 5% B in 0.1 minutes, | | |
| | | | | | then stop | | |
| H | Shim-pack | Water/0.05% | ACN/0.05% | 1 | 5% to 100% B in 2.20 | 250 | 0.95 |
| | XR-ODS | TFA | TFA | | minutes, 100% B for | | |
| | 2.2 μm | | | | 1.00 minutes, 100% to | | |
| | 3.0 × 50 mm | | | | 5% B in 0.1 minutes, | | |
| | | | | | then stop | | |
| I | Shim-pack | Water/0.05% | ACN/0.05% | 1 | 5% to 100% B in 1.20 | 250 | 0.95 |
| | XR-ODS | TFA | TFA | | minutes, 100% B for | | |
| | 2.2 μm | | | | 1.00 minutes, 100% to | | |
| | 3.0 × 50 mm | | | | 5% B in 0.1 minutes, | | |
| | | | | | then stop | | |
| J | Shim-pack | Water/0.05% | ACN/0.05% | 1 | 5% to 70% B in 3.20 | 250 | 0.95 |
| | XR-ODS | TFA | TFA | | minutes, 70% B for | | |
| | 2.2 μm | | | | 0.75 minutes, 70% to | | |
| | 3.0 × 50 mm | | | | 5% B in 0.35 minutes, | | |
| | | | | | then stop | | |
| K | Shim-pack | Water/0.05% | ACN/0.05% | 1 | 5% to 80% B in 3.00 | 250 | 1.5 |
| | XR-ODS | TFA | TFA | | minutes, 80% B for 0.8 | | |
| | 2.2 μm | | | | minutes, 80% to 5% B | | |
| | 3.0 × 50 mm | | | | in 0.1 minutes, then | | |
| | | | | | stop | | |
| L | Shim-pack | Water/0.05% | ACN/0.05% | 1 | 5% to 100% B in 3.00 | 250 | 1.5 |
| | XR-ODS | TFA | TFA | | minutes, 100% B for | | |
| | 2.2 μm | | | | 0.8 minutes, 100% to | | |
| | 3.0 × 50 mm | | | | 5% B in 0.1 minutes, | | |
| | | | | | then stop | | |
| M | Shim-pack | Water/0.05% | ACN/0.05% | 1 | 5% to 100% B in 2.20 | 250 | 1.5 |
| | XR-ODS | TFA | TFA | | minutes, 100% B for | | |
| | 2.2 μm | | | | 1.00 minutes, 100% to | | |
| | 3.0 × 50 mm | | | | 5% B in 0.1 minutes, | | |
| | | | | | then stop | | |
| N | Shim-pack | Water/0.05% | ACN/0.05% | 1 | 5% to 80% B in 2.20 | 250 | 1.5 |
| | XR-ODS | TFA | TFA | | minutes, 80% B for 1.0 | | |
| | 2.2 μm | | | | minutes, 80% to 5% B | | |
| | 3.0 × 50 mm | | | | in 0.1 minutes, then | | |
| | | | | | stop | | |
| O | Zorbax | Water/0.05% | ACN/0.05% | 1 | 5% to 70 B in 8.00 | 250 | 1.5 |
| | Eclipse Plus | TFA | TFA | | minutes, 70% B for 2.0 | | |
| | C18 | | | | minutes, then stop | | |
| | 4.6 × 100 mm | | | | | | |
| P | Shim-pack | Water/0.05% | ACN/0.05% | 1 | 5% to 65% B in 3.00 | 250 | 1.5 |
| | XR-ODS | TFA | TFA | | minutes, 65% B for | | |
| | 2.2 μm | | | | 0.80 minutes, 100% to | | |
| | 3.0 × 50 mm | | | | 5% B in 0.1 minutes, | | |
| | | | | | then stop | | |
| Q | Shim-pack | Water/0.05% | ACN/0.05% | 1 | 5% to 60% B in 2.50 | 250 | 0.95 |
| | XR-ODS | TFA | TFA | | minutes, 60% B for 0.7 | | |
| | 2.2 μm | | | | minutes, 60% to 5% B | | |
| | 3.0 × 50 mm | | | | in 0.1 minutes, then | | |
| | | | | | stop | | |
| R | Shim-pack | Water/0.05% | ACN/0.05% | 1 | 5% to 50% B in 2.50 | 250 | 0.95 |
| | XR-ODS | TFA | TFA | | minutes, 50% B for 0.7 | | |
| | 2.2 μm | | | | minutes, 50% to 5% B | | |
| | 3.0 × 50 mm | | | | in 0.1 minutes, then | | |
| | | | | | stop | | |
| S | XBridge | Water/0.05% | ACN/0.05% | 1 | 5% to 95% B in 2.20 | 250 | 0.9 |
| | C18 3.5 μm | TFA | TFA | | minutes, 95% B for | | |
| | 3.0 × 50 mm | | | | 1.00 minutes, 95% to | | |
| | | | | | 5% B in 0.1 minutes, | | |
| | | | | | then stop | | |
| T | Shim-pack | Water/0.05% | ACN/0.05% | 0.7 | 5% to 100% B in 2.0 | 250 | 0.85 |
| | XR-ODS | FA | FA | | minutes, 100% B for | | |
| | 1.6 μm | | | | 1.1 minutes, 100% to | | |
| | 2.0 × 50 mm | | | | 5% B in 0.1 minutes, | | |
| | | | | | then stop | | |
| U | Shim-pack | Water/0.05% | ACN/0.05% | 1 | 5% to 40% B in 2.50 | 250 | 0.95 |
| | XR-ODS | TFA | TFA | | minutes, 40% B for 0.7 | | |
| | 2.2 μm | | | | minutes, 40% to 5% B | | |
| | 3.0 × 50 mm | | | | in 0.1 minutes, then | | |
| | | | | | stop | | |
| V | Shim-pack | Water/0.05% | ACN/0.05% | 1 | 5% to 60% B in 4.20 | 200 | 1.05 |
| | XR-ODS | TFA | TFA | | minutes, 60% B for 1.0 | | |
| | 2.2 μm | | | | minutes, 60% to 5% B | | |
| | 3.0 × 50 mm | | | | in 0.1 minutes, then | | |
| | | | | | stop | | |
| W | Shim-pack | Water/0.05% | ACN/0.05% | 1 | 5% to 100% B in 2.20 | 200 | 0.95 |
| | XR-ODS | TFA | TFA | | minutes, 100% B for | | |
| | 2.2 μm | | | | 1.00 minutes, 100% to | | |
| | 3.0 × 50 mm | | | | 5% B in 0.1 minutes, | | |
| | | | | | then stop | | |
| X | Shim-pack | Water/0.05% | ACN/0.05% | 0.7 | 5% to 100% B in 2.0 | 200 | 0.85 |
| | XR-ODS | FA | FA | | minutes, 100% B for | | |
| | 1.6 μm | | | | 1.1 minutes, 100% to | | |
| | 2.0 × 50 mm | | | | 5% B in 0.1 minutes, | | |
| | | | | | then stop | | |
| Y | Ecliplis Plus | Water/0.05% | ACN | 1 | 5% to 100% B in 2.0 | 250 | 1 |
| | C18 3.5 μm | TFA | | | minutes, 100% B for | | |
| | 4.6 × 50 mm | | | | 1.0 minutes, 100% to | | |
| | | | | | 5% B in 0.1 minutes, | | |
| | | | | | then stop | | |
| Z | Ecliplis Plus | Water/10 mM | ACN/5% | 1 | 5% to 100% B in 2.0 | 250 | 1.1 |
| | C18 3.5 μm | ammonium | water | | minutes, 100% B for | | |
| | 4.6 × 50 mm | carbonate | | | 1.0 minutes, 100% to | | |
| | | | | | 5% B in 0.1 minutes, | | |
| | | | | | then stop | | |
| A1 | Shim-pack | Water/0.05% | ACN | 1 | 5% to 100% B in 2.0 | 250 | 1 |
| | XR-ODS | TFA | | | minutes, 100% B for | | |
| | 2.2 μm | | | | 1.0 minutes, 100% to | | |
| | 3.0 × 50 mm | | | | 5% B in 0.1 minutes, | | |
| | | | | | then stop | | |
| A2 | Ecliplis Plus | Water/10 mM | ACN | 1 | 5% to 100% B in 2.0 | 250 | 0.95 |
| | C18 3.5 μm | ammonium | | | minutes, 100% B for | | |
| | 4.6 × 50 mm | acetate | | | 1.4 minutes, 100% to | | |
| | | | | | 5% B in 0.1 minutes, | | |
| | | | | | then stop | | |
| A3 | Acquity | Water/5 mM | ACN/0.1% | 0.55 | 5% B at 0.01 min up to | | |
| | BEH C18 | ammonium | FA | | 0.4 min, 35% B at 0.8 min, | | |
| | 1.7 μm 2.1 × | acetate/ | | | 55% B at 1.2 min, | | |
| | 50 mm | 0.1% FA | | | 100% B in 1.3 minutes, | | |
| | | | | | at 2.5 min up to 3.30 min, | | |
| | | | | | 5% B at 3.31 min | | |
| | | | | | up to 4.0 min, then | | |
| | | | | | stop | | |
| A4 | Shim-pack | Water/0.05% | ACN/0.05% | 1 | 5% to 30% B in 8.0 | 250 | 1.5 |
| | XR-ODS | TFA | TFA | | minutes, 30% B for 2.0 | | |
| | 3.0 × 50 mm | | | | minutes, then stop | | |
| A5 | Shim-pack | Water/0.05% | ACN/0.05% | 1 | 5% to 100% B in 2.2 | 250 | 1.5 |
| | XR-ODS | TFA | TFA | | minutes, 100% B for | | |
| | 3.0 × 50 mm | | | | 1.0 minutes, 100% to | | |
| | | | | | 5% B in 0.1 minutes, | | |
| | | | | | then stop | | |
| A6 | Atlantis | Water/0.05% | ACN/0.05% | 0.8 | 95% to 60% B in 4.0 | 250 | 1.5 |
| | HILIC | TFA | TFA | | minutes, 60% B for 4.0 | | |
| | 3.0 × 100 mm | | | | minutes, then stop | | |
| A7 | Shim-pack | Water/0.05% | ACN/0.05% | 1 | 5% B for 0.5 minutes, | 250 | 1.5 |
| | XR-ODS | TFA | TFA | | 5% to 75% B at 2.2 | | |
| | 3.0 × 50 mm | | | | minutes, 100% B for | | |
| | | | | | 1.0 minutes, 100% to | | |
| | | | | | 5% B in 0.1 minutes, | | |
| | | | | | then stop | | |
| A8 | Zorbax SB- | Water/0.05% | ACN/0.05% | 1.2 | 5% to 70% B in 10.0 | 250 | 1.05 |
| | C18 | TFA | TFA | | minutes, 70% B for 5.0 | | |
| | 5 μm | | | | minutes, then stop | | |
| | 4.6 × 150 mm | | | | | | |
| A9 | Shim-pack | Water/0.05% | ACN/0.05% | 1 | 5% to 40% B in 4.4 | 250 | 0.95 |
| | XR-ODS | TFA | TFA | | minutes, 40% B for 0.9 | | |
| | 3.0 × 50 mm | | | | minutes, then stop | | |
| A10 | Atlantis T3 | Water/0.05% | ACN/0.05% | 1 | 5% to 50% B in 8.0 | 200 | 1.05 |
| | 3 μm | TFA | TFA | | minutes, 50% B for 2.0 | | |
| | 4.6 × 100 mm | | | | minutes, then stop | | |
| A11 | Shim-pack | Water/0.05% | ACN/0.05% | 1 | 5% B for 0.5 minutes, | 250 | 1.50 |
| | XR-ODS | TFA | TFA | | 5% to 100% B in 1.7 | | |
| | 3.0 × 50 mm | | | | minutes, 100% B for | | |
| | | | | | 1.0 minute, 100% to | | |
| | | | | | 5% B in 0.1 minute, | | |
| | | | | | then stop | | |

Compound structure confirmations were carried out using standard 300 or 400 MHz NMR spectrometers with NOe's conducted whenever necessary.

The following abbreviations are used herein:

| Abbreviation | Meaning |
|---|---|
| ACN | acetonitrile |
| atm. | atmosphere |
| DCM | dichloromethane |
| DHP | dihydropyran |
| DIBAL | diisobutyl aluminum hydride |
| DIEA | diisopropyl ethylamine |
| DMF | dimethyl formamide |
| DMF-DMA | dimethyl formamide dimethyl acetal |
| DMSO | dimethyl sulfoxide |
| dppf | 1,1′-bis(diphenylphosphino)ferrocene |
| EA | ethyl acetate |
| ESI | electrospray ionization |
| EtOH | ethanol |
| FA | formic acid |
| GC | gas chromatography |
| h | hour |
| Hex | hexanes |
| HMDS | hexamethyl disilazide |
| HPLC | high performance liquid chromatography |
| IPA | isopropanol |
| LCMS | liquid chromatography/mass spectrometry |
| MeOH | methanol |
| min | minutes |
| NBS | N-bromo succinimide |
| NCS | N-chloro succinimide |
| NIS | N-iodo succinimide |
| NMR | nuclear magnetic resonance |
| NOe | nuclear Overhauser effect |
| Prep. | preparative |
| PTSA | para-toluene sulfonic acid |
| Rf | retardation factor |
| rt | room temperature |
| RT | retention time |
| sat. | saturated |
| SGC | silica gel chromatography |
| TBAF | tetrabutyl ammonium fluoride |
| TEA | triethylamine |
| TFA | trifluoroacetic acid |
| THF | tetrahydrofuran |
| TLC | thin layer chromatography |
| UPLC | ultra performance liquid chromatography |
| LiHMDS | lithium hexamethyldisilazide |
| TMAD | tetramethyl azocarboxamide |

### Exemplified Synthesis

### Example 1. Preparation of 1-isopropyl-6-(3-((methyl(2-(methylamino)ethyl)amino)methyl) phenyl)-N-(tetrahydro-2H-pyran-4-yl)-1H-pyrazolo[3,4-b]pyridine-4-carboxamide (Compound 233)

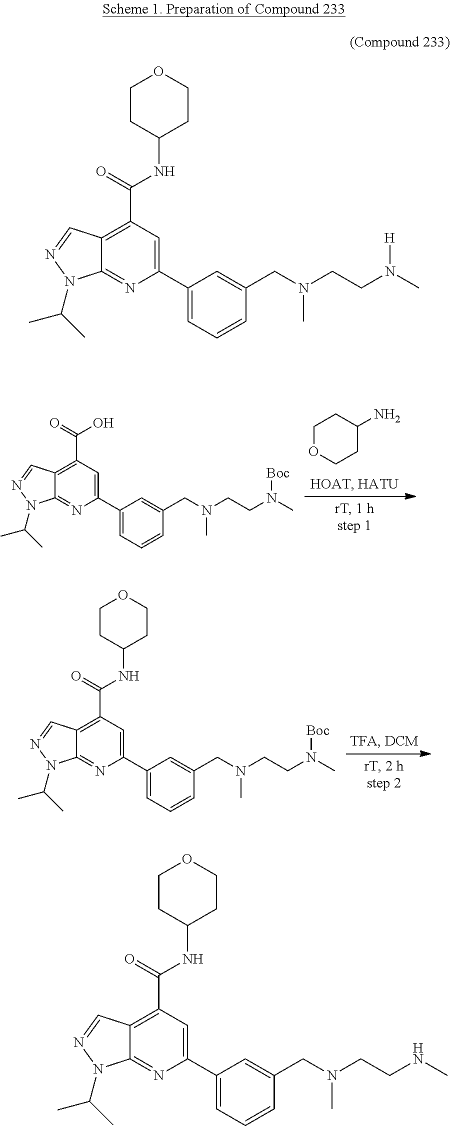

### Step 1: tert-butyl 2-((3-(1-isopropyl-4-(tetrahydro-2H-pyran-4-ylcarbamoyl)-1H-pyrazolo[3,4-b]pyridin-6-yl)benzyl)(methyl)amino)ethyl(methyl)carbamate

To a solution of 6-(3-(((2-(tert-butoxycarbonyl(methyl)amino)ethyl)(methyl)amino) methyl)phenyl)-1-isopropyl-1H-pyrazolo[3,4-b]pyridine-4-carboxylic acid (120 mg, 0.25 mmol) in DCM (2 mL) was added HOAT (60 mg, 0.38 mmol) and HATU (150 mg, 0.38 mmol); the mixture was stirred at room temperature for 5 min. and treated with slow addition of tetrahydro-2H-pyran-4-amine (50 mg, 0.5 mmol). The reaction mixture was further stirred at the same temperature for 1 h, it was then diluted with DCM (10 mL) and washed with water (5 mL×2). The organic layer was dried over Na₂SO₄, filtered and concentrated to render a residue which was purified by chromatographic column on silicagel to give tert-butyl 2-((3-(1-isopropyl-4-(tetrahydro-2H-pyran-4-yl carbamoyl)-1H-pyrazolo[3,4-b]pyridin-6-yl)benzyl)(methyl)amino) ethyl(methyl)carbamate as a white solid (110 mg, 78% yield). ESI-LCMS (m/z): 565 [M+1]⁺

### Step 2: Synthesis of 1-isopropyl-6-(3-((methyl(2-(methylamino)ethyl)amino) methyl)phenyl)-N-(tetrahydro-2H-pyran-4-yl)-1H-pyrazolo[3,4-b]pyridine-4-carboxamide

A solution of tert-Butyl 2-((3-(1-isopropyl-4-(tetrahydro-2H-pyran-4-yl carbamoyl)-1H-pyrazolo[3,4-b]pyridin-6-yl)benzyl)(methyl)amino)ethyl(methyl) carbamate (110 mg, 0.19 mmol) in 3:4 TFA:DCM (3.5 ml) was stirred at room temperature for 2 h. The solvent was then removed in vacuo and the residue was dissolved in MeOH (3 ml), treated with ammonia till pH 7-8 and concentrated. The residue was purified by preparative HPLC to give 1-isopropyl-6-(3-((methyl(2-(methylamino)ethyl)amino) methyl)phenyl)-N-(tetrahydro-2H-pyran-4-yl)-1H-pyrazolo[3,4-b]pyridine-4-carboxamide as a yellow solid (25 mg, 27% yield). ESI-LCMS (m/z): 465.4 found for [M+H]+. ¹HNMR (500 MHz, CD₃OD) δ ppm: 8.32 (s, 1H), 8.24 (s, 1H), 8.18 (d, J=7.5, 1H), 8.07 (s, 1H), 7.52-7.49 (m, 2H), 5.47-5.44 (m, 1H), 4.23 (m, 1H), 4.05-4.02 (m, 2H), 3.69 (s, 2H), 3.60-3.56 (m, 2H), 2.81-2.79 (m, 2H), 2.64-2.62 (m, 2H), 2.41 (s, 3H), 2.31 (s, 3H), 2.02-1.99 (m, 2H), 1.78-1.75 (m, 2H), 1.63-1.62 (m, 6H).

### Example 2. Preparation of N1-(3-(1-isopropyl-3-methyl-4-morpholino-1H-pyrazolo[3,4-b]pyridin-6-yl)benzyl)-N1-methylethane-1,2-diamine (Compound 22)

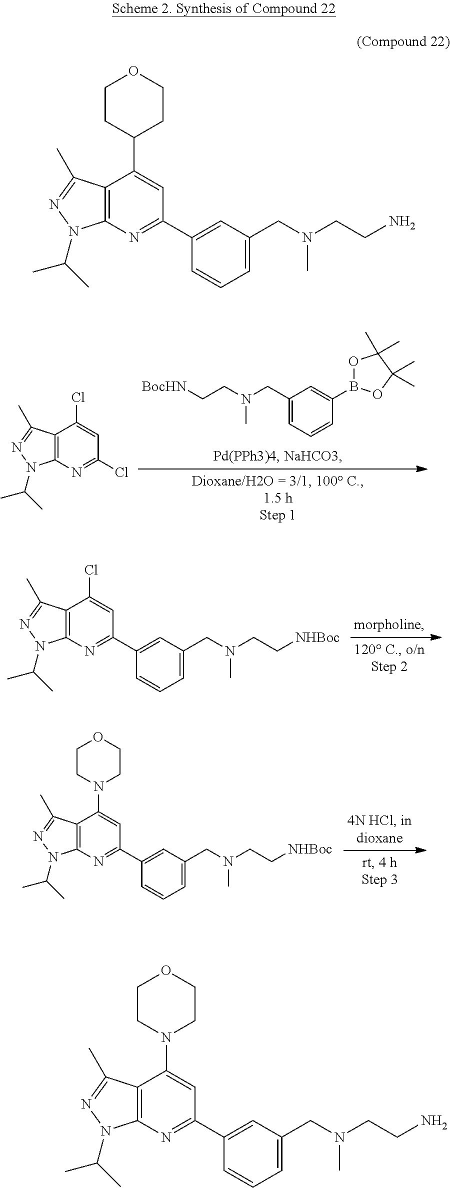 [SMILES: Cc1nn(C(C)C)c2nc(-c3cccc(CN(C)CCN)c3)cc(C3CCOCC3)c12 ; Cc1nn(C(C)C)c2nc(Cl)cc(Cl)c12 ; CN(CCNC(=O)OC(C)(C)C)Cc1cccc(B2OC(C)(C)C(C)(C)O2)c1 ; Cc1nn(C(C)C)c2nc(-c3cccc(CN(C)CCNC(=O)OC(C)(C)C)c3)cc(Cl)c12 ; Cc1nn(C(C)C)c2nc(-c3cccc(CN(C)CCNC(=O)OC(C)(C)C)c3)cc(N3CCOCC3)c12 ; Cc1nn(C(C)C)c2nc(-c3cccc(CN(C)CCN)c3)cc(N3CCOCC3)c12]

### Step 1: Synthesis of tert-butyl 2-((3-(4-chloro-1-isopropyl-3-methyl-1H-pyrazolo[3,4-b]pyridin-6-yl)benzyl)(methyl)amino)ethylcarbamate

A solution of 4,6-dichloro-1-isopropyl-3-methyl-1H-pyrazolo[3,4-b]pyridine (500 mg, 2.05 mmol) in degassed dioxane (9 mL) and water (3 mL) was added tert-butyl 2-(methyl(3-(4,4,5,5-tetramethyl-1,3,2-dioxaborolan-2-yl)benzyl)amino)ethylcarbamate (1 g, 2.56 mmol), NaHCO₃ (344 mg, 4.1 mmol) and Pd(PPh₃)₄ (118 mg, 0.1 mmol) and the mixture was stirred at 100° C. for 1 h under N₂ atmosphere. After being cooled down to room temperature the mixture was concentrated under reduced pressure, diluted with water (10 mL) and the resulting aqueous solution was extracted with DCM (40 mL×3). The combined organic layer were washed with brine (50 mL), dried over Na₂SO₄, filtered and concentrated in vacuo. The residue was purified by chromatographic column on silicagel eluted with 10% EtOAc in petroleum ether to give tert-butyl 2-((3-(4-chloro-1-isopropyl-3-methyl-1H-pyrazolo[3,4-b]pyridin-6-yl)benzyl)(methyl)amino)ethylcarbamate (0.97 g, 100% yield) as a colorless oil. ESI-LCMS (m/z): 472.2 found for [M+1]+.

### Step 2: Synthesis of tert-butyl 2-((3-(1-isopropyl-3-methyl-4-morpholino-1H-pyrazolo[3,4-b]pyridin-6-yl)benzyl)(methyl)amino)ethylcarbamate

A solution of tert-butyl 2-((3-(4-chloro-1-isopropyl-3-methyl-1H-pyrazolo[3,4-b]pyridin-6-yl)benzyl)(methyl)amino)ethylcarbamate (0.99 g, 2.1 mmol) in neat morpholine (10 mL) was stirred at 120° C. for 16 h. The mixture was cooled down to room temperature and concentrated to reneder a residue that was purified by chromatographic column on silicagel eluted with 1% to 10% EtOAc in petroleum ether to give tert-butyl 2-((3-(1-isopropyl-3-methyl-4-morpholino-1H-pyrazolo[3,4-b]pyridin-6-yl)benzyl)(methyl)amino)ethylcarbamate (0.8 g, yield 73%) as a yellow solid. ESI-LCMS (m/z): 523.4 found for [M+1]+.

### Step 3: Synthesis of N1-(3-(1-isopropyl-3-methyl-4-morpholino-1H-pyrazolo[3,4-b]pyridin-6-yl)benzyl)-N1-methylethane-1,2-diamine

A solution of tert-butyl 2-((3-(1-isopropyl-3-methyl-4-morpholino-1H-pyrazolo[3,4-b]pyridin-6-yl)benzyl)(methyl)amino)ethylcarbamate (0.72 g, 1.38 mmol) in 4 N HCl in dioxane (20 mL) was stirred at room temperature for 4 h and then concentrated under reduced pressure. The residue was dissolved in water, the pH adjusted to 8-9 by addition of ammonia and the mixture was extracted with DCM (50 mL×3). The combined organic layer were washed with brine (80 mL), dried over Na₂SO₄, filtered and concentrated. The residue was purified by preparative HPLC to give N1-(3-(1-isopropyl-3-methyl-4-morpholino-1H-pyrazolo[3,4-b]pyridin-6-yl)benzyl)-N1-methylethane-1,2-diamine (33 mg, 81%) as white solid. ESI-LCMS (m/z): 423.4 found for [M+1]⁺¹. HNMR (500 MHz, MeOD) δ ppm: 8.08 (s, 1H), 8.00 (d, J=3 Hz, 1H), 7.47-7.41 (m, 2H), 7.02 (s, 1H), 5.33-5.30 (m, 1H), 3.92 (m, 4H), 3.64 (s, 2H), 3.28 (m, 4H), 2.82-2.80 (m, 2H), 2.64 (s, 3H), 2.56-2.53 (m, 2H), 2.28 (s, 3H), 1.56-1.55 (m, 6H).

### Example 3. Preparation of N1-(3-(1-isopropyl-4-(1-methyl-1H-pyrazol-4-yl)-1H-pyrazolo[3, 4-b]pyridin-6-yl)benzyl)-N1,N2-dimethylethane-1,2-diamine (Compound 49)

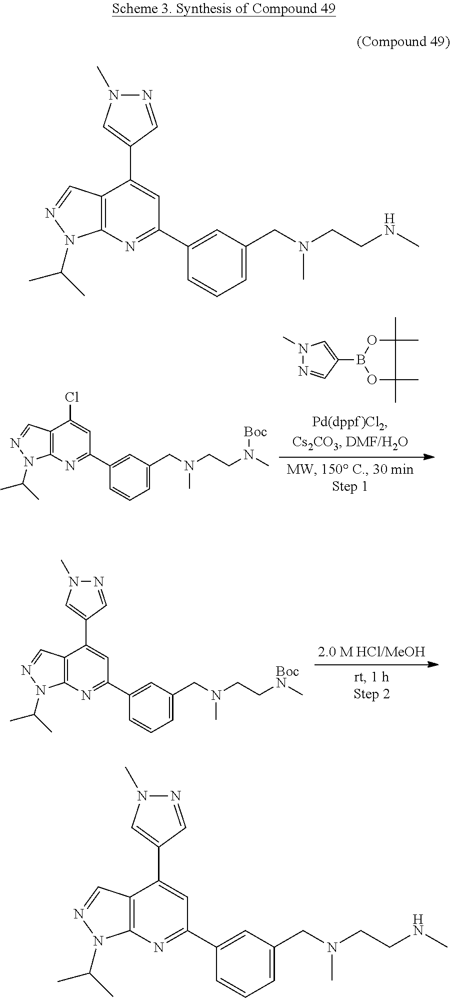

### Step 1: Synthesis of tert-butyl 2-((3-(1-isopropyl-4-(1-methyl-1H-pyrazol-4-yl)-1H-pyrazolo[3,4-b]pyridin-6-yl)benzyl)(methyl)amino)ethyl(methyl)carbamate

To a solution of tert-butyl 2-(((3-(4-chloro-1-isopropyl-1H-pyrazolo[3,4-b]pyridin-6-yl)benzyl)(methyl)amino)ethyl(methyl)carbamate (110 mg, 0.23 mmol) in degassed DMF (2 mL) and H₂O (0.5 ml) was added 1-methyl-4-(4,4,5,5-tetramethyl-1,3,2-dioxaborolan-2-yl)-1H-pyrazole (100 mg, 0.46 mmol), Pd(dppf)Cl₂ (20 mg, 0.023 mmol) and Cs₂CO₃ (225 mg, 0.69 mmol). The reaction vessel was capped, placed in a microwave reactor and irradiated for 30 min at external temperature of 150° C. The reaction mixture was cooled down to room temperature, diluted with dichloromethane (40 mL) and washed with water (50 mL×3). The organic layer was concentrated in vacuo and the residue was purified by preparative TLC on silicagel developed with 50% EtOAc in petroleum ether to give tert-butyl 2-((3-(1-isopropyl-4-(1-methyl-1H-pyrazol-4-yl)-1H-pyrazolo[3,4-b]pyridin-6-yl)benzyl)(methyl)amino)ethyl(methyl)carbamate as a yellow oil (90 mg, 75% yield). ESI-LCMS (m/z): 518.4 found for [M+1]+.

### Step 2: Synthesis of N1-(3-(1-isopropyl-4-(1-methyl-1H-pyrazol-4-yl)-1H-pyrazolo[3,4-b]pyridin-6-yl)benzyl)-N1,N2-dimethylethane-1,2-diamine

A solution of tert-butyl 2-((3-(1-isopropyl-4-(1-methyl-1H-pyrazol-4-yl)-1H-pyrazolo[3,4-b]pyridin-6-yl)benzyl)(methyl)amino)ethyl(methyl)carbamate (90 mg, 0.17 mmol) in 4.0 M HCl in dioxane solution (8 mL) was stirred at room temperature for 1 h; the volatiles were removed in vacuo and the resulting residue was purified by preparative HPLC to give N1-(3-(1-isopropyl-4-(1-methyl-1H-pyrazol-4-yl)-1H-pyrazolo[3,4-b]pyridin-6-yl)benzyl)-N1,N2-dimethylethane-1,2-diamine as a white solid (60 mg, 83% yield). ESI-LCMS (m/z): 418.3 found for [M+H]+. ¹HNMR (500 MHz, CD₃OD) δ ppm: 8.47 (s, 1H), 8.35 (s, 1H), 8.25 (s, 1H), 8.19 (s, 1H), 8.12 (d, J=5 Hz, 1H), 7.86 (s, 1H), 7.51-7.44 (m, 2H), 5.45-5.42 (m, 1H), 4.02 (s, 3H), 3.67 (s, 2H), 2.82-2.80 (m, 2H), 2.64-2.62 (m, 2H), 2.41 (s, 3H), 2.31 (s, 3H), 1.63-1.61 (d, 6H).

### Example 4. Preparation of N1-(3-(1-isopropyl-4-morpholino-1H-pyrazolo[3,4-b]pyridin-6-yl)benzyl)-N1-methylethane-1,2-diamine (Compound 61)

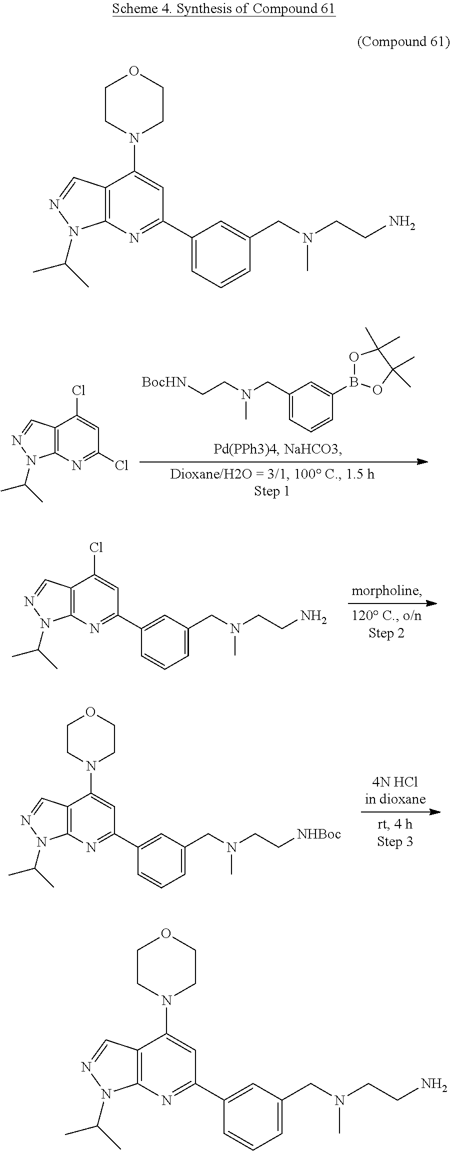 [SMILES: CC(C)n1ncc2c(N3CCOCC3)cc(-c3cccc(CN(C)CCN)c3)nc21 ; CC(C)n1ncc2c(Cl)cc(Cl)nc21 ; CN(CCNC(=O)OC(C)(C)C)Cc1cccc(B2OC(C)(C)C(C)(C)O2)c1 ; CC(C)n1ncc2c(Cl)cc(-c3cccc(CN(C)CCN)c3)nc21 ; CC(C)n1ncc2c(N3CCOCC3)cc(-c3cccc(CN(C)CCNC(=O)OC(C)(C)C)c3)nc21 ; CC(C)n1ncc2c(N3CCOCC3)cc(-c3cccc(CN(C)CCN)c3)nc21]

### Step 1: Synthesis of tert-butyl 2-((3-(4-chloro-1-isopropyl-1H-pyrazolo[3,4-b]pyridin-6-yl)benzyl)(methyl)amino) ethylcarbamate

To a solution of 4,6-dichloro-1-isopropyl-1H-pyrazolo[3,4-b]pyridine (1 g, 4.37 mmol) and tert-butyl 2-(methyl(3-(4,4,5,5-tetramethyl-1,3,2-dioxaborolan-2-yl)benzyl)amino)ethylcarbamate (2 g, 5.13 mmol) in degassed dioxane (15 mL) and water (5 mL) was added NaHCO₃ (0.73 g, 8.7 mmol) and Pd(PPh₃)₄ (252 mg, 0.22 mmol) and the mixture was stirred at 100° C. for 1.5 h under N₂ atmosphere. After being cooled down to room temperature, the reaction mixture was concentrated under reduced pressure, diluted with water (10 mL) and the resulting aqueous solution was extracted with DCM (50 mL×3). The combined organic layer were washed with brine (50 mL), dried over Na₂SO₄, filtered and concentrated in vacuo. The residue was purified by chromatographic column on silicagel eluted with 1% to 10% EtOAc in petroleum ether to give tert-butyl 2-((3-(4-chloro-1-isopropyl-1H-pyrazolo[3,4-b]pyridin-6-yl)benzyl)(methyl)amino) ethylcarbamate as a colorless oil. (1.9 g, 95% yield). ESI-LCMS (m/z): 458.2 found for [M+1]+.

### Step 2: Synthesis of tert-butyl 2-((3-(1-isopropyl-4-morpholino-1H-pyrazolo[3,4-b]pyridin-6-yl)benzyl)(methyl)amino)ethylcarbamate

A solution of tert-butyl 2-((3-(4-chloro-1-isopropyl-1H-pyrazolo[3,4-b]pyridin-6-yl)benzyl)(methyl)amino) ethylcarbamate (1.9 g, 4.13 mmol) in neat morpholine (20 mL) was stirred at 120° C. for 16 h. The mixture was cooled down to room temperature, concentrated under vacuo and the resulting residue was purified by chromatographic column on silicagel eluted with 0% to 10% EtOAc in petroleum ether to give tert-butyl 2-((3-(1-isopropyl-4-morpholino-1H-pyrazolo[3,4-b]pyridin-6-yl)benzyl)(methyl)amino)ethylcarbamate (1.7 g, 81% yield) as a yellow solid. ESI-LCMS (m/z): 509.4 found for [M+1]+.

### Step 3: Synthesis of N1-(3-(1-isopropyl-4-morpholino-1H-pyrazolo[3,4-b]pyridin-6-yl)benzyl)-N1-methylethane-1,2-diamine

A solution of tert-butyl 2-((3-(1-isopropyl-4-morpholino-1H-pyrazolo[3,4-b]pyridin-6-yl)benzyl)(methyl)amino)ethylcarbamate (1.7 g, 3.34 mmol) in 4N HCl in dioxane (35 mL) was stirred at room temperature for 4 h. The reaction mixture was then concentrated under reduced pressure and the residue was dissolved in water and treated with ammonia till pH 8˜9. This solution was extracted with DCM (50 mL×3), the combined organic layer were washed with brine (80 mL), dried over Na₂SO₄, filtered and concentrated to give N1-(3-(1-isopropyl-4-morpholino-1H-pyrazolo[3,4-b]pyridin-6-yl)benzyl)-N1-methylethane-1,2-diamine (1.1 g, 81% yield) as yellow solid. ESI-LCMS (m/z): 409.2 found for [M+1]+. ¹HNMR (400 MHz, MeOD) δ ppm: 8.17 (s, 1H), 8.06 (s, 1H), 7.99-7.98 (m, 2H), 7.48-7.42 (m, 2H), 5.38-5.35 (m, 1H), 3.93-3.91 (m, 4H), 3.72-3.66 (m, 6H), 2.84-2.81 (m, 2H), 2.58-2.55 (m, 2H), 2.30 (s, 3H), 1.58-1.55 (m, 6H).

### Example 5. Preparation of N1-(3-(1-cyclopropyl-4-(methyl(tetrahydro-2H-pyran-4-yl)amino)-1H-pyrazolo[3,4-d]pyrimidin-6-yl)benzyl)-N1,N2-dimethylethane-1,2-diamine (Compound 109)

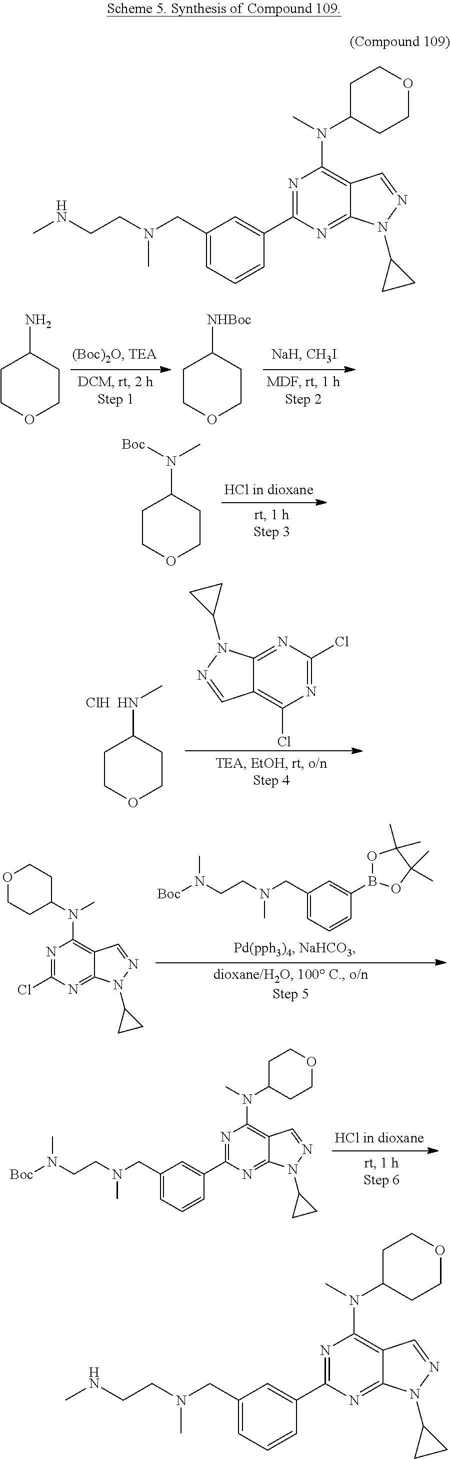

### Step 1: Synthesis of tert-butyl tetrahydro-2H-pyran-4-ylcarbamate

To a solution of tetrahydro-2H-pyran-4-amine (2 g, 19.8 mmol) in DCM (24 mL) was added di-tert-butyl dicarbonate (6.5 g, 29.8 mmol) and triethylamine (4 g, 39.6 mmol). The reaction mixture was stirred at room temperature for 2 h.; diluted with diethyl ether (150 mL) and then washed with saturated NH₄Cl solution (70 mL×2) and brine (70 mL×2). The organic layer was dried over anhydrous MgSO₄, filtered, and concentrated in vacuo to give tert-butyl tetrahydro-2H-pyran-4-ylcarbamate as a white solid (5.1 g, crude), which was used into next step without further purification. ¹HNMR (500 MHz, CD₃OD) δ ppm: 3.94-3.91 (m, 2H), 3.56-3.53 (m, 1H), 3.48-3.43 (m, 2H), 1.83-1.80 (m, 2H), 1.52-1.51 (m, 2H), 1.45 (s, 9H).

### Step 2: Synthesis of tert-butyl methyl (tetrahydro-2H-pyran-4-yl)carbamate

A solution of tert-butyl tetrahydro-2H-pyran-4-ylcarbamate (1 g, 5 mmol) in DMF (20 mL) stirred at 0° C. under N₂ (balloon) was treated sodium hydride (60% in oil, 1 g, 25 mmol), the suspension was stirred for 5 min and then treated with slow addition of neat CH₃I (1.4 g, 10 mmol, 0.61 ml) at 0° C. The cooling bath was removed and the reaction mixture was further stirred at room temperature for 1 h.; diluted with EtOAc (150 mL), and washed with water (100 ml×4). The organic layer was concentrated in vacuo to give tert-butyl methyl(tetrahydro-2H-pyran-4-yl) carbamate as a brown oil (1.6 g, crude), which was used into next step without further purification. ¹HNMR (500 MHz, CDCl₃) δ ppm: 4.02-3.99 (m, 2H), 3.46-3.41 (m, 2H), 2.74 (s, 3H), 1.76-1.71 (m, 1H), 1.59-1.56 (m, 2H), 1.46 (S, 9H), 1.33-1.25 (m, 1H).

### Step 3: Synthesis of N-methyl-tetrahydro-2H-pyran-4-amine hydrochloride

A solution of tert-butyl methyl (tetrahydro-2H-pyran-4-yl)carbamate (1.6 g) in 4N HCl in dioxane (33 mL) was stirred at room temperature for 1 h.; the volatiles were removed in rotary evaporator and the residue was dissolved in water (50 mL) and treated with ammonia till pH 8˜9. This solution was extracted with ether (50 ml×5), the organic layers were combined, and concentrated in vacuo to give N-methyl-tetrahydro-2H-pyran-4-amine hydrochloride as a white solid (430 mg, 57% yield for two steps); which was used as such for the next step without further purification. ¹HNMR (500 MHz, DMSO) δ ppm: 3.92-3.89 (m, 2H), 3.31-3.26 (m, 2H), 3.16-3.15 (m, 1H), 2.50-2.48 (s, 3H), 1.95-1.92 (m, 2H), 1.60-1.56 (m, 2H).

### Step 4: Synthesis of 6-chloro-1-cyclopropyl-N-methyl-N-(tetrahydro-2H-pyran-4-yl)-1H-pyrazolo[3,4-d]pyrimidin-4-amine

To a solution of 4,6-dichloro-1-cyclopropyl-1H-pyrazolo[3,4-d]pyrimidine (335 mg, 1.47 mmol) in EtOH (8 mL) was added N-methyl-tetrahydro-2H-pyran-4-amine hydrochloride (344 mg, 2.27 mmol) and triethylamine (312 mg, 3.08 mmol). The reaction mixture was stirred at room temperature for 16 h. and then the mixture was concentrated in vacuo. The residue was dissolved in EtOAc (70 mL) and washed with water (50 mL×3). The organic layer was concentrated in vacuo to give 6-chloro-1-cyclopropyl-N-methyl-N-(tetrahydro-2H-pyran-4-yl)-1H-pyrazolo[3,4-d]pyrimidin-4-amine as a light yellow solid (440 mg, 98% yield), which was used directly for the next step without further purification. ESI-LCMS: 308.1 found for [M+1]⁺.

### Step 5: Synthesis of tert-butyl 2-((3-(1-cyclopropyl-4-(methyl(tetrahydro-2H-pyran-4-yl)amino)-1H-pyrazolo[3,4-d]pyrimidin-6-yl)benzyl)(methyl)amino) ethyl(methyl)carbamate

To a solution of 6-chloro-1-cyclopropyl-N-methyl-N-(tetrahydro-2H-pyran-4-yl)-1H-pyrazolo[3,4-d]pyrimidin-4-amine (220 mg, 0.72 mmol) in degassed 1,4-dioxane (9 mL) and H₂O (3 ml) was added tert-butyl methyl(2-(methyl(3-(4,4,5,5-tetramethyl-1,3,2-dioxaborolan-2-yl)benzyl)amino)ethyl) carbamate (350 mg, 0.86 mmol), NaHCO₃ (182 mg, 2.16 mmol) and Pd(PPh₃)₄ (84 mg, 0.072 mmol) and the mixture was stirred at 100° C. for 16 h under N₂ atmosphere. The reaction mixture was then concentrated in vacuo, diluted with water (80 mL) and the resulting mixture was extracted with EtOAc (50 mL×3). The organic layers were combined and concentrated in vacuo. The residue was purified by chromatographic column on silicagel eluted with 15% petroleum ether in ether to give tert-butyl 2-((3-(1-cyclopropyl-4-(methyl(tetrahydro-2H-pyran-4-yl)amino)-1H-pyrazolo[3,4-d]pyrimidin-6-yl)benzyl)(methyl)amino)ethyl(methyl)carbamate as a light yellow solid (300 mg, 76% yield). ESI-LCMS: 550.3 found for [M+1]⁺.

### Step 6: Synthesis of N1-(3-(1-cyclopropyl-4-(methyl(tetrahydro-2H-pyran-4-yl)amino)-1H-pyrazolo[3,4-d]pyrimidin-6-yl)benzyl)-N1,N2-dimethylethane-1,2-diamine

A solution of tert-butyl 2-((3-(1-cyclopropyl-4-(methyl(tetrahydro-2H-pyran-4-yl)amino)-1H-pyrazolo[3,4-d]pyrimidin-6-yl)benzyl)(methyl)amino)ethyl(methyl) carbamate (300 mg) in 4.0 M HCl solution in dioxane (10 mL) was stirred at room temperature for 1 h.; the solvent was then removed in rotary evaporator and the resulting residue was purified by preparative HPLC to give N1-(3-(1-cyclopropyl-4-(methyl(tetrahydro-2H-pyran-4-yl)amino)-1H-pyrazolo[3,4-d]pyrimidin-6-yl)benzyl)-N1,N2-dimethylethane-1,2-diamine as a light yellow solid (33 mg, 13% yield). ESI-LCMS (m/z): 450.4 found for [M+H]⁺. ¹HNMR (500 MHz, CD₃OD) δ ppm: 8.47 (s, 1H), 8.39 (t, 1H), 8.03 (s, 1H), 7.46 (d, J=4.5 Hz, 2H), 5.35 (m, 1H), 4.13 (dd, J=4.5 and 4.0 Hz, 2H), 3.93 (m, 1H), 3.69-3.65 (m, 4H), 3.33 (s, 3H), 2.73-2.70 (t, 2H), 2.62-2.59 (t, 2H), 2.35 (s, 3H), 2.30 (s, 3H), 2.09-1.97 (m, 2H), 1.80-1.77 (m, 2H), 1.32-1.31 (m, 2H), 1.17-1.16 (m, 2H).

### Example 6. Preparation of N1-(3-(1-tert-butyl-4-(tetrahydro-2H-pyran-4-ylamino)-1H-pyrazolo[3,4-d]pyrimidin-6-yl)benzyl)-N1,N2-dimethylethane-1,2-diamine (Compound 27)

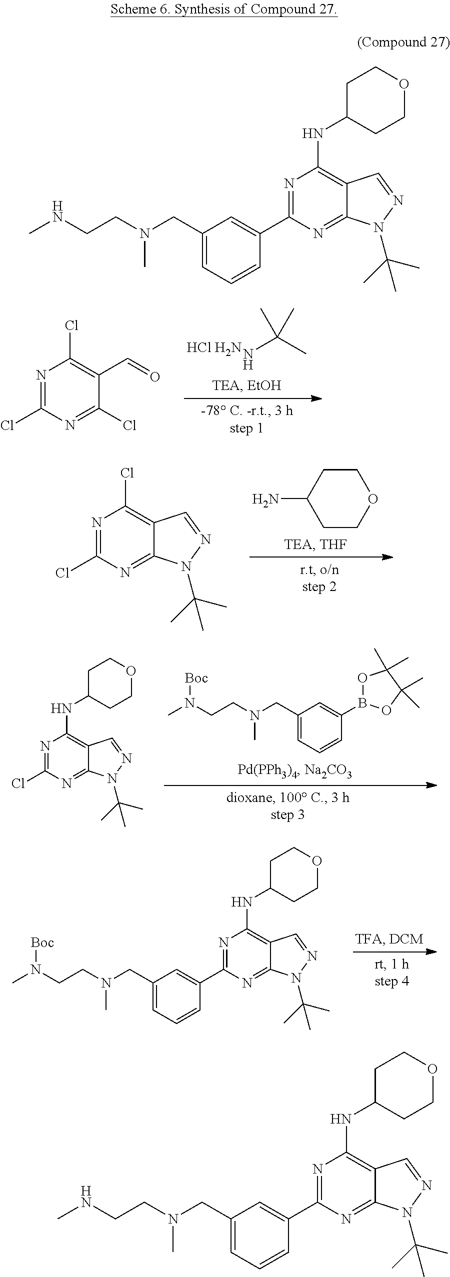

### Step 1: Synthesis of 1-tert-butyl-4,6-dichloro-1H-pyrazolo[3,4-d]pyrimidine

To a solution of 2,4,6-trichloropyrimidine-5-carbaldehyde (5.0 g, 23.8 mmol) in EtOH (100 mL), stirred at −78° C. was added Et₃N (7.18 g, 71.1 mmol) followed by tert-butylhydrazine hydrochloride (2.97 g, 23.8 mmol) and the mixture was further stirred at the same temperature for 1 h.; the cooling bath was then removed and the mixture allowed to warm to room temperature for over 2 h. The reaction mixture was concentrated and the resulting residue was purified by chromatographic column on silicagel eluted with 15% EtOAc in petroleum ether to give 1-tert-butyl-4,6-dichloro-1H-pyrazolo[3,4-d]pyrimidine (5 g, 86% yield) as white solid. ESI-LCMS (m/z): 245.1 found for [M+H]⁺.

### Step 2: Synthesis of 1-tert-butyl-6-chloro-N-(tetrahydro-2H-pyran-4-yl)-1H-pyrazolo[3,4-d]pyrimidin-4-amine

A solution of 1-tert-butyl-4,6-dichloro-1H-pyrazolo[3,4-d]pyrimidine (1 g, 4.08 mmol); Et₃N (1.442 g, 14.28 mmol) and tetrahydro-2H-pyran-4-amine (618 mg, 6.12 mmol) in THF (40 mL) was stirred at room temperature for 2 h; the mixture was then concentrated and the residue was purified by chromatographic column on silicagel eluted with 33% EtOAc in petroleum ether to give 1-tert-butyl-6-chloro-N-(tetrahydro-2H-pyran-4-yl)-1H-pyrazolo[3,4-d]pyrimidin-4-amine (1 g, 78% yield) as a white solid. ESI-LCMS (m/z): 310.1 found for [M+H]⁺.

### Step 3: tert-butyl 2-((3-(1-tert-butyl-4-(tetrahydro-2H-pyran-4-ylamino)-1H-pyrazolo[3,4-d]pyrimidin-6-yl)benzyl)(methyl)amino)ethyl(methyl)carbamate

A solution of 1-tert-butyl-6-chloro-N-(tetrahydro-2H-pyran-4-yl)-1H-pyrazolo[3,4-d]pyrimidin-4-amine (310 mg, 1 mmol) in degassed dioxane (5 mL) and water (1 mL) was treated with tert-butyl methyl(2-(methyl(3-(4,4,5,5-tetramethyl-1,3,2-dioxaborolan-2-yl)benzyl)amino)ethyl) carbamate (485 mg, 1.2 mmol), Pd(PPh₃)₄ (115 mg, 0.1 mmol), and Na₂CO₃ (318 mg, 3 mmol) and the reaction mixture was stirred at 100° C. for 6 h. under N₂ atmosphere. The reaction mixture was then cooled down to room temperature, diluted with water (100 mL) and the resulting mixture was extracted with EtOAc (50 mL×3). The combined organic layers were dried over Na₂SO₄, concentrated and the resulting residue was purified by chromatographic column on silicagel eluted with 25% petroleum ether in EtOAc to give tert-butyl 2-((3-(1-tert-butyl-4-(tetrahydro-2H-pyran-4-ylamino)-1H-pyrazolo[3,4-d]pyrimidin-6-yl)benzyl)(methyl)amino) ethyl(methyl)carbamate (496 mg, 90% yield) as a white solid. ESI-LCMS (m/z): 552.3 found for [M+H]⁺.

### Step 4: Synthesis of N1-(3-(1-tert-butyl-4-(tetrahydro-2H-pyran-4-ylamino)-1H-pyrazolo[3,4-d]pyrimidin-6-yl)benzyl)-N1,N2-dimethylethane-1,2-diamine

A solution of tert-butyl 2-((3-(1-tert-butyl-4-(tetrahydro-2H-pyran-4-ylamino)-1H-pyrazolo[3,4-d]pyrimidin-6-yl)benzyl)(methyl)amino)ethyl(methyl)carbamate (496 mg, 0.9 mmol) in 1:1 DCM:TFA (20 mL) was stirred at room temperature for 16 h.; the mixture was then concentrated under vacuum, the resulting residue was dissolved in MeOH (10 ml) and the solution was adjusted to pH 7-8 with ammonia. The mixture was concentrated and the residue was purified by preparative HPLC to give N1-(3-(1-tert-butyl-4-(tetrahydro-2H-pyran-4-ylamino)-1H-pyrazolo[3,4-d]pyrimidin-6-yl)benzyl)-N1,N2-dimethylethane-1,2-diamine (325 mg, 80% yield) as a white solid. ESI-LCMS (m/z): 452.4 found for [M+H]+. ¹HNMR (400 MHz, CDCl₃) δ ppm: 8.41-8.39 (m, 2H), 7.99 (s, 1H), 7.52-7.45 (m, 2H), 4.55-4.54 (m, 1H), 4.05 (d, J=9.6, 2H), 3.73 (s, 2H), 3.66-3.62 (m, 2H), 3.15-3.13 (m, 2H), 2.74 (s, 2H), 2.64 (s, 3H), 2.35 (s, 3H); 2.13-2.10 (m, 2H), 1.84 (s, 9H), 1.78-1.70 (m, 2H).

### Example 7. Preparation of N1-(3-(3-isopropyl-7-morpholinopyrazolo[1,5-a]pyrimidin-5-yl)benzyl)-N1,N2-dimethylethane-1,2-diamine formate (Compound 62)

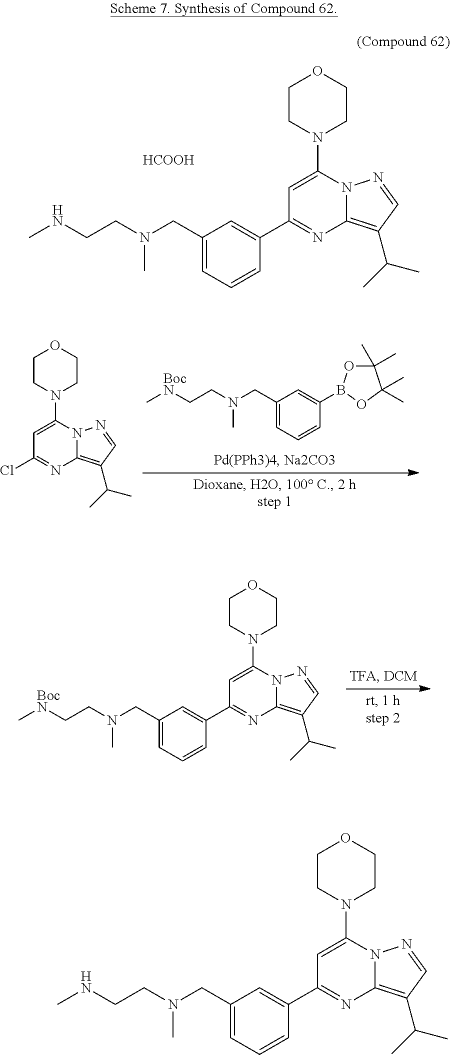

### Step 1: tert-butyl 2-((3-(3-isopropyl-7-morpholinopyrazolo[1,5-a]pyrimidin-5-yl)benzyl)(methyl)amino)ethyl(methyl)carbamate

A solution of 4-(5-chloro-3-isopropylpyrazolo[1,5-a]pyrimidin-7-yl)morpholine (150 mg, 0.54 mmol) in degassed dioxane (12 mL) and H₂O (4 mL) was treated with Na₂CO₃ (172 mg, 1.6 mmol), Pd(PPh₃)₄ (180 mg, 0.16 mmol) and tert-butyl methyl(2-(methyl(3-(4,4,5,5-tetramethyl-1,3,2-dioxaborolan-2-yl)benzyl)amino)ethyl)carbamate (324 mg, 0.8 mmol). The system was purged with N₂ stream and stirred at 100° C. for 2 h, then cooled down to room temperature, concentrated and diluted with water (10 mL). The mixture was extracted with EtOAc (20 mL×2), the combined organic layers were dried over Na₂SO₄, filtered and concentrate to render a residue which was purified by chromatographic column on silicagel eluted with 20% EtOAc in petroleum ether to give tert-butyl 2-((3-(3-isopropyl-7-morpholinopyrazolo[1,5-a]pyrimidin-5-yl)benzyl)(methyl)amino)ethyl(methyl)carbamate (142 mg, 50% yield) as a white solid. ESI-LCMS (m/z): 523.4 [M+1]⁺.

### Step 2: Synthesis of N1-(3-(3-isopropyl-7-morpholinopyrazolo[1,5-a]pyrimidin-5-yl)benzyl)-N1,N2-dimethylethane-1,2-diamine formate

A solution of tert-butyl 2-((3-(3-isopropyl-7-morpholinopyrazolo[1,5-a]pyrimidin-5-yl)benzyl)(methyl)amino)ethyl(methyl)carbamate (142 mg, 0.27 mmol) in 50% TFA in DCM (4 mL) was stirred at room temperature for 1 h. The solvent was then removed in vacuo, the resulting residue was dissolved in MeOH (3 ml) and the solution pH was adjusted 7-8 with ammonia. After removal of volatiles under vacuo, the resulting residue was purified by preparative HPLC to give N1-(3-(3-isopropyl-7-morpholinopyrazolo[1,5-a]pyrimidin-5-yl)benzyl)-N1,N2-dimethylethane-1,2-diamine formate as a white solid (62 mg, 35% yield). ESI-LCMS (m/z): 423.3 found for [M+H]+. ¹HNMR (500 MHz, CD₃OD) δ ppm: 8.42 (bs, 1H), 8.12 (s, 1H), 8.08-8.05 (m, 1H), 7.99 (s, 1H), 7.56-7.50 (m, 2H), 6.73 (s, 1H), 3.96-3.95 (m, 4H), 3.80-3.75 (m, 4H), 3.73 (s, 2H), 3.43-3.36 (m, 1H), 3.18-3.15 (m, 2H), 2.77-2.74 (m, 2H), 2.67 (s, 3H), 2.35 (s, 3H), 1.45-1.42 (m, 6H).

### Biological Methods

### PRMT1 Biochemical Assay

General Materials.

S-adenosylmethionine (SAM), S-adenosylhomocysteine (SAH), bicine, Tween20, dimethylsulfoxide (DMSO), bovine skin gelatin (BSG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 36-50 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-RLARRGGVKRISGLI-amide (SEQ ID NO.: 1).

Molecular Biology:

Full-length human PRMT1 isoform 1 (NM_001536.5) transcript clone was amplified from an HEK 293 cDNA library, incorporating flanking 5′ sequence encoding a FLAG tag (DYKDDDDK) (SEQ ID NO.: 2) fused directly to Met 1 of PRMT1. The amplified gene was subcloned into pFastBacI (Life Technologies) modified to encode an N-terminal GST tag and a TEV cleavage sequence (MSPILGYWKIKGLVQPTRLLLEYLEEKYEEHLYERDEGDKWRNKKFELGLEFPNLP YYIDGDVKLTQSMAIIRYIADKHNMLGGCPKERAEISMLEGAVLDIRYGVSRIAYSK DFETLKVDFLSKLPEMLKMFEDRLCHKTYLNGDHVTHPDFMLYDALDVVLYMDPM CLDAFPKLVCFKKRIEAIPQIDKYLKSSKYIAWPLQGWQATFGGGDHPPKSDENLYF QGGNS)(SEQ ID NO.: 3) fused to Asp of the Flag tag of PRMT1.

Protein Expression.

Recombinant baculovirus were generated according to Bac-to-Bac kit instructions (Life Technologies). Protein over-expression was accomplished by infecting exponentially growing High Five insect cell culture at 1.5×10⁶ cell/ml with 1:100 ratio of virus. Infections were carried out at 27° C. for 48 hours, harvested by centrifugation, and stored at −80° C. for purification.

Protein Purification.

Expressed full-length human GST-tagged PRMT1 protein was purified from cell paste by glutathione sepharose affinity chromatography after equilibration of the resin with 50 mM phosphate buffer, 200 mM NaCl, 5% glycerol, 5 mM β-mercaptoethanol, pH7.8 (Buffer A). GST-tagged PRMT1 was eluted with 50 mM Tris, 2 mM glutathione, pH 7.8, dialysed in buffer A and concentrated to 1 mg/mL. The purity of recovered protein was 73%. Reference: Wasilko, D. J. and S. E. Lee: “TIPS: titerless infected-cells preservation and scale-up” Bioprocess J., 5 (2006), pp. 29-32.

Predicted Translations:

GST-tagged PRMT1
(SEQ ID NO.: 4)
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDENLYFQGGNSDYKDDDDKMAAAEAANCIME
NFVATLANGMSLQPPLEEVSCGQAESSEKPNAEDMTSKDYYFDSYAHFGI
HEEMLKDEVRTLTYRNSMFHNRHLFKDKVVLDVGSGTGILCMFAAKAGAR
KVIGIECSSISDYAVKIVKANKLDHVVTIIKGKVEEVELPVEKVDIIISE
WMGYCLFYESMLNTVLYARDKWLAPDGLIFPDRATLYVTAIEDRQYKDYK
IHWWENVYGFDMSCIKDVAIKEPLVDVVDPKQLVTNACLIKEVDIYTVKV
EDLTFTSPFCLQVKRNDYVHALVAYFNIEFTRCHKRTGFSTSPESPYTHW
KQTVFYMEDYLTVKTGEEIFGTIGMRPNAKNNRDLDFTIDLDFKGQLCEL
SCSTDYRMR

General Procedure for PRMT1 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT1, was added to columns 11, 12, 23, 24, rows I-P for the minimum signal control. A cocktail (40 ul) containing the PRMT1 enzyme was added by Multidrop Combi (Thermo-Fisher). The compounds were allowed to incubate with PRMT1 for 30 min at room temperature, then a cocktail (10 ul) containing SAM and peptide was added to initiate the reaction (final volume=51 ul). The final concentrations of the components were as follows: PRMT1 was 0.5 nM, ³H-SAM was 200 nM, non-radiolabeled SAM was 1.5 uM, peptide was 20 nM, SAH in the minimum signal control wells was 1 mM, and the DMSO concentration was 2%. The assays were stopped by the addition of non-radiolabeled SAM (10 ul) to a final concentration of 300 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT6 Biochemical Assay

General Materials.

S-adenosylmethionine (SAM), S-adenosylhomocysteine (SAH), bicine, Tween20, dimethylsulfoxide (DMSO), bovine skin gelatin (BSG), sodium butyrate and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 36-50 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-RLARRGGVKRISGLI-amide and contained a monomethylated lysine at position 44 (SEQ ID NO.: 5).

Molecular Biology:

Full-length human PRMT6 (NM_018137.2) transcript clone was amplified from an HEK 293 cDNA library, incorporating a flanking 5′ sequence encoding a FLAG tag (MDYKDDDDK) (SEQ ID NO.: 6) fused directly to Ser 2 of PRMT6 and a 3′ sequence encoding a hexa His sequence (HHHHHH) (SEQ ID NO.: 17) fused directly to Asp 375. The amplified gene was subcloned into pFastBacMam (Viva Biotech).

Protein Expression.

Recombinant baculovirus were generated according to Bac-to-Bac kit instructions (Life Technologies). Protein over-expression was accomplished by infecting exponentially growing HEK 293F cell culture at 1.3×10⁶ cell/ml with virus (MOI=10) in the presence of 8 mM sodium butyrate. Infections were carried out at 37° C. for 48 hours, harvested by centrifugation, and stored at −80° C. for purification.

Protein Purification.

Expressed full-length human Flag- and His-tagged PRMT6 protein was purified from cell paste by NiNTA agarose affinity chromatography after equilibration of the resin with buffer containing 50 mM Tris, 300 mM NaCl, 10% glycerol, pH 7.8 (Buffer Ni-A). Column was washed with 20 mM imidazole in the same buffer and Flag-PRMT6-His was eluted with 150 mM imidazole. Pooled fractions were dialysed against buffer Ni-A and further purified by anti-flag M2 affinity chromatography. Flag-PRMT6-His was eluted with 200 ug/ml FLAG peptide in the same buffer. Pooled fractions were dialysed in 20 mM Tris, 150 mM NaCl, 10% glycerol and 5 mM β-mercaptoethanol, pH 7.8. The purity of recovered protein was 95%.

Predicted Translations:

Flag-PRMT6-His
(SEQ ID NO.: 7)
MDYKDDDDKSQPKKRKLESGGGGEGGEGTEEEDGAEREAALERPRRTKRE
RDQLYYECYSDVSVHEEMIADRVRTDAYRLGILRNWAALRGKTVLDVGAG
TGILSIFCAQAGARRVYAVEASAIWQQAREVVRFNGLEDRVHVLPGPVET
VELPEQVDAIVSEWMGYGLLHESMLSSVLHARTKWLKEGGLLLPASAELF
IAPISDQMLEWRLGFWSQVKQHYGVDMSCLEGFATRCLMGHSEIVVQGLS
GEDVLARPQRFAQLELSRAGLEQELEAGVGGRFRCSCYGSAPMHGFAIWF
QVTFPGGESEKPLVLSTSPFHPATHWKQALLYLNEPVQVEQDTDVSGEIT
LLPSRDNPRRLRVLLRYKVGDQEEKTKDFAMEDHHHHHH

General Procedure for PRMT6 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT6, was added to columns 11, 12, 23, 24, rows I-P for the minimum signal control. A cocktail (40 ul) containing the PRMT6 enzyme was added by Multidrop Combi (Thermo-Fisher). The compounds were allowed to incubate with PRMT6 for 30 min at room temperature, then a cocktail (10 ul) containing SAM and peptide was added to initiate the reaction (final volume=51 ul). The final concentrations of the components were as follows: PRMT6 was 1 nM, ³H-SAM was 200 nM, non-radiolabeled SAM was 250 nM, peptide was 75 nM, SAH in the minimum signal control wells was 1 mM, and the DMSO concentration was 2%. The assays were stopped by the addition of non-radiolabeled SAM (10 ul) to a final concentration of 400 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT8 Biochemical Assay

General Materials.

S-adenosylmethionine (SAM), S-adenosylhomocysteine (SAH), bicine, Tween20, dimethylsulfoxide (DMSO), bovine skin gelatin (BSG), isopropyl-β-D-thiogalactopyranoside (IPTG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 31-45 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-KPAIRRLARRGGVKR-amide (SEQ ID NO.: 8).

Molecular Biology:

Full-length human PRMT8 (NM_019854.4) isoform 1 transcript clone was amplified from an HEK 293 cDNA library and subcloned into pGEX-4T-1 (GE Life Sciences). The resulting construct encodes an N-terminal GST tag and a thrombin cleavage sequence (MSPILGYWKIKGLVQPTRLLLEYLEEKYEEHLYERDEGDKWRNKKFELGLEFPNLP YYIDGDVKLTQSMAIIRYIADKHNMLGGCPKERAEISMLEGAVLDIRYGVSRIAYSK DFETLKVDFLSKLPEMLKMFEDRLCHKTYLNGDHVTHPDFMLYDALDVVLYMDPM CLDAFPKLVCFKKRIEAIPQIDKYLKSSKYIAWPLQGWQATFGGGDHPPKSDLVPRG SPEF) (SEQ ID NO.: 9) fused directly to Met 1 of PRMT8.

Protein Expression.

E. coli (BL21(DE3) Gold, Stratagene) made competent by the CaCl₂ method were transformed with the PRMT8 construct and ampicillin selection. Protein over-expression was accomplished by growing the PRMT8 expressing E. coli clone and inducing expression with 0.3 mM IPTG at 16° C. The culture was grown for 12 hours, harvested by centrifugation, and stored at −80° C. for purification.

Protein Purification.

Expressed full-length human GST-tagged PRMT8 protein was purified from cell paste by glutathione sepharose affinity chromatography after the resin was equilibrated with 50 mM phosphate buffer, 200 mM NaCl, 5% glycerol, 5 mM β-mercaptoethanol, pH7.8 (Buffer A). GST-tagged PRMT8 was eluted with 50 mM Tris, 2 mM glutathione, pH 7.8. Pooled fractions were cleaved by thrombin (10U) and dialysed in buffer A. GST was removed by reloading the cleaved protein sample onto glutathione sepharose column and PRMT8 was collected in the flow-through fractions. PRMT8 was purified further by ceramic hydroxyapatite chromatography. The column was washed with 50 mM phosphate buffer, 100 mM NaCl, 5% glycerol, 5 mM β-mercaptoethanol, pH 7.8 and PRMT8 was eluted by 100 mM phosphate in the same buffer. Protein was concentrated and buffer was exchanged to 50 mM Tris, 300 mM NaCl, 10% glycerol, 5 mM β-mercaptoethanol, pH 7.8 by ultrafiltration. The purity of recovered protein was 89%.

Predicted Translations:

GST-tagged PRMT8
(SEQ ID NO.: 10)
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLVPRGSPEFMGMKHSSRCLLLRRKMAENAA
ESTEVNSPPSQPPQPVVPAKPVQCVHHVSTQPSCPGRGKMSKLLNPEEMT
SRDYYFDSYAHFGIHEEMLKDEVRTLTYRNSMYHNKHVFKDKVVLDVGSG
TGILSMFAAKAGAKKVFGIECSSISDYSEKIIKANHLDNIITIFKGKVEE
VELPVEKVDIIISEWMGYCLFYESMLNTVIFARDKWLKPGGLMFPDRAAL
YVVAIEDRQYKDFKIHWWENVYGFDMTCIRDVAMKEPLVDIVDPKQVVTN
ACLIKEVDIYTVKTEELSFTSAFCLQIQRNDYVHALVTYFNIEFTKCHKK
MGFSTAPDAPYTHWKQTVFYLEDYLTVRRGEEIYGTISMKPNAKNVRDLD
FTVDLDFKGQLCETSVSNDYKMR

General Procedure for PRMT8 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT8, was added to columns 11, 12, 23, 24, rows I-P for the minimum signal control. A cocktail (40 ul) containing the PRMT8 enzyme was added by Multidrop Combi (Thermo-Fisher). The compounds were allowed to incubate with PRMT8 for 30 min at room temperature, then a cocktail (10 ul) containing ³H-SAM and peptide was added to initiate the reaction (final volume=51 ul). The final concentrations of the components were as follows: PRMT8 was 1.5 nM, ³H-SAM was 50 nM, non-radiolabeled SAM was 550 nM, peptide was 150 nM, SAH in the minimum signal control wells was 1 mM, and the DMSO concentration was 2%. The assays were stopped by the addition of non-radiolabeled SAM (10 ul) to a final concentration of 400 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### PRMT3 Biochemical Assay

General Materials.

S-adenosylmethionine (SAM), S-adenosylhomocysteine (SAH), bicine, Tween20, dimethylsulfoxide (DMSO), bovine skin gelatin (BSG),), isopropyl-β-D-thiogalactopyranoside (IPTG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide containing the classic RMT substrate motif was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-GGRGGFGGRGGFGGRGGFG-amide (SEQ ID NO.: 11).

Molecular Biology:

Full-length human PRMT3 (NM_005788.3) isoform 1 transcript clone was amplified from an HEK 293 cDNA library and subcloned into pGEX-KG (GE Life Sciences). The resulting construct encodes an N-terminal GST tag and a thrombin cleavage sequence (MSPILGYWKIKGLVQPTRLLLEYLEEKYEEHLYERDEGDKWRNKKFELGLEFPNLP YYIDGDVKLTQSMAIIRYIADKHNMLGGCPKERAEISMLEGAVLDIRYGVSRIAYSK DFETLKVDFLSKLPEMLKMFEDRLCHKTYLNGDHVTHPDFMLYDALDVVLYMDPM CLDAFPKLVCFKKRIEAIPQIDKYLKSSKYIAWPLQGWQATFGGGDHPPKSDLVPRG S) (SEQ ID NO.: 12) fused directly to Cys 2 of PRMT3.

Protein Expression.

E. coli (BL21(DE3) Gold, Stratagene) made competent by the CaCl₂ method were transformed with the PRMT3 construct and ampicillin selection. Protein over-expression was accomplished by growing the PRMT3 expressing E. coli clone and inducing expression with 0.3 mM IPTG at 16° C. The culture was grown for 12 hours, harvested by centrifugation, and stored at −80° C. for purification.

Protein Purification.

Expressed full-length human GST-tagged PRMT3 protein was purified from cell paste by glutathione sepharose affinity chromatography after equilibration of the resin with 50 mM phosphate buffer, 200 mM NaCl, 5% glycerol, 1 mM EDTA, 5 mM β-mercaptoethanol, pH6.5 (Buffer A). GST-tagged PRMT3 was eluted with 50 mM Tris, 2 mM glutathione, pH 7.1 and 50 mM Tris, 20 mM glutathione, pH 7.1. Pooled fractions were dialysed in 20 mM Tris, 50 mM NaCl, 5% glycerol, 1 mM EDTA, 1 mM DTT, pH7.5 (Buffer B) and applied to a Q Sepharose Fast Flow column. GST-tagged PRMT3 was eluted by 500 mM NaCl in buffer B. Pooled fractions were dialyzed in 25 mM phosphate buffer, 100 mM NaCl, 5% glycerol, 2 mM DTT, pH 6.8 (Buffer C) and loaded on to a ceramic hydroxyapatite column. GST-tagged PRMT3 eluted with 25-400 mM phosphate in buffer C. Protein was concentrated and buffer was exchanged to 20 mM Tris, 150 mM NaCl, 5% glycerol, 5 mM β-mercaptoethanol, pH7.8 by ultrafiltration. The purity of recovered protein was 70%.

Predicted Translations:

GST-tagged PRMT3
(SEQ ID NO.: 13)
MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLVPRGSCSLASGATGGRGAVENEEDLPELS
DSGDEAAWEDEDDADLPHGKQQTPCLFCNRLFTSAEETFSHCKSEHQFNI
DSMVHKHGLEFYGYIKLINFIRLKNPTVEYMNSIYNPVPWEKEEYLKPVL
EDDLLLQFDVEDLYEPVSVPFSYPNGLSENTSVVEKLKHMEARALSAEAA
LARAREDLQKMKQFAQDFVMHTDVRTCSSSTSVIADLQEDEDGVYFSSYG
HYGIHEEMLKDKIRTESYRDFIYQNPHIFKDKVVLDVGCGTGILSMFAAK
AGAKKVLGVDQSEILYQAMDIIRLNKLEDTITLIKGKIEEVHLPVEKVDV
IISEWMGYFLLFESMLDSVLYAKNKYLAKGGSVYPDICTISLVAVSDVNK
HADRIAFWDDVYGFKMSCMKKAVIPEAVVEVLDPKTLISEPCGIKHIDCH
TTSISDLEFSSDFTLKITRTSMCTAIAGYFDIYFEKNCHNRVVFSTGPQS
TKTHWKQTVFLLEKPFSVKAGEALKGKVTVHKNKKDPRSLTVTLTLNNST
QTYGLQ

General Procedure for PRMT3 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT3, was added to columns 11, 12, 23, 24, rows I-P for the minimum signal control. A cocktail (40 ul) containing the PRMT3 enzyme was added by Multidrop Combi (Thermo-Fisher). The compounds were allowed to incubate with PRMT3 for 30 min at room temperature, then a cocktail (10 ul) containing SAM and peptide was added to initiate the reaction (final volume=51 ul). The final concentrations of the components were as follows: PRMT3 was 0.5 nM, ³H-SAM was 100 nM, non-radiolabeled SAM was 1.8 uM, peptide was 330 nM, SAH in the minimum signal control wells was 1 mM, and the DMSO concentration was 2%. The assays were stopped by the addition of potassium chloride (10 ul) to a final concentration of 100 mM. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### CARM1 Biochemical Assay

General Materials.

S-adenosylmethionine (SAM), S-adenosylhomocysteine (SAH), bicine, Tween20, dimethylsulfoxide (DMSO), bovine skin gelatin (BSG), sodium butyrate and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H3 residues 16-30 was synthesized with an N-terminal linker-affinity tag motif and a C-terminal amide cap by 21^{st} Century Biochemicals. The peptide was purified by high-performance liquid chromatography (HPLC) to greater than 95% purity and confirmed by liquid chromatography mass spectrometry (LC-MS). The sequence was Biot-Ahx-PRKQLATKAARKSAP-amide and contained a monomethylated arginine at position 26 (SEQ ID NO.: 14).

Molecular Biology:

Human CARM1 (PRMT4) (NM_199141.1) transcript clone was amplified from an HEK 293 cDNA library, incorporating a flanking 5′ sequence encoding a FLAG tag (MDYKDDDDK) (SEQ ID NO.: 6) fused directly to Ala 2 of CARM1 and 3′ sequence encoding a hexa His sequence (EGHHHHHH) (SEQ ID NO.: 15) fused directly to Ser 608. The gene sequence encoding isoform1 containing a deletion of amino acids 539-561 was amplified subsequently and subcloned into pFastBacMam (Viva Biotech).

Protein Expression.

Recombinant baculovirus were generated according to Bac-to-Bac kit instructions (Life Technologies). Protein over-expression was accomplished by infecting exponentially growing HEK 293F cell culture at 1.3×10⁶ cell/ml with virus (MOI=10) in the presence of 8 mM sodium butyrate. Infections were carried out at 37° C. for 48 hours, harvested by centrifugation, and stored at −80° C. for purification.

Protein Purification.

Expressed full-length human Flag- and His-tagged CARM1 protein was purified from cell paste by anti-flag M2 affinity chromatography with resin equilibrated with buffer containing 20 mM Tris, 150 mM NaCl, 5% glycerol, pH 7.8. Column was washed with 500 mM NaCl in buffer A and Flag-CARM1-His was eluted with 200 ug/ml FLAG peptide in buffer A. Pooled fractions were dialyzed in 20 mM Tris, 150 mM NaCl, 5% glycerol and 1 mM DTT, pH 7.8. The purity of recovered protein was 94.

Predicted Translations:

Flag-CARM1-His
(SEQ ID NO.: 16)
MDYKDDDDKAAAAAAVGPGAGGAGSAVPGGAGPCATVSVFPGARLLTIGD
ANGEIQRHAEQQALRLEVRAGPDSAGIALYSHEDVCVFKCSVSRETECSR
VGKQSFIITLGCNSVLIQFATPNDFCSFYNILKTCRGHTLERSVFSERTE
ESSAVQYFQFYGYLSQQQNMMQDYVRTGTYQRAILQNHTDFKDKIVLDVG
CGSGILSFFAAQAGARKIYAVEASTMAQHAEVLVKSNNLTDRIVVIPGKV
EEVSLPEQVDIIISEPMGYMLFNERMLESYLHAKKYLKPSGNMFPTIGDV
HLAPFTDEQLYMEQFTKANFWYQPSFHGVDLSALRGAAVDEYFRQPVVDT
FDIRILMAKSVKYTVNFLEAKEGDLHRIEIPFKFHMLHSGLVHGLAFWFD
VAFIGSIMTVWLSTAPTEPLTHWYQVRCLFQSPLFAKAGDTLSGTCLLIA
NKRQSYDISIVAQVDQTGSKSSNLLDLKNPFFRYTGTTPSPPPGSHYTSP
SENMWNTGSTYNLSSGMAVAGMPTAYDLSSVIASGSSVGHNNLIPLGSSG
AQGSGGGSTSAHYAVNSQFTMGGPAISMASPMSIPTNTMHYGSEGHHHHH
H

General Procedure for CARM1 Enzyme Assays on Peptide Substrates.

The assays were all performed in a buffer consisting of 20 mM Bicine (pH=7.6), 1 mM TCEP, 0.005% BSG, and 0.002% Tween 20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of CARM1, was added to columns 11, 12, 23, 24, rows I-P for the minimum signal control. A cocktail (40 ul) containing the CARM1 enzyme was added by Multidrop Combi (Thermo-Fisher). The compounds were allowed to incubate with CARM1 for 30 min at room temperature, then a cocktail (10 ul) containing ³H-SAM and peptide was added to initiate the reaction (final volume=51 ul). The final concentrations of the components were as follows: CARM1 was 0.25 nM, ³H-SAM was 30 nM, peptide was 250 nM, SAH in the minimum signal control wells was 1 mM, and the DMSO concentration was 2%. The assays were stopped by the addition of non-radiolabeled SAM (10 ul) to a final concentration of 300 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed once with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

$$\% inhibition calculation$$

$$\% inh = 100 - ( \frac{{dpm}_{cmpd} - {dpm}_{min}}{{dpm}_{max} - {dpm}_{min}} ) \times 100$$

Where dpm=disintegrations per minute, cmpd=signal in assay well, and min and max are the respective minimum and maximum signal controls.

$$Four \text{-} parameter IC 50 fit$$

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

RKO methylation assay

RKO adherent cells were purchased from ATCC (American Type Culture Collection), Manassas, Va., USA. DMEM/Glutamax medium, penicillin-streptomycin, heat inactivated fetal bovine serum, 0.05% trypsin and D-PBS were purchased from Life Technologies, Grand Island, N.Y., USA. Odyssey blocking buffer, 800CW goat anti-rabbit IgG (H+L) antibody, and Licor Odyssey infrared scanner were purchased from Licor Biosciences, Lincoln, Nebr., USA. Mono-methyl arginine antibody was purchased from Cell Signaling Technology, Danvers, Mass., USA. Methanol was purchased from VWR, Franklin, Mass., USA. 10% Tween 20 was purchased from KPL, Inc., Gaithersburg, Md., USA. DRAQS was purchased from Biostatus Limited, Leicestershire, UK.

RKO adherent cells were maintained in growth medium (DMEM/Glutamax medium supplemented with 10% v/v heat inactivated fetal bovine serum and 100 units/mL penicillin-streptomycin) and cultured at 37° C. under 5% CO₂.

Cell Treatment, in Cell Western (ICW) for Detection of Mono-Methyl Arginine and DNA Content.

RKO cells were seeded in assay medium at a concentration of 20,000 cells per mL to a poly-D-lysine coated 384 well culture plate (BD Biosciences 356697) with 50 μL per well. Compound (100 nL) from a 96-well source plate was added directly to 384 well cell plate. Plates were incubated at 37° C., 5% CO₂ for 72 hours. After three days of incubation, plates were brought to room temperature outside of the incubator for ten minutes and blotted on paper towels to remove cell media. 50 μL of ice cold 100% methanol was added directly to each well and incubated for 30 min at room temperature. After 30 min, plates were transferred to a Biotek EL406 plate washer and washed 2 times with 100 μL per well of wash buffer (1×PBS). Next 60 μL per well of Odyssey blocking buffer (Odyssey Buffer with 0.1% Tween 20 (v/v)) were added to each plate and incubated 1 hour at room temperature. Blocking buffer was removed and 20 μL per well of primary antibody was added (mono-methyl arginine diluted 1:200 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:200 800CW goat anti-rabbit IgG (H+L) antibody, 1:1000 DRAQS (Biostatus limited) in Odyssey buffer with 0.1% Tween 20 (v/v)) and incubated for 1 hour at room temperature. The plates were washed 5 times with 100 μL per well wash buffer then 2 times with 100 μL per well of water. Plates were allowed to dry at room temperature then imaged on the Licor Odyssey machine which measures integrated intensity at 700 nm and 800 nm wavelengths. Both 700 and 800 channels were scanned.

Calculations:

First, the ratio for each well was determined by:

$$( \frac{monomethyl Arginine 800 nm value}{DRAQ 5 \; 700 nm value} )$$

Each plate included fourteen control wells of DMSO only treatment (minimum activation) as well as fourteen control wells for maximum activation treated with 20 μM of a reference compound. The average of the ratio values for each control type was calculated and used to determine the percent activation for each test well in the plate. Reference compound was serially diluted three-fold in DMSO for a total of nine test concentrations, beginning at 20 μM. Percent activation was determined and EC₃₀ curves were generated using triplicate wells per concentration of compound.

$$Percent Activation = 100 - ( ( \frac{( ( Individual Test Sample Ratio ) - ( Minimum Activation Ratio ) )}{( ( Maximum Activation Ratio ) - ( Minimum Activation Ratio ) )} ) * 100 )$$

### Cell Treatment, in Cell Western (ICW) for Detection of Asymmetric Di-Methyl PABP1 and DNA Content.

RKO cells were seeded in assay medium at a concentration of 30,000 cells per mL to a poly-D-lysine coated 384 well culture plate (BD Biosciences 356697) with 50 μL per well. Compound (100 nL) from a 96-well source plate was added directly to 384 well cell plate. Plates were incubated at 37° C., 5% CO2 for 48 hours. After two days of incubation, plates were brought to room temperature outside of the incubator for ten minutes and blotted on paper towels to remove cell media. Cells were fixed for 20 minutes at room temperature by adding 50 μl of 8% PFA followed by aspiration of supernatant with the Biotek EL406 plate washer. Cells were then permeabilized by addition of 50 μL of ice cold 100% methanol directly to each well and incubated for 30 min at room temperature. After 30 min, plates were transferred to a Biotek EL406 plate washer and washed 2 times with 100 μL per well of wash buffer (1×PBS). Next 60 μL per well of Odyssey blocking buffer (Odyssey Buffer with 0.1% Tween 20 (v/v)) were added to each plate and incubated 1 hour at room temperature. Blocking buffer was removed and 20 μL per well of primary antibody was added (asymmetric-methyl PABP1) diluted 1:400 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:800 800CW goat anti-rabbit IgG (H+L) antibody, 1:2000 DRAQS in Odyssey buffer with 0.1% Tween 20 (v/v)) and incubated for 1 hour at room temperature. The plates were washed 5 times with 100 μL per well wash buffer then 2 times with 100 μL per well of water. Plates were allowed to dry at room temperature then imaged on the Licor Odyssey machine which measures integrated intensity at 700 nm and 800 nm wavelengths. Both 700 and 800 channels were scanned.

Calculations:

First, the ratio for each well was determined by:

$$( \frac{asymmetric di \text{-} methyl PABP 1 \; 800 nm value}{DRAQ 5 \; 700 nm value} )$$

Each plate included fourteen control wells of DMSO only treatment (minimum inhibition) as well as fourteen control wells for maximum inhibition treated with 20 μM of a reference compound. The average of the ratio values for each control type was calculated and used to determine the percent activation for each test well in the plate. Reference compound was serially diluted three-fold in DMSO for a total of nine test concentrations, beginning at 20 μM. Percent inhibition was determined and IC50 curves were generated using triplicate wells per concentration of compound.

$$Percentage Inhibition = 100 - ( \frac{( Minimum Inhibition Ratio ) - ( Individual Test Sample Ratio )}{( Minimum Inhibition Ratio ) \times ( Maximum Inhibition Ratio )} ) \times 100$$

### OTHER EMBODIMENTS

The foregoing has been a description of certain non-limiting embodiments of the invention. Those of ordinary skill in the art will appreciate that various changes and modifications to this description may be made without departing from the spirit or scope of the present invention, as defined in the following claims.

What is claimed is:

1. A compound of Formula (I): 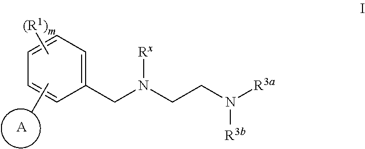
or a pharmaceutically acceptable salt thereof,
wherein:
Ring A is optionally substituted aryl, optionally substituted pyridinyl, optionally substituted bicyclic heteroaryl with one, three, or four nitrogen ring atoms, optionally substituted indazolyl, optionally substituted azaindolyl, or optionally substituted benzoimidazolyl;
m is 0, 1, 2, 3, or 4;
R^{x} is optionally substituted C₁₋₄ alkyl or optionally substituted C₃₋₄ cycloalkyl; and
each of R^{3a} and R^{3b} is independently hydrogen, optionally substituted C₁₋₄ alkyl, or optionally substituted C₃₋₄ cycloalkyl;
each instance of R¹ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;
each instance of R^{A} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group; and
each instance of R^{B} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
or two R^{B} taken together with the intervening nitrogen form optionally substituted heterocyclyl;
or R^{A} and R^{B} taken together with the intervening atoms form optionally substituted heterocyclyl; and
provided that the optional substituent on Ring A is not an optionally substituted pyridone.

2. The compound of claim 1, wherein the compound is of Formula (I-a):

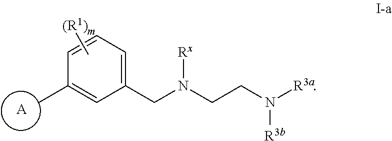

3. The compound of claim 1, wherein the compound is of Formula (I-al): 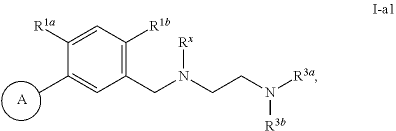
or a pharmaceutically acceptable salt thereof; wherein each instance of R^{1a} and R^{1b} is independently hydrogen, halogen, or optionally substituted C₁₋₆ alkyl.

4. The compound of claim 3, wherein R^{1a} is hydrogen.

5. The compound of claim 3, wherein R^{1a} is halogen.

6. The compound of claim 3, wherein R^{1a} is F.

7. The compound of claim 3, wherein R^{1a} is unsubstituted C₁₋₆ alkyl.

8. The compound of claim 3, wherein R^{1a} is methyl.

9. The compound of any one of claims 3-8, wherein R^{1b} is hydrogen.

10. The compound of any one of claims 3-8, wherein R^{1b} is halogen.

11. The compound of any one of claim 10, wherein R^{1b} is F.

12. The compound of any one of claims 3-8, wherein R^{1b} is unsubstituted C₁₋₆ alkyl.

13. The compound of claim 12, wherein R^{1b} is methyl.

14. The compound of claim 1, wherein the compound is of Formula (I-b): 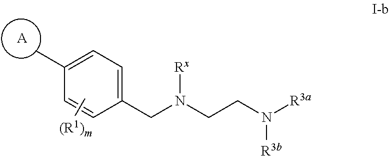
or a pharmaceutically acceptable salt thereof.

15. The compound of claim 14, wherein m is 0.

16. The compound of claim 14, wherein m is 1.

17. The compound of claim 14 or 16, wherein R¹ is hydrogen.

18. The compound of claim 14 or 16, wherein R¹ is unsubstituted C₁₋₆ alkyl.

19. The compound of claim 18, wherein R¹ is methyl.

20. The compound of any one of claims 1-19, wherein Ring A is an optionally substituted aryl.

21. The compound of claim 20, wherein Ring A is optionally substituted phenyl of Formula (A-1): 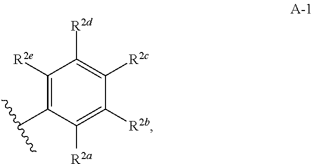
wherein each of R^{2a}, R^{2b}, R^{2c}, R^{2d}, and R^{2e} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂.

22. The compound of claim 21, wherein Ring A is of one of the following formulae:

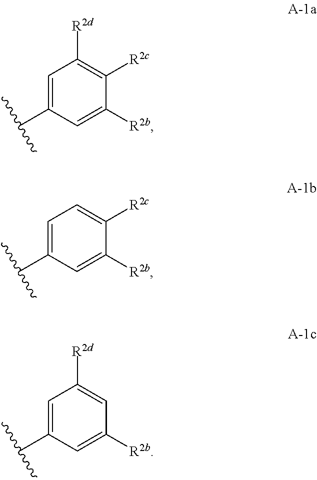

23. The compound of any one of claims 21-22, wherein R^{2b} is —N(R^{B})₂ or —C(═O)N(R^{B})₂.

24. The compound of any one of claims 21-23, wherein R^{2b} is —N(R^{B})₂; and each instance of R^{B} is independently hydrogen or optionally substituted alkyl.

25. The compound of any one of claims 21-24, wherein R^{2b} is —N(CH₃)R^{B}, wherein R^{B} is hydrogen or optionally substituted alkyl.

26. The compound of claim 25, wherein R^{B} is —CH₂-cyclopropyl.

27. The compound of claim 23, wherein R^{2b} is —C(═O)N(R^{B})₂, wherein each instance of R^{B} is independently hydrogen, optionally substituted alkyl, or optionally substituted heterocyclyl.

28. The compound of claim 27, wherein R^{2b} is —C(═O)NHR^{B}.

29. The compound of claim 28, wherein R^{B} is tetrahydropyranyl.

30. The compound of claim 28, wherein R^{B} is substituted alkyl.

31. The compound of claim 30, wherein R^{B} is —C₁₋₆alkyl-heterocyclyl.

32. The compound of claim 31, wherein R^{B} is —CH₂-tetrahydropyranyl.

33. The compound of any one of claims 21-32, wherein R^{2c} is optionally substituted alkyl or —C(═O)N(R^{B})₂.

34. The compound of claim 33, wherein R^{2c} is unsubstituted C₁₋₆ alkyl.

35. The compound of claim 34, wherein R^{2c} is methyl.

36. The compound of claim 33, wherein R^{2c} is —C(═O)N(R^{B})₂; and each instance of R^{B} is independently hydrogen, optionally substituted alkyl, or optionally substituted heterocyclyl.

37. The compound of claim 36, wherein R^{2c} is —C(═O)NHR^{B}; and R^{B} is optionally substituted C₁₋₆ alkyl or optionally substituted heterocyclyl.

38. The compound of claim 37, wherein R^{B} is substituted C₁₋₆ alkyl.

39. The compound of claim 38, wherein R^{B} is —CH₂-tetrahydropyranyl.

40. The compound of claim 37, wherein R^{B} is unsubstituted C₁₋₆ alkyl.

41. The compound of claim 40, wherein R^{B} is methyl.

42. The compound of claim 37, wherein R^{B} is unsubstituted heterocyclyl.

43. The compound of claim 42, wherein R^{B} is tetrahydropyranyl.

44. The compound of any one of claims 21-43, wherein R^{2d} is —N(R^{B})₂ or —C(═O)N(R^{B})₂.

45. The compound of claim 44, wherein R^{2d} is —N(R^{B})₂; and each instance of R^{B} is independently hydrogen, optionally substituted alkyl, optionally substituted heterocyclyl.

46. The compound of claim 45, wherein R^{2d} is —N(CH₃)R^{B} or —N(C₂H₅)R^{B}.

47. The compound of claim 46, wherein R^{B} is substituted alkyl.

48. The compound of claim 46, wherein R^{B} is —C₁₋₄alkyl-carbocyclyl.

49. The compound of claim 46, wherein R^{B} is —CH₂-cyclopropyl.

50. The compound of claim 46, wherein R^{B} is optionally substituted heterocyclyl.

51. The compound of claim 46, wherein R^{B} is tetrahydropyranyl.

52. The compound of claim 44, wherein R^{2d} is —C(═O)N(R^{B})₂; and each instance of R^{B} is independently hydrogen, optionally substituted alkyl, or optionally substituted heterocyclyl.

53. The compound of claim 48, wherein R^{B} is substituted alkyl.

54. The compound of claim 53, wherein R^{B} is —C₁₋₄alkyl-heterocyclyl.

55. The compound of claim 54, wherein R^{B} is —CH₂-tetrahydropyranyl.

56. The compound of claim 52, wherein R^{B} is unsubstituted heterocyclyl.

57. The compound of claim 56, wherein R^{B} is tetrahydropyranyl.

58. The compound of claim 44, wherein two R^{B} taken together with the intervening nitrogen form optionally substituted heterocyclyl.

59. The compound of any one of claims 21-58, wherein each of R^{2b}, R^{2c}, and R^{2d} is independently of one of the following formulae:

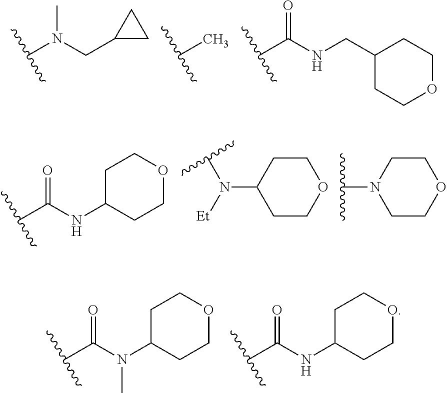

60. The compound of claim 22, wherein Ring A is of the formula: 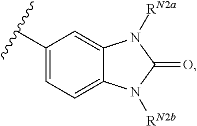
wherein each of R^{N2a} and R^{N2b} is independently hydrogen, optionally substituted alkyl, or optionally substituted aryl.

61. The compound of claim 60, wherein R^{N2a} is hydrogen.

62. R^{N2b} The compound of any one of claims 60-61, wherein R is optionally substituted phenyl.

63. The compound of claim 62, wherein Ring A is of the formula:

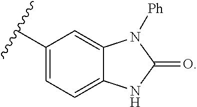 [SMILES: *c1ccc2[nH]c(=O)n(-c3ccccc3)c2c1]

64. The compound of claim 22, wherein Ring A is of the formula: 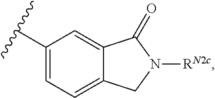
wherein R^{N2c} is optionally substituted alkyl.

65. The compound of claim 64, wherein R^{N2C} is unsubstituted C₁₋₆ alkyl.

66. The compound of any one of claims 65, wherein R^{N2C} is methyl or ethyl.

67. The compound of any one of claims 1-19, wherein Ring A is of Formula (A-3) 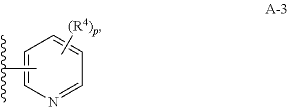
wherein:
each instance of R⁴ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and
p is 0, 1, 2, or 3;
or R^{B} and another R⁴ taken together with the intervening atoms form optionally substituted heterocyclyl.

68. The compound of claim 67, wherein p is 1.

69. The compound of claim 67, wherein p is 2.

70. The compound of claim 67, wherein Ring A is of one of the following formulae: 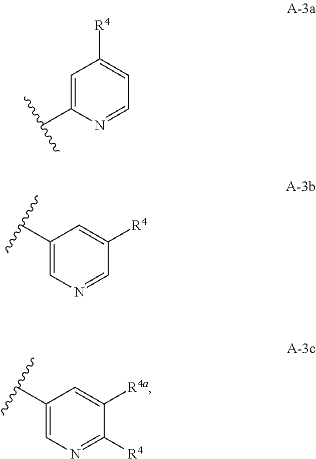
wherein:
R^{4a} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂.

71. The compound of claim 70, wherein each instance of R⁴ and R^{4a} is independently hydrogen, optionally substituted C₁₋₆ alkyl, —OR^{A}, —NR^{B}C(═O)R^{A}, or —C(═O)N(R^{B})₂.

72. The compound of claim 71, wherein R^{B} is hydrogen or optionally substituted alkyl.

73. The compound of claim 70, wherein R⁴ and R^{4a} is independently hydrogen, optionally substituted C₁₋₆ alkyl, —OR^{A}, —NHC(═O)R^{A}, —C(═O)NHR^{B}, or —C(═O)N(CH₃)R^{B}.

74. The compound of claim 73, wherein R^{A} is hydrogen or optionally substituted alkyl; and R^{B} is optionally substituted —C₁₋₄alkyl-heteroaryl or optionally substituted —C₁₋₄alkyl-phenyl.

75. The compound of any one of claims 67-74, wherein R^{A} and R^{B} taken together with the intervening atoms form optionally substituted heterocyclyl.

76. The compound of any one of claims 67-74, wherein R^{B} and another R⁴ taken together with the intervening atoms form optionally substituted heterocyclyl.

77. The compound of any one of claims 67-76, wherein Ring A is one of the following formulae:

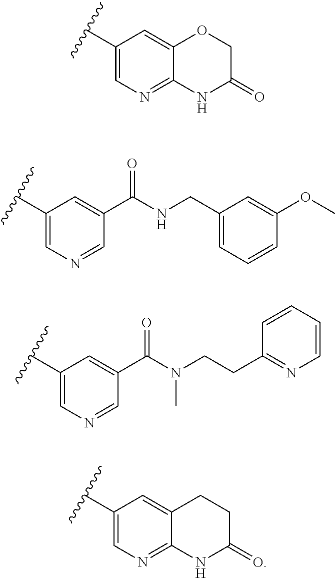 [SMILES: *c1cnc2c(c1)OCC(=O)N2 ; *c1cncc(C(=O)NCc2cccc(OC)c2)c1 ; *c1cncc(C(=O)N(C)CCc2ccccn2)c1 ; *c1cnc2c(c1)CCC(=O)N2]

78. The compound of any one of claims 1-19, wherein Ring A is of Formula (A-4): 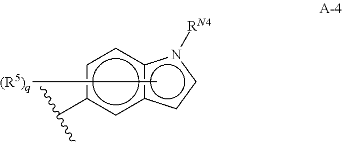
wherein:
each instance of R⁵ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;
q is 0, 1, 2, 3, 4, or 5; and
each instance of R^{N4} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
or R^{B} and another R⁵ taken together with the intervening atoms form optionally substituted heterocyclyl.

79. The compound of claim 78, wherein Ring A is of Formula (A-4a):

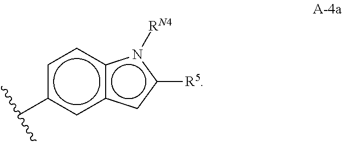

80. The compound of claim 79, wherein R⁵ is optionally substituted C₁₋₆ alkyl.

81. The compound of claim 80, wherein R⁵ is unsubstituted C₁₋₆ alkyl.

82. The compound of claim 82, wherein R⁵ is methyl.

83. The compound of any one of claims 1-19, wherein Ring A is one of the following formulae: 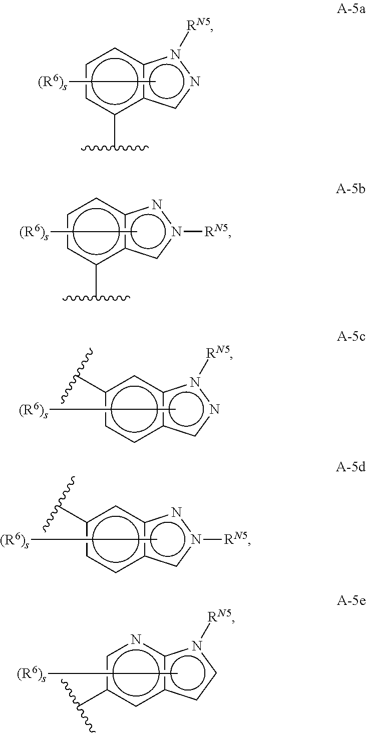
wherein:
each instance of R⁶ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and
s is 0, 1, 2, 3, or 4;
each instance of R^{N5} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

84. The compound of claim 83, wherein s is 1.

85. The compound of claim 83, wherein s is 2.

86. The compound of any one of claims 83-85, wherein Ring A is of one of the following formulae:

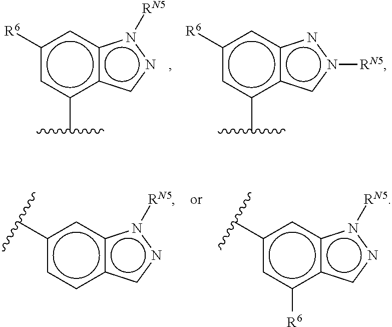

87. The compound of any one of claims 83-86, wherein R^{N5} is hydrogen, optionally substituted alkyl, or —C(═O)NHR^{B}; and R^{B} is hydrogen or optionally substituted alkyl.

88. The compound of claim 87, wherein R^{N5} is hydrogen.

89. The compound of claim 87, wherein R^{N5} is unsubstituted C₁₋₆ alkyl.

90. The compound of claim 89, wherein R^{N5} is isopropyl.

91. The compound of claim 89, wherein R^{N5} is substituted alkyl.

92. The compound of claim 91, wherein R^{N5} is of Formula (i): 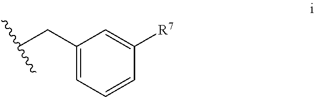
wherein R⁷ is hydrogen, optionally substituted alkyl, —OR^{A}, —C(═O)R^{A}, or —C(═O)N(R^{B})₂.

93. The compound of claim 92, wherein:
each instance of R^{A} is independently optionally substituted alkyl, optionally substituted phenyl, or optionally substituted heterocyclyl; and
each instance of R^{B} is independently optionally substituted alkyl;
or two R^{B} taken together with the intervening nitrogen form optionally substituted heterocyclyl.

94. The compound of claim 92 or 93, wherein R^{N5} is of one of the following formulae:

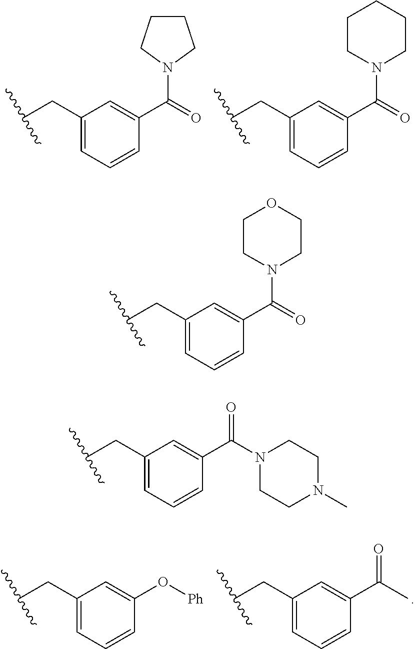

95. The compound of claim 91, wherein R^{N5} is of Formula (ii): 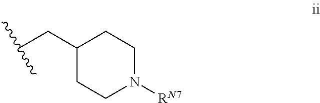
wherein R^{N7} is hydrogen, optionally substituted alkyl, or a nitrogen protecting group.

96. The compound of claim 95, wherein R^{N7} is hydrogen.

97. The compound of claim 95, wherein R^{N7} is a nitrogen protecting group.

98. The compound of claim 95, wherein R^{N7} is acyl.

99. The compound of claim 95, wherein R^{N7} is of one of the following formulae:

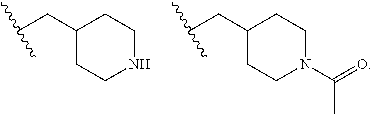 [SMILES: *CC1CCNCC1 ; *CC1CCN(C(C)=O)CC1]

100. The compound of any one of claims 83-99, wherein R⁶ is —C(═O)N(R^{B})₂.

101. The compound of claim 100, wherein each instance of R^{B} is independently hydrogen or optionally substituted alkyl.

102. The compound of claim 101, wherein R⁶ is —C(═O)NHR^{B}.

103. The compound of claim 102, wherein R⁶ is —C(═O)N(CH₃)R^{B}.

104. The compound of any one of claims 100-103, wherein R^{B} is substituted alkyl.

105. The compound of claim 104, wherein R^{B} is optionally substituted —C₁₋₆alkyl-phenyl.

106. The compound of claim 105, wherein R^{B} is of one of the following formulae:

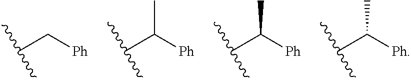

107. The compound of claim 104, wherein R^{B} is optionally substituted —C₁₋₆alkyl-heteroaryl.

108. The compound of claim 83, wherein Ring A is of one of the following formulae:

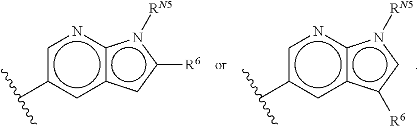

109. The compound of claim 108, wherein R^{N5} is hydrogen.

110. The compound of any one of claims 108-109, wherein R⁶ is unsubstituted C₁₋₆ alkyl.

111. The compound of claim 110, wherein R⁶ is methyl.

112. The compound of any one of claims 1-19, wherein Ring A is of one of the following formulae: 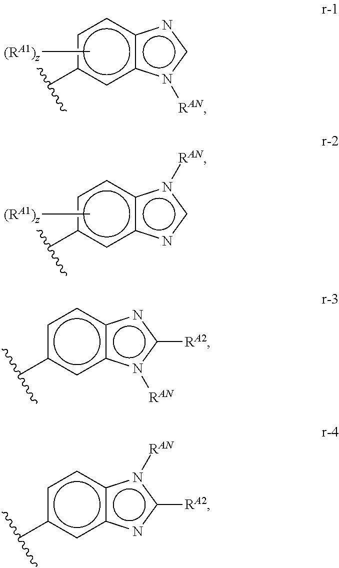
wherein:
each instance of R^{A1} and R^{A2} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;
z is 0, 1, 2, or 3;
each instance of R^{A} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group; and
each instance of R^{AN} and R^{B} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
or two R^{B} taken together with the intervening nitrogen form optionally substituted heterocyclyl;
or R^{A} and R^{B} taken together with the intervening atoms form optionally substituted heterocyclyl.

113. The compound of claim 112, wherein R^{AN} is hydrogen, optionally substituted alkyl, optionally substituted aryl, optionally substituted carbocyclyl, or optionally substituted heterocyclyl.

114. The compound of claim 113, wherein R^{AN} is hydrogen.

115. The compound of any one of claims 112-113, wherein R^{AN} is optionally substituted alkyl.

116. The compound of claim 115, wherein R^{AN} is unsubstituted alkyl.

117. The compound of claim 116, wherein R^{AN} is methyl, ethyl, n-propyl, or isopropyl.

118. The compound of claim 117, wherein R^{AN} is substituted alkyl.

119. The compound of claim 118, wherein R^{AN} is of Formula 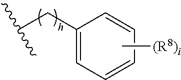
wherein:
h is 0, 1, 2, 3, or 4;
i is 0, 1, 2, 3, 4, or 5; and
each instance of R⁸ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂.

120. The compound of claim 119, wherein i is 0.

121. The compound of claim 119, wherein i is 1.

122. The compound of claim 119, wherein i is 2.

123. The compound of any one of claims 119-122, wherein his 1.

124. The compound of any one of claims 119-122, wherein his 2.

125. The compound of claim 112-124, wherein R^{AN} is of one of the following formulae:

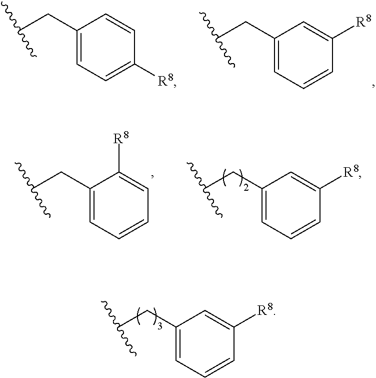

126. The compound of any one of claims 119-125, wherein R⁸ is CN.

127. The compound of any one of claims 119-125, wherein R⁸ is halogen.

128. The compound of claim 127, wherein R⁸ is Cl.

129. The compound of any one of claims 119-125, wherein R⁸ is unsubstituted alkyl.

130. The compound of claim 129, wherein R⁸ is methyl.

131. The compound of any one of claims 119-125, wherein R⁸ is —OR^{A}; and R^{A} is optionally substituted alkyl.

132. The compound of claim 131, wherein R⁸ is —OCH₃.

133. The compound of any one of claims 119-125, wherein R⁸ is —SO₂R^{A}; and R^{A} is optionally substituted alkyl.

134. The compound of claim 133, wherein R⁸ is —SO₂CH₃.

135. The compound of any one of claims 119-125, wherein R⁸ is —C(═O)N(R^{B})₂.

136. The compound of claim 135, wherein:
each instance of R^{B} is independently hydrogen, optionally substituted alkyl, optionally substituted carbocyclyl, optionally substituted heterocyclyl, optionally substituted aryl, optionally substituted heteroaryl, or a nitrogen protecting group;
or two R^{B} taken together with the intervening nitrogen form optionally substituted heterocyclyl.

137. The compound of claim 135, wherein R⁸ is —C(═O)NHR^{B} or —C(═O)N(CH₃)R^{B}.

138. The compound of any one of claims 135-137, wherein R^{B} is hydrogen or optionally substituted alkyl.

139. The compound of claim 138, wherein R^{B} is unsubstituted alkyl.

140. The compound of claim 139, wherein R^{B} is methyl.

141. The compound of claim 138, wherein R^{B} is substituted alkyl.

142. The compound of claim 141, wherein R^{B} is optionally substituted —C₁₋₄alkyl-alkoxy, optionally substituted —C₁₋₄alkyl-aryl, optionally substituted —C₁₋₄alkyl-heterocyclyl, or optionally substituted —C₁₋₄alkyl-acyl.

143. The compound of claim 142, wherein R^{B} is of one of the following formulae:

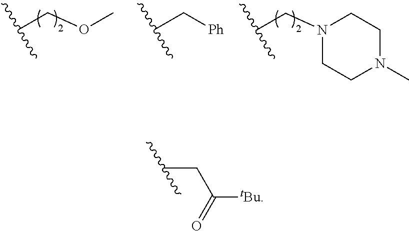

144. The compound of any one of claims 135-137, wherein R^{B} is optionally substituted carbocyclyl.

145. The compound of claim 144, wherein R^{B} is cyclopropyl, cyclopentyl, cyclohexyl, or

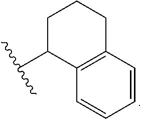 [SMILES: *C1CCCc2ccccc21]

146. The compound of any one of claims 135-137, wherein R^{B} is optionally substituted phenyl.

147. The compound of claim 146, wherein R^{B} is of one of the following formulae:

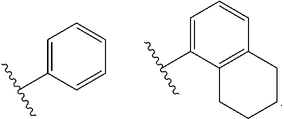 [SMILES: *c1ccccc1 ; *c1cccc2c1CCCC2]

148. The compound of any one of claims 135-137, wherein R^{B} is optionally substituted heterocyclyl.

149. The compound of claim 148, wherein R^{B} is tetrahydropyranyl.

150. The compound of any one of claims 135-137, wherein two R^{B} taken together with the intervening nitrogen form optionally substituted heterocyclyl.

151. The compound of claim 150, wherein R⁸ is one of the following formulae:

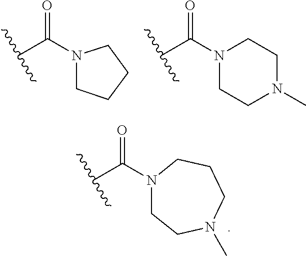

152. The compound of claim 118, wherein R^{AN} is of the formula 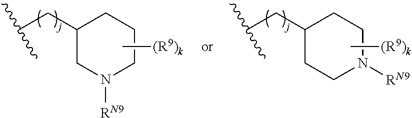
wherein:
each instance of j is 0, 1, 2, 3, or 4;
each instance of k is 0, 1, 2, 3, 4, 5, or 6, as valency permits;
each instance of R⁹ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;
each instance of R^{N9} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl.

153. The compound of claim 152, wherein R^{AN} is of one of the following formulae:

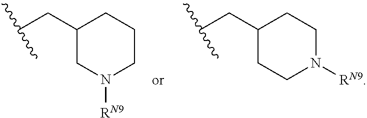

154. The compound of any one of claims 52-153, wherein R^{N9} is hydrogen, optionally substituted alkyl, or a nitrogen protecting group.

155. The compound of claim 154, wherein R^{N9} is hydrogen.

156. The compound of claim 154, wherein R^{N9} is unsubstituted alkyl.

157. The compound of claim 156, wherein R^{N9} is methyl.

158. The compound of claim 154, wherein R^{N9} is a nitrogen protecting group.

159. The compound of claim 158, wherein R^{N9} is acetyl.

160. The compound of claim 158, wherein R^{N9} is —SO₂—CH₃.

161. The compound of claim 118, wherein R^{AN} is optionally substituted carbocyclyl.

162. The compound of claim 161, wherein R^{AN} is cyclopentyl.

163. The compound of claim 118, wherein R^{AN} is optionally substituted heterocyclyl.

164. The compound of claim 163, wherein R^{AN} is tetrahydropyranyl or piperidine.

165. The compound of any one of claims 112-164, wherein R^{A1} is hydrogen, optionally substituted alkyl, optionally substituted aryl, or optionally substituted heteroaryl.

166. The compound of claim 165, wherein R^{A2} is hydrogen.

167. The compound of claim 165, wherein R^{A2} is substituted C₁₋₆ alkyl.

168. The compound of claim 167, wherein R^{A2} is optionally substituted —C₁₋₄alkyl-aryl.

169. The compound of claim 168, wherein R^{A2} is optionally substituted —CH₂-monosubstituted-phenyl.

170. The compound of claim 169, wherein R^{A2} is

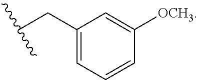 [SMILES: *Cc1cccc(OC)c1]

171. The compound of claim 165, wherein R^{A2} is substituted phenyl.

172. The compound of claim 171, wherein R^{A2} is o-methoxy-phenyl.

173. The compound of claim 165, wherein R^{A2} is optionally substituted heteroaryl.

174. The compound of claim 173, wherein R^{A2} is quinolinyl.

175. The compound of claim 114, wherein z is 0.

176. The compound of claim 114, wherein z is 1.

177. The compound of any one of claims 1-19, wherein Ring A is one of the following formulae: 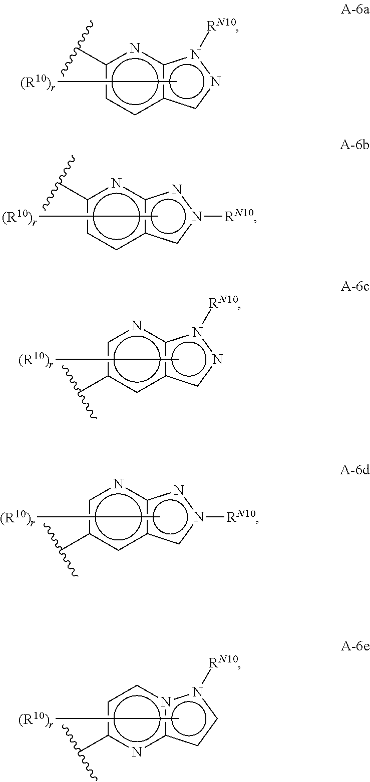
wherein:
each instance of R¹⁰ is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;
r is 0, 1, 2, 3, or 4, as valency permits; and
each instance of R^{N10} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

178. The compound of claim 177, wherein Ring A is of the formula:

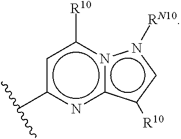

179. The compound of claim 178, wherein each instance of R¹⁰ is independently optionally substituted C₁₋₆ alkyl or optionally substituted six-membered heterocyclyl.

180. The compound of claim 179, wherein each instance of R¹⁰ is independently unsubstituted C₁₋₆ alkyl or unsubstituted six-membered heterocyclyl.

181. The compound of claim 180, wherein R¹⁰ is isopropyl or morpholinyl.

182. The compound of any one of claims 178-181, wherein R^{1\110} is hydrogen.

183. The compound of claim 177, wherein Ring A is of the formula:

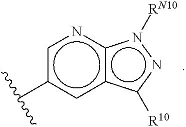

184. The compound of claim 183, wherein R^{N10} is optionally substituted C₁₋₆ alkyl.

185. The compound of claim 184, wherein R^{N10} is substituted C₁₋₆ alkyl.

186. The compound of claim 185, wherein R^{N10} is —C₁₋₆alkyl-heterocyclyl.

187. The compound of claim 186, wherein R^{N10} is —CH₂-tetrahydropyranyl.

188. The compound of any one of claims 183-187, wherein R¹⁰ is halogen or optionally substituted carbocyclyl.

189. The compound of any one of claims 183-187, wherein R¹⁰ is halogen or optionally substituted C₃₋₆ carbocyclyl.

190. The compound of claim 189, wherein R¹⁰ is cyclopropyl.

191. The compound of claim 189, wherein R¹⁰ is Br.

192. The compound of claim 177, wherein Ring A is of one of the following formulae: 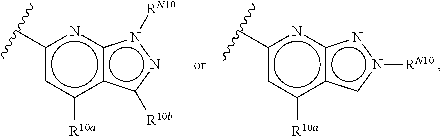
wherein:
each of R^{10a} and R^{10b} is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂; and
each instance of R^{N10} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

193. The compound of claim 192, wherein each instance of R^{N10} is independently hydrogen, optionally substituted alkyl, optionally substituted carbocyclyl, optionally substituted heterocyclyl, optionally substituted aryl, optionally substituted heteroaryl.

194. The compound of any one of claims 192-193, wherein R^{N10} is hydrogen.

195. The compound of any one of claims 192-193, wherein R^{N10} is optionally substituted C₁₋₆ alkyl.

196. The compound of claim 195, wherein R^{N10} is unsubstituted C₁₋₆ alkyl.

197. The compound of claim 196, wherein R^{N10} is methyl, ethyl, isopropyl, or tert-butyl.

198. The compound of claim 195, wherein R^{N10} is substituted C₁₋₆ alkyl.

199. The compound of claim 198, wherein R^{N10} is C₁₋₆ haloalkyl, —C₁₋₄alkyl-cyano, —C₁₋₄ alkyl-alkenyl, —C₁₋₄alkyl-aryl, —C₁₋₄alkyl-carbocyclyl, or —C₁₋₄alkyl-heteroaryl.

200. The compound of claim 199, wherein R^{N10} is —CH₂—CF₃, —CH₂—CN, —CH₂—CH═CH₂, —CH₂-pyridinyl, —CH₂-cyclopropyl, —CH₂-phenyl, or —CH₂-o-F-phenyl.

201. The compound of claims 192-193, wherein R^{N10} is optionally substituted carbocyclyl.

202. The compound of claim 201, wherein R^{N10} is cyclobutyl or cyclopropyl.

203. The compound of claims 192-193, wherein R^{N10} is optionally substituted heterocyclyl.

204. The compound of claim 203, wherein R^{N10} is tetrahydropyranyl.

205. The compound of claims 192-193, wherein R^{N10} is optionally substituted heteroaryl.

206. The compound of claim 205, wherein R^{N10} is pyridinyl.

207. The compound of any one of claims 192-206, wherein R^{10a} is hydrogen, halogen, —CN, optionally substituted alkyl, optionally substituted alkenyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)N(R^{B})₂, —NR^{B}C(═O)R^{A}, or —NR^{B}C(═O)N(R^{B})₂.

208. The compound of claim 207, wherein R^{10a} is hydrogen.

209. The compound of claim 207, wherein R^{10a} is halogen.

210. The compound of claim 209, wherein R^{10a} is Cl.

211. The compound of claim 207, wherein R^{10a} is CN.

212. The compound of claim 207, wherein R^{10a} is optionally substituted C₁₋₆ alkyl.

213. The compound of claim 212, wherein R^{10a} is unsubstituted C₁₋₆ alkyl.

214. The compound of claim 213, wherein R^{10a} is methyl, ethyl, n-propyl, or isopropyl.

215. The compound of claim 212, wherein R^{10a} is substituted C₁₋₆ alkyl.

216. The compound of claim 215, wherein R^{10a} is C₁₋₆ haloalkyl.

217. The compound of claim 216, wherein R^{10a} is CF₃.

218. The compound of claim 215, wherein R^{10a} is —C₁₋₆alkyl-OH.

219. The compound of claim 218, wherein R^{10a} is —CH₂OH.

220. The compound of claim 215, wherein R^{10a} is of the formula: 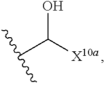
wherein X^{10a} is optionally substituted alkyl, optionally substituted alkenyl, optionally substituted alkynyl, optionally substituted carbocyclyl, optionally substituted heterocyclyl, optionally substituted aryl, or optionally substituted heteroaryl.

221. The compound of claim 220, wherein X^{10a} is unsubstituted C₁₋₆ alkyl.

222. The compound of claim 221, wherein X^{10a} is ethyl.

223. The compound of claim 222, wherein X^{10a} is unsubstituted six-membered heteroaryl.

224. The compound of claim 223, wherein X^{10a} is pyridinyl.

225. The compound of claim 220, wherein X^{10a} is unsubstituted heterocyclyl.

226. The compound of claim 225, wherein X^{10a} is tetrahydropyranyl.

227. The compound of claim 220, wherein R^{10a} is optionally substituted alkenyl.

228. The compound of claim 227, wherein R^{10a} is of the formula

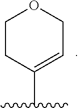 [SMILES: *C1=CCOCC1]

229. The compound of claim 220, wherein R^{10a} is optionally substituted aryl.

230. The compound of claim 229, wherein R^{10a} is optionally substituted phenyl.

231. The compound of claim 230, wherein R^{10a} is p-OH-phenyl or p-F-phenyl.

232. The compound of claim 220, wherein R^{10a} is optionally substituted heterocyclyl.

233. The compound of claim 232, wherein R^{10a} is optionally substituted four-membered, five-membered, or six-membered heterocyclyl.

234. The compound of claim 233, wherein R^{10a} is of one of the following formulae: 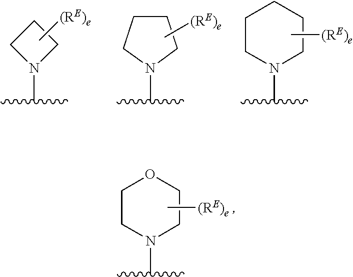
wherein:
e is 0, 1, 2, 3, or 4, as valency permits; and
each instance of R^{E} is independently hydrogen, halogen, —CN, —NO₂, —N₃, —OH, optionally substituted alkyl, optionally substituted alkenyl, optionally substituted alkynyl, optionally substituted carbocyclyl, optionally substituted aryl, optionally substituted heterocyclyl, optionally substituted heteroaryl, optionally substituted alkoxy, or optionally substituted amino.

235. The compound of claim 234, wherein e is 0.

236. The compound of claim 234, wherein e is 1.

237. The compound of any one of claims 234-236, wherein R^{E} is CN or optionally substituted alkyl.

238. The compound of claim 237, wherein R^{E} is CN.

239. The compound of claim 237, wherein R^{E} is substituted alkyl.

240. The compound of claim 239, wherein R^{E} is CF₃ or methoxy.

241. The compound of any one of claims 234-240, wherein R^{10a} is one of the following formulae:

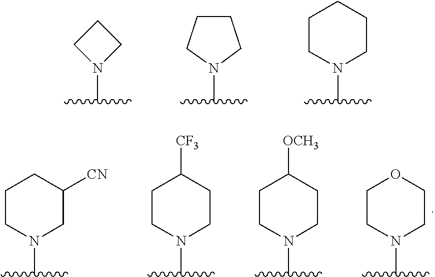

242. The compound of claim 207, wherein R^{10a} is optionally substituted heteroaryl.

243. The compound of claim 242, wherein R^{10a} is optionally substituted five-membered heteroaryl.

244. The compound of claim 243, wherein R^{10a} is thiophenyl, furanyl, thiazolyl, or pyrazolyl.

245. The compound of claim 244, wherein R^{10a} is one of the following formulae:

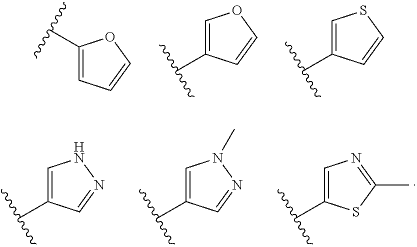

246. The compound of claim 242, wherein R^{10a} is optionally substituted six-membered heteroaryl.

247. The compound of claim 246, wherein R^{10a} is pyridinyl.

248. The compound of claim 207, wherein R^{10a} is —OR^{A}; and R^{A} is optionally substituted alkyl, optionally substituted carbocyclyl, optionally substituted heterocyclyl, optionally substituted aryl, or an oxygen protecting group.

249. The compound of claim 248, wherein R^{A} is unsubstituted C₁₋₆ alkyl.

250. The compound of claim 249, wherein R^{A} is methyl or ethyl.

251. The compound of claim 248, wherein R^{A} is substituted C₁₋₆ alkyl.

252. The compound of claim 251, wherein R^{A} is C₁₋₆ haloalkyl or —C₁₋₆ alkyl-carbocyclyl.

253. The compound of claim 252, wherein R^{A} is —CH₂—CF₃ or —CH₂-cyclopropyl.

254. The compound of claim 248, wherein R^{A} is optionally substituted phenyl.

255. The compound of claim 254, wherein R^{A} is unsubstituted phenyl.

256. The compound of claim 248, wherein R^{A} is optionally substituted heterocyclyl.

257. The compound of claim 248, wherein R^{A} is unsubstituted five-membered heterocyclyl or unsubstituted six-membered heterocyclyl.

258. The compound of claim 257, wherein R^{A} is tetrahydrofuranyl or tetrahydropyranyl.

259. The compound of claim 207, wherein R^{10a} is —N(R^{B})₂; and each instance of R^{B} is independently hydrogen, optionally substituted alkyl, optionally substituted carbocyclyl, optionally substituted aryl, optionally substituted heteroaryl, or a nitrogen protecting group.

260. The compound of claim 259, wherein R^{10a} is —NHR^{B}.

261. The compound of claim 259, wherein R^{10a} is —N(CH₃)R^{B}.

262. The compound of any one of claims 259-261, wherein R^{B} is hydrogen.

263. The compound of any one of claims 259-261, wherein R^{B} is unsubstituted C₁₋₆ alkyl.

264. The compound of claim 263, wherein R^{B} is methyl or ethyl.

265. The compound of any one of claims 259-261, wherein R^{B} is substituted C₁₋₆ alkyl.

266. The compound of claim 265, wherein R^{B} is optionally substituted —C₁₋₄alkyl-carbocyclyl, optionally substituted —C₁₋₄alkyl-heteroaryl, optionally substituted —C₁₋₄alkyl-heterocyclyl, or optionally substituted —C₁₋₄alkyl-CO₂X^{10B}; and X^{10B} is hydrogen or optionally substituted alkyl.

267. The compound of claim 266, wherein R^{B} is one of the following formulae:

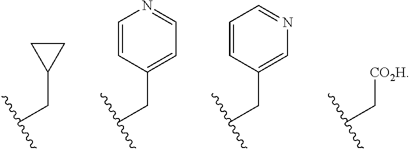 [SMILES: *CC1CC1 ; *Cc1ccncc1 ; *Cc1cccnc1 ; *CC(=O)O]

268. The compound of any one of claims 259-261, wherein R^{B} is optionally substituted C₃₋₆ carbocyclyl.

269. The compound of claim 268, wherein R^{B} is cyclopropyl.

270. The compound of any one of claims 259-261, wherein R^{B} is optionally substituted heterocyclyl.

271. The compound of claim 270, wherein R^{B} is tetrahydropyranyl or oxetanyl.

272. The compound of any one of claims 259-261, wherein R^{B} is optionally substituted heteroaryl.

273. The compound of claim 272, wherein R^{B} is optionally substituted thiazolyl.

274. The compound of claim 273, wherein R^{B} is of the formula

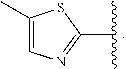 [SMILES: *c1ncc(C)s1]

275. The compound of any one of claims 259-261, wherein R^{B} is a nitrogen protecting group.

276. The compound of claim 275, wherein:
R^{B} is —SO₂—X^{10S};
X^{10S} is optionally substituted alkyl or —N(R^{SB})₂; and
each instance of R^{SB} is hydrogen or optionally substituted alkyl.

277. The compound of claim 276, wherein R^{B} is —SO₂—N(CH₃)₂, —SO₂—CH₃, —SO₂—C₂H₅, or —SO₂—CF₃.

278. The compound of claim 207, wherein R^{10a} is —C(═O)N(R^{B})₂; and each instance of R^{B} is independently hydrogen, optionally substituted alkyl, optionally substituted carbocyclyl, optionally substituted aryl, optionally substituted heteroaryl, or a nitrogen protecting group; or two R^{B} taken together with the intervening nitrogen form optionally substituted heterocyclyl.

279. The compound of claim 278, wherein R^{10a} is —C(═O)NHR^{B}.

280. The compound of claim 78, wherein R^{10a} is —C(═O)N(CH₃)R^{B}.

281. The compound of any one of claims 278-280, wherein R^{B} is hydrogen.

282. The compound of any one of claims 278-280, wherein R^{B} is optionally substituted C₁₋₆ alkyl.

283. The compound of claim 282, wherein R^{B} is unsubstituted C₁₋₆ alkyl.

284. The compound of claim 283, wherein R^{B} is methyl or ethyl.

285. The compound of claim 282, wherein R^{B} is substituted C₁₋₆ alkyl.

286. The compound of claim 285, wherein R^{B} is C₁₋₆ haloalkyl.

287. The compound of claim 286, wherein R^{B} is —CH₂—CF₃.

288. The compound of claim 285, wherein R^{B} is optionally substituted —C₁₋₄alkyl-carbocyclyl.

289. The compound of claim 288, wherein R^{B} is —CH₂-cyclopropyl.

290. The compound of claim 285, wherein R^{B} is optionally substituted —C₁₋₄alkyl-heteroaryl.

291. The compound of claim 290, wherein R^{B} is one of the following formulae:

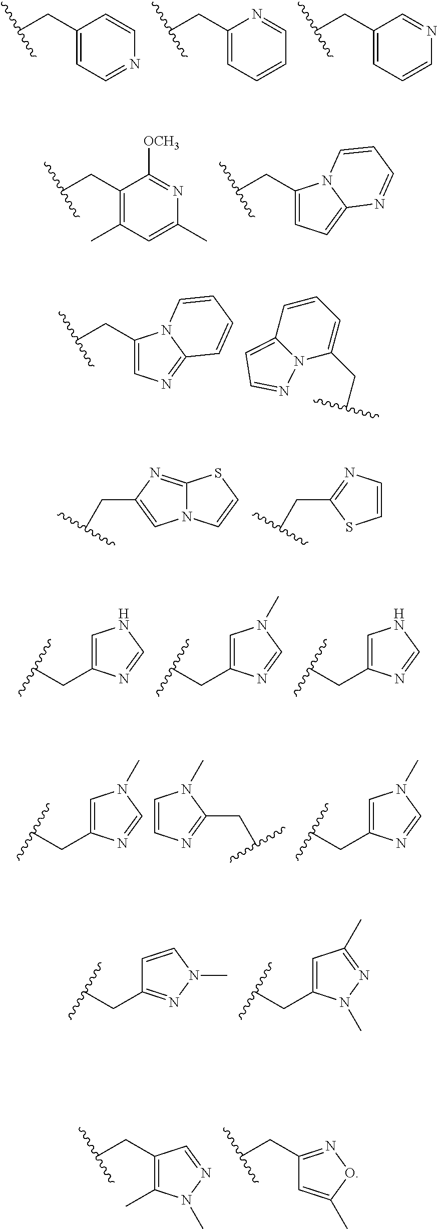

292. The compound of claim 285, wherein R^{B} is optionally substituted —C₁₋₄alkyl-heterocyclyl.

293. The compound of claim 92, wherein R^{B} is one of the following formulae:

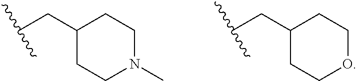

294. The compound of claim 285, wherein R^{B} is optionally substituted —C₁₋₄alkyl-phenyl.

295. The compound of claim 294, wherein R^{B} is one of the following formulae:

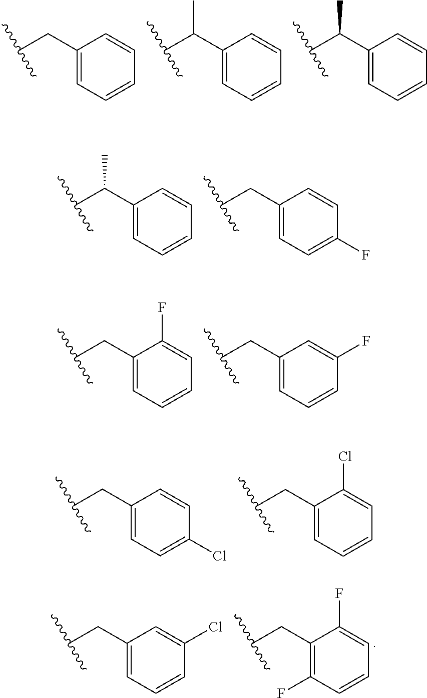

296. The compound of claims 278-280, wherein R^{B} is optionally substituted carbocyclyl.

297. The compound of claim 297, wherein R^{B} is cyclopropyl.

298. The compound of claims 278-280, wherein R^{B} is optionally substituted heterocyclyl.

299. The compound of claim 298, wherein R^{B} is tetrahydrofuranyl or tetrahydropyranyl.

300. The compound of claim 299, wherein two R^{B} taken together with the intervening nitrogen form an optionally substituted heterocyclyl.

301. The compound of claim 300, wherein R^{10a} is one of the following formulae:

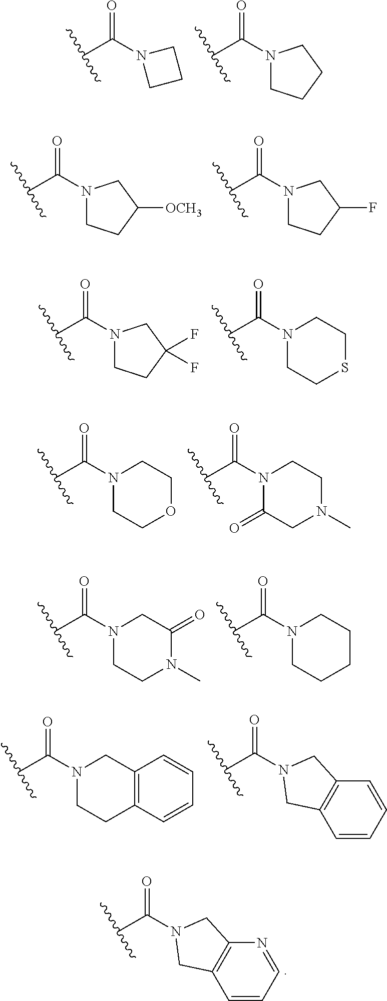

302. The compound of claim 207, wherein R^{10a} is —C(═O)OR^{A}; and R^{A} is optionally substituted alkyl.

303. The compound of claim 302, wherein lea is —C(═O)OC₂H₅.

304. The compound of claim 207, wherein R^{10a} is —C(═O)R^{A}; and R^{A} is optionally substituted alkyl.

305. The compound of claim 304, wherein R^{10a} is —C(═O)C₂H₅.

306. The compound of claim 207, wherein R^{10a} is —NR^{B}C(═O)OR^{A}; R^{A} is optionally substituted alkyl; and R^{B} is hydrogen or optionally substituted alkyl.

307. The compound of claim 306, wherein R^{B} is hydrogen.

308. The compound of claim 306, wherein R^{A} is unsubstituted C₁₋₆ alkyl.

309. The compound of claim 308, wherein R^{A} is methyl or ethyl.

310. The compound of claim 306, wherein R^{A} is substituted C₁₋₆ alkyl.

311. The compound of claim 310, wherein R^{A} is C₁₋₆ alkyl-aryl.

312. The compound of claim 311, wherein R^{A} is —CH₂-Ph.

313. The compound of claim 207, wherein R^{10a} is —NR^{B}C(═O)N(R^{B})₂; and each instance of R^{B} is hydrogen; optionally substituted alkyl; or optionally substituted heterocyclyl.

314. The compound of claim 313, wherein R^{10a} is —NHC(═O)N(R^{B})₂.

315. The compound of claim 314, wherein R^{10a} is —NHC(═O)NHR^{B} or —NHC(═O)N(CH₃)R^{B}.

316. The compound of any one of claims 313-315, wherein R^{B} is unsubstituted C₁₋₆ alkyl.

317. The compound of claim 316, wherein R^{B} is methyl, ethyl, or isopropyl.

318. The compound of any one of claims 313-315, wherein R^{B} is optionally substituted heterocyclyl.

319. The compound of claim 318, wherein R^{B} is tetrahydropyranyl.

320. The compound of claim 207, wherein R^{10a} is —C(═O)N(R^{B})₂; and each instance of R^{B} is hydrogen or optionally substituted alkyl.

321. The compound of claim 320, wherein R^{10a} is —C(═O)NHR^{B}.

322. The compound of claim 321, wherein R^{B} is optionally substituted C₁₋₆alkyl-heteroaryl or optionally substituted C₁₋₆alkyl-heterocyclyl.

323. The compound of any one of claims 320-322, wherein R^{B} is of the formula: 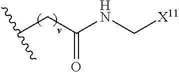
wherein:
v is 1, 2, 3, 4, 5, 6, or 7; and
X¹¹ is optionally substituted heterocyclyl or optionally substituted five-membered heteroaryl.

324. The compound of claim 323, wherein R^{B} is of one of the following formulae:

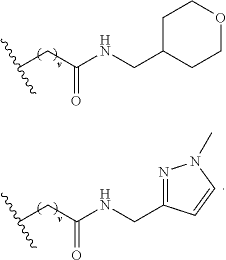

325. The compound of any one of claims 323-324, wherein v is 2.

326. The compound of claims 323-324, wherein v is 3.

327. The compound of claims 323-324, wherein v is 4.

328. The compound of claims 323-324, wherein v is 5.

329. The compound of any one of claims 207-328, wherein R^{10b} is hydrogen or optionally substituted C₁₋₆ alkyl.

330. The compound of claim 329, wherein R^{10b} is hydrogen.

331. The compound of claim 329, wherein R^{10b} is unsubstituted C₁₋₆ alkyl.

332. The compound of claim 331, wherein R^{10b} is methyl.

333. The compound of any one of claims 1-19, wherein Ring A is one of the following formulae: 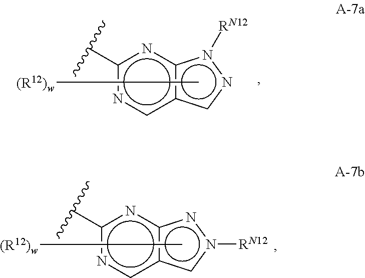
wherein:
each instance of R¹² is independently hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —C(═O)R^{A}, —C(═O)OR^{A}, —OC(═O)R^{A}, —C(═O)N(R^{B})₂, —NR^{B}C(═O)R^{A}, —OC(═O)N(R^{B})₂, —NR^{B}C(═O)OR^{A}, —NR^{B}C(═O)N(R^{B})₂, —S(═O)R^{A}, —OS(═O)₂R^{A}, —SO₂R^{A}, —NR^{B}SO₂R^{A}, or —SO₂N(R^{B})₂;
w is 0, 1, or 2; and
each instance of R^{N12} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group.

334. The compound of claim 333, wherein Ring A is one of the following formulae

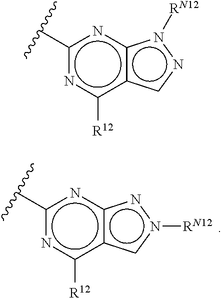

335. The compound of any one of claims 333-334, wherein R^{N12} is hydrogen or optionally substituted alkyl.

336. The compound of claim 335, wherein R^{N12} is hydrogen.

337. The compound of claim 336, wherein R^{N12} is optionally substituted C₁₋₆ alkyl.

338. The compound of claim 337, wherein R^{N12} is unsubstituted C₁₋₆ alkyl.

339. The compound of claim 338, wherein R^{N12} is isopropyl or tert-butyl.

340. The compound of any one of claims 333-339, wherein R′² is —N(R^{B})₂ or —C(═O)N(R^{B})₂, wherein each instance of R^{B} is independently hydrogen, optionally substituted alkyl, optionally substituted carbocyclyl, optionally substituted heterocyclyl, optionally substituted aryl, or optionally substituted heteroaryl.

341. The compound of any one of claims 333-340, wherein R′² is —NHR^{B}, —N(CH₃)R^{B}, —C(═O)NHR^{B}, or, —C(═O)N(CH₃)R^{B}.

342. The compound of any one of claims 333-341, wherein R^{B} is hydrogen.

343. The compound of any one of claims 333-341, wherein R^{B} is optionally substituted C₁₋₆ alkyl.

344. The compound of claim 343, wherein R^{B} is unsubstituted C₁₋₆ alkyl.

345. The compound of claim 344, wherein R^{B} is methyl.

346. The compound of claim 343, wherein R^{B} is substituted C₁₋₆ alkyl.

347. The compound of claim 346, wherein R^{B} is optionally substituted —C₁₋₆ alkyl-heterocyclyl or optionally substituted —C₁₋₆ alkyl-heteroaryl.

348. The compound of any one of claim 347, wherein R^{B} is one of the following formulae:

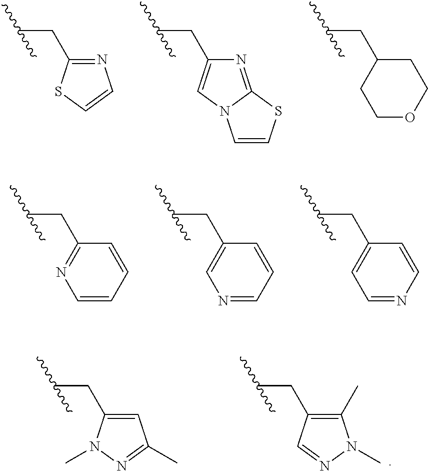

349. The compound of any one of claims 334-341, wherein R^{B} is optionally substituted carbocyclyl.

350. The compound of claim 349, wherein R^{B} is cyclopropyl.

351. The compound of any one of claims 334-341, wherein R^{B} is optionally substituted heterocyclyl.

352. The compound of claim 351, wherein R^{B} is unsubstituted heterocyclyl.

353. The compound of claim 352, wherein R^{B} is tetrahydropyranyl.

354. The compound of any one of claims 1-353, wherein IV is optionally substituted C₁₋₄ alkyl.

355. The compound of claim 354, wherein IV is unsubstituted C₁₋₄ alkyl.

356. The compound of claim 355, wherein IV is methyl.

357. The compound of any one of claims 1-356, wherein R^{3a} is hydrogen.

358. The compound of any one of claims 1-357, wherein R^{3a} is optionally substituted C₁₋₄ alkyl.

359. The compound of claim 358, wherein R^{3a} is unsubstituted C₁₋₄ alkyl.

360. The compound of claim 359, wherein R^{3a} is methyl.

361. The compound of any one of claims 1-360, wherein R^{3b} is hydrogen.

362. The compound of any one of claims 1-361, wherein R^{3b} is optionally substituted C₁₋₄ alkyl.

363. The compound of claim 362, wherein R^{3b} is methyl.

364. The compound of any one of claims 1-363, wherein R^{3b} is optionally substituted C₃₋₄ cyclcoalkyl.

365. The compound of claim 364, wherein R^{3b} is cyclopropyl.

366. The compound of claim 1, wherein the compound is selected from the group consisting of the compounds depicted in Table 1, and pharmaceutically acceptable salts thereof.

367. The compound of claim 1, wherein the compound is not one of the compounds depicted in Table 2.

368. A pharmaceutical composition comprising a compound of any one of claims 1-367 or a pharmaceutically acceptable salt thereof, and a pharmaceutically acceptable excipient.

369. A kit or packaged pharmaceutical comprising a compound of any one of claims 1-367 or a pharmaceutically acceptable salt thereof, and instructions for use thereof.

370. A method of inhibiting an arginine methyl tranferase (RMT) comprising contacting a cell with an effective amount of a compound of any one of claims 1-367 or a pharmaceutically acceptable salt thereof.

371. The method of claim 370, wherein the arginine methyl transferase is PRMT1.

372. The method of claim 370, wherein the arginine methyl transferase is PRMT6.

373. The method of claim 370, wherein the arginine methyl transferase is PRMT3.

374. The method of claim 370, wherein the arginine methyl transferase is PRMT8.

375. The method of claim 70, wherein the arginine methyl transferase is CARM1.

376. A method of modulating gene expression comprising contacting a cell with an effective amount of a compound of any one of claims 1-367 or a pharmaceutically acceptable salt thereof.

377. A method of modulating transcription comprising contacting a cell with an effective amount of a compound of any one of claims 1-367 or a pharmaceutically acceptable salt thereof.

378. The method of any one of claims 370-377, wherein the cell is in vitro.

379. The method of any one of claims 370-377, wherein the cell is in a subject.

380. A method of treating a RMT-mediated disorder, comprising administering to a subject in need thereof a therapeutically effective amount of a compound of any one of claims 1-367, or a pharmaceutically acceptable salt thereof, or a pharmaceutical composition of claim 368.

381. The method of claim 380, wherein the RMT-mediated disorder is a PRMT1-mediated disorder.

382. The method of claim 380, wherein the RMT-mediated disorder is a PRMT6-mediated disorder.

383. The method of claim 380, wherein the RMT-mediated disorder is a PRMT3-mediated disorder.

384. The method of claim 380, wherein the RMT-mediated disorder is a PRMT8-mediated disorder.

385. The method of claim 380, wherein the RMT-mediated disorder is a CARM1-mediated disorder.

386. The method of claim 380, wherein the disorder is a proliferative disorder.

387. The method of claim 386, wherein the disorder is cancer.

388. The method of claim 380, wherein the disorder is a neurological disorder.

389. The method of claim 388, wherein the disorder is amyotrophic lateral sclerosis.

390. The method of claim 380, wherein the disorder is a muscular dystrophy.

391. The method of claim 380, wherein the disorder is an autoimmune disorder.

392. The method of claim 380, wherein the disorder is a vascular disorder.

393. The method of claim 380, wherein the disorder is a metabolic disorder.


SEQUENCE LISTING (17 sequences)

SEQ ID NO: 1 - protein, 15 aa, Artificial Sequence
  Synthetic Polypeptide
RLARRGGVKRISGLI

SEQ ID NO: 2 - protein, 8 aa, Artificial Sequence
  Synthetic Polypeptide
DYKDDDDK

SEQ ID NO: 3 - protein, 230 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDENLYFQGGNS

SEQ ID NO: 4 - protein, 609 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDENLYFQGGNSDYKDDDDKMAAAEAANCIMENFVATLANGMSLQPPLEEVSCGQAESSEKPNAEDMTSKDYYFDSYAHFGIHEEMLKDEVRTLTYRNSMFHNRHLFKDKVVLDVGSGTGILCMFAAKAGARKVIGIECSSISDYAVKIVKANKLDHVVTIIKGKVEEVELPVEKVDIIISEWMGYCLFYESMLNTVLYARDKWLAPDGLIFPDRATLYVTAIEDRQYKDYKIHWWENVYGFDMSCIKDVAIKEPLVDVVDPKQLVTNACLIKEVDIYTVKVEDLTFTSPFCLQVKRNDYVHALVAYFNIEFTRCHKRTGFSTSPESPYTHWKQTVFYMEDYLTVKTGEEIFGTIGMRPNAKNNRDLDFTIDLDFKGQLCELSCSTDYRMR

SEQ ID NO: 5 - protein, 15 aa, Artificial Sequence
  Synthetic Polypeptide
RLARRGGVKRISGLI

SEQ ID NO: 6 - protein, 9 aa, Artificial Sequence
  Synthetic Polypeptide
MDYKDDDDK

SEQ ID NO: 7 - protein, 389 aa, Artificial Sequence
  Synthetic Polypeptide
MDYKDDDDKSQPKKRKLESGGGGEGGEGTEEEDGAEREAALERPRRTKRERDQLYYECYSDVSVHEEMIADRVRTDAYRLGILRNWAALRGKTVLDVGAGTGILSIFCAQAGARRVYAVEASAIWQQAREVVRFNGLEDRVHVLPGPVETVELPEQVDAIVSEWMGYGLLHESMLSSVLHARTKWLKEGGLLLPASAELFIAPISDQMLEWRLGFWSQVKQHYGVDMSCLEGFATRCLMGHSEIVVQGLSGEDVLARPQRFAQLELSRAGLEQELEAGVGGRFRCSCYGSAPMHGFAIWFQVTFPGGESEKPLVLSTSPFHPATHWKQALLYLNEPVQVEQDTDVSGEITLLPSRDNPRRLRVLLRYKVGDQEEKTKDFAMEDHHHHHH

SEQ ID NO: 8 - protein, 15 aa, Artificial Sequence
  Synthetic Polypeptide
KPAIRRLARRGGVKR

SEQ ID NO: 9 - protein, 229 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEF

SEQ ID NO: 10 - protein, 623 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFMGMKHSSRCLLLRRKMAENAAESTEVNSPPSQPPQPVVPAKPVQCVHHVSTQPSCPGRGKMSKLLNPEEMTSRDYYFDSYAHFGIHEEMLKDEVRTLTYRNSMYHNKHVFKDKVVLDVGSGTGILSMFAAKAGAKKVFGIECSSISDYSEKIIKANHLDNIITIFKGKVEEVELPVEKVDIIISEWMGYCLFYESMLNTVIFARDKWLKPGGLMFPDRAALYVVAIEDRQYKDFKIHWWENVYGFDMTCIRDVAMKEPLVDIVDPKQVVTNACLIKEVDIYTVKTEELSFTSAFCLQIQRNDYVHALVTYFNIEFTKCHKKMGFSTAPDAPYTHWKQTVFYLEDYLTVRRGEEIYGTISMKPNAKNVRDLDFTVDLDFKGQLCETSVSNDYKMR

SEQ ID NO: 11 - protein, 19 aa, Artificial Sequence
  Synthetic Polypeptide
GGRGGFGGRGGFGGRGGFG

SEQ ID NO: 12 - protein, 226 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGS

SEQ ID NO: 13 - protein, 756 aa, Artificial Sequence
  Synthetic Polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSCSLASGATGGRGAVENEEDLPELSDSGDEAAWEDEDDADLPHGKQQTPCLFCNRLFTSAEETFSHCKSEHQFNIDSMVHKHGLEFYGYIKLINFIRLKNPTVEYMNSIYNPVPWEKEEYLKPVLEDDLLLQFDVEDLYEPVSVPFSYPNGLSENTSVVEKLKHMEARALSAEAALARAREDLQKMKQFAQDFVMHTDVRTCSSSTSVIADLQEDEDGVYFSSYGHYGIHEEMLKDKIRTESYRDFIYQNPHIFKDKVVLDVGCGTGILSMFAAKAGAKKVLGVDQSEILYQAMDIIRLNKLEDTITLIKGKIEEVHLPVEKVDVIISEWMGYFLLFESMLDSVLYAKNKYLAKGGSVYPDICTISLVAVSDVNKHADRIAFWDDVYGFKMSCMKKAVIPEAVVEVLDPKTLISEPCGIKHIDCHTTSISDLEFSSDFTLKITRTSMCTAIAGYFDIYFEKNCHNRVVFSTGPQSTKTHWKQTVFLLEKPFSVKAGEALKGKVTVHKNKKDPRSLTVTLTLNNSTQTYGLQ

SEQ ID NO: 14 - protein, 15 aa, Artificial Sequence
  Synthetic Polypeptide
PRKQLATKAARKSAP

SEQ ID NO: 15 - protein, 8 aa, Artificial Sequence
  Synthetic Polypeptide
EGHHHHHH

SEQ ID NO: 16 - protein, 601 aa, Artificial Sequence
  Synthetic Polypeptide
MDYKDDDDKAAAAAAVGPGAGGAGSAVPGGAGPCATVSVFPGARLLTIGDANGEIQRHAEQQALRLEVRAGPDSAGIALYSHEDVCVFKCSVSRETECSRVGKQSFIITLGCNSVLIQFATPNDFCSFYNILKTCRGHTLERSVFSERTEESSAVQYFQFYGYLSQQQNMMQDYVRTGTYQRAILQNHTDFKDKIVLDVGCGSGILSFFAAQAGARKIYAVEASTMAQHAEVLVKSNNLTDRIVVIPGKVEEVSLPEQVDIIISEPMGYMLFNERMLESYLHAKKYLKPSGNMFPTIGDVHLAPFTDEQLYMEQFTKANFWYQPSFHGVDLSALRGAAVDEYFRQPVVDTFDIRILMAKSVKYTVNFLEAKEGDLHRIEIPFKFHMLHSGLVHGLAFWFDVAFIGSIMTVWLSTAPTEPLTHWYQVRCLFQSPLFAKAGDTLSGTCLLIANKRQSYDISIVAQVDQTGSKSSNLLDLKNPFFRYTGTTPSPPPGSHYTSPSENMWNTGSTYNLSSGMAVAGMPTAYDLSSVIASGSSVGHNNLIPLGSSGAQGSGGGSTSAHYAVNSQFTMGGPAISMASPMSIPTNTMHYGSEGHHHHHH

SEQ ID NO: 17 - protein, 6 aa, Artificial Sequence
  Synthetic Polypeptide
HHHHHH